(19) United States
(12) Patent Application Publication
(10) Pub. No.: US 2003/0152926 A1
(43) Pub. Date: Aug. 14, 2003

(54) Novel methods of diagnosis of angiogenesis, compositions and methods of screening for angiogenesis modulators

(75) Inventors: Richard Murray, Cupertino, CA (US); Richard Glynne, Palo Alto, CA (US); Susan R. Watson, El Cerrito, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eos Biotechnology, Inc., South San Francisco, CA (US)

(21) Appl. No.: 10/021,660

(22) Filed: Dec. 6, 2001

Related U.S. Application Data
(63) Continuation of application No. 09/784,356, filed on Feb. 14, 2001.
(63) Continuation-in-part of application No. 09/637,977, filed on Aug. 11, 2000.
(60) Provisional application No. 60/148,425, filed on Aug. 11, 1999.

(51) Int. Cl. C12Q 1/68; C07H 21/04; C12P 19/34

(52) U.S. Cl. 435/6; 536/23.1; 435/91.2

(57) ABSTRACT

Described herein are methods and compositions that can be used for diagnosis and treatment of angiogenic phenotypes and angiogenesis-associated diseases. Also described herein are methods that can be used to identify modulators of angiogenesis.

### CROSS-REFERENCES TO RELATED APPLICATIONS

The present application is a continuation-in-part (CIP) of co-pending U.S. patent application “Novel Methods Of Diagnosis Of Angiogenesis, Compositions And Methods Of Screening For Angiogenesis Modulators”, Attorney Docket No. A651 10-1, filed on Aug. 11, 2000, which claims the benefit of priority to U.S. Ser. No. 60/148,425 filed Aug. 11, 1999, both of which are incorporated herein by reference.

### FIELD OF THE INVENTION

The invention relates to the identification of nucleic acid and protein expression profiles and nucleic acids, products, and antibodies thereto that are involved in angiogenesis; and to the use of such expression profiles and compositions in diagnosis and therapy of angiogenesis. The invention further relates to methods for identifying and using agents and/or targets that modulate angiogenesis.

### BACKGROUND OF THE INVENTION

Both vasculogenesis, the development of an interactive vascular system comprising arteries and veins, and angiogenesis, the generation of new blood vessels, play a role in embryonic development. In contrast, angiogenesis is limited in a normal adult to the placenta, ovary, endometrium and sites of wound healing. However, angiogenesis, or its absence, plays an important role in the maintenance of a variety of pathological states. Some of these states are characterized by neovascularization, e.g., cancer, diabetic retinopathy, glaucoma, and age related macular degeneration. Others, e.g., stroke, infertility, heart disease, ulcers, and scleroderma, are diseases of angiogenic insufficiency. Angiogenesis has a number of stages (see, e.g., Folkman, J. Natl Cancer Inst. 82.4-6, 1990; Firestein, J Clin Invest.103:3-4, 1999; Koch, Arthritis Rheum.41:951-62, 1998; Carter, Oncologist 5(Suppl 1):51-4, 2000; Browder et al., Cancer Res. 60:1878-86, 2000; and Zhu and Witte, Invest New Drugs 17:195-212, 1999). The early stages of angiogenesis include endothelial cell protease production, migration of cells, and proliferation. The early stages also appear to require some growth factors, with VEGF, TGF-A, angiostatin, and selected chemokines all putatively playing a role. Later stages of angiogenesis include population of the vessels with mural cells (pericytes or smooth muscle cells), basement membrane production, and the induction of vessel bed specializations. The final stages of vessel formation include what is known as “remodeling”, wherein a forming vasculature becomes a stable, mature vessel bed. Thus, the process is highly dynamic, often requiring coordinated spatial and temporal waves of gene expression.

Conversely, the complex process may be subject to disruption by interfering with one or more critical steps. Thus, the lack of understanding of the dynamics of angiogenesis prevents therapeutic intervention in serious diseases such as those indicated. It is an object of the invention to provide methods that can be used to screen compounds for the ability to modulate angiogenesis. Additionally, it is an object to provide molecular targets for therapeutic intervention in disease states which either have an undesirable excess or a deficit in angiogenesis. The present invention provides solutions to both.

### SUMMARY OF THE INVENTION

The present invention provides compositions and methods for detecting or modulating angiogenesis associated sequences.

In one aspect, the invention provides a method of detecting an angiogenesis-associated transcript in a cell in a patient, the method comprising contacting a biological sample from the patient with a polynucleotide that selectively hybridized to a sequence at least 80% identical to a sequence as shown in Table 1. In one embodiment, the biological sample is a tissue sample. In another embodiment, the biological sample comprises isolated nucleic acids, which are often mRNA.

In another embodiment, the method further comprises the step of amplifying nucleic acids before the step of contacting the biological sample with the polynucleotide. Often, the polynucleotide comprises a sequence as shown in Table 1. The polynucleotide can be labeled, for example, with a fluorescent label and can be immobilized on a solid surface.

In other embodiments the patient is undergoing a therapeutic regimen to treat a disease associated with angiogenesis or the patient is suspected of having an angiogenesis-associated disorder.

In another aspect, the invention comprises an isolated nucleic acid molecule consisting of a polynucleotide sequence as shown in Table 1. The nucleic acid molecule can be labeled, for example, with a fluorescent label.

In other aspects, the invention provides an expression vector comprising an isolated nucleic acid molecule consisting of a polynucleotide sequence as shown in Table 1 or a host cell comprising the expression vector.

In another embodiment, the isolated nucleic acid molecule encodes a polypeptide having an amino acid sequence as shown in Table 2.

In another aspect, the invention provides an isolated polypeptide which is encoded by a nucleic acid molecule having polynucleotide sequence as shown in Table 1. In one embodiment, the isolated polypeptide has an amino acid sequence as shown in Table 2.

In another embodiment, the invention provides an antibody that specifically binds a polypeptide that has an amino acid sequence as shown in Table 2. The antibody can be conjugated to an effector component such as a fluorescent label, a toxin, or a radioisotope. In some embodiments, the antibody is an antibody fragment or a humanized antibody.

In another aspect, the invention provides a method of detecting a cell undergoing angiogenesis in a biological sample from a patient, the method comprising contacting the biological sample with an antibody that specifically binds to a polypeptide that has an amino acid sequence as shown in Table 2. In some embodiment, the antibody is further conjugated to an effector component, for example, a fluorescent label.

In another embodiment, the invention provides a method of detecting antibodies specific to angiogenesis in a patient, the method comprising contacting a biological sample from the patient with a polypeptide comprising a sequence as shown in Table 2.

The invention also provides a method of identifying a compound that modulates the activity of an angiogenesis-associated polypeptide, the method comprising the steps of: (i) contacting the compound with a polypeptide that comprises at least 80% identity to an amino acid sequence as shown in Table 2; and (ii) detecting an increase or a decrease in the activity of the polypeptide. In one embodiment, the polypeptide has an amino acid sequence as shown in Table 2. In another embodiment, the polypeptide is expressed in a cell.

The invention also provides a method of identifying a compound that modulates angiogenesis, the method comprising steps of: (i) contacting the compound with a cell undergoing angiogenesis; and (ii) detecting an increase or a decrease in the expression of a polypeptide sequence as shown in Table 2. In one embodiment, the detecting step comprises hybridizing a nucleic acid sample from the cell with a polynucleotide that selectively hybridizes to a sequence at least 80% identical to a sequence as shown in Table 1. In another embodiment, the method further comprises detecting an increase or decrease in the expression of a second sequence as shown in Table 2.

In another embodiment, the invention provides a method of inhibiting angiogenesis in a cell that expresses a polypeptide at least 80% identical to a sequence as shown in Table 2, the method comprising the step of contacting the cell with a therapeutically effective amount of an inhibitor of the polypeptide. In one embodiment, the polypeptide has an amino acid sequence shown in Table 2. In another embodiment, the inhibitor is an antibody.

In other embodiments, the invention provides a method of activating angiogenesis in a cell that expresses a polypeptide at least 80% identical to a sequence as shown in Table 2, the method comprising the step of contacting the cell with a therapeutically effective amount of an activator of the polypeptide. In one embodiment, the polypeptide has an amino acid sequence shown in Table 2.

Other aspects of the invention will become apparent to the skilled artisan by the following description of the invention.

Table 1 provides nucleotide sequence of genes that exhibit changes in expression levels as a function of time in tissue undergoing angiogenesis compared to tissue that is not.

Table 2 provides polypeptide sequence of proteins that exhibit changes in expression levels as a function of time in tissue undergoing angiogenesis compared to tissue that is not.

### DESCRIPTION OF THE SPECIFIC EMBODIMENTS

In accordance with the objects outlined above, the present invention provides novel methods for diagnosis and treatment of disorders associated with angiogenesis (sometimes referred to herein as angiogenesis disorders or AD), as well as methods for screening for compositions which modulate angiogenesis. By “disorder associated with angiogenesis” or “disease associated with angiogenesis” herein is meant a disease state which is marked by either an excess or a deficit of vessel development. Angiogenesis disorders asociated with increased angiogenesis include, but are not limited to, cancer and proliferative diabetic retinopathy. Pathological states for which it may be desirable to increase angiogenesis include stroke, heart disease, infertility, ulcers, and scleradoma. Also provided are methods for treating AD.

Definitions

The term “angiogenesis protein” or “angiogenesis polynucleotide” refers to nucleic acid and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an angiogenesis protein sequence of Table 2; (2) bind to antibodies, e.g. polyclonal antibodies, raised against an immunogen comprising an amino acid sequence of Table 2, and conservatively modified variants thereof; (3) specifically hybridize under stringent hybridization conditions to an anti-sense strand corresponding to a nucleic acid sequence of Table 1 and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a sense sequence corresponding to one set out in Table 1. A polynucleotide or polypeptide sequence is typically from a mammal including, but not limited to, primate, e.g., human; rodent, e.g., rat, mouse, hamster; cow, pig, horse, sheep, or any mammal. An “angiogenesis polypeptide” and an “angiogenesis polynucleotide,” include both naturally occurring or recombinant.

A “full length” angiogenesis protein or nucleic acid refers to an agiogenesis polypeptide or polynucleotide sequence, or a variant thereof, that contains all of the elements normally contained in one or more naturally occurring, wild type angiogenesis polynucleotide or polypeptide sequences. The “full length” may be prior to, or after, various stages of post-translation processing.

“Biological sample” as used herein is a sample of biological tissue or fluid that contains nucleic acids or polypeptides, e.g., of an angiogenic protein. Such samples include, but are not limited to, tissue isolated from primates, e.g., humans, or rodents, e.g., mice, and rats. Biological samples may also include sections of tissues such as biopsy and autopsy samples, and frozen sections taken for histologic purposes. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome histroy, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., SEQ ID NOS:1-4), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4 and a comparison of both strands. For amino acid sequences, the BLASTN program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=-4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and a-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, for example, detection moieties including radioactive compounds, fluroescent compounds, an enzyme or substrate, tags such as epitope tags, a toxin; a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijseen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5× SSC, and 1% SDS, incubating at 42° C., or, 5× SSC, 1% SDS, incubating at 65° C., with wash in 0.2× SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of an angiogenesis protein includes the determination of a parameter that is indirectly or directly under the influence of the angiogenesis protein, e.g., a functional, physical, or chemical effect, such as the ability to increase or decrease angiogenesis. It includes binding activity, the ability of cells to proliferate, expression in cells undergoing angiogenesis, and other characteristics of angiogenic cells. “Functional effects” include in vitro, in vivo, and ex vivo activities.

By “determining the functional effect” is meant assaying for a compound that increases or decreases a parameter that is indirectly or directly under the influence of an angiogenesis protein sequence, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the angiogenesis protein; measuring binding activity or binding assays, e.g. binding to antibodies, and measuring cellular proliferation, particularly endothelial cell proliferation. Determination of the functional effect of a compound on angiogenesis can also be performed using angiogenesis assays known to those of skill in the art such as an in vitro assays, e.g., in vitro endothelial cell tube formation assays, and other assays such as the chick CAM assay, the mouse corneal assay, and assays that assess vascularization of an implanted tumor. The functional effects can be evaluated by many means known to those skilled in the art, e.g., microscopy for quantitative or qualitative measures of alterations in morphological features, e.g., tube or blood vessel formation, measurement of changes in RNA or protein levels for angiogenesis-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Inhibitors”, “activators”, and “modulators” of angiogenic polynucleotide and polypeptide sequences are used to refer to activating, inhibitory, or modulating molecules identified using in vitro and in vivo assays of angiogenic polynucleotide and polypeptide sequences. Inhibitors are compounds that, e.g., bind to, partially or totally block activity, decrease, prevent, delay activation, inactivate, desensitize, or down regulate the activity or expression of angiogenesis proteins, e.g., antagonists. “Activators” are compounds that increase, open, activate, facilitate, enhance activation, sensitize, agonize, or up regulate angiogenesis protein activity. Inhibitors, activators, or modulators also include genetically modified versions of angiogenesis proteins, e.g., versions with altered activity, as well as naturally occurring and synthetic ligands, antagonists, agonists, antibodies, small chemical molecules and the like. Such assays for inhibitors and activators include, e.g., expressing the angiogenic protein in vitro, in cells, or cell membranes, applying putative modulator compounds, and then determining the functional effects on activity, as described above. Activators and inhibitors of angiogenesis can also be identified by incubating angiogenic cells with the test compound and determining increases or decreases in the expression of 1 or more angiogenesis proteins, e.g., 1, 2, 3, 4, 5, 10, 15, 20, 25, 30, 40, 50 or more angiogenesis proteins, such as angiogenesis proteins comprising the sequences set out in Table 2.

Samples or assays comprising angiogenesis proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of an angiogenesis polypeptide is achieved when the activity value relative to the control (untreated with activators) is 110%, more preferably 150%, more preferably 200-500% (i.e., two to five fold higher relative to the control), more preferably 1000-3000% higher.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclorial, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The present application may be related to U.S. Ser. No. 09/437,702, filed Nov. 10, 1999; U.S. Ser. No. 09/437,528, filed Nov. 10, 1999; U.S. Ser. No. 09/434,197, filed Nov. 4, 1999; U.S. Ser. No. 60/183,926, filed Feb. 22, 2000; U.S. Ser. No. 09/440,493, filed Nov. 15, 1999; U.S. Ser. No. 09/520,478, filed Mar. 8, 2000; U.S. Ser. No. 09/440,369, filed Nov. 12, 1999; Attorney Docket number A68928, filed Dec. 15, 2000; Attorney Docket number A69789, filed Jan. 22, 2001; and Attorney Docket number A69806, filed Dec. 15, 2000.

The detailed description of the invention includes discussion of the following aspects of the invention:

Expression of angiogenesis-associated sequences

Informatics

Angiogenesis-associated sequences

Detection of angiogenesis sequence for diagnostic and therapeutic applications

Modulators of angiogenesis

Methods of identifying variant angiogenesis-associated sequences

Administration of pharmaceutical and vaccine compositions

Kits for use in diagnostic and/or prognostic applications.

Expression of Angiogenesis-associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is unique to the state of the cell. That is, normal tissue may be distinguished from AD tissue. By comparing expression profiles of tissue in known different angiogenesis states, information regarding which genes are important (including both up- and down-regulation of genes) in each of these states is obtained. The identification of sequences that are differentially expressed in angiogenic versus non-angiogenic tissue allows the use of this information in a number of ways. For example, a particular treatment regime may be evaluated: does a chemotherapeutic drug act to down-regulate angiogenesis, and thus tumor growth or recurrence, in a particular patient. Similarly, diagnosis and treatment outcomes may be done or confirmed by comparing patient samples with the known expression profiles. Angiogenic tissue can also be analyzed to determine the stage of angiogenesis in the tissue. Furthermore, these gene expression profiles (or individual genes) allow screening of drug candidates with an eye to mimicking or altering a particular expression profile; for example, screening can be done for drugs that suppress the angiogenic expression profile. This may be done by making biochips comprising sets of the important angiogenesis genes, which can then be used in these screens. These methods can also be done on the protein basis; that is, protein expression levels of the angiogenic proteins can be evaluated for diagnostic purposes or to screen candidate agents. In addition, the angiogenic nucleic acid sequences can be administered for gene therapy purposes, including the administration of antisense nucleic acids, or the angiogenic proteins (including antibodies and other modulators thereof) administered as therapeutic drugs.

Thus the present invention provides nucleic acid and protein sequences that are differentially expressed in angiogenesis, herein termed “angiogenesis sequences”. As outlined below, angiogenesis sequences include those that are up-regulated (i.e. expressed at a higher level) in disorders associated with angiogenesis, as well as those that are down-regulated (i.e. expressed at a lower level). In a preferred embodiment, the angiogenesis sequences are from humans; however, as will be appreciated by those in the art, angiogenesis sequences from other organisms may be useful in animal models of disease and drug evaluation; thus, other angiogenesis sequences are provided, from vertebrates, including mammals, including rodents (rats, mice, hamsters, guinea pigs, etc.), primates, farm animals (including sheep, goats, pigs, cows, horses, etc). Angiogenesis sequences from other organisms may be obtained using the techniques outlined below.

Angiogenesis sequences can include both nucleic acid and amino acid sequences. In a preferred embodiment, the angiogenesis sequences are recombinant nucleic acids. By the term “recombinant nucleic acid” herein is meant nucleic acid, originally formed in vitro, in general, by the manipulation of nucleic acid e.g., using polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an angiogenesis protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the angiogenesis sequences are nucleic acids. As will be appreciated by those in the art and is more fully outlined below, angiogenesis sequences are useful in a variety of applications, including diagnostic applications, which will detect naturally occurring nucleic acids, as well as screening applications; for example, biochips comprising nucleic acid probes to the angiogenesis sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, nucleic acid analogs are included that may have alternate backbones, comprising, for example, phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids. Modifications of the ribose-phosphate backbone may be done for a variety of reasons, for example to increase the stability and half-life of such molecules in physiological environments or as probes on a biochip.

As will be appreciated by those in the art, nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An angiogenesis sequence can be initially identified by substantial nucleic acid and/or amino acid sequence homology to the angiogenesis sequences outlined herein. Such homology can be based upon the overall nucleic acid or amino acid sequence, and is generally determined as outlined below, using either homology programs or hybridization conditions.

For identifying angiogenesis-associated sequences, the angiogenesis screen typically includes comparing genes identified in a modification of an in vitro model of angiogenesis as described in Hiraoka, Cell 95:365 (1998) with genes identified in controls. Samples of normal tissue and tissue undergoing angiogenesis are applied to biochips comprising nucleic acid probes. The samples are first microdissected, if applicable, and treated as is known in the art for the preparation of mRNA. Suitable biochips are commercially available, for example from Affymetrix. Gene expression profiles as described herein are generated and the data analyzed.

In a preferred embodiment, the genes showing changes in expression as between normal and disease states are compared to genes expressed in other normal tissues, including, but not limited to lung, heart, brain, liver, breast, kidney, muscle, prostate, small intestine, large intestine, spleen, bone and placenta. In a preferred embodiment, those genes identified during the angiogenesis screen that are expressed in any significant amount in other tissues are removed from the profile, although in some embodiments, this is not necessary. That is, when screening for drugs, it is usually preferable that the target be disease specific, to minimize possible side effects.

In a preferred embodiment, angiogenesis sequences are those that are up-regulated in angiogenesis disorders; that is, the expression of these genes is higher in the disease tissue as compared to normal tissue. “Up-regulation” as used herein means at least about a two-fold change, preferably at least about a three fold change, with at least about five-fold or higher being preferred. All accession numbers herein are for the GenBank sequence database and the sequences of the accession numbers are hereby expressly incorporated by reference. GenBank is known in the art, see, e.g., Benson, DA, et al., Nucleic Acids Research 26:1-7 (1998) and http://www.ncbi.nlm.nih.gov/. Sequences are also avialable in other databases, e.g., European Molecular Biology Laboratory (EMBL) and DNA Database of Japan (DDBJ). In addition, most preferred genes were found to be expressed in a limited amount or not at all in heart, brain, lung, liver, breast, kidney, prostate, small intestine and spleen.

In another preferred embodiment, angiogenesis sequences are those that are down-regulated in the angiogenesis disorder; that is, the expression of these genes is lower in angiogenic tissue as compared to normal tissue. “Down-regulation” as used herein means at least about a two-fold change, preferably at least about a three fold change, with at least about five-fold or higher being preferred.

Angiogenesis sequences according to the invention may be classified into discrete clusters of sequences based on common expression profiles of the sequences. Expression levels of angiogenesis sequences may increase or decrease as a function of time in a manner that correlates with the induction of angiogenesis. Alternatively, expression levels of angiogenesis sequences may both increase and decrease as a function of time. For example, expression levels of some angiogenesis sequences are temporarily induced or diminished during the switch to the angiogenesis phenotype, followed by a return to baseline expression levels. Table 1 provides genes, the mRNA expression of which varies as a function of time in angiogenesis tissue when compared to normal tissue.

Table 2 provides protein sequences corresponding to the coding regions of the sequences that undergo changes in expression as a function of time in tissue undergoing angiogenesis.

In a particularly preferred embodiment, angiogenesis sequences are those that are induced for a period of time, typically by positive angiogenic factors, followed by a return to the baseline levels. Sequences that are temporarily induced provide a means to target angiogenesis tissue, for example neovascularized tumors, at a particular stage of angiogenesis, while avoiding rapidly growing tissue that require perpetual vascularization. Such positive angiogenic factors include αFGF, βFGF, VEGF, angiogenin and the like.

Induced angiogenesis sequences also are further categorized with respect to the timing of induction. For example, some angiogenesis genes may be induced at an early time period, such as within 10 minutes of the induction of angiogenesis. Others may be induced later, such as between 5 and 60 minutes, while yet others may be induced for a time period of about two hours or more followed by a return to baseline expression levels.

In another preferred embodiment are angiogenesis sequences that are inhibited or reduced as a function of time followed by a return to “normal” expression levels. Inhibitors of angiogenesis are examples of molecules that have this expression profile. These sequences also can be further divided into groups depending on the timing of diminished expression. For example, some molecules may display reduced expression within 10 minutes of the induction of angiogenesis. Others may be diminished later, such as between 5 and 60 minutes, while others may be diminished for a time period of about two hours or more followed by a return to baseline. Examples of such negative angiogenic factors include thrombospondin and endostatin to name a few.

In yet another preferred embodiment are angiogenesis sequences that are induced for prolonged periods. These sequences are typically associated with induction of angiogenesis and may participate in induction and/or maintenance of the angiogenesis phenotype.

In another preferred embodiment are angiogenesis sequences, the expression of which is reduced or diminished for prolonged periods in angiogenic tissue. These sequences are typically angiogenesis inhibitors and their diminution is correlated with an increase in angiogenesis.

Informatics

The ability to identify genes that undergo changes in expression with time during angiogenesis can additionally provide high-resolution, high-sensitivity datasets which can be used in the areas of diagnostics, therapeutics, drug development, biosensor development, and other related areas. For example, the expression profiles can be used in diagnostic or prognostic evaluation of patients with angiogenesis-associated disease. Or as another example, subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g. nucleic acids, saccharides, lipids, drugs, and the like).

Thus, in another embodiment, the present invention provides a database that includes at least one set of data assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for any assay data acquired using an assay of the invention.

The compositions and methods for identifying and/or quantitating the relative and/or absolute abundance of a variety of molecular and macromolecular species from a biological sample undergoing angiogenesis, i.e., the identification of angiogenesis-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining fill-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g. a neoplastic lesion or another tissue specimen to be analyzed for angiogenesis. In another variation, the assay records cross-tabulate one or more of the following parameters for each target species in a sample: (1) a unique identification code, which can include, e.g., a target molecular structure and/or characteristic separation coordinate (e.g. electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10 BaseT), telephone line, ISDN line, wireless network, optical fiber, or other suitable signal tranmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g. DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Angiogenesis-associated Sequences

Angiogenesis proteins of the present invention may be classified as secreted proteins, transmembrane proteins or intracellular proteins. In one embodiment, the angiogenesis protein is an intracellular protein. Intracellular proteins may be found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Molecular Biology of the Cell, 3rd Edition, Alberts, Ed., Garland Pub., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In another embodiment, the angiogenesis sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted (see, e.g. PSORT web site http://psort.nibb.acjp/).

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

Angiogenesis proteins that are transmembrane are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide acess to intracellular proteins.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In another embodiment, the angiogenesis proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Angiogenesis proteins that are secreted proteins are particularly preferred in the present invention as they serve as good targets for diagnostic markers, e.g., for blood or serum tests.

An angiogenesis sequence is initially identified by substantial nucleic acid and/or amino acid sequence homology or linkage to the angiogenesis sequences outlined herein. Such homology can be based upon the overall nucleic acid or amino acid sequence, and is generally determined as outlined below, using either homology programs or hybridization conditions. Typically, linked sequences on a mRNA are found on the same molecule.

As detailed in the definitions, percent identity can be determined using an algorithm such as BLAST. A preferred method utilizes the BLASTN module of WU-BLAST-2 set to the default parameters, with overlap span and overlap fraction set to 1 and 0.125, respectively. The alignment may include the introduction of gaps in the sequences to be aligned. In addition, for sequences which contain either more or fewer nucleotides than those of the nucleic acids of the figure it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, e.g., nucleic acids which hybridize under high stringency to a nucleic acid of Table 1, or its complement, or is also found on naturally occurring mRNAs is considered an angiogenesis sequence. In another embodiment, less stringent hybridization conditions are used; for example, moderate or low stringency conditions may be used, as are known in the art; see Ausubel, supra, and Tijssen, supra.

In addition, the angiogenesis nucleic acid sequences of the invention, e.g, the sequence in Table 1, are fragments of larger genes, i.e. they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the angiogenesis genes can be obtained, using techniques well known in the art for cloning either longer sequences or the full length sequences; see Ausubel, et al., supra. Much can be done by informatics and many sequences can be clustered to include multiple sequences, e.g., systems such as UniGene (see, http://www.ncbi.nlm.nih.gov/UniGene/).

Once the angiogenesis nucleic acid is identified, it can be cloned and, if necessary, its constituent parts recombined to form the entire angiogenesis nucleic acid coding regions or the entire mRNA sequence. Once isolated from its natural source, e.g., contained within a plasmid or other vector or excised therefrom as a linear nucleic acid segment, the recombinant angiogenesis nucleic acid can be further-used as a probe to identify and isolate other angiogenesis nucleic acids, for example extended coding regions. It can also be used as a “precursor” nucleic acid to make modified or variant angiogenesis nucleic acids and proteins.

The angiogenesis nucleic acids of the present invention are used in several ways. In a first embodiment, nucleic acid probes to the angiogenesis nucleic acids are made and attached to biochips to be used in screening and diagnostic methods, as outlined below, or for administration, for example for gene therapy, vaccine, and/or antisense applications. Alternatively, the angiogenesis nucleic acids that include coding regions of angiogenesis proteins can be put into expression vectors for the expression of angiogenesis proteins, again for screening purposes or for administration to a patient.

In a preferred embodiment, nucleic acid probes to angiogenesis nucleic acids (both the nucleic acid sequences outlined in the figures and/or the complements thereof) are made. The nucleic acid probes attached to the biochip are designed to be substantially complementary to the angiogenesis nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate. In some cases, PCR primers may be used to amplify signal for higher sensitivity.

As will be appreciated by those in the art, nucleic acids can be attached or immobilized to a solid support in a wide variety of ways. By “inunobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluorescese. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of angiogenesis-associated sequences. These assays are typically performed in conjunction with reverse transcription. In such assays, an angiogenesis-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of angiogenesis-associated RNA Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. Then the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

In a preferred embodiment, angiogenesis nucleic acids, e.g., encoding angiogenesis proteins are used to make a variety of expression vectors to express angiogenesis proteins which can then be used in screening assays, as described below. Expression vectors and recombinant DNA technology are well known to those of skill in the art (see, e.g., Ausubel, supra, and Gene Expression Systems, Fernandez & Hoeffler, Eds, Academic Press, 1999) and are used to express proteins. The expression vectors may be either self-replicating extrachromosomal vectors or vectors which integrate, into a host genome. Generally, these expression vectors include transcriptional and translational regulatory nucleic acid operably linked to the nucleic acid encoding the angiogenesis protein. The term “control sequences” refers to DNA sequences used for the expression of an operably linked coding sequence in a particular host organism. Control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the angiogenesis protein; for example, transcriptional and translational regulatory nucleic acid sequences from Bacillus are preferably used to express the angiogenesis protein in Bacillus. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The angiogenesis proteins of the present invention are produced by culturing a host cell transformed with an expression vector containing nucleic acid encoding an angiogenesis protein, under the appropriate conditions to induce or cause expression of the angiogenesis protein. Conditions appropriate for angiogenesis protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THP1 cells (a macrophage cell line) and various other human cells and cell lines.

In a preferred embodiment, the angiogenesis proteins are expressed in mammalian cells. Mammalian expression systems are also known in the art, and include retroviral and adenoviral systems. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, angiogenesis proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the angiogenesis protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, angiogenesis proteins are produced in insect cells. Expression vectors for the transformation of insect cells, and in particular, baculovirus-based expression vectors, are well known in the art.

In a preferred embodiment, angiogenesis protein is produced in yeast cells. Yeast expression systems are well known in the art, and include expression vectors for Saccharomyces cerevisiae, Candida albicans and C. maltosa, Hansenula polymorpha, Kluyveromyces fragilis and K lactis, Pichia guillerimondii and P. pastoris, Schizosaccharomyces pombe, and Yarrowia lipolytica.

The angiogenesis protein may also be made as a fusion protein, using techniques well known in the art. Thus, for example, for the creation of monoclonal antibodies, if the desired epitope is small, the angiogenesis protein may be fused to a carrier protein to form an immunogen. Alternatively, the angiogenesis protein may be made as a fusion protein to increase expression, or for other reasons. For example, when the angiogenesis protein is an angiogenesis peptide, the nucleic acid encoding the peptide may be linked to other nucleic acid for expression purposes.

In one embodiment, the angiogenesis nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the angiogenesis nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides angiogenesis protein sequences. An angiogenesis protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the angiogenesis protein has an identifiable motif or homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “Query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of angiogenesis proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques well known in the art as are outlined above for the nucleic acid homologies.

Angiogenesis proteins of the present invention may be shorter or longer than the wild type amino acid sequences. Thus, in a preferred embodiment, included within the definition of angiogenesis proteins are portions or fragments of the wild type sequences. herein. In addition, as outlined above, the angiogenesis nucleic acids of the invention may be used to obtain additional coding regions, and thus additional protein sequence, using techniques known in the art.

In a preferred embodiment, the angiogenesis proteins are derivative or variant angiogenesis proteins as compared to the wild-type sequence. That is, as outlined more fully below, the derivative angiogenesis peptide will often contain at least one amino acid substitution, deletion or insertion, with amino acid substitutions being particularly preferred. The amino acid substitution, insertion or deletion may occur at any residue within the angiogenesis peptide.

Also included within one embodiment of angiogenesis proteins of the present invention are amino acid sequence variants. These variants typically fall into one or more of three classes: substitutional, insertional or deletional variants. These variants ordinarily are prepared by site specific mutagenesis of nucleotides in the DNA encoding the angiogenesis protein, using cassette or PCR mutagenesis or other techniques well known in the art, to produce DNA encoding the variant, and thereafter expressing the DNA in recombinant cell culture as outlined above. However, variant angiogenesis protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the angiogenesis protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed angiogenesis variants screened for the optimal combination of desired activity. Techniques for making substitution mutations at predetermined sites in DNA having a known sequence are well known, for example, M13 primer mutagenesis and PCR mutagenesis. Screening of the mutants is done using assays of angiogenesis protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the angiogenesis protein are desired, substitutions are generally made in accordance with the amino acid substitution chart provided in the definition section.

Substantial changes in function or immunological identity are made by selecting substitutions that are less conservative than those provided in the definition of “conservative substitution”. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analog, although variants also are selected to modify the characteristics of the angiogenesis proteins as needed. Alternatively, the variant may be designed such that the biological activity of the angiogenesis protein is altered. For example, glycosylation sites may be altered or removed.

Covalent modifications of angiogenesis polypeptides are included within the scope of this invention. One type of covalent modification includes reacting targeted amino acid residues of an angiogenesis polypeptide with an organic derivatizing agent that is capable of reacting with selected side chains or the N-or C-terminal residues of an angiogenesis polypeptide. Derivatization with bifunctional agents is useful, for instance, for crosslinking angiogenesis polypeptides to a water-insoluble support matrix or surface for use in the method for purifying anti-angiogenesis polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the γ-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the angiogenesis polypeptide included within the scope of this invention comprises altering the native glycosylation pattern of the polypeptide. “Altering the native glycosylation pattern” is intended for purposes herein to mean deleting one or more carbohydrate moieties found in native sequence angiogenesis polypeptide, and/or adding one or more glycosylation sites that are not present in the native sequence angiogenesis polypeptide. Glycosylation patterns can be altered in many ways. For example the use of different cell types to express angiogenesis-associated sequences can result in different glycosylation patterns.

Addition of glycosylation sites to angiogenesis polypeptides may also be accomplished by altering the amino acid sequence thereof. The alteration may be made, for example, by the addition of, or substitution by, one or more serine or threonine residues to the native sequence angiogenesis polypeptide (for O-linked glycosylation sites). The angiogenesis amino acid sequence may optionally be altered through changes at the DNA level, particularly by mutating the DNA encoding the angiogenesis polypeptide at preselected bases such that codons are generated that will translate into the desired amino acids.

Another means of increasing the number of carbohydrate moieties on the angiogenesis polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the angiogenesis polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of angiogenesis comprises linking the angiogenesis polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Angiogenesis polypeptides of the present invention may also be modified in a way to form chimeric molecules comprising an angiogenesis polypeptide fused to another, heterologous polypeptide or amino acid sequence. In one embodiment, such a chimeric molecule comprises a fusion of an angiogenesis polypeptide with a tag polypeptide which provides an epitope to which an anti-tag antibody can selectively bind. The epitope tag is generally placed at the amino-or carboxyl-terminus of the angiogenesis polypeptide. The presence of such epitope-tagged forms of an angiogenesis polypeptide can be detected using an antibody against the tag polypeptide. Also, provision of the epitope tag enables the angiogenesis polypeptide to be readily purified by affinity purification using an anti-tag antibody or another type of affinity matrix that binds to the epitope tag. In an alternative embodiment, the chimeric molecule may comprise a fusion of an angiogenesis polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his).or poly-histidine-glycine (poly-his-gly) tags; HIS6 and metal chelation tags, the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al. Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with an embodiment of angiogenesis protein are other angiogenesis proteins of the angiogenesis family, and angiogenesis proteins from other organisms, which are cloned and expressed as outlined below. Thus, probe or degenerate polymerase chain reaction (PCR) primer sequences may be used to find other related angiogenesis proteins from humans or other organisms. As will be appreciated by those in the art, particularly useful probe and/or PCR primer sequences include the unique areas of the angiogenesis nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art (e.g., Innis, PCR Protocols, supra).

In addition, as is outlined herein, angiogenesis proteins can be made that are longer than those encoded by the nucleic acids of the figures, e.g., by the elucidation of extended sequences, the addition of epitope or purification tags, the addition of other fusion sequences, etc.

Angiogenesis proteins may also be identified as being encoded by angiogenesis nucleic acids. Thus, angiogenesis proteins are encoded by nucleic acids that will hybridize to the sequences of the sequence listings, or their complements, as outlined herein.

In a preferred embodiment, when the angiogenesis protein is to be used to generate antibodies, e.g., for immunotherapy or immunodiagnosis, the angiogenesis protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller angiogenesis protein will be able to bind to the full-length protein, particularly linear epitopes. In a preferred embodiment, the epitope is unique; that is, antibodies generated to a unique epitope show little or no cross-reactivity. In a preferred embodiment, the epitope is selected from a protein sequence set out in Table 2.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Table 1, or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment; the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for a protein encoded by a nucleic acid Table 1 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to angiogenesis protein are capable of reducing or eliminating a biological function of an angiogenesis protein, as is described below. That is, the addition of anti-angiogenesis protein antibodies (either polyclonal or preferably monoclonal) to angiogenic tissue (or cells containing angiogenesis) may reduce or eliminate the angiogenesis activity. Generally, at least a 25% decrease in activity is preferred, with at least about 50% being particularly preferred and about a 95-100% decrease being especially preferred.

In a preferred embodiment the antibodies to the angiogenesis proteins are humanized antibodies (e.g., Xenerex Biosciences, Mederex, Inc., Abgenix, Inc., Protein Design Labs,Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of angiogenesis with an antibody raised against angiogenesis proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the angiogenesis proteins against which antibodies are raised are secreted proteins as described above. Without being bound by theory, antibodies used for treatment, bind and prevent the secreted protein from binding to its receptor, thereby inactivating the secreted angiogenesis protein.

In another preferred embodiment, the angiogenesis protein to which antibodies are raised is a transmembrane protein. Without being bound by theory, antibodies used for treatment, bind the extracellular domain of the angiogenesis protein and prevent it from binding to other proteins, such as circulating ligands or cell-associated molecules. The antibody may cause down-regulation of the transmembrane angiogenesis protein. As will be appreciated by one of ordinary skill in the art, the antibody may be a competitive, non-competitive or uncompetitive inhibitor of protein binding to the extracellular domain of the angiogenesis protein. The antibody is also an antagonist of the angiogenesis protein. Further, the antibody prevents activation of the transmembrane angiogenesis protein. In one aspect, when the antibody prevents the binding of other molecules to the angiogenesis protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, angiogenesis is treated by administering to a patient antibodies directed against the transmembrane angiogenesis protein. Antibody-labeling may activate a co-toxin, localize a toxin payload, or otherwise provide means to locally ablate cells.

In another preferred embodiment, the antibody is conjugated to an effector moiety. The effector moiety can be any number of molecules, including labelling moieties such as radioactive labels or fluorescent labels, or can be a therapeutic moiety. In one aspect the therapeutic moiety is a small molecule that modulates the activity of the angiogenesis protein. In another aspect the therapeutic moiety modulates the activity of molecules associated with or in close proximity to the angiogenesis protein. The therapeutic moiety may inhibit enzymatic activity such as protease or collagenase activity associated with angiogenesis.

In a preferred embodiment, the therapeutic moiety can also be a cytotoxic agent. In this method, targeting the cytotoxic agent to angiogenesis tissue or cells, results in a reduction in the number of afflicted cells, thereby reducing symptoms associated with angiogenesis. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against angiogenesis proteins, or binding of a radionuclide to a chelating agent that has been covalently attached to the antibody. Targeting the therapeutic moiety to transmembrane angiogenesis proteins not only serves to increase the local concentration of therapeutic moiety in the angiogenesis afflicted area, but also serves to reduce deleterious side effects that may be associated with the therapeutic moiety.

In another preferred embodiment, the angiogenesis protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the angiogenesis protein can be targeted within a cell, i.e., the nucleus, an antibody thereto contains a signal for that target localization, i.e., a nuclear localization signal.

The angiogenesis antibodies of the invention specifically bind to angiogenesis proteins. By “specifically bind” herein is meant that the antibodies bind to the protein with a K_{d} of at least about 0.1 mM, more usually at least about 1 μ M, preferably at least about 0.1 μM or better, and most preferably, 0.01 μM or better. Selectivity of binding is also important.

In a preferred embodiment, the angiogenesis protein is purified or isolated after expression. Angiogenesis proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the angiogenesis protein may be purified using a standard anti-angiogenesis protein antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the angiogenesis protein. In some instances no purification will be necessary.

Once expressed and purified if necessary, the angiogenesis proteins and nucleic acids are useful in a number of applications. They may be used as immunoselection reagents, as vaccine reagents, as screening agents, etc.

Detection of Angiogenesis Sequence for Diagnostic and Therapeutic Applications

In one aspect, the RNA expression levels of genes are determined for different cellular states in the angiogenesis phenotype. Expression levels of genes in normal tissue (i.e., not undergoing angiogenesis) and in angiogenesis tissue (and in some cases, for varying severities of angiogenesis that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or angiogenesic tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus angiogenic tissue. Genes may be turned on or turned off in a particular state, relative to another state thus permitting comparison of two or more statese. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the angiogenesis protein and standard immunoassays (ELISAs, etc.) or other techniques, including mass spectroscopy assays, 2D gel electrophoresis assays, etc. Proteins corresponding to angiogenesis genes, i.e., those identified as being important in an angiogenesis phenotype, can be evaluated in an angiogenesis diagnostic test.

In a preferred embodiment, gene expression monitoring is performed simultaneously on a number of genes. Multiple protein expression monitoring can be performed as well. Similarly, these assays may be performed on an individual basis as well.

In this embodiment, the angiogenesis nucleic acid probes are attached to biochips as outlined herein for the detection and quantification of angiogenesis sequences in a particular cell. The assays are further described below in the example. PCR techniques can be used to provide greater sensitivity.

In a preferred embodiment nucleic acids encoding the angiogenesis protein are detected. Although DNA or RNA encoding the angiogenesis protein may be detected, of particular interest are methods wherein an mRNA encoding an angiogenesis protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding an angiogenesis protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The angiogenesis proteins, antibodies, nucleic acids, modified proteins and cells containing angiogenesis sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, angiogenesis proteins, including intracellular, transmembrane or secreted proteins, find use as markers of angiogenesis. Detection of these proteins in putative angiogenesis tissue allows for detection or diagnosis of angiogenesis. In one embodiment, antibodies are used to detect angiogenesis proteins. A preferred method separates proteins from a sample by electrophoresis on a gel (typically a denaturing and reducing protein gel, but may be another type of gel, including isoelectric focusing gels and the like). Following separation of proteins, the angiogenesis protein is detected, e.g., by immunoblotting with antibodies raised against the angiogenesis protein. Methods of immunoblotting are well known to those of ordinary skill in the art.

In another preferred method, antibodies to the angiogenesis protein find use in in situ imaging techniques, e.g., in histology (e.g., Methods in Cell Biology: Antibodies in Cell Biology, volume 37 (Asai, ed. 1993)). In this method cells are contacted with from one to many antibodies to the angiogenesis protein(s). Following washing to remove non-specific antibody binding, the presence of the antibody or antibodies is detected. In one embodiment the antibody is detected by incubating with a secondary antibody that contains a detectable label. In another method the primary antibody to the angiogenesis protein(s) contains a detectable label, for example an enzyme marker that can act on a substrate. In another preferred embodiment each one of multiple primary antibodies contains a distinct and detectable label. This method finds particular use in simultaneous screening for a plurality of angiogenesis proteins. As will be appreciated by one of ordinary skill in the art, many other histological imaging techniques are alsoprovided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing angiogenesis from blood samples. As previously described, certain angiogenesis proteins are secreted/circulating molecules. Blood samples, therefore, are useful as samples to be probed or tested for the presence of secreted angiogenesis proteins. Antibodies can be used to detect an angiogenesis protein by previously described immunoassay techniques including ELISA, immunoblotting (Western blotting), immunoprecipitation, BIACORE technology and the like. Conversely, the presence of antibodies may indicate an immune response against an endogenous angiogenesis protein.

In a preferred embodiment, in situ hybridization of labeled angiogenesis nucleic acid probes to tissue arrays is done. For example, arrays of tissue samples, including angiogenesis tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the angiogenesis proteins, antibodies, nucleic acids, modified proteins and cells containing angiogenesis sequences are used in prognosis assays. As above, gene expression profiles can be generated that correlate to angiogenesis severity, in terms of long term prognosis. Again, this may be done on either a protein or gene level, with the use of genes being preferred. As above, angiogenesis probes may be attached to biochips for the detection and quantification of angiogenesis sequences in a tissue or patient. The assays proceed as outlined above for diagnosis. PCR method may provide more sensitive and accurate quantification.

In a preferred embodiment members of the three classes of proteins as described herein are used in drug screening assays. The angiogenesis proteins, antibodies, nucleic acids, modified proteins and cells containing angiogenesis sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279, 84-8 (1998); Heid, Genome Res 6:986-94, 1996).

In a preferred embodiment, the angiogenesis proteins, antibodies, nucleic acids, modified proteins and cells containing the native or modified angiogenesis proteins are used in screening assays. That is, the present invention provides novel methods for screening for compositions which modulate the angiogenesis phenotype or an identified physiological function of an angiogenesis protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in angiogenesis, test compounds can be screened for the ability to modulate gene expression or for binding to the angiogenic protein. “Modulation” thus includes both an increase and a decrease in gene expression. The preferred amount of modulation will depend on the original change of the gene expression in normal versus tissue undergoing angiogenesis, with changes of at least 10%, preferably 50%, more preferably 100-300%, and in some embodiments 300-1000% or greater. Thus, if a gene exhibits a 4-fold increase in angiogenic tissue compared to normal tissue, a decrease of about four-fold is often desired; similarly, a 10-fold decrease in angiogenic tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the angiogenesis protein and standard immunoassays. Proteomics and separation techniques may also allow quantification of expression.

In a preferred embodiment, gene expression or protein monitoring of a number of entitites, i.e., an expression profile, is monitored simultaneously. Such profiles will typically invove a plurality of those entitites described herein.

In this embodiment, the angiogenesis nucleic acid probes are attached to biochips as outlined herein for the detection and quantification of angiogenesis sequences in a particular cell. Alternatively, PCR may be used. Thus, a series, e.g., of microtiter plate, may be used with dispensed primers in desired wells. A PCR reaction can then be performed and analyzed for each well.

Modulators of Angiogenesis

Expression monitoring can be performed to identify compounds that modify the expression of one or more angiogenesis-associated sequences, e.g., a polynucleotide sequence set out in Table 1. Generally, in a preferred embodiment, a test modulator is added to the cells prior to analysis. Moreover, screens are also provided to identify agents that modulate angiogenesis, modulate angiogenesis proteins, bind to an angiogenesis protein, or interfere with the binding of an angiogenesis protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the angiogenesis phenotype or the expression of an angiogenesis sequence, e.g., a nucleic acid or protein sequence. In preferred embodiments, modulators alter expression profiles, or expression profile nucleic acids or proteins provided herein. In one embodiment, the modulator suppresses an angiogenesis phenotype, for example to a normal tissue fingerprint. In another embodiment, a modulator induced an angiogenesis phenotype. Generally, a plurality of assay mixtures are run in parallel with different agent concentrations to obtain a differential response to the various concentrations. Typically, one of these concentrations serves as a negative control, i.e., at zero concentration or below the level of detection.

In one aspect, a modulator will neutralize the effect of an angiogenesis protein. By “neutralize” is meant that activity of a protein is inhibited or blocked and thereby has substantially no effect on a cell.

In certain embodiments, combinatorial libraries of potential modulators will be screened for an ability to bind to an angiogenesis polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) method are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al. (1994) J. Med. Chem. 37(9): 1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88), peptoids (PCT Publication No WO 91/19735, 26 Dec. 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083), antibody libraries (see, e.g. Vaughn et al. (1996) Nature Biotechnology, 14(3): 309-314), and PCT/US96/10287), carbohydrate libraries (see, e.g., Liang et al., (1996) Science, 274: 1520-1522, and U.S. Pat. No. 5,593,853), and small organic molecule libraries (see, e.g., benzodiazepines, Baum (1993) C&EN, January 18, page 25; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Mattek Biosciences, Columbia, Md., etc.).

The assays to identify modulators are amenable to high throughput screening. Preferred assays thus detect enhancement or inhibition of angiogenesis gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Paticularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of angiogenesis can also be nucleic acids, as defined above.

As described above generally for proteins, nucleic acid modulating agents may be naturally occurring nucleic acids, random nucleic acids, or “biased” random nucleic acids. For example, digests of procaryotic or eucaryotic genomes may be used as is outlined above for proteins.

In a preferred embodiment, the candidate compounds are organic chemical moieties, a wide variety of which are available in the literature.

After the candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, for example, a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g. albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the angiogenesis phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress an angiogenesis expression pattern leading to a normal expression pattern, or to modulate a single angiogenesis gene expression profile so as to mimic the expression of the gene from normal tissue, a screen as described above can be performed to identify genes that are specifically modulated in response to the agent. Comparing expression profiles between normal tissue and agent treated angiogenesis tissue reveals genes that are not expressed in normal tissue or angiogenesis tissue, but are expressed in agent treated tissue. These agent-specific sequences can be identified and used by methods described herein for angiogenesis genes or proteins. In particular these sequences and the proteins they encode find use in marking or identifying agent treated cells. In addition, antibodies can be raised against the agent induced proteins and used to target novel therapeutics to the treated angiogenesis tissue sample.

Thus, in one embodiment, a test compound is administered to a population of angiogenic cells, that have an associated angiogenesis expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, for example, angiogenesis tissue may be screened for agents that modulate, e.g., induce or suppress the angiogenesis phenotype. A change in at least one gene, preferably many, of the expression profile indicates that the agent has an effect on angiogenesis activity. By defining such a signature for the angiogenesis phenotype, screens for new drugs that alter the phenotype can be devised. With this approach, the drug target need not be known and need not be represented in the original expression screening platform, nor does the level of transcript for the target protein need to change.

Measure of angiogenesis polypeptide activity, or of angiogenesis or the angiogenic phenotype can be performed using a variety of assays. For example, the effects of the test compounds upon the function of the anagiogenesis polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of angiogenesis associated with tumors, tumor growth, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian angiogenesis polypeptide is typically used, e.g., mouse, preferably human.

A variety of angiogenesis assays are known to those of skill in the art. Various models have been employed to evaluate angiogenesis (e.g., Croix et al., Science 289:1197-1202, 2000 and Kahn et al., Amer. J. Pathol. 156:1887-1900). Assessement of angiogenesis in the presence of a potential modulator of angiogenesis can be performed using cell-cultre-based angiogenesis assays, e.g., endothelial cell tube formation assays, as well as other bioassays such as the chick CAM assay, the mouse corneal assay, and assays measuring the effect of administering potential modulators on implanted tumors. The chick CAM assay is described by O'Reilly, et al. Cell 79: 315-328, 1994. Briefly, 3 day old chicken embryos with intact yolks are separated from the egg and placed in a petri dish. After 3 days of incubation, a methylcellulose disc containing the protein to be tested is applied to the CAM of individual embryos. After about 48 hours of incubation, the embryos and CAMs are observed to determine whether endothelial growth has been inhibited. The mouse corneal assay involves implanting a growth factor-containing pellet, along with another pellet containing the suspected endothelial growth inhibitor, in the cornea of a mouse and observing the pattern of capillaries that are elaborated in the cornea. Angiogenesis can also be measured by determining the extent of neovascularization of a tumor. For example, carcinoma cells can be subcutaneously inoculated into athymic nude mice and tumor growth then monitored. The cancer cells are treated with an angiogenesis inhibitor, such as an antibody, or other compound that is exogenously administered, or can be transfected prior to inoculation with a polynucleotide inhibitor of angiogenesis. Immunoassays using endothelial cell-specific antibodies are typically used to stain for vascularization of tumor and the number of vessels in the tumor.

Assays to identify compounds with modulating activity can be performed in vitro. For example, an angiogenesis polypeptide is first contacted with a potential modulator and incubated for a suitable amount of time, e.g., from 0.5 to 48 hours. In one embodiment, the angiogenesis polypeptide levels are determined in vitro by measuring the level of protein or mRNA. The level of protein is measured using immunoassays such as western blotting, ELISA and the like with an antibody that selectively binds to the angiogenesis polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the angiogenesis protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “angiogenesis proteins”. In preferred embodiments the angiogenesis protein comprises a sequence shown in Table 2. The angiogenesis protein may be a fragment, or alternatively, be the full length protein to a fragment shown herein.

Preferably, the angiogenesis protein is a fragment of approximately 14 to 24 amino acids long. More preferably the fragment is a soluble fragment. In one embodiment an angiogenesis protein is conjugated to an immunogenic agent or BSA.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate strucutre activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the angiogenesis proteins can be used in the assays.

Thus, in a preferred embodiment, the methods comprise combining an angiogenesis protein and a candidate compound, and determining the binding of the compound to the angiogenesis protein. Preferred embodiments utilize the human angiogenesis protein, although other mammalian proteins may also be used, for example for the development of animal models of human disease. In some embodiments, as outlined herein, variant or derivative angiogenesis proteins may be used.

Generally, in a preferred embodiment of the methods herein, the angiogenesis protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the angiogenesis protein is bound to the support, and a test compound is added to the assay. Alternatively, the candidate agent is bound to the support and the angiogenesis protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the angiogenesis protein may be done in a number of ways. In a preferred embodiment, the compound is labelled, and binding determined directly, e.g., by attaching all or a portion of the angiogenesis protein to a solid support, adding a labelled candidate agent (e.g. a fluorescent label), washing off excess reagent, and determining whether the label is present on the solid support. Various blocking and washing steps may be utilized as appropriate.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin, etc. For the specific binding members, the complementary member would normally be labeled with a molecule which provides for detection, in accordance with known procedures, as outlined above. The label can directly or indirectly provide a detectable signal.

In some embodiments, only one of the components is labeled, e.g., the proteins (or proteinaceous candidate compounds) can be labeled. Alternatively, more than one component can be labeled with different labels, e.g., 125 for the proteinsand a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e. an angiogenesis protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the angiogenesis protein and thus is capable of binding to, and potentially modulating, the activity of the angiogenesis protein. In this embodiment, either component can be labeled. Thus, for example, if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the angiogenesis protein with a higher affinity. Thus, if the test compound is labeled, the presence of the label on the support, coupled with a lack of competitor binding, may indicate that the test compound is capable of binding to the angiogenesis protein.

In a preferred embodiment, the methods comprise differential screening to identity agents that are capable of modulating the activitity of the angiogenesis proteins. In this embodiment, the methods comprise combining an angiogenesis protein and a competitor in a first sample. A second sample comprises a test compound, an angiogenesis protein, and a competitor. The binding of the competitor is determined for both samples, and a change, or difference in binding between the two samples indicates the presence of an agent capable of binding to the angiogenesis protein and potentially modulating its activity. That is, if the binding of the competitor is different in the second sample relative to the first sample, the agent is capable of binding to the angiogenesis protein.

Alternatively, differential screening is used to identify drug candidates that bind to the native angiogenesis protein, but cannot bind to modified angiogenesis proteins. The structure of the angiogenesis protein may be modeled, and used in rational drug design to synthesize agents that interact with that site. Drug candidates that affect the activity of an angiogenesis protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of an angiogenesis protein. The methods comprise adding a test compound, as defined above, to a cell comprising angiogenesis proteins. Preferred cell types include almost any cell. The cells contain a recombinant nucleic acid that encodes an angiogenesis protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate angiogenesis agents are identified. Compounds with pharmacological activity are able to enhance or interfere with the activity of the angiogenesis protein. Once identified, similar structures are evaluated to identify critical structural feature of the compound.

In one embodiment, a method of inhibiting angiogenic cell division is provided. The method comprises administration of an angiogenesis inhibitor. In another embodiment, a method of inhibiting angiogenesis is provided. The method comprises administration of an angiogenesis inhibitor. In a further embodiment, methods of treating cells or individuals with angiogenesis are provided. The method comprises administration of an angiogenesis inhibitor.

In one embodiment, an angiogenesis inhibitor is an antibody as discussed above. In another embodiment, the angiogenesis inhibitor is an antisense molecule.

Polynucleotide Modulators of Angiogenesis

Antisense Polynucleotides

In certain embodiments, the activity of an angiogenesis-associated protein is downregulated, or entirely inhibited, by the use of antisense polynucleotide, i.e., a nucleic acid complementary to, and which can preferably hybridize specifically to, a coding mRNA nucleic acid sequence, e.g. in angiogenesis protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the angiogenesis protein mRNA. See, e.g., Isis Pharmaceuticals, Carlsbad, CA; Sequitor, Inc., Natick, MA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block trancription by binding to the anti-sense strand. The antisense and sense oligonucleotide comprise a single-stranded nucleic acid sequence (either RNA or DNA) capable of binding to target mRNA (sense) or DNA (antisense) sequences for angiogenesis molecules. A preferred antisense molecule is for an angiogenesis sequences in Table 1, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Ribozymes

In addition to antisense polynucleotides, ribozymes can be used to target and inhibit transcription of angiogenesis-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al. (1994) Adv. in Pharmacology 25: 289-317 for a general review of the properties of different ribozymes).

The general features of hairpin ribozymes are described, e.g., in Hampel et al. (1990) Nucl. Acids Res. 18: 299-304; Hampel et al. (1990) European Patent Publication No. 0 360 257; U.S. Pat. No. 5,254,678. Methods of preparing are well known to those of skill in the art (see, e.g., Wong-Staal et al., WO 94/26877; Ojwang et al. (1993) Proc. Natl. Acad. Sci. USA 90: 6340-6344; Yamada et al. (1994) Human Gene Therapy 1: 39-45; Leavitt et al. (1995) Proc. Natl. Acad. Sci. USA 92: 699-703; Leavitt et al. (1994) Human Gene Therapy 5: 1151-120; and Yamada et al. (1994) Virology 205: 121-126).

Polynucleotide modulators of angiogenesis may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of angiogenesis may be introduced into a cell containing the target nucleic acid sequence, e.g., by formation of an polynucleotide-lipid complex, as described in WO 90/10448. It is understood that the use of aitisense molecules or knock out and knock in models may also be used in screening assays as discussed above, in addition to methods of treatment.

Thus, in one embodiment, methods of modulating angiogenesis in cells or organisms are provided. In one embodiment, the methods comprise administering to a cell an anti-angiogenesis antibody that reduces or eliminates the biological activity of an endogeneous angiogenesis protein. Alternatively, the methods comprise administering to a cell or organism a recombinant nucleic acid encoding an angiogenesis protein. This may be accomplished in any number of ways. In a preferred embodiment, for example when the angiogenesis sequence is down-regulated in angiogenesis, such state may be reversed by increasing the amount of angiogenesis gene product in the cell. This can be accomplished, e.g., by overexpressing the endogeneous angiogenesis gene or administering a gene encoding the angiogenesis sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entireity. Alternatively, for example when the angiogenesis sequence is up-regulated in angiogenesis, the activity of the endogeneous angiogenesis gene is decreased, for example by the administration of a angiogenesis antisense nucleic acid.

In one embodiment, the angiogenesis proteins of the present invention may be used to generate polyclonal and monoclonal antibodies to angiogenesis proteins. Similarly, the angiogenesis proteins can be coupled, using standard technology, to affinity chromatography columns. These columns may then be used to purify angiogenesis antibodies useful for production, diagnostic, or therapeutic purposes. In a preferred embodiment, the antibodies are generated to epitopes unique to a angiogenesis protein; that is, the antibodies show little or no cross-reactivity to other proteins. The angiogenesis antibodies may be coupled to standard affinity chromatography columns and used to purify angiogenesis proteins. The antibodies may also be used as blocking polypeptides, as outlined above, since they will specifically bind to the angiogenesis protein.

Methods of Identifying Variant Angiogenesis-associated Sequences

Without being bound by theory, expression of various angiogenesis sequences is correlated with angiogenesis. Accordingly, disorders based on mutant or variant angiogenesis genes may be determined. In one embodiment, the invention provides methods for identifying cells containing variant angiogenesis genes, e.g., determining all or part of the sequence of at least one endogeneous angiogenesis genes in a cell. This may be accomplished using any number of sequencing techniques. In a preferred embodiment, the invention provides methods of identifying the angiogenesis genotype of an individual, e.g., determining all or part of the sequence of at least one angiogenesis gene of the individual. This is generally done in at least one tissue of the individual, and may include the evaluation of a number of tissues or different samples of the same tissue. The method may include comparing the sequence of the sequenced angiogenesis gene to a known angiogenesis gene, i.e., a wild-type gene.

The sequence of all or part of the angiogenesis gene can then be compared to the sequence of a known angiogenesis gene to determine if any differences exist. This can be done using any number of known homology programs, such as Bestfit, etc. In a preferred embodiment, the presence of a a difference in the sequence between the angiogenesis gene of the patient and the known angiogenesis gene correlates with a disease state or a propensity for a disease state, as outlined herein.

In a preferred embodiment, the angiogenesis genes are used as probes to determine the number of copies of the angiogenesis gene in the genome.

In another preferred embodiment, the angiogenesis genes are used as probes to determine the chromosomal localization of the angiogenesis genes. Information such as chromosomal localization finds use in providing a diagnosis or prognosis in particular when chromosomal abnormalities such as translocations, and the like are identified in the angiogenesis gene locus.

Administration of Pharmaceutical and Vaccine Compositions

In one embodiment, a therapeutically effective dose of an angiogenesis protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceuitcal Dosage Forms and Drug Delivery, Lippincott, Williams & Wilkins Publishers, ISBN:0683305727; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding, Amer. Pharmacutical Assn, ISBN 0917330889; and Pickar (1999) Dosage Calculations, Delmar Pub, ISBN 0766805042). As is known in the art, adjustments for angiogenesis degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the angiogenesis proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the angiogenesis proteins and modulators may be directly applied as a solution or spray.

The pharmaceutical compositions of the present invention comprise an angiogenesis protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that angiogenesis protein modulators (e.g. antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are well known in the art.

The compositions for administration will commonly comprise an angiogenesis protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science, 15th ed., Mack Publishing Company, Easton, Pa. (1980) and Goodman and Gillman, The Pharmacologial Basis of Therapeutics,(Hardman, J. G, Limbird, L. E, Molinoff, P. B., Ruddon, R. W, and Gilman, A. G., eds) TheMcGraw-Hill Companies, Inc.,1996).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacologial Basis of Therapeutics, supra.

The compositions containing modulators of angiogenesis proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present angiogenesis protein-modulating compounds can be administered alone or in combination with additional angiogenesis modulating compounds or with other therapeutic agent, e.g., other anti-cancer agents or treatments.

In numerous embodiments, one or more nucleic acids, e.g., polynucleotides comprising nucleic acid sequences set forth in Table 1, such as antisense polynucleotides or ribozyrnes, will be introduced into cells, in vitro or in vivo. The present invention provides methods, reagents, vectors, and cells useful for expression of angiogenesis-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger and Kimmel, Guide to Molecular Cloning Techniques, Methods in Enzymology volume 152 Academic Press, Inc., San Diego, Calif. (Berger), F. M. Ausubel et al., eds., Current Protocols, a joint venture between Greene Publishing Associates, Inc. and John Wiley & Sons, Inc., (supplemented through 1999), and Sambrook et al., Molecular Cloning—A Laboratory Manual (2nd Ed.), Vol. 1-3, Cold Spring Harbor Laboratory, Cold Spring Harbor, N.Y., 1989.

In a preferred embodiment, angiogenesis proteins and modulators are administered as therapeutic agents, and can be formulated as outlined above. Similarly, angiogenesis genes (including both the full-length sequence, partial sequences, or regulatory sequences of the angiogenesis coding regions) can be administered in a gene therapy application. These angiogenesis genes can include antisense applications, either as gene therapy (i.e. for incorporation into the genome) or as antisense compositions, as will be appreciated by those in the art.

Angiogenesis polypeptides and polynucleotides can also be administered as vaccine compositions to stimulate HTL, CTL and antibody responses. Such vaccine compositions can include, for example, lipidated peptides (e.g.,Vitiello, A. et al., J. Clin. Invest. 95:341, 1995), peptide compositions encapsulated in poly(DL-lactide-co-glycolide) (“PLG”) microspheres (see, e.g., Eldridge, et al., Milec. Immunol. 28:287-294, 1991: Alonso et al., Vaccine 12:299-306, 1994; Jones et al., Vaccine 13:675-681, 1995), peptide compositions contained in immune stimulating complexes (ISCOMS) (see, e.g. Takahashi et al., Nature 344:873-875, 1990; Hu et al., Clin Exp Immunol. 113:235-243, 1998), multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996), peptides formulated as multivalent peptides; peptides for use in ballistic delivery systems, typically crystallized peptides, viral delivery vectors (Perkus, M. E. et al., In: Concepts in vaccine development, Kaufinann, S. H. E., ed., p. 379, 1996; Chakrabarti, S. et al., Nature 320:535, 1986; Hu, S. L. et al., Nature 320:537, 1986; Kieny, M. -P. et al., AIDS Bio/Technology 4:790, 1986; Top, F. H. et al., J. Infect. Dis. 124:148, 1971; Chanda, P. K. et al., Virology 175:535, 1990), particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995), adjuvants (Warren, H. S., Vogel, F. R., and Chedid, L. A. Annu. Rev. Immunol. 4:369, 1986; Gupta, R. K. et al., Vaccine 11:293, 1993), liposomes (Reddy, R. et al., J. Immunol. 148:1585, 1992; Rock, K. L., Immunol. Today 17:131, 1996), or, naked or particle absorbed cDNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable inicrospheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, for example, as a vector to express nucleotide sequences that encode angiogenic polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization e.g. adeno and adeno-associated virus vectors, retroviral vectors, Salmonella typhi vectors, detoxified anthrax toxin vectors, and the like, will be apparent to those skilled in the art from the description herein (see, e.g., Shata et al. (2000) Mol Med Today, 6: 66-71; Shedlock et al., J. Leukoc Biol 68,:793-806, 2000; Hipp et al., In Vivo 14:571-85, 2000).

Methods for the use of genes as DNA vaccines are well known, and include placing an angiogenesis gene or portion of an angiogenesis gene under the control of a regulatable promoter or a tissue-specific promoter for expression in an angiogenesis patient. The angiogenesis gene used for DNA vaccines can encode full-length angiogenesis proteins, but more preferably encodes portions of the angiogenesis proteins including peptides derived from the angiogenesis protein. In one embodiment, a patient is immunized with a DNA vaccine comprising a plurality of nucleotide sequences derived from an angiogenesis gene. For example, angiogenesis-associated genes or sequence encoding subfragments of an angiogenesis protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the angiogenesis polypeptide encoded by the DNA vaccine. Additional or alternative adjuvants are available.

In another preferred embodiment angiogenesis genes find use in generating animal models of angiogenesis. When the angiogenesis gene identified is repressed or diminished in angiogenesic tissue, gene therapy technology, e.g., wherein antisense RNA directed to the angiogenesis gene will also diminish or repress expression of the gene. Animal models of angiogenesis find use in screening for modulators of an angiogenesis-associated sequence or modulators of angiogenesis. Similarly, transgenic animal technology including gene knockout technology, for example as a result of homologous recombination with an appropriate gene targeting vector, will result in the absence or increased expression of the angiogenesis protein. When desired, tissue-specific expression or knockout of the angiogenesis protein may be necessary.

It is also possible that the angiogenesis protein is overexpressed in angiogenesis. As such, transgenic animals can be generated that overexpress the angiogenesis protein. Depending on the desired expression level, promoters of various strengths can be employed to express the transgene. Also, the number of copies of the integrated transgene can be determined and compared for a determination of the expression level of the transgene. Animals generated by such methods find use as animal models of angiogenesis and are additionally useful in screening for modulators to treat angiogenesis.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, angiogenesis-specific nucleic acids or antibodies, hybridization probes and/or primers, antisense polynucleotides, ribozymes, dominant negative angiogenesis polypeptides or polynucleotides, small molecules inhibitors of angiogenesis-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of angiogenesis-associated sequences. Such kits can be prepared from readily available materials and reagents. For example, such kits can comprise one or more of the following materials: an angiogenesis-associated polypeptide or polynucleotide, reaction tubes, and instructions for testing angiogenic-associated activity. Optionally, the kit contains biologically active angiogenesis protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

It is understood that the examples described above in no way serve to limit the true scope of this invention, but rather are presented for illustrative purposes. All publications, sequences of accession numbers, and Patent applications cited in this specification are herein incorporated by reference as if each individual publication or Patent application were specifically and individually indicated to be incorporated by reference.

### EXAMPLES

### Example 1

### Tissue Preparation, Labeling Chips, and Fingerprints

Purify Total RNA from Tissue Using TRIzol Reagent

Homogenize tissue samples in 1 ml of TRIzol per 50 mg of tissue using a Polytron 3100 homogenizer. The generator/probe used depends upon the tissue size. A generator that is too large for the amount of tissue to be homogenized will cause a loss of sample and lower RNA yield. TRIzol is added directly to frozen tissue, which is then homogenize. Following homogenization, insoluble material is removed by centrifugation at 7500× g for 15 min in a Sorvall superspeed or 12,000× g for 10 min. in an Eppendorf centrifuge at 4° C. The clear homogenate is transferred to a new tube for use. The samples may be frozen now at −60° to −70° C. (and kept for at least one month). The homogenate is mixed with 0.2 ml of chloroform per 1 ml of TRIzol reagent used in the original homogenization and incubated at room temp. for 2-3 minutes. The aqueous phase is then separated by centrifugation and transferred to a fresh tube and the RNA precipitated using isopropyl alcohol. The pellet is isolated by centrifugation, washed, air-dried, resuspended in an appropriate volume of DEPC H₂O, and the absorbance measured.

Purification of poly A+ mRNA from total RNA is performed as follows. Heat an oligotex suspension to 37° C. and mixing immediately before adding to RNA. The Elution Buffer is heated at 70° C. Warm up 2× Binding Buffer at 65° C. if there is precipitate in the buffer. Mix total RNA with DEPC-treated water, 2× Binding Buffer, and Oligotex according to Table 2 on page 16 of the Oligotex Handbook. Incubate for 3 minutes at 65° C. Incubate for 10 minutes at room temperature. Centrifuge for 2 minutes at 14,000 to 18,000 g. Remove supernatant without disturbing Oligotex pellet. A little bit of solution can be left behind to reduce the loss of Oligotex. Gently resuspend in Wash Buffer OW2 and pipet onto spin column. Centrifuge the spin column at full speed for 1 minute. Transfer spin column to a new collection tube and gently resuspend in Wash Buffer OW2 and centrifuge as describe herein. Transfer spin column to a new tube and elute with 20 to 100 ul of preheated (70° C.) Elution Buffer. Gently resuspend Oligotex resin by pipetting up and down. Centrifuge as above. Repeat elution with fresh elution buffer or use first eluate to keep the elution volume low. Read absorbance, using diluted Elution Buffer as the blank. Before proceeding with cDNA synthesis, precipitate the mRNA as follows: add 0.4 vol. of 7.5 M NH4OAc+2.5 vol. of cold 100% ethanol. Precipitate at −20° C. 1 hour to overnight (or 20-30 min. at −70° C.). Centrifuge at 14,000-16,000× g for 30 minutes at 4° C. Wash pellet with 0.5 ml of 80%ethanol (−20° C.) then centrifuge at 14,000-16,000× g for 5 minutes at room temperature. Repeat 80% ethanol wash. Air dry the ethanol from the pellet in the hood. Suspend pellet in DEPC H₂O at 1 ug/ul concentration.

To further Clean up total RNA using Qiagen's RNeasy kit, add no more than 100 ug to an RNeasy column. Adjust sample to a volume of 100 ul with RNase-free water. Add 350 ul Buffer RLT then 250 ul ethanol (100%) to the sample. Mix by pipetting (do not centrifuge) then apply sample to an RNeasy mini spin column. Centrifuge for 15 sec at >10,00 rpm. Transfer column to a new 2-ml collection tube. Add 500 ul Buffer RPE and centrifuge for 15 sec at >10,00 rpm. Discard flowthrough. Add 500 ul Buffer RPE and centrifuge for 15 sec at >10,000 rpm. Discard flowthrough then centrifuge for 2 min at maximum speed to dry column membrane. Transfer column to a new 1.5-ml collection tube and apply 30-50 ul of RNase-free water directly onto column membrane. Centrifuge 1 min at >10,000 rpm. Repeat elution. and read absorbance.

cDNA Synthesis Using Gibco's “SuperScript Choice System for cDNA Synthesis” Kit

First Strand cDNA synthesis is performed as follows. Use 5 ug of total RNA or 1 ug of polyA+ mRNA as starting material. For total RNA, use 2 ul of SuperScript RT. For polyA+ mRNA, use 1 ul of SuperScript RT. Final volume of first strand synthesis mix is 20 ul. RNA must be in a volume no greater than 10 ul. Incubate RNA with 1 ul of 100 pmol T7-T24 oligo for 10 min at 70 C. On ice, add 7 ul of: 4 ul 5× 1st Strand Buffer, 2 ul of 0.1M DTT, and 1 ul of 10 nM dNTP mix. Incubate at 37 C. for 2 min then add SuperScript RT. Incubate at 37 C. for 1 hour.

For the second strand synthesis, place 1st strand reactions on ice and add: 91 ul DEPC H₂O; 30 ul 5× 2nd Strand Buffer; 3 ul 10 mM dNTP mix; 1 ul 10 U/ul E. coli DNA Ligase; 4 ul 10 U/ul E. coli DNA Polymerase; and 1 ul 2 U/ul RNase H. Mix and incubate 2 hours at 16 C. Add 2 ul T4 DNA Polymerase. Incubate 5 min at 16 C. Add 10 ul of 0.5M EDTA. A further clean-up of DNA is performed using phenol:chloroform:isoamyl Alcohol (25:24:1) purification.

In vitro Transcription (IVT) and labeling with biotin is performed as follows: Pipet 1.5 ul of cDNA into a thin-wall PCR tube. Make NTP labeling mix by combining 2 ul T7 10× ATP (75 mM) (Ambion); 2 ul T7 10× GTP (75 mM) (Ambion); 1.5 ul T7 10× CTP (75 mM) (Ambion); 1.5 ul T7 10× UTP (75 mM) (Ambion); 3.75 ul 10 mM Bio-11-UTP (Boehringer-Mannheim/Roche or Enzo); 3.75 ul 10 nM Bio-16-CTP (Enzo); 2 ul 10× T7 transcription buffer (Ambion); and 2 ul 10× T7 enzyme mix (Ambion). The final volume is 20 ul. Incubate 6 hours at 37° C. in a PCR machine. The RNA can be furthered cleaned.

Fragmentation is performed as follows. 15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer. Fragment RNA by incubation at 94 C. for 35 minutes in 1× Fragmentation buffer (5× Fragmentation buffer is 200 mM Tris-acetate, pH 8.1; 500 mM KOAc; 150 mM MgOAc). The labeled RNA transcript can be analyzed before and after fragmentation. Samples can be heated to 65° C. for 15 minutes and electrophoresed on 1% agarose/TBE gels to get an approximate idea of the transcript size range.

For hybridization, 200 ul (10 ug cRNA) of a hybridization mix is put on the chip. If multiple hybridizations are to be done (such as cycling through a 5 chip set), then it is recommended that an initial hybridization mix of 300 ul or more be made. The hybridization mix is: fragment labeled RNA (50 ng/ul final conc.); 50 pM 948-b control oligo; 1.5 pM BioB; 5 pM BioC; 25 pM BioD; 100 pM CRE; 0.1 mg/ml herring sperm DNA; 0.5 mg/ml acetylated BSA; and 300 ul with 1× MES hyb buffer.

Labeling is performed as follows: The hybridization reaction includes non-biotinylated IVT (purified by RNeasy columns); IVT antisense RNA 4 μg:μl; random Hexamers (1 μg/μl) 4 μl and water to 14 ul. The reaciton is incubated at 70° C., 10 min. Reverse transcriptionis performed in the following reaction: 5× First Strand (BRL) buffer, 6 μl; 0.1 M DTT, 3 μl; 50× dNTP mix, 0.6 μl; H₂O, 2.4 μl; Cy3 or CyS dUTP (lmM), 3 pL; SS RT II (BRL), 1 μl in a final volume of 16 μl. Add to hybridization reaction. Incubate 30 min., 42° C. Add 1 μl SSII and incubate another hour. Put on ice. 50× dNTP mix (25 mM of cold dATP, dCTP, and dGTP, 10 mM of dTTP: 25 μl each of 100 mM dATP, dCTP, and dGTP; 10 μl of 100 mM dTTP to 15 μl H₂O. dNTPs from Pharmacia). RNA degradation is performed as follows. Add 86 μl H₂O, 1.5 μl 1M NaOH/2 mM EDTA and incubate at 65° C., 10 min. For U-Con 30, 500 μl TE/sample spin at 7000 g for 10 min, save flow through for purification. For Qiagen purification, suspend u-con recovered material in 500 μl buffer PB and proceed using Qiagen protocol. For DNAse digestion, add 1 ul of 1/100dil of DNAse/30 ul Rx and incubate at 37° C. for 15 min. Incubate at 5 min 95° C. to denature the DNAse/.

For sample preparation, add Cot-1 DNA, 10 μl; 50× dNTPs, 1 μl; 20× SSC, 2.3 μl; Na pyro phosphate, 7.5 μl; 10 mg/ml Herring sperm DNA; 1 ul of 1/10 dilution to 21.8 final vol. Dry in speed vac. Resuspend in 15 μl H₂O. Add 0.38 μl 10% SDS. Heat 95° C., 2 min and slow cool at room temp. for 20 min. Put on slide and hybridize overnight at 64° C. Washing after the hybridization: 3× SSC0.03% SDS: 2 min., 37.5 mls 20× SSC+0.75 mls 10% SDS in 250 mls H2O; 1× SSC: 5 min., 12.5 mls 20× SSC in 250 mls H2O; 0.2× SSC: 5 min., 2.5 mls 20× SSC in 250 mls H2O. Dry slides and scan at appropiate PMT's and channels.

### Example 2

### A Model of Angiogenesis is Used to Determine Expression in Angiogenesis

In the model of angiogenesis used to determine expression of angiogenesis-associated sequences, human umbilical vein endothelial cells (HUVEC) were obtained, e.g., as passage 1 (p1) frozen cells from Cascade Biologics (Oregon) and grown in maintenance medium: Medium 199 (Life Technologies) supplemented with 20% pooled human serum, 100 mg/ml heparin and 75 mg/ml endothelial cell growth supplements (Sigma) and gentamicin (Life Technologies). An in vitro cell system model was used in which 2×10⁵ HUVECs were cultured in 0.5 ml 3 mgs/ml plasminogen-depleted fibrinogen (Calbiochem, San Diego, Calif.) that was polymerized by the addition of 1 unit of maintenance medium supplemented with 100 ng/ml VEGF and HGF and 10 ng/ml TGF-a (R&D Systems, Minneapolis, Minn.) added (growth medium). The growth medium was replaced every 2 days. Samples for RNA were collected, e.g., at 0, 2, 6, 15, 24, 48, and 96 hours of culture. The fibrin clots were placed in Trizol (Life Technologies) and disrupted using a Tissuemizer. Thereafter standard procedures were used for extracting the RNA (e.g., Example 1).

Angiogenesis associated sequences thus identified are shown in Table 1. As indicated, some of the Accession numbers include expression sequence tags (ESTs). Thus, in one embodiment herein, genes within an expression profile, also termed expression profile genes, include ESTs and are not necessarily full length.

TABLE 1

AAA4 DNA sequence
Gene name: CGI-100 protein
Unigene number: Hs.275253
Probeset Accession #: AA089688
Nucleic Acid Accession #: NM_016040 cluster
Coding sequence: 142-831 (predicted start/stop codons underlined)

| | |
|---|---|
| GTTCGCCGCC GCCGCGCCGG CCACCTGGAG TTTTTTCAGA CTCCAGATTT CCCTGTCAAC | 60 |
| CACGAGGAGT CCAGAGAGGA AACGCGGAGC GGAGACAACA GTACCTGACG CCTCTTTCAG | 120 |
| CCCGGGATCG CCCCAGCAGG GATGGGCGAC AAGATCTGGC TGCCCTTCCC CGTGCTCCTT | 180 |
| CTGGCCGCTC TGCCTCCGGT GCTGCTGCCT GGGGCGGCCG GCTTCACACC TTCCCTCGAT | 240 |
| AGCGACTTCA CCTTTACCCT TCCCGCCGGC CAGAAGGAGT GCTTCTACCA GCCCATGCCC | 300 |
| CTGAAGGCCT CGCTGGAGAT CGAGTACCAA GTTTTAGATG GAGCAGGATT AGATATTGAT | 360 |
| TTCCATCTTG CCTCTCCAGA AGGCAAAACC TTAGTTTTTG AACAAAGAAA ATCAGATGGA | 420 |
| GTTCACACTG TAGAGACTGA AGTTGGTGAT TACATGTTCT GCTTTGACAA TACATTCAGC | 480 |
| ACCATTTCTG AGAAGGTGAT TTTCTTTGAA TTAATCCTGG ATAATATGGG AGAACAGGCA | 540 |
| CAAGAACAAG AAGATTGGAA GAAATATATT ACTGGCACAG ATATATTGGA TATGAAACTG | 600 |
| GAAGACATCC TGGAATCCAT CAACAGCATC AAGTCCAGAC TAAGCAAAAG TGGGCACATA | 660 |
| CAAACTCTGC TTAGAGCATT TGAAGCTCGT GATCGAAACA TACAAGAAAG CAACTTTGAT | 720 |
| AGAGTCAATT TCTGGTCTAT GGTTAATTTA GTGGTCATGG TGGTGGTGTC AGCCATTCAA | 780 |
| GTTTATATGC TGAAGAGTCT GTTTGAAGAT AAGAGGAAAA GTAGAACTTA AAACTCCAAA | 840 |
| CTAGAGTACG TAACATTGAA AAATGAGGCA TAAAAATGCA ATAAACTGTT ACAGTCAAGA | 900 |
| CCATTAATGG TCTTCTCCAA AATATTTTGA GATATAAAAG TAGGAAACAG GTATAATTTT | 960 |
| AATGTGAAAA TTAAGTCTTC ACTTTCTGTG CAAGTAATCC TGCTGATCCA GTTGTACTTA | 1020 |
| AGTGTGTAAC AGGAATATTT TGCAGAATAT AGGTTTAACT GAATGAAGCC ATATTAATAA | 1080 |
| CTGCATTTTC CTAACTTTGA AAAATTTTGC AAATGTCTTA GGTGATTTAA ATAAATGAGT | 1140 |
| ATTGGGCCTA AA | |

AAA7 DNA sequence
Gene name: Endothelial differentiation, sphingolipid G-protein-coupled receptor, 1
(EDG1)
Unigene number: Hs.154210
Probeset Accession #: M31210
Nucleic Acid Accession #: NM_001400 cluster
Coding sequence: 251-1396 (predicted start/stop codons underlined)

| | |
|---|---|
| TCTAAAGGTC GGGGGCAGCA GCAAGATGCG AAGCGAGCCG TACAGATCCC GGGCTCTCCG | 60 |
| AACGCAACTT CGCCCTGCTT GAGCGAGGCT GCGGTTTCCG AGGCCCTCTC CAGCGAAGGA | 120 |
| AAAGCTACAC AAAAAGCCTG GATCACTCAT CGAACCACCC CTGAAGCCAG TGAAGGCTCT | 180 |
| CTCGCCTCGC CCTCTAGCGT TCGTCTGGAG TAGCGCCACC CCGGCTTCCT GGGGACACAG | 240 |
| GGTTGGCACC ATGGGGCCCA CCAGCGTCCC GCTGGTCAAG GCCCACCGCA GCTCGGTCTC | 300 |
| TGACTACGTC AACTATGATA TCATCGTCCG GCATTACAAC TACACGGGAA AGCTGAATAT | 360 |
| CAGCGCGGAC AAGGAGAACA GCATTAAACT GACCTCGGTG GTGTTCATTC TCATCTGCTG | 420 |
| CTTTATCATC CTGGAGAACA TCTTTGTCTT GCTGACCATT TGGAAAACCA AGAAATTCCA | 480 |
| CCGACCCATG TACTATTTTA TTGGCAATCT GGCCCTCTCA GACCTGTTGG CAGGAGTAGC | 540 |
| CTACACAGCT AACCTGCTCT TGTCTGGGGC CACCACCTAC AAGCTCACTC CCGCCCAGTG | 600 |
| GTTTCTGCGG GAAGGGAGTA TGTTTGTGGC CCTGTCAGCC TCCGTGTTCA GTCTCCTCGC | 660 |
| CATCGCCATT GAGCGCTATA TCACAATGCT GAAAATGAAA CTCCACAACG GGAGCAATAA | 720 |
| CTTCCGCCTC TTCCTGCTAA TCAGCGCCTG CTGGGTCATC TCCCTCATCC TGGGTGGCCT | 780 |
| GCCTATCATG GGCTGGAACT GCATCAGTGC GCTGTCCAGC TGCTCCACCG TGCTGCCGCT | 840 |
| CTACCACAAG CACTATATCC TCTTCTGCAC CACGGTCTTC ACTCTGCTTC TGCTCTCCAT | 900 |
| CGTCATTCTG TACTGCAGAA TCTACTCCTT GGTCAGGACT CGGAGCCGCC GCCTGACGTT | 960 |
| CCGCAAGAAC ATTTCCAAGG CCAGCCGCAG CTCTGAGAAT GTGGCGCTGC TCAAGACCGT | 1020 |
| AATTATCGTC CTGAGCGTCT TCATCGCCTG CTGGGCACCG CTCTTCATCC TGCTCCTGCT | 1080 |
| GGATGTGGGC TGCAAGGTGA AGACCTGTGA CATCCTCTTC AGAGCGGAGT ACTTCCTGGT | 1140 |
| GTTACCTGTG CTCAACTCCG GCACCAACCC CATCATTTAC ACTCTGACCA ACAAGGAGAT | 1200 |
| GCGTCGGGCC TTCATCCGGA TCATGTCCTG CTGCAAGTGC CCGAGCGGAG ACTCTGCTGG | 1260 |
| CAAATTCAAG CGACCCATCA TCGCCGGCAT GGAATTCAGC CGCAGCAAAT CGGACAATTC | 1320 |
| CTGGCACCCC CAGAAAGACG AAGGGGACAA CCCAGAGACC ATTATGTCTT CTGGAAACGT | 1380 |
| CAACTCTTCT TCCTAGAACT GGAAGCTGTC CACCCACCGG AAGCGCTCTT TACTTGGTCG | 1440 |
| CTGGCCACCC CAGTGTTTGG AAAAAAATCT CTGGGCTTCG ACTGCTGCCA GGGAGGAGCT | 1500 |
| GCTGCAAGCC AGAGGGAGGA AGGGGGAGAA TACGAACAGC CTGGTGGTGT CGGGTGTTGG | 1560 |
| TGGGTAGAGT TAGTTCCTGT GAACAATGCA CTGGGAAGGG TGGAGATCAG GTCCCGGCCT | 1620 |
| GGAATATATA TTCTACCCCC CTGGAGCTTT GATTTTGCAC TGAGCCAAAG GTCTAGCATT | 1680 |
| GTCAAGCTCC TAAAGGGTTC ATTTGGCCCC TCCTCAAAGA CTAATGTCCC CATGTGAAAG | 1740 |
| CGTCTCTTTG TCTGGAGCTT TGAGGAGATG TTTTCCTTCA CTTTAGTTTC AAACCCAAGT | 1800 |
| GAGTGTGTGC ACTTCTGCTT CTTTAGGGAT GCCCTGTACA TCCCACACCC CACCCTCCCT | 1860 |
| TCCCTTCATA CCCCTCCTCA ACGTTCTTTT ACTTTATACT TTAACTACCT GAGAGTTATC | 1920 |
| AGAGCTGGGG TTGTGGAATG ATCGATCATC TATAGCAAAT AGGCTATGTT GAGTACGTAG | 1980 |
| GCTGTGGGAA GATGAAGATG GTTTGGAGGT GTAAAACAAT GTCCTTCGCT GAGGCCAAAG | 2040 |
| TTTCCATGTA AGCGGGATCC GTTTTTTGGA ATTTGGTTGA AGTCACTTTG ATTTCTTTAA | 2100 |
| AAAACATCTT TTCAATGAAA TGTGTTACCA TTTCATATCC ATTGAAGCCG AAATCTGCAT | 2160 |
| AAGGAAGCCC ACTTTATCTA AATGATATTA GCCAGGATCC TTGGTGTCCT AGGAGAAACA | 2220 |
| GACAAGCAAA ACAAAGTGAA AACCGAATGG ATTAACTTTT GCAAACCAAG GGAGATTTCT | 2280 |
| TAGCAAATGA GTCTAACAAA TATGACATCC GTCTTTCCCA CTTTTGTTGA TGTTTATTTC | 2340 |
| AGAATCTTGT GTGATTCATT TCAAGCAACA ACATGTTGTA TTTTGTTGTG TTAAAAGTAC | 2400 |
| TTTTCTTGAT TTTTGAATGT ATTTGTTTCA GGAAGAAGTC ATTTTATGGA TTTTTCTAAC | 2460 |
| CCGTGTTAAC TTTTCTAGAA TCCACCCTCT TGTGCCCTTA AGCATTACTT TAACTGGTAG | 2520 |
| GGAACGCCAG AACTTTTAAG TCCAGCTATT CATTAGATAG TAATTGAAGA TATGTATAAA | 2580 |
| TATTACAAAG AATAAAAATA TATTACTGTC TCTTTAGTAT GGTTTTCAGT GCAATTAAAC | 2640 |
| CGAGAGATGT CTTGTTTTTT TAAAAAGAAT AGTATTTAAT AGGTTTCTGA CTTTTGTGGA | 2700 |
| TCATTTTGCA CATAGCTTTA TCAACTTTTA AACATTAATA AACTGATTTT TTTAAAG | |

AAB3 DNA sequence
Gene name: Solute carrier family 20 (phosphate transporter), member 1, Human
leukaemia virus receptor 1 (GLVR1)
Unigene number: Hs.78452
Probeset Accession #: L20859
Nucleic Acid Accession #: NM_005415 cluster
Coding sequence: predicted 371-2410 (predicted start/stop codons underlined)

| | |
|---|---|
| GAGCTGTCCC CGGTGCCGCC GACCCGGGCC GTGCCGTGTG CCCGTGGCTC CAGCCGCTGC | 60 |
| CGCCTCGATC TCCTCGTCTC CCGCTCCGCC CTCCCTTTTC CCTGGATGAA CTTGCGTCCT | 120 |
| TTCTCTTCTC CGCCATGGAA TTCTGCTCCG TGCTTTTAGC CCTCCTGAGC CAAAGAAACC | 180 |
| CCAGACAACA GATGCCCATA CGCAGCGTAT AGCAGTAACT CCCCAGCTCG GTTTCTGTGC | 240 |
| CGTAGTTTAC AGTATTTAAT TTTATATAAT ATATATTATT TATTATAGCA TTTTTGATAC | 300 |
| CTCATATTCT GTTTACACAT CTTGAAAGGC GCTCAGTAGT TCTCTTACTA AACAACCACT | 360 |
| ACTCCAGAGA ATGGCAACGC TGATTACCAG TACTACAGCT GCTACCGCCG CTTCTGGTCC | 420 |
| TTTGGTGGAC TACCTATGGA TGCTCATCCT GGGCTTCATT ATTGCATTTG TCTTGGCATT | 480 |
| CTCCGTGGGA GCCAATGATG TAGCAAATTC TTTTGGTACA GCTGTGGGCT CAGGTGTAGT | 540 |
| GACCCTGAAG CAAGCCTGCA TCCTAGCTAG CATCTTTGAA ACAGTGGGCT CTGTCTTACT | 600 |
| GGGGGCCAAA GTGAGCGAAA CCATCCGGAA GGGCTTGATT GACGTGGAGA TGTACAACTC | 660 |
| GACTCAAGGG CTACTGATGG CCGGCTCAGT CAGTGCTATG TTTGGTTCTG CTGTGTGGCA | 720 |
| ACTCGTGGCT TCGTTTTTGA AGCTCCCTAT TTCTGGAACC CATTGTATTG TTGGTGCAAC | 780 |
| TATTGGTTTC TCCCTCGTGG CAAAGGGGCA GGAGGGTGTC AAGTGGTCTG AACTGATAAA | 840 |
| AATTGTGATG TCTTGGTTCG TGTCCCCACT GCTTTCTGGA ATTATGTCTG GAATTTTATT | 900 |
| CTTCCTGGTT CGTGCATTCA TCCTCCATAA GGCAGATCCA GTTCCTAATG GTTTGCGAGC | 960 |
| TTTGCCAGTT TTCTATGCCT GCACAGTTGG AATAAACCTC TTTTCCATCA TGTATACTGG | 1020 |
| AGCACCGTTG CTGGGCTTTG ACAAACTTCC TCTGTGGGGT ACCATCCTCA TCTCGGTGGG | 1080 |
| ATGTGCAGTT TTCTGTGCCC TTATCGTCTG GTTCTTTGTA TGTCCCAGGA TGAAGAGAAA | 1140 |
| AATTGAACGA GAAATAAAGT GTAGTCCTTC TGAAAGCCCC TTAATGGAAA AAAAGAATAG | 1200 |
| CTTGAAAGAA GACCATGAAG AAACAAAGTT GTCTGTTGGT GATATTGAAA ACAAGCATCC | 1260 |
| TGTTTCTGAG GTAGGGCCTG CCACTGTGCC CCTCCAGGCT GTGGTGGAGG AGAGAACAGT | 1320 |
| CTCATTCAAA CTTGGAGATT TGGAGGAAGC TCCAGAGAGA GAGAGGCTTC CCAGCGTGGA | 1380 |
| CTTGAAAGAG GAAACCAGCA TAGATAGCAC CGTGAATGGT GCAGTGCAGT TGCCTAATGG | 1440 |
| GAACCTTGTC CAGTTCAGTC AAGCCGTCAG CAACCAAATA AACTCCAGTG GCCACTCCCA | 1500 |
| GTATCACACC GTGCATAAGG ATTCCGGCCT GTACAAAGAG CTACTCCATA AATTACATCT | 1560 |
| TGCCAAGGTG GGAGATTGCA TGGGAGACTC CGGTGACAAA CCCTTAAGGC GCAATAATAG | 1620 |
| CTATACTTCC TATACCATGG CAATATGTGG CATGCCTCTG GATTCATTCC GTGCCAAAGA | 1680 |
| AGGTGAACAG AAGGGCGAAG AAATGGAGAA GCTGACATGG CCTAATGCAG ACTCCAAGAA | 1740 |
| GCGAATTCGA ATGGACAGTT ACACCAGTTA CTGCAATGCT GTGTCTGACC TTCACTCAGC | 1800 |
| ATCTGAGATA GACATGAGTG TCAAGGCAGC GATGGGTCTA GGTGACAGAA AAGGAAGTAA | 1860 |
| TGGCTCTCTA GAAGAATGGT ATGACCAGGA TAAGCCTGAA GTCTCTCTCC TCTTCCAGTT | 1920 |
| CCTGCAGATC CTTACAGCCT GCTTTTGGTC ATTCGCCCAT GGTGGCAATG ACGTAAGCAA | 1980 |
| TGCCATTGGG CCTCTGGTTG CTTTATATTT GGTTTATGAC ACAGGAGATG TTTCTTCAAA | 2040 |
| AGTGGCAACA CCAATATGGC TTCTACTCTA TGGTGGTGTT GGTATCTGTG TTGGTCTGTG | 2100 |
| GGTTTGGGGA AGAAGAGTTA TCCAGACCAT GGGGAAGGAT CTGACACCGA TCACACCCTC | 2160 |
| TAGTGGCTTC AGTATTGAAC TGGCATCTGC CCTCACTGTG GTGATTGCAT CAAATATTGG | 2220 |
| CCTTCCCATC AGTACAACAC ATTGTAAAGT GGGCTCTGTT GTGTCTGTTG GCTGGCTCCG | 2280 |
| GTCCAAGAAG GCTGTTGACT GGCGTCTCTT TCGTAACATT TTTATGGCCT GGTTTGTCAC | 2340 |
| AGTCCCCATT TCTGGAGTTA TCAGTGCTGC CATCATGGCA ATCTTCAGAT ATGTCATCCT | 2400 |
| CAGAATGTGA AGCTGTTTGA GATTAAAATT TGTGTCAATG TTTGGGACCA TCTTAGGTAT | 2460 |
| TCCTGCTCCC CTGAAGAATG ATTACAGTGT TAACAGAAGA CTGACAAGAG TCTTTTTATT | 2520 |
| TGGGAGCAGA GGAGGGAAGT GTTACTTGTG CTATAACTGC TTTTGTGCTA AATATGAATT | 2580 |
| GTCTCAAAAT TAGCTGTGTA AAATAGCCCG GGTTCCACTG GCTCCTGCTG AGGTCCCCTT | 2640 |
| TCCTTCTGGG CTGTGAATTC CTGTACATAT TTCTCTACTT TTTGTATCAG GCTTCAATTC | 2700 |
| CATTATGTTT TAATGTTGTC TCTGAAGATG ACTTGTGATT TTTTTTTCTT TTTTTTAAAC | 2760 |
| CATGAAGAGC CGTTTGACAG AGCATGCTCT GCGTTGTTGG TTTCACCAGC TTCTGCCCTC | 2820 |
| ACATGCACAG GGATTTAACA ACAAAAATAT AACTACAACT TCCCTTGTAG TCTCTTATAT | 2880 |
| AAGTAGAGTC CTTGGTACTC TGCCCTCCTG TCAGTAGTGG CAGGATCTAT TGGCATATTC | 2940 |
| GGGAGCTTCT TAGAGGGATG AGGTTCTTTG AACACAGTGA AAATTTAAAT TAGTAACTTT | 3000 |
| TTTGCAAGCA GTTTATTGAC TGTTATTGCT AAGAAGAAGT AAGAAAGAAA AAGCCTGTTG | 3060 |
| GCAATCTTGG TTATTTCTTT AAGATTTCTG GCAGTGTGGG ATGGATGAAT GAAGTGGAAT | 3120 |
| GTGAACTTTG GGCAAGTTAA ATGGGACAGC CTTCCATGTT CATTTGTCTA CCTCTTAACT | 3180 |
| GAATAAAAAA GCCTACAGTT TTTAGAAAAA ACCCGAATTC | |

AAB4 DNA sequence
Gene name: Matrix metalloproteinase 10 (stromelysin 2)
Unigene number: Hs.2258
Probeset Accession #: X07820
Nucleic Acid Accession #: NM_002425
Coding sequence: predicted 23-1453 (predicted start/stop codons underlined)

| | |
|---|---|
| AAAGAAGGTA AGGGCAGTGA GAATGATGCA TCTTGCATTC CTTGTGCTGT TGTGTCTGCC | 60 |
| AGTCTGCTCT GCCTATCCTC TGAGTGGGGC AGCAAAAGAG GAGGACTCCA ACAAGGATCT | 120 |
| TGCCCAGCAA TACCTAGAAA AGTACTACAA CCTCGAAAAG GATGTGAAAC AGTTTAGAAG | 180 |
| AAAGGACAGT AATCTCATTG TTAAAAAAAT CCAAGGAATG CAGAAGTTCC TTGGGTTGGA | 240 |
| GGTGACAGGG AAGCTAGACA CTGACACTCT GGAGGTGATG CGCAAGCCCA GGTGTGGAGT | 300 |
| TCCTGACGTT GGTCACTTCA GCTCCTTTCC TGGCATGCCG AAGTGGAGGA AAACCCACCT | 360 |
| TACATACAGG ATTGTGAATT ATACACCAGA TTTGCCAAGA GATGCTGTTG ATTCTGCCAT | 420 |
| TGAGAAAGCT CTGAAAGTCT GGGAAGAGGT GACTCCACTC ACATTCTCCA GGCTGTATGA | 480 |
| AGGAGAGGCT GATATAATGA TCTCTTTCGC AGTTAAAGAA CATGGAGACT TTTACTCTTT | 540 |
| TGATGGCCCA GGACACAGTT TGGCTCATGC CTACCCACCT GGACCTGGGC TTTATGGAGA | 600 |
| TATTCACTTT GATGATGATG AAAAATGGAC AGAAGATGCA TCAGGCACCA ATTTATTCCT | 660 |
| CGTTGCTGCT CATGAACTTG GCCACTCCCT GGGGCTCTTT CACTCAGCCA ACACTGAAGC | 720 |
| TTTGATGTAC CCACTCTACA ACTCATTCAC AGAGCTCGCC CAGTTCCGCC TTTCGCAAGA | 780 |
| TGATGTGAAT GGCATTCAGT CTCTCTACGG ACCTCCCCCT GCCTCTACTG AGGAACCCCT | 840 |
| GGTGCCCACA AAATCTGTTC CTTCGGGATC TGAGATGCCA GCCAAGTGTG ATCCTGCTTT | 900 |
| GTCCTTCGAT GCCATCAGCA CTCTGAGGGG AGAATATCTG TTCTTTAAAG ACAGATATTT | 960 |
| TTGGCGAAGA TCCCACTGGA ACCCTGAACC TGAATTTCAT TTGATTTCTG CATTTTGGCC | 1020 |
| CTCTCTTCCA TCATATTTGG ATGCTGCATA TGAAGTTAAC AGCAGGGACA CCGTTTTTAT | 1080 |
| TTTTAAAGGA AATGAGTTCT GGGCCATCAG AGGAAATGAG GTACAAGCAG GTTATCCAAG | 1140 |
| AGGCATCCAT ACCCTGGGTT TTCCTCCAAC CATAAGGAAA ATTGATGCAG CTGTTTCTGA | 1200 |
| CAAGGAAAAG AAGAAAACAT ACTTCTTTGC AGCGGACAAA TACTGGAGAT TTGATGAAAA | 1260 |
| TAGCCAGTCC ATGGAGCAAG GCTTCCCTAG ACTAATAGCT GATGACTTTC CAGGAGTTGA | 1320 |
| GCCTAAGGTT GATGCTGTAT TACAGGCATT TGGATTTTTC TACTTCTTCA GTGGATCATC | 1380 |
| ACAGTTTGAG TTTGACCCCA ATGCCAGGAT GGTGACACAC ATATTAAAGA GTAACAGCTG | 1440 |
| GTTACATTGC TAGGCGAGAT AGGGGGAAGA CAGATATGGG TGTTTTTAAT AAATCTAATA | 1500 |
| ATTATTCATC TAATGTATTA TGAGCCAAAA TGGTTAATTT TTCCTGCATG TTCTGTGACT | 1560 |
| GAAGAAGATG AGCCTTGCAG ATATCTGCAT GTGTCATGAA GAATGTTTCT GGAATTCTTC | 1620 |
| ACTTGCTTTT GAATTGCACT GAACAGAATT AAGAAATACT CATGTGCAAT AGGTGAGAGA | 1680 |
| ATGTATTTTC ATAGATGTGT TATTACTTCC TCAATAAAAA GTTTTATTTT GGGCCTGTTC | 1740 |
| CTT | |

AAB6 DNA sequence
Gene name: Podocalyxin-like
Unigene number: Hs.16426
Probeset Accession #: U97519
Nucleic Acid Accession #: NM_005397 cluster
Coding sequence: 251-1837 (predicted start/stop codons underlined)

| | |
|---|---|
| AAACGCCGCC CAGGACGCAG CCGCCGCCGC CGCCGCTCCT CTGCCACTGG CTCTGCGCCC | 60 |
| CAGCCCGGCT CTGCTGCAGC GGCAGGGAGG AAGAGCCGCC GCAGCGCGAC TCGGGAGCCC | 120 |
| CGGGCCACAG CCTGGCCTCC GGAGCCACCC ACAGGCCTCC CCGGGCGGCG CCCACGCTCC | 180 |
| TACCGCCCGG ACGCGCGGAT CCTCCGCCGG CACCGCAGCC ACCTGCTCCC GGCCCAGAGG | 240 |
| CGACGACACG ATGCGCTGCG CGCTGGCGCT CTCGGCGCTG CTGCTACTGT TGTCAACGCC | 300 |
| GCCGCTGCTG CCGTCGTCGC CGTCGCCGTC GCCGTCGCCG TCGCCCTCCC AGAATGCAAC | 360 |
| CCAGACTACT ACGGACTCAT CTAACAAAAC AGCACCGACT CCAGCATCCA GTGTCACCAT | 420 |
| CATGGCTACA GATACAGCCC AGCAGAGCAC AGTCCCCACT TCCAAGGCCA ACGAAATCTT | 480 |
| GGCCTCGGTC AAGGCGACCA CCCTTGGTGT ATCCAGTGAC TCACCGGGGA CTACAACCCT | 540 |
| GGCTCAGCAA GTCTCAGGCC CAGTCAACAC TACCGTGGCT AGAGGAGGCG GCTCAGGCAA | 600 |
| CCCTACTACC ACCATCGAGA GCCCCAAGAG CACAAAAAGT GCAGACACCA CTACAGTTGC | 660 |
| AACCTCCACA GCCACAGCTA AACCTAACAC CACAAGCAGC CAGAATGGAG CAGAAGATAC | 720 |
| AACAAACTCT GGGGGGAAAA GCAGCCACAG TGTGACCACA GACCTCACAT CCACTAAGGC | 780 |
| AGAACATCTG ACGACCCCTC ACCCTACAAG TCCACTTAGC CCCCGACAAC CCACTTTGAC | 840 |
| GCATCCTGTG GCCACCCCAA CAAGCTCGGG ACATGACCAT CTTATGAAAA TTTCAAGCAG | 900 |
| TTCAAGCACT GTGGCTATCC CTGGCTACAC CTTCACAAGC CCGGGGATGA CCACCACCCT | 960 |
| ACCGTCATCG GTTATCTCGC AAAGAACTCA ACAGACCTCC AGTCAGATGC CAGCCAGCTC | 1020 |
| TACGGCCCCT TCCTCCCAGG AGACAGTGCA GCCCACGAGC CCGGCAACGG CATTGAGAAC | 1080 |
| ACCTACCCTG CCAGAGACCA TGAGCTCCAG CCCCACAGCA GCATCAACTA CCCACCGATA | 1140 |
| CCCCAAAACA CCTTCTCCCA CTGTGGCTCA TGAGAGTAAC TGGGCAAAGT GTGAGGATCT | 1200 |
| TGAGACACAG ACACAGAGTG AGAAGCAGCT CGTCCTGAAC CTCACAGGAA ACACCCTCTG | 1260 |
| TGCAGGGGGC GCTTCGGATG AGAAATTGAT CTCACTGATA TGCCGAGCAG TCAAAGCCAC | 1320 |
| CTTCAACCCG GCCCAAGATA AGTGCGGCAT ACGGCTGGCA TCTGTTCCAG GAAGTCAGAC | 1380 |
| CGTGGTCGTC AAAGAAATCA CTATTCACAC TAAGCTCCCT GCCAAGGATG TGTACGAGCG | 1440 |
| GCTGAAGGAC AAATGGGATG AACTAAAGGA GGCAGGGGTC AGTGACATGA AGCTAGGGGA | 1500 |
| CCAGGGGCCA CCGGAGGAGG CCGAGGACCG CTTCAGCATG CCCCTCATCA TCACCATCGT | 1560 |
| CTGCATGGCG TCATTCCTGC TCCTCGTGGC GGCCCTCTAT GGCTGCTGCC ACCAGCGCCT | 1620 |
| CTCCCAGAGG AAGGACCAGC AGCGGCTAAC AGAGGAGCTG CAGACAGTGG AGAATGGTTA | 1680 |
| CCATGACAAC CCAACACTGG AAGTGATGGA GACCTCTTCT GAGATGCAGG AGAAGAAGGT | 1740 |
| GGTCAGCCTC AACGGGGAGC TGGGGGACAG CTGGATCGTC CCTCTGGACA ACCTGACCAA | 1800 |
| GGACGACCTG GATGAGGAGG AAGACACACA CCTCTAGTCC GGTCTGCCGG TGGCCTCCAG | 1860 |
| CAGCACCACA GAGCTCCAGA CCAACCACCC CAAGTGCCGT TTGGATGGGG AAGGGAAAGA | 1920 |
| CTGGGGAGGG AGAGTGAACT CCGAGGGGTG TCCCCTCCCA ATCCCCCCAG GGCCTTAATT | 1980 |
| TTTCCCTTTT CAACCTGAAC AAATCACATT CTGTCCAGAT TCCTCTTGTA AAATAACCCA | 2040 |
| CTAGTGCCTG AGCTCAGTGC TGCTGGATGA TGAGGGAGAT CAAGAAAAAG CCACGTAAGG | 2100 |
| GACTTTATAG ATGAACTAGT GGAATCCCTT CATTCTGCAG TGAGATTGCC GAGACCTGAA | 2160 |
| GAGGGTAAGT GACTTGCCCA AGGTCAGAGC CACTTGGTGA CAGAGCCAGG ATGAGAACAA | 2220 |
| AGATTCCATT TGCACCATGC CACACTGCTG TGTTCACATG TGCCTTCCGT CCAGAGCAGT | 2280 |
| CCCGGGCAGG GGTGAAACTC CAGCAGGTGG CTGGGCTGGA AAGGAGGGCA GGGCTACATC | 2340 |
| CTGGCTCGGT GGGATCTGAC GACCTGAAAG TCCAGCTCCC AAGTTTTCCT TCTCCTACCC | 2400 |
| CAGCCTCGTG TACCCATCTT CCCACCCTCT ATGTTCTTAC CCCTCCCTAC ACTCAGTGTT | 2460 |
| TGTTCCCACT TACTCTGTCC TGGGGCCTCT GGGATTAGCA CAGGTTATTC ATAACCTTGA | 2520 |
| ACCCCTTGTT CTGGATTCGG ATTTTCTCAC ATTTGCTTCG TGAGATGGGG GCTTAACCCA | 2580 |
| CACAGGTCTC CGTGCGTGAA CCAGGTCTGC TTAGGGGACC TGCGTGCAGG TGAGGAGAGA | 2640 |
| AGGGGACACT CGAGTCCAGG CTGGTATCTC AGGGCAGCTG ATGAGGGGTC AGCAGGAACA | 2700 |
| CTGGCCCATT GCCCCTGGCA CTCCTTGCAG AGGCCACCCA CGATCTTCTT TGGGCTTCCA | 2760 |
| TTTCCACCAG GGACTAAAAT CTGCTGTAGC TAGTGAGAGC AGCGTGTTCC TTTTGTTGTT | 2820 |
| CACTGCTCAG CTGATGGGAG TGATTCCCTG AGACCCAGTA TGAAAGAGCA GTGGCTGCAG | 2880 |
| GAGAGGCCTT CCCGGGGCCC CCCATCAGCG ATGTGTCTTC AGAGACAATC CATTAAAGCA | 2940 |
| GCCAGGAAGG ACAGGCTTTC CCCTGTATAT CATAGGAAAC TCAGGGACAT TTCAAGTTGC | 3000 |
| TGAGAGTTTT GTTATAGTTG TTTTCTAACC CAGCCCTCCA CTGCCAAAGG CCAAAAGCTC | 3060 |
| AGACAGTTGG CAGACGTCCA GTTAGCTCAT CTCACTCACT CTGATTCTCC TGTGCCACAG | 3120 |
| GAAAAGAGGG CCTGGAAAGC GCAGTGCATG CTGGGTGCAT GAAGGGCAGC CTGGGGGACA | 3180 |
| GACTGTTGTG GGAACGTCCC ACTGTCCTGG CCTGGAGCTA GGCCTTGCTG TTCCTCTTCT | 3240 |
| CTGTGAGCCT AGTGGGGCTG CTGCGGTTCT CTTGCAGTTT CTGGTGGCAT CTCAGGGGAA | 3300 |
| CACAAAAGCT ATGTCTATTC CCCAATATAG GACTTTTATG GGCTCGGCAG TTAGCTGCCA | 3360 |
| TGTAGAAGGC TCCTAAGCAG TGGGCATGGT GAGGTTTCAT CTGATTGAGA AGGGGGAATC | 3420 |
| CTGTGTGGAA TGTTGAACTT TCGCCATGGT CTCCATCGTT CTGGGCGTAA ATTCCCTGGG | 3480 |
| ATCAAGTAGG AAAATGGGCA GAACTGCTTA GGGGAATGAA ATTGCCATTT TTCGGGTGAA | 3540 |
| ACGCCACACC TCCAGGGTCT TAAGAGTCAG GCTCCGGCTG TAGTAGCTCT GATGAAATAG | 3600 |
| GCTATCCACT CGGGATGGCT TACTTTTTAA AAGGGTAGGG GGAGGGGCTG GGGAAGATCT | 3660 |
| GTCCTGCACC ATCTGCCTAA TTCCTTCCTC ACAGTCTGTA GCCATCTGAT ATCCTAGGGG | 3720 |
| GAAAAGGAAG GCCAGGGGTT CACATAGGGC CCCAGCGAGT TTCCCAGGAG TTAGAGGGAT | 3780 |
| GCGAGGCTAA CAAGTTCCAA AAACATCTGC CCCGATGCTC TAGTGTTTGG AGGTGGGCAG | 3840 |
| GATGGAGAAC AGTGCCTGTT TGGGGGAAAA CAGGAAATCT TGTTAGGCTT GAGTGAGGTG | 3900 |
| TTTGCTTCCT TCTTGCCCAG CGCTGGGTTC TCTCCACCCA GTAGGTTTTC TGTTGTGGTC | 3960 |
| CCGTGGGAGA GGCCAGACTG GATTATTCCT CCTTTGCTGA TCCTGGGTCA CACTTCACCA | 4020 |
| GCCAGGGCTT TTGACGGAGA CAGCAAATAG GCCTCTGCAA ATCAATCAAA GGCTGCAACC | 4080 |
| CTATGGCCTC TTGGAGACAG ATGATGACTG GCAAGGACTA GAGAGCAGGA GTGCCTGGCC | 4140 |
| AGGTCGGTCC TGACTCTCCT GACTCTCCAT CGCTCTGTCC AAGGAGAACC CGGAGAGGCT | 4200 |
| CTGGGCTGAT TCAGAGGTTA CTGCTTTATA TTCGTCCAAA CTGTGTTAGT CTAGGCTTAG | 4260 |
| GACAGCTTCA GAATCTGACA CCTTGCCTTG CTCTTGCCAC CAGGACACCT ATGTCAACAG | 4320 |
| GCCAAACAGC CATGCATCTA TAAAGGTCAT CATCTTCTGC CACCTTTACT GGGTTCTAAA | 4380 |
| TGCTCTCTGA TAATTCAGAG AGCATTGGGT CTGGGAAGAG GTAAGAGGAA CACTAGAAGC | 4440 |
| TCAGCATGAC TTAAACAGGT TGTAGCAAAG ACAGTTTATC ATCAACTCTT TCAGTGGTAA | 4500 |
| ACTGTGGTTT CCCCAAGCTG CACAGGAGGC CAGAAACCAC AAGTATGATG ACTAGGAAGC | 4560 |
| CTACTGTCAT GAGAGTGGGG AGACAGGCAG CAAAGCTTAT GAAGGAGGTA CAGAATATTC | 4620 |
| TTTGCGTTGT AAGACAGAAT ACGGGTTTAA TCTAGTCTAG GCRCCAGATT TTTTTCCCGC | 4680 |
| TTGATAAGGA AAGCTAGCAG AAAGTTTATT TAAACCACTT CTTGAGCTTT ATCTTTTTTG | 4740 |
| ACAATATACT GGAGAAACTT TGAAGAACAA GTTCAAACTG ATACATATAC ACATATTTTT | 4800 |
| TTGATAATGT AAATACAGTG ACCATGTTAA CCTACCCTGC ACTGCTTTAA GTGAACATAC | 4860 |
| TTTGAAAAAG CATTATGTTA GCTGAGTGAT GGCCAAGTTT TTTCTCTGGA CAGGAATGTA | 4920 |
| AATGTCTTAC TGGAAATGAC AAGTTTTTGC TTGATTTTTT TTTTTAAACA AAAAATGAAA | 4980 |
| TATAACAAGA CAAACTTATG ATAAAGTATT TGTCTTGTAG ATCAGGTGTT TTGTTTTGTT | 5040 |
| TTTTTAATTT TAAAATGCAA CCCTGCCCCC TCCCCAGCAA AGTCACAGCT CCATTTCAGT | 5100 |
| AAAGGTTGGA GTCAATATGC TCTGGTTGGC AGGCAACCCT GTAGTCATGG AGAAAGGTAT | 5160 |
| TTCAAGATCT AGTCCAATCT TTTTCTAGAG AAAAAGATAA TCTGAAGCTC ACAAAGATGA | 5220 |
| AGTGACTTCC TCAAAATCAC ATGGTTCAGG ACAGAAACAA GATTAAAACC TGGATCCACA | 5280 |
| GACTGTGCGC CTCAGAAGGA ATAATCGGTA AATTAAGAAT TGCTACTCGA AGGTGCCAGA | 5340 |
| ATGACACAAA GGACAGAATT CCTTTCCCAG TTGTTACCCT AGCAAGGCTA GGGAGGGCAT | 5400 |
| GAACACAAAC ATAAGAACTG GTCTTCTCAC ACTTTCTCTG AATCATTTAG GTTTAAGATG | 5460 |
| TAAGTGAACA ATTCTTTCTT TCTGCCAAGA AACAAAGTTT TGGATGAGCT TTTATATATG | 5520 |
| GAACTTACTC CAACAGGACT GAGGGACCAA GGAAACATGA TGGGGGAGGC AAGAGAGGGC | 5580 |
| AAAGAGTAAA ACTGTAGCAT AGCTTTTGTC ACGGTCACTA GCTGATCCCT CAGGTCTGCT | 5640 |
| GCAAACACAG CATGGAGGAC ACAGATGACT CTTTGGTGTT GGTCTTTTTG TCTGCAGTGA | 5700 |
| ATGTTCAACA GTTTGCCCAG GAACTGGGGG ATCATATATG TCTTAGTGGA CAGGGGTCTG | 5760 |
| AAGTACACTG GAATTTACTG AGAAACTTGT TTGTAAAAAC TATAGTTAAT AATTATTGCA | 5820 |
| TTTTCTTACA AAAATATATT TTGGAAAATT GTATACTGTC AATTAAAGT | |

AAB8 DNA sequence
Gene name: EGF-containing fibulin-like extracellular matrix protein 1
Unigene number: Hs.76224
Probeset Accession #: U03877
Nucleic Acid Accession #: NM_004105 Transcript variant 1
Coding sequence: 150-1631 (predicted start/stop codons underlined)

| | |
|---|---|
| CTAGTATTCT ACTAGAACTG GAAGATTGCT CTCCGAGTTT TTTTTTTGTT ATTTTGTTAA | 60 |
| AAAATAAAAA GCTTGAGCAG CAATTCATAT TACTGTCACA GGTATTTTTG CTGTGCTGTG | 120 |
| CAAGGTAACT CTGCTAGCTA AGATTCACAA TGTTGAAAGC CCTTTTCCTA ACTATGCTGA | 180 |
| CTCTGGCGCT GGTCAAGTCA CAGGACACCG AAGAAACCAT CACGTACACG CAATGCACTG | 240 |
| ACGGATATGA GTGGGATCCT GTGAGACAGC AATGCAAAGA TATTGATGAA TGTGACATTG | 300 |
| TCCCAGACGC TTGTAAAGGT GGAATGAAGT GTGTCAACCA CTATGGAGGA TACCTCTGCC | 360 |
| TTCCGAAAAC AGCCCAGATT ATTGTCAATA ATGAACAGCC TCAGCAGGAA ACACAACCAG | 420 |
| CAGAAGGAAC CTCAGGGGGA ACCACCGGGG TTGTAGCTGC CAGCAGCATG GCAACCAGTG | 480 |
| GAGTGTTGCC CGGGGGTGGT TTTGTGGCCA GTGCTGCTGC AGTCGCAGGC CCTGAAATGC | 540 |
| AGACTGGCCG AAATAACTTT GTCATCCGGC GGAACCCAGC TGACCCTCAG CGCATTCCCT | 600 |
| CCAACCCTTC CCACCGTATC CAGTGTGCAG CAGGCTACGA GCAAAGTGAA CACAACGTGT | 660 |
| GCCAAGACAT AGACGAGTGC ACTGCAGGGA CGCACAACTG TAGAGCAGAC CAAGTGTGCA | 720 |
| TCAATTTACG GGGATCCTTT GCATGTCAGT GCCCTCCTGG ATATCAGAAG CGAGGGGAGC | 780 |
| AGTGCGTAGA CATAGATGAA TGTACCATCC CTCCATATTG CCACCAAAGA TGCGTGAATA | 840 |
| CACCAGGCTC ATTTTATTGC CAGTGCAGTC CTGGGTTTCA ATTGGCAGCA AACAACTATA | 900 |
| CCTGCGTAGA TATAAATGAA TGTGATGCCA GCAATCAATG TGCTCAGCAG TGCTACAACA | 960 |
| TTCTTGGTTC ATTCATCTGT CAGTGCAATC AAGGATATGA GCTAAGCAGT GACAGGCTCA | 1020 |
| ACTGTGAAGA CATTGATGAA TGCAGAACCT CAAGCTACCT GTGTCAATAT CAATGTGTCA | 1080 |
| ATGAACCTGG GAAATTCTCA TGTATGTGCC CCCAGGGATA CCAAGTGGTG AGAAGTAGAA | 1140 |
| CATGTCAAGA TATAAATGAG TGTGAGACCA CAAATGAATG CCGGGAGGAT GAAATGTGTT | 1200 |
| GGAATTATCA TGGCGGCTTC CGTTGTTATC CACGAAATCC TTGTCAAGAT CCCTACATTC | 1260 |
| TAACACCAGA GAACCGATGT GTTTGCCCAG TCTCAAATGC CATGTGCCGA GAACTGCCCC | 1320 |
| AGTCAATAGT CTACAAATAC ATGAGCATCC GATCTGATAG GTCTGTGCCA TCAGACATCT | 1380 |
| TCCAGATACA GGCCACAACT ATTTATGCCA ACACCATCAA TACTTTTCGG ATTAAATCTG | 1440 |
| GAAATGAAAA TGGAGAGTTC TACCTACGAC AAACAAGTCC TGTAAGTGCA ATGCTTGTGC | 1500 |
| TCGTGAAGTC ATTATCAGGA CCAAGAGAAC ATATCGTGGA CCTGGAGATG CTGACAGTCA | 1560 |
| GCAGTATAGG GACCTTCCGC ACAAGCTCTG TGTTAAGATT GACAATAATA GTGGGGCCAT | 1620 |
| TTTCATTTTA GTCTTTTCTA AGAGTCAACC ACAGGCATTT AAGTCAGCCA AAGAATATTG | 1680 |
| TTACCTTAAA GCACTATTTT ATTTATAGAT ATATCTAGTG CATCTACATC TCTATACTGT | 1740 |
| ACACTCACCC ATAACAAACA ATTACACCAT GGTATAAAGT GGGCATTTAA TATGTAAAGA | 1800 |
| TTCAAAGTTT GTCTTTATTA CTATATGTAA ATTAGACATT AATCCACTAA ACTGGTCTTC | 1860 |
| TTCAAGAGAG CTAAGTATAC ACTATCTGGT GAAACTTGGA TTCTTTCCTA TAAAAGTGGG | 1920 |
| ACCAAGCAAT GATGATCTTC TGTGGTGCTT AAGGAAACTT ACTAGAGCTC CACTAACAGT | 1980 |
| CTCATAAGGA GGCAGCCATC ATAACCATTG AATAGCATGC AAGGGTAAGA ATGAGTTTTT | 2040 |
| AACTGCTTTG TAAGAAAATG GAAAAGGTCA ATAAAGATAT ATTTCTTTAG AAAATGGGGA | 2100 |
| TCTGCCATAT TTGTGTTGGT TTTTATTTTC ATATCCAGCC TAAAGGTGGT TGTTTATTAT | 2160 |
| ATAGTAATAA ATCATTGCTG TACAACATGC TGGTTTCTGT AGGGTATTTT TAATTTTGTC | 2220 |
| AGAAATTTTA GATTGTGAAT ATTTTGTAAA AAACAGTAAG CAAAATTTTC CAGAATTCCC | 2280 |
| AAAATGAACC AGATACCCCC TAGAAAATTA TACTATTGAG AAATCTATGG GGAGGATATG | 2340 |
| AGAAAATAAA TTCCTTCTAA ACCACATTGG AACTGACCTG AAGAAGCAAA CTCGGAAAAT | 2400 |
| ATAATAACAT CCCTGAATTC AGGCATTCAC AAGATGCAGA ACAAAATGGA TAAAAGGTAT | 2460 |
| TTCACTGGAG AAGTTTTAAT TTCTAAGTAA AATTTAAATC CTAACACTTC ACTAATTTAT | 2520 |
| AACTAAAATT TCTCATCTTC GTACTTGATG CTCACAGAGG AAGAAAATGA TGATGGTTTT | 2580 |
| TATTCCTGGC ATCCAGAGTG ACAGTGAACT TAAGCAAATT ACCCTCCTAC CCAATTCTAT | 2640 |
| GGAATATTTT ATACGTCTCC TTGTTTAAAA TCTGACTGCT TTACTTTGAT GTATCATATT | 2700 |
| TTTAAATAAA AATAAATATT CCTTTAGAAG ATCACTCTAA AA | |

AAB9 DNA sequence
Gene name: Melanoma adhesion molecule, MUC 18 glycoprotein
Unigene number: Hs.211579
Probeset Accession #: M28882
Nucleic Acid Accession #: NM_006500 cluster
Coding sequence: 27-1967 (predicted start/stop codons underlined)

| | |
|---|---|
| ACTTGCGTCT CGCCCTCCGG CCAAGCATGG GGCTTCCCAG GCTGGTCTGC GCCTTCTTGC | 60 |
| TCGCCGCCTG CTGCTGCTGT CCTCGCGTCG CGGGTGTGCC CGGAGAGGCT GAGCAGCCTG | 120 |
| CGCCTGAGCT GGTGGAGGTG GAAGTGGGCA GCACAGCCCT TCTGAAGTGC GGCCTCTCCC | 180 |
| AGTCCCAAGG CAACCTCAGC CATGTCGACT GGTTTTCTGT CCACAAGGAG AAGCGGACGC | 240 |
| TCATCTTCCG TGTGCGCCAG GGCCAGGGCC AGAGCGAACC TGGGGAGTAC GAGCAGCGGC | 300 |
| TCAGCCTCCA GGACAGAGGG GCTACTCTGG CCCTGACTCA AGTCACCCCC CAAGACGAGC | 360 |
| GCATCTTCTT GTGCCAGGGC AAGCGCCCTC GGTCCCAGGA GTACCGCATC CAGCTCCGCG | 420 |
| TCTACAAAGC TCCGGAGGAG CCAAACATCC AGGTCAACCC CCTGGGCATC CCTGTGAACA | 480 |
| GTAAGGAGCC TGAGGAGGTC GCTACCTGTG TAGGGAGGAA CGGGTACCCC ATTCCTCAAG | 540 |
| TCATCTGGTA CAAGAATGGC CGGCCTCTGA AGGAGGAGAA GAACCGGGTC CACATTCAGT | 600 |
| CGTCCCAGAC TGTGGAGTCG AGTGGTTTGT ACACCTTGCA GAGTATTCTG AAGGCACAGC | 660 |
| TGGTTAAAGA AGACAAAGAT GCCCAGTTTT ACTGTGAGCT CAACTACCGG CTGCCCAGTG | 720 |
| GGAACCACAT GAAGGAGTCC AGGGAAGTCA CCGTCCCTGT TTTCTACCCG ACAGAAAAAG | 780 |
| TGTGGCTGGA AGTGGAGCCC GTGGGAATGC TGAAGGAAGG GGACCGCGTG GAAATCAGGT | 840 |
| GTTTGGCTGA TGGCAACCCT CCACCACACT TCAGCATCAG CAAGCAGAAC CCCAGCACCA | 900 |
| GGGAGGCAGA GGAAGAGACA ACCAACGACA ACGGGGTCCT GGTGCTGGAG CCTGCCCGGA | 960 |
| AGGAACACAG TGGGCGCTAT GAATGTCAGG CCTGGAACTT GGACACCATG ATATCGCTGC | 1020 |
| TGAGTGAACC ACAGGAACTA CTGGTGAACT ATGTGTCTGA CGTCCGAGTG AGTCCCGCAG | 1080 |
| CCCCTGAGAG ACAGGAAGGC AGCAGCCTCA CCCTGACCTG TGAGGCAGAG AGTAGCCAGG | 1140 |
| ACCTCGAGTT CCAGTGGCTG AGAGAAGAGA CAGACCAGGT GCTGGAAAGG GGGCCTGTGC | 1200 |
| TTCAGTTGCA TGACCTGAAA CGGGAGGCAG GAGGCGGCTA TCGCTGCGTG GCGTCTGTGC | 1260 |
| CCAGCATACC CGGCCTGAAC CGCACACAGC TGGTCAAGCT GGCCATTTTT GGCCCCCCTT | 1320 |
| GGATGGCATT CAAGGAGAGG AAGGTGTGGG TGAAAGAGAA TATGGTGTTG AATCTGTCTT | 1380 |
| GTGAAGCGTC AGGGCACCCC CGGCCCACCA TCTCCTGGAA CGTCAACGGC ACGGCAAGTG | 1440 |
| AACAAGACCA AGATCCACAG CGAGTCCTGA GCACCCTGAA TGTCCTCGTG ACCCCGGAGC | 1500 |
| TGTTGGAGAC AGGTGTTGAA TGCACGGCCT CCAACGACCT GGGCAAAAAC ACCAGCATCC | 1560 |
| TCTTCCTGGA GCTGGTCAAT TTAACCACCC TCACACCAGA CTCCAACACA ACCACTGGCC | 1620 |
| TCAGCACTTC CACTGCCAGT CCTCATACCA GAGCCAACAG CACCTCCACA GAGAGAAAGC | 1680 |
| TGCCGGAGCC GGAGAGCCGG GGCGTGGTCA TCGTGGCTGT GATTGTGTGC ATCCTGGTCC | 1740 |
| TGGCGGTGCT GGGCGCTGTC CTCTATTTCC TCTATAAGAA GGGCAAGCTG CCGTGCAGGC | 1800 |
| GCTCAGGGAA GCAGGAGATC ACGCTGCCCC CGTCTCGTAA GACCGAACTT GTAGTTGAAG | 1860 |
| TTAAGTCAGA TAAGCTCCCA GAAGAGATGG GCCTCCTGCA GGGCAGCAGC GGTGACAAGA | 1920 |
| GGGCTCCGGG AGACCAGGGA GAGAAATACA TCGATCTGAG GCATTAGCCC CGAATCACTT | 1980 |
| CAGCTCCCTT CCCTGCCTGG ACCATTCCCA GCTCCCTGCT CACTCTTCTC TCAGCCAAAG | 2040 |
| CCTCCAAAGG GACTAGAGAG AAGCCTCCTG CTCCCCTCAC CTGCACACCC CCTTTCAGAG | 2100 |
| GGCCACTGGG TTAGGACCTG AGGACCTCAC TTGGCCCTGC AAGCCGCTTT TCAGGGACCA | 2160 |
| GTCCACCACC ATCTCCTCCA CGTTGAGTGA AGCTCATCCC AAGCAAGGAG CCCCAGTCTC | 2220 |
| CCGAGCGGGT AGGAGAGTTT CTTGCAGAAC GTGTTTTTTC TTTACACACA TTATGGCTGT | 2280 |
| AAATACCTGG CTCCTGCCAG CAGCTGAGCT GGGTAGCCTC TCTGAGCTGG TTTCCTGCCC | 2340 |
| CAAAGGCTGG CTTCCACCAT CCAGGTGCAC CACTGAAGTG AGGACACACC GGAGCCAGGC | 2400 |
| GCCTGCTCAT GTTGAAGTGC GCTGTTCACA CCCGCTCCGG AGAGCACCCC AGCGGCATCC | 2460 |
| AGAAGCAGCT GCAGTGTTGC TGCCACCACC CTUCTGCTCG CCTCTTCAAA GTCTCCTGTG | 2520 |
| ACATTTTTTC TTTGGTCAGA AGCCAGGAAC TGGTGTCATT CCTTAAAAGA TACGTGCCGG | 2580 |
| GGCCAGGTGT GGTGGCTCAC GCCTGTAATC CCAGCACTTT GGGAGGCCGA GGCGGGCGGA | 2640 |
| TCACAAAGTC AGGACGAGAC CATCCTGGCT AACACGGTGA AACCCTGTCT CTACTAAAAA | 2700 |
| TACAAAAAAA AATTAGCTAG GCGTAGTGGT TGGCACCTAT AGTCCCAGCT ACTCGGAAGG | 2760 |
| CTGAAGCAGG AGAATGGTAT GAATCCAGGA GGTGGAGCTT GCAGTGAGCC GAGACCGTGC | 2820 |
| CACTGCACTC CAGCCTGGGC AACACAGCGA GACTCCGTCT CGAGGAAAAA AAAAGAAAAG | 2880 |
| ACGCGTACCT GCGGTGAGGA AGCTGGGCGC TGTTTTCGAG TTCAGGTGAA TTAGCCTCAA | 2940 |
| TCCCCGTGTT CACTTGCTCC CATAGCCCTC TTGATGGATC ACGTAAAACT GAAAGGCAGC | 3000 |
| GGGGAGCAGA CAAAGATGAG GTCTACACTG TCCTTCATGG GGATTAAAGC TATGGTTATA | 3060 |
| TTAGCACCAA ACTTCTACAA ACCAAGCTCA GGGCCCCAAC CCTAGAAGGG CCCAAATGAG | 3120 |
| AGAATGGTAC TTAGGGATGG AAAACGGGGC CTGGCTAGAG CTTCGGGTGT GTGTGTCTGT | 3280 |
| CTGTGTGTAT GCATACATAT GTGTGTATAT ATGGTTTTGT CAGGTGTGTA AATTTGCAAA | 3240 |
| TTGTTTCCTT TATATATGTA TGTATATATA TATATGAAAA TATATATATA TATGAAAAAT | 3300 |
| AAAGCTTAAT TGTCCCAGAA AATCATACAT TGCTTTTTTA TTCTACATGG GTACCACAGG | 3360 |
| AACCTGGGGG CCTGTGAAAC TACAACCAAA AGGCACACAA AACCGTTTCC AGTTGGCAGC | 3420 |
| AGAGATCAGG GGTTACCTCT GCTTCTGAGC AAATGGCTCA AGCTCTACCA GAGCAGACAG | 3480 |
| CTACCCTACT TTTCAGCAGC AAAACGTCCC GTATGACGCA GCACGAAGGG CCTGGCAGGC | 3540 |
| TGTTAGCAGG AGCTATGTCC CTTCCTATCG TTTCCGTCCA CTT | |

AAC1 DNA sequence
Gene name: Matrix metalloproteinase 1 (interstitial collagenase)
Unigene number: Hs.83169
Probeset Accession #: X54925
Nucleic Acid Accession #: NM_002421 cluster
Coding sequence: 69-1478 (predicted start/stop codons underlined)

| | |
|---|---|
| ATATTGGAGT AGCAAGAGGC TGGGAAGCCA TCACTTACCT TGCACTGAGA AAGAAGACAA | 60 |
| AGGCCAGTAT GCACAGCTTT CCTCCACTGC TGCTGCTGCT GTTCTGGGGT GTGGTGTCTC | 120 |
| ACAGCTTCCC AGCGACTCTA GAAACACAAG AGCAAGATGT GGACTTAGTC CAGAAATACC | 180 |
| TGGAAAAATA CTACAACCTG AAGAATGATG GGAGGCAAGT TGAAAAGCGG AGAAATAGTG | 240 |
| GCCCAGTGGT TGAAAAATTG AAGCAAATGC AGGAATTCTT TGGGCTGAAA GTGACTGGGA | 300 |
| AACCAGATGC TGAAACCCTG AAGGTGATGA AGCAGCCCAG ATGTGGAGTG CCTGATGTGG | 360 |
| CTCAGTTTGT CCTCACTGAG GGGAACCCTC GCTGGGAGCA AACACATCTG ACCTACAGGA | 420 |
| TTGAAAATTA CACGCCAGAT TTGCCAAGAG CAGATGTGGA CCATGCCATT GAGAAAGCCT | 480 |
| TCCAACTCTG GAGTAATGTC ACACCTCTGA CATTCACCAA GGTCTCTGAG GGTCAAGCAG | 540 |
| ACATCATGAT ATCTTTTGTC AGGGGAGATC ATCGGGACAA CTCTCCTTTT GATGGACCTG | 600 |
| GAGGAAATCT TGCTCATGCT TTTCAACCAG GCCCAGGTAT TGGAGGGGAT GCTCATTTTG | 660 |
| ATGAAGATGA AAGGTGGACC AACAATTTCA GAGAGTACAA CTTACATCGT GTTGCGGCTC | 720 |
| ATGAACTCGG CCATTCTCTT GGACTCTCCC ATTCTACTGA TATCGGGGCT TTGATGTACC | 780 |
| CTAGCTACAC CTTCAGTGGT GATGTTCAGC TAGCTCAGGA TGACATTGAT GGCATCCAAG | 840 |
| CCATATATGG ACGTTCCCAA AATCCTGTCC AGCCCATCGG CCCACAAACC CCAAAAGCAT | 900 |
| GTGACAGTAA GCTAACCTTT GATGCTATAA CTACGATTCG GGGAGAAGTG ATGTTCTTTA | 960 |
| AAGACAGATT CTACATGCGC ACAAATCCCT TCTACCCGGA AGTTGAGCTC AATTTCATTT | 1020 |
| CTGTTTTCTG GCCACAACTG CCAAATGGGC TTGAAGCTGC TTACGAATTT GCCGACAGAG | 1080 |
| ATGAAGTCCG GTTTTTCAAA GGGAATAAGT ACTGGGCTGT TCAGGGACAG AATGTGCTAC | 1140 |
| ACGGATACCC CAAGGACATC TACAGCTCCT TTGGCTTCCC TAGAACTGTG AAGCATATCG | 1200 |
| ATGCTGCTCT TTCTGAGGAA AACACTGGAA AAACCTACTT CTTTGTTGCT AACAAATACT | 1260 |
| GGAGGTATGA TGAATATAAA CGATCTATGG ATCCAGGTTA TCCCAAAATG ATAGCACATG | 1320 |
| ACTTTCCTGG AATTGGCCAC AAAGTTGATG CAGTTTTCAT GAAAGATGGA TTTTTCTATT | 1380 |
| TCTTTCATGG AACAAGACAA TACAAATTTG ATCCTAAAAC GAAGAGAATT TTGACTCTCC | 1440 |
| AGAAAGCTAA TAGCTGGTTC AACTGCAGGA AAAATTGAAC ATTACTAATT TGAATGGAAA | 1500 |
| ACACATGGTG TGAGTCCAAA GAAGGTGTTT TCCTGAAGAA CTGTCTATTT TCTCAGTCAT | 1560 |
| TTTTAACCTC TAGAGTCACT GATACACAGA ATATAATCTT ATTTATACCT CAGTTTGCAT | 1620 |
| ATTTTTTTAC TATTTAGAAT GTAGCCCTTT TTGTACTGAT ATAATTTAGT TCCACAAATG | 1680 |
| GTGGGTACAA AAAGTCAAGT TTGTGGCTTA TGGATTCATA TAGGCCAGAG TTGCAAAGAT | 1740 |
| CTTTTCCAGA GTATGCAACT CTGACGTTGA TCCCAGAGAG CAGCTTCAGT GACAAACATA | 1800 |
| TCCTTTCAAG ACAGAAAGAG ACAGGAGACA TGAGTCTTTG CCGGAGGAAA AGCAGCTCAA | 1860 |
| GAACACATGT GCAGTCACTG GTGTCACCCT GGATAGGCAA GGGATAACTC TTCTAACACA | 1920 |
| AAATAAGTGT TTTATGTTTG GAATAAAGTC AACCTTGTTT CTACTGTTTT | |

AAC3 DNA sequence
Gene name: Branched chain aminotransferase 1, cytosolic
Unigene number: Hs.157205
Probeset Accession #: AA423987
Nucleic Acid Accession #: NM_005504 cluster
Coding sequence: 1-1155 (predicted start/stop codons underlined)

| | |
|---|---|
| ATGGATTGCA GTAACGGATC GGCAGAGTGT ACCGGAGAAG GAGGATCAAA AGAGGTGGTG | 60 |
| GGGACTTTTA AGGCTAAAGA CCTAATAGTC ACACCAGCTA CCATTTTAAA GGAAAAACCA | 120 |
| GACCCCAATA ATCTGGTTTT TGGAACTGTG TTCACGGATC ATATGCTGAC GGTGGAGTGG | 180 |
| TCCTCAGAGT TTGGATGGGA GAAACCTCAT ATCAAGCCTC TTCAGAACCT GTCATTGCAC | 240 |
| CCTGGCTCAT CAGCTTTGCA CTATGCAGTG GAATTATTTG AAGGATTGAA GGCATTTCGA | 300 |
| GGAGTAGATA ATAAAATTCG ACTGTTTCAG CCAAACCTCA ACATGGATAG AATGTATCGC | 360 |
| TCTGCTGTGA GGGCAACTCT GCCGGTATTT GACAAAGAAG AGCTCTTAGA GTGTATTCAA | 420 |
| CAGCTTGTGA AATTGGATCA AGAATGGGTC CCATATTCAA CATCTGCTAG TCTGTATATT | 480 |
| CGTCCTGCAT TCATTGGAAC TGAGCCTTCT CTTGGAGTCA AGAAGCCTAC CAAAGCCCTG | 540 |
| CTCTTTGTAC TCTTGAGCCC AGTGGGACCT TATTTTTCAA GTGGAACCTT TAATCCAGTG | 600 |
| TCCCTGTGGG CCAATCCCAA GTATGTAAGA GCCTGGAAAG GTGGAACTGG GGACTGCAAG | 660 |
| ATGGGAGGGA ATTACGGCTC ATCTCTTTTT GCCCAATGTG AAGACGTAGA TAATGGGTGT | 720 |
| CAGCAGGTCC TGTGGCTCTA TGGCAGAGAC CATCAGATCA CTGAAGTGGG AACTATGAAT | 780 |
| CTTTTTCTTT ACTGGATAAA TGAAGATGGA GAAGAAGAAC TGGCAACTCC TCCACTAGAT | 840 |
| GGCATCATTC TTCCAGGAGT GACAAGGCGG TGCATTCTGG ACCTGGCACA TCAGTGGGGT | 900 |
| GAATTTAAGG TGTCAGAGAG ATACCTCACC ATGGATGACT TGACAACAGC CCTGGAGGGG | 960 |
| AACAGAGTGA GAGAGATGTT TAGCTCTGGT ACAGCCTGTG TTGTTTGCCC AGTTTCTGAT | 1020 |
| ATACTGTACA AAGGCGAGAC AATACACATT CCAACTATGG AGAATGGTCC TAAGCTGGCA | 1080 |
| AGCCGCATCT TGAGCAAATT AACTGATATC CAGTATGGAA GAGAAGAGAG CGACTGGACA | 1140 |
| ATTGTGCTAT CCTGA | |

ACG4 DNA sequence:
Gene name: Pentaxin-related gene, rapidly induced by IL-1 beta
Unigene number: Hs.2050
Probeset Accession #: M31166
Nucleic Acid Accession #: NM_002852 cluster
Coding sequence: 68-1213 (predicted start/stop codons underlined)

| | |
|---|---|
| CTCAAACTCA GCTCACTTGA GAGTCTCCTC CCGCCAGCTG TGGAAAGAAC TTTGCGTCTC | 60 |
| TCCAGCAATG CATCTCCTTG CGATTCTGTT TTGTGCTCTC TGGTCTGCAG TGTTGGCCGA | 120 |
| GAACTCGGAT GATTATGATC TCATGTATGT GAATTTGGAC AACGAAATAG ACAATGGACT | 180 |
| CCATCCCACT GAGGACCCCA CGCCGTGCGA CTGCGGTCAG GAGCACTCGG AATGGGACAA | 240 |
| GCTCTTCATC ATGCTGGAGA ACTCGCAGAT GAGAGAGCGC ATGCTGCTGC AAGCCACGGA | 300 |
| CGACGTCCTG CGGGGCGAGC TGCAGAGGCT GCGGGAGGAG CTGGGCCGGC TCGCGGAAAG | 360 |
| CCTGGCGAGG CCGTGCGCGC CGGGGGCTCC CGCAGAGGCC AGGCTGACCA GTGCTCTGGA | 420 |
| CGAGCTGCTG CAGGCGACCC GCGACGCGGG CCGCAGGCTG GCGCGTATGG AGGGCGCGGA | 480 |
| GGCGCAGCGC CCAGAGGAGG CGGGGCGCGC CCTGGCCGCG GTGCTAGAGG AGCTGCGGCA | 540 |
| GACGCGAGCC GACCTGCACG CGGTGCAGGG CTGGGCTGCC CGGAGCTGGC TGCCGGCAGG | 600 |
| TTGTGAAACA GCTATTTTAT TCCCAATGCG TTCCAAGAAG ATTTTTGGAA GCGTGCATCC | 660 |
| AGTGAGACCA ATGAGGCTTG AGTCTTTTAG TGCCTGCATT TGGGTCAAAG CCACAGATGT | 720 |
| ATTAAACAAA ACCATCCTGT TTTCCTATGG CACAAAGAGG AATCCATATG AAATCCAGCT | 780 |
| GTATCTCAGC TACCAATCCA TAGTGTTTGT GGTGGGTGGA GAGGAGAACA AACTGGTTGC | 840 |
| TGAAGCCATG GTTTCCCTGG GAAGGTGGAC CCACCTGTGC GGCACCTGGA ATTCAGAGGA | 900 |
| AGGGCTCACA TCCTTGTGGG TAAATGGTGA ACTGGCGGCT ACCACTGTTG AGATGGCCAC | 960 |
| AGGTCACATT GTTCCTGAGG GAGGAATCCT GCAGATTGGC CAAGAAAAGA ATGGCTGCTG | 1020 |
| TGTGGGTGGT GGCTTTGATG AAACATTAGC CTTCTCTGGG AGACTCACAG GCTTCAATAT | 1080 |
| CTGGGATAGT GTTCTTAGCA ATGAAGAGAT AAGAGAGACC GGAGGAGCAG AGTCTTGTCA | 1140 |
| CATCCGGGGG AATATTGTTG GGTGGGGAGT CACAGAGATC CAGCCACATG GAGGAGCTCA | 1200 |
| GTATGTTTCA TAAATGTTGT GAAACTCCAC TTGAAGCCAA AGAAAGAAAC TCACACTTAA | 1260 |
| AACACATGCC AGTTGGGAAG GTCTGAAAAC TCAGTGCATA ATAGGAACAC TTGAGACTAA | 1320 |
| TGAAAGAGAG AGTTGAGACC AATCTTTATT TGTACTGGCC AAATACTGAA TAAACAGTTG | 1380 |
| AAGGAAAGAC ATTGGAAAAA GCTTTTGAGG ATAATGTTAC TAGACTTTAT GCCATGGTGC | 1440 |
| TTTCAGTTTA ATGCTGTGTC TCTGTCAGAT AAACTCTCAA ATAATTAAAA AGGACTGTAT | 1500 |
| TGTTGAACAG AGGGACAATT GTTTTACTTT TCTTTGGTTA ATTTTGTTTT GGCCAGAGAT | 1560 |
| GAATTTTACA TTGGAAGAAT AACAAAATAA GATTTGTTGT CCATTGTTCA TTGTTATTGG | 1620 |
| TATGTACCTT ATTACAAAAA AAATGATGAA AACATATTTA TACTACAAGG TGACTTAACA | 1680 |
| ACTATAAATG TAGTTTATGT GTTATAATCG AATGTCACGT TTTTGAGAAG ATAGTCATAT | 1740 |
| AAGTTATATT GCAAAAGGGA TTTGTATTAA TTTAAGACTA TTTTTGTAAA GCTCTACTGT | 1800 |
| AAATAAAATA TTTTATAAAA CTAAAAAAAA AAAAAAA | |

ACK5 DNA sequence
Gene name: Von Willebrand factor; Coagulation factor VIII
Unigene number: Hs.110802
Probeset Accession #: M10321
Nucleic Acid Accession #: NM_000552
Coding sequence: 311-8752 (predicted start/stop codons underlined)

| | |
|---|---|
| AGCTCACAGC TATTGTGGTG GGAAAGGGAG GGTGGTTGGT GGATGTCACA GCTTGGGCTT | 60 |
| TATCTCCCCC AGCAGTGGGG ACTCCACAGC CCCTGGGCTA CATAACAGCA AGACAGTCCG | 120 |
| GAGCTGTAGC AGACCTGATT GAGCCTTTGC AGCAGCTGAG AGCATGGCCT AGGGTGGGCG | 180 |
| GCACCATTGT CCAGCAGCTG AGTTTCCCAG GGACCTTGGA GATAGCCGCA GCCCTCATTT | 240 |
| GCAGGGGAAG GCACCATTGT CCAGCAGCTG AGTTTCCCAG GGACCTTGGA GATAGCCGCA | 300 |
| GCCCTCATTT ATGATTCCTG CCAGATTTGC CGGGGTGCTG CTTGCTCTGG CCCTCATTTT | 360 |
| GCCAGGGACC CTTTGTGCAG AAGGAACTCG CGGCAGGTCA TCCACGGCCC GATGCAGCCT | 420 |
| TTTCGGAAGT GACTTCGTCA ACACCTTTGA TGGGAGCATG TACAGCTTTG CGGGATACTG | 480 |
| CAGTTACCTC CTGGCAGGGG GCTGCCAGAA ACGCTCCTTC TCGATTATTG GGGACTTCCA | 540 |
| GAATGGCAAG AGAGTGAGCC TCTCCGTGTA TCTTGGGGAA TTTTTTGACA TCCATTTGTT | 600 |
| TGTCAATGGT ACCGTGACAC AGGGGGACCA AAGAGTCTCC ATGCCCTATG CCTCCAAAGG | 660 |
| GCTGTATCTA GAAACTGAGG CTGGGTACTA CAAGCTGTCC GGTGAGGCCT ATGGCTTTGT | 720 |
| GGCCAGGATC GATGGCAGCG GCAACTTTCA AGTCCTGCTG TCAGACAGAT ACTTCAACAA | 780 |
| GACCTGCGGG CTGTGTGGCA ACTTTAACAT CTTTGCTGAA GATGACTTTA TGACCCAAGA | 840 |
| AGGGACCTTG ACCTCGGACC CTTATGACTT TGCCAACTCA TGGGCTCTGA GCAGTGGAGA | 900 |
| ACAGTGGTGT GAACGGGCAT CTCCTCCCAG CAGCTCATGC AACATCTCCT CTGGGGAAAT | 960 |
| GCAGAAGGGC CTGTGGGAGC AGTGCCAGCT TCTGAAGAGC ACCTCGGTGT TTGCCCGCTG | 1020 |
| CCACCCTCTG GTGGACCCCG AGCCTTTTGT GGCCCTGTGT GAGAAGACTT TGTGTGAGTG | 1080 |
| TGCTGGGGGG CTGGAGTGCG CCTGCCCTGC CCTCCTGGAG TACGCCCGGA CCTGTGCCCA | 1140 |
| GGAGGGAATG GTGCTGTACG GCTGGACCGA CCACAGCGCG TGCAGCCCAG TGTGCCCTGC | 1200 |
| TGGTATGGAG TATAGGCAGT GTGTGTCCCC TTGCGCCAGG ACCTGCCAGA GCCTGCACAT | 1260 |
| CAATGAAATG TGTCAGGAGC GATGCGTGGA TGGCTGCAGC TGCCCTGAGG GACAGCTCCT | 1320 |
| GGATGAAGGC CTCTGCGTGG AGAGCACCGA GTGTCCCTGC GTGCATTCCG GAAAGCGCTA | 1380 |
| CCCTCCCGGC ACCTCCCTCT CTCGAGACTG CAACACCTGC ATTTGCCGAA ACAGCCAGTG | 1440 |
| GATCTGCAGC AATGAAGAAT GTCCAGGGGA GTGCCTTGTC ACTGGTCAAT CCCACTTCAA | 1500 |
| GAGCTTTGAC AACAGATACT TCACCTTCAG TGGGATCTGC CAGTACCTGC TGGCCCGGGA | 1560 |
| TTGCCAGGAC CACTCCTTCT CCATTGTCAT TGAGACTGTC CAGTGTGCTG ATGACCGCGA | 1620 |
| CGCTGTGTGC ACCCGCTCCG TCACCGTCCG GCTGCCTGGC CTGCACAACA GCCTTGTGAA | 1680 |
| ACTGAAGCAT GGGGCAGGAG TTGCCATGGA TGGCCAGGAC ATCCAGCTCC CCCTCCTGAA | 1740 |
| AGGTGACCTC CGCATCCAGC ATACAGTGAC GGCCTCCGTG CGCCTCAGCT ACGGGGAGGA | 1800 |
| CCTGCAGATG GACTGGGATG GCCGCGGGAG GCTGCTGGTG AAGCTGTCCC CCGTCTACGC | 1860 |
| CGGGAAGACC TGCGGCCTGT GTGGGAATTA CAATGGCAAC CAGGGCGACG ACTTCCTTAC | 1920 |
| CCCCTCTGGG CTGGCAGAGC CCCGGGTGGA GGACTTCGGG AACGCCTGGA AGCTGCACGG | 1980 |
| GGACTGCCAG GACCTGCAGA AGCAGCACAG CGATCCCTGC GCCCTCAACC CGCGCATGAC | 2040 |
| CAGGTTCTCC GAGGAGGCGT GCGCGGTCCT GACGTCCCCC ACATTCGAGG CCTGCCATCG | 2100 |
| TGCCGTCAGC CCGCTGCCCT ACCTGCGGAA CTGCCGCTAC GACGTGTGCT CCTGCTCGGA | 2160 |
| CGGCCGCGAG TGCCTGTGCG GCGCCCTGGC CAGCTATGCC GCGGCCTGCG CGGGGAGAGG | 2220 |
| CGTGCGCGTC GCGTGGCGCG AGCCAGGCCG CTGTGAGCTG AACTGCCCGA AAGGCCAGGT | 2280 |
| GTACCTGCAG TGCGGGACCC CCTGCAACCT GACCTGCCGC TCTCTCTCTT ACCCGGATGA | 2340 |
| GGAATGCAAT GAGGCCTGCC TGGAGGGCTG CTTCTGCCCC CCAGGGCTCT ACATGGATGA | 2400 |
| GAGGGGGGAC TGCGTGCCCA AGGCCCAGTG CCCCTGTTAC TATGACGGTG AGATCTTCCA | 2460 |
| GCCAGAAGAC ATCTTCTCAG ACCATCACAC CATGTGCTAC TGTGAGGATG GCTTCATGCA | 2520 |
| CTGTACCATG AGTGGAGTCC CCGGAAGCTT GCTGCCTGAC GCTGTCCTCA GCAGTCCCCT | 2580 |
| GTCTCATCGC AGCAAAAGGA GCCTATCCTG TCGGCCCCCC ATGGTCAAGC TGGTGTGTCC | 2640 |
| CGCTGACAAC CTGCGGGCTG AAGGGCTCGA GTGTACCAAA ACGTGCCAGA ACTATGACCT | 2700 |
| GGAGTGCATG AGCATGGGCT GTGTCTCTGG CTGCCTCTGC CCCCCGGGCA TGGTCCGGCA | 2760 |
| TGAGAACAGA TGTGTGGCCC TGGAAAGGTG TCCCTGCTTC CATCAGGGCA AGGAGTATGC | 2820 |
| CCCTGGAGAA ACAGTGAAGA TTGGCTGCAA CACTTGTGTC TGTCGGGACC GGAAGTGGAA | 2880 |
| CTGCACAGAC CATGTGTGTG ATGCCACGTG CTCCACGATC GGCATGGCCC ACTACCTCAC | 2940 |
| CTTCGACGGG CTCAAATACC TGTTCCCCGG GGAGTGCCAG TACGTTCTGG TGCAGGATTA | 3000 |
| CTGCGGCAGT AACCCTGGGA CCTTTCGGAT CCTAGTGGGG AATAAGGGAT GCAGCCACCC | 3060 |
| CTCAGTGAAA TGCAAGAAAC GGGTCACCAT CCTGGTGGAG GGAGGAGAGA TTGAGCTGTT | 3120 |
| TGACGGGGAG GTGAATGTGA AGAGGCCCAT GAAGGATGAG ACTCACTTTG AGGTGGTGGA | 3180 |
| GTCTGGCCGG TACATCATTC TGCTGCTGGG CAAAGCCCTC TCCGTGGTCT GGGACCGCCA | 3240 |
| CCTGAGCATC TCCGTGGTCC TGAAGCAGAC ATACCAGGAG AAAGTGTGTG GCCTGTGTGG | 3300 |
| GAATTTTGAT GGCATCCAGA ACAATGACCT CACCAGCAGC AACCTCCAAG TGGAGGAAGA | 3360 |
| CCCTGTGGAC TTTGGGAACT CCTGGAAAGT GAGCTCGCAG TGTGCTGACA CCAGAAAAGT | 3420 |
| GCCTCTGGAC TCATCCCCTG CCACCTGCCA TAACAACATC ATGAAGCAGA CGATGGTGGA | 3480 |
| TTCCTCCTGT AGAATCCTTA CCAGTGACGT CTTCCAGGAC TGCAACAAGC TGGTGGACCC | 3540 |
| CGAGCCATAT CTGGATGTCT GCATTTACGA CACCTGCTCC TGTGAGTCCA TTGGGGACTG | 3600 |
| CGCCTGCTTC TGCGACACCA TTGCTGCCTA TGCCCACGTG TGTGCCCAGC ATGGCAAGGT | 3660 |
| GGTGACCTGG AGGACGGCCA CATTGTGCCC CCAGAGCTGC GAGGAGAGGA ATCTCCGGGA | 3720 |
| GAACGGGTAT GAGTGTGAGT GGCGCTATAA CAGCTGTGCA CCTGCCTGTC AAGTCACGTG | 3780 |
| TCAGCACCCT GAGCCACTGG CCTGCCCTGT GCAGTGTGTG GAGGGCTGCC ATGCCCACTG | 3840 |
| CCCTCCAGGG AAAATCCTGG ATGAGCTTTT GCAGACCTGC GTTGACCCTG AAGACTGTCC | 3900 |
| AGTGTGTGAG GTGGCTGGCC GGCGTTTTGC CTCAGGAAAG AAAGTCACCT TGAATCCCAG | 3960 |
| TGACCCTGAG CACTGCCAGA TTTGCCACTG TGATGTTGTC AACCTCACCT GTGAAGCCTG | 4020 |
| CCAGGAGCCG GGAGGCCTGG TGGTGCCTCC CACAGATGCC CCGGTGAGCC CCACCACTCT | 4080 |
| GTATGTGGAG GACATCTCGG AACCGCCGTT GCACGATTTC TACTGCAGCA GGCTACTGGA | 4140 |
| CCTGGTCTTC CTGCTGGATG GCTCCTCCAG GCTGTCCGAG GCTGAGTTTG AAGTGCTGAA | 4200 |
| GGCCTTTGTG GTGGACATGA TGGAGCGGCT GCGCATCTCC CAGAAGTGGG TCCGCGTGGC | 4260 |
| CGTGGTGGAG TACCACGACG GCTCCCACGC CTACATCGGG CTCAAGGACC GGAAGCGACC | 4320 |
| GTCAGAGCTG CGGCGCATTG CCAGCCAGGT GAAGTATGCG GGCAGCCAGG TGGCCTCCAC | 4380 |
| CAGCGAGGTC TTGAAATACA CACTGTTCCA AATCTTCAGC AAGATCGACC GCCCTGAAGC | 4440 |
| CTCCCGCATC GCCCTGCTCC TGATGGCCAG CCAGGAGCCC CAACGGATGT CCCGGAACTT | 4500 |
| TGTCCGCTAC GTCCAGGGCC TGAAGAAGAA GAAGGTCATT GTGATCCCGG TGGGCATTGG | 4560 |
| GCCCCATGCC AACCTCAAGC AGATCCGCCT CATCGAGAAG CAGGCCCCTG AGAACAAGGC | 4620 |
| CTTCGTGCTG AGCAGTGTGG ATGAGCTGGA GCAGCAAAGG GACGAGATCG TTAGCTACCT | 4680 |
| CTGTGACCTT GCCCCTGAAG CCCCTCCTCC TACTCTGCCC CCCCACATGG CACAAGTCAC | 4740 |
| TGTGGGCCCG GGGCTCTTGG GGGTTTCGAC CCTGGGGCCC AAGAGGAACT CCATGGTTCT | 4800 |
| GGATGTGGCG TTCGTCCTGG AAGGATCGGA CAAAATTGGT GAAGCCGACT TCAACAGGAG | 4860 |
| CAAGGAGTTC ATGGAGGAGG TGATTCAGCG GATGGATGTG GGCCAGGACA GCATCCACGT | 4920 |
| CACGGTGCTG CAGTACTCCT ACATGGTGAC CGTGGAGTAC CCCTTCAGCG AGGCACAGTC | 4980 |
| CAAAGGGGAC ATCCTGCAGC GGGTGCGAGA GATCCGCTAC CAGGGCGGCA ACAGGACCAA | 5040 |
| CACTGGGCTG GCCCTGCGGT ACCTCTCTGA CCACAGCTTC TTGGTCAGCC AGGGTGACCG | 5100 |
| GGAGCAGGCG CCCAACCTGG TCTACATGGT CACCGGAAAT CCTGCCTCTG ATGAGATCAA | 5160 |
| GAGGCTGCCT GGAGACATCC AGGTGGTGCC CATTGGAGTG GGCCCTAATG CCAACGTGCA | 5220 |
| GGAGCTGGAG AGGATTGGCT GGCCCAATGC CCCTATCCTC ATCCAGGACT TTGAGACGCT | 5280 |
| CCCCCGAGAG GCTCCTGACC TGGTGCTGCA GAGGTGCTGC TCCGGAGAGG GGCTGCAGAT | 5340 |
| CCCCACCCTC TCCCCTGCAC CTGACTGCAG CCAGCCCCTG GACGTGATCC TTCTCCTGGA | 5400 |
| TGGCTCCTCC AGTTTCCCAG CTTCTTATTT TGATGAAATG AAGAGTTTCG CCAAGGCTTT | 5460 |
| CATTTCAAAA GCCAATATAG GGCCTCGTCT CACTCAGGTG TCAGTGCTGC AGTATGGAAG | 5520 |
| CATCACCACC ATTGACGTGC CATGGAACGT GGTCCCGGAG AAAGCCCATT TGCTGAGCCT | 5580 |
| TGTGGACGTC ATGCAGCGGG AGGGAGGCCC CAGCCAAATC GGGGATGCCT TGGGCTTTGC | 5640 |
| TGTGCGATAC TTGACTTCAG AAATGCATGG TGCCAGGCCG GGAGCCTCAA AGGCGGTGGT | 5700 |
| CATCCTGGTC ACGGACGTCT CTGTGGATTC AGTGGATGCA GCAGCTGATG CCGCCAGGTC | 5760 |
| CAACAGAGTG ACAGTGTTCC CTATTGGAAT TGGAGATCGC TACGATGCAG CCCAGCTACG | 5820 |
| GATCTTGGCA GGCCCAGCAG GCGACTCCAA CGTGGTGAAG CTCCAGCGAA TCGAAGACCT | 5880 |
| CCCTACCATG GTCACCTTGG GCAATTCCTT CCTCCACAAA CTGTGCTCTG GATTTGTTAG | 5940 |
| GATTTGCATG GATGAGGATG GGAATGAGAA GAGGCCCGGG GACGTCTGGA CCTTGCCAGA | 6000 |
| CCAGTGCCAC ACCGTGACTT GCCAGCCAGA TGGCCAGACC TTGCTGAAGA GTCATCGGGT | 6060 |
| CAACTGTGAC CGGGGGCTGA GGCCTTCGTG CCCTAACAGC CAGTCCCCTG TTAAAGTGGA | 6120 |
| AGAGACCTGT GGCTGCCGCT GGACCTGCCC CTGCGTGTGC ACAGGCAGCT CCACTCGGCA | 6180 |
| CATCGTGACC TTTGATGGGC AGAATTTCAA GCTGACTGGC AGCTGTTCTT ATGTCCTATT | 6240 |
| TCAAAACAAG GAGCAGGACC TGGAGGTGAT TCTCCATAAT GGTGCCTGCA GCCCTGGAGC | 6300 |
| AAGGCAGGGC TGCATGAAAT CCATCGAGGT GAAGCACAGT GCCCTCTCCG TCGAGCTGCA | 6360 |
| CAGTGACATG GAGGTGACGG TGAATGGGAG ACTGGTCTCT GTTCCTTACG TGGGTGGGAA | 6420 |
| CATGGAAGTC AACGTTTATG GTGCCATCAT GCATGAGGTC AGATTCAATC ACCTTGGTCA | 6480 |
| CATCTTCACA TTCACTCCAC AAAACAATGA GTTCCAACTG CAGCTCAGCC CCAAGACTTT | 6540 |
| TGCTTCAAAG ACGTATGGTC TGTGTGGGAT CTGTGATGAG AACGGAGCCA ATGACTTCAT | 6600 |
| GCTGAGGGAT GGCACAGTCA CCACAGACTG GAAAACACTT GTTCAGGAAT GGACTGTGCA | 6660 |
| GCGGCCAGGG CAGACGTGCC AGCCCATCCT GGAGGAGCAG TGTCTTGTCC CCGACAGCTC | 6720 |
| CCACTGCCAG GTCCTCCTCT TACCACTGTT TGCTGAATGC CACAAGGTCC TGGCTCCAGC | 6780 |
| CACATTCTAT GCCATCTGCC AGCAGGACAG TTGCCACCAG GAGCAAGTGT GTGAGGTGAT | 6840 |
| CGCCTCTTAT GCCCACCTCT GTCGGACCAA CGGGGTCTGC GTTGACTGGA GGACACCTGA | 6900 |
| TTTCTGTGCT ATGTCATGCC CACCATCTCT GGTCTACAAC CACTGTGAGC ATGGCTGTCC | 6960 |
| CCGGCACTGT GATGGCAACG TGAGCTCCTG TGGGGACCAT CCCTCCGAAG GCTGTTTCTG | 7020 |
| CCCTCCAGAT AAAGTCATGT TGGAAGGCAG CTGTGTCCCT GAAGAGGCCT GCACTCAGTG | 7080 |
| CATTGGTGAG GATGGAGTCC AGCACCAGTT CCTGGAAGCC TGGGTCCCGG ACCACCAGCC | 7140 |
| CTGTCAGATC TGCACATGCC TCAGCGGGCG GAAGGTCAAC TGCACAACGC AGCCCTGCCC | 7200 |
| CACGGCCAAA GCTCCCACGT GTGGCCTGTG TGAAGTAGCC CGCCTCCGCC AGAATGCAGA | 7260 |
| CCAGTGCTGC CCCGAGTATG AGTGTGTGTG TGACCCAGTG AGCTGTGACC TGCCCCCAGT | 7320 |
| GCCTCACTGT GAACGTGGCC TCCAGCCCAC ACTGACCAAC CCTGGCGAGT GCAGACCCAA | 7380 |
| CTTCACCTGC GCCTGCAGGA AGGAGGAGTG CAAAAGAGTG TCCCCACCCT CCTGCCCCCC | 7440 |
| GCACCGTTTG CCCACCCTTC GGAAGACCCA GTGCTGTGAT GAGTATGAGT GTGCCTGCAA | 7500 |
| CTGTGTCAAC TCCACAGTGA GCTGTCCCCT TGGGTACTTG GCCTCAACCG CCACCAATGA | 7560 |
| CTGTGGCTGT ACCACAACCA CCTGCCTTCC CGACAAGGTG TGTGTCCACC GAAGCACCAT | 7620 |
| CTACCCTGTG GGCCAGTTCT GGGAGGAGGG CTGCGATGTG TGCACCTGCA CCGACATGGA | 7680 |
| GGATGCCGTG ATGGGCCTCC GCGTGGCCCA GTGCTCCCAG AAGCCCTGTG AGGACAGCTG | 7740 |
| TCGGTCGGGC TTCACTTACG TTCTGCATGA AGGCGAGTGC TGTGGAAGGT GCCTGCCATC | 7800 |
| TGCCTGTGAG GTGGTGACTG GCTCACCGCG GGGGGACTCC CAGTCTTCCT GGAAGAGTGT | 7860 |
| CGGCTCCCAG TGGGCCTCCC CGGAGAACCC CTGCCTCATC AATGAGTGTG TCCGAGTGAA | 7920 |
| GGAGGAGGTC TTTATACAAC AAAGGAACGT CTCCTGCCCC GAGCTGGAGG TCCCTGTCTG | 7980 |
| CCCCTCGGGC TTTCAGCTGA GCTGTAAGAC CTCAGCGTGC TGCCCAAGCT GTCGCTGTGA | 8040 |
| GCGCATGGAG GCCTGCATGC TCAATGGCAC TGTCATTGGG CCCGGGAAGA CTGTGATGAT | 8100 |
| CGATGTGTGC ACGACCTGCC GCTGCATGGT GCAGGTGGGG GTCATCTCTG GATTCAAGCT | 8160 |
| GGAGTGCAGG AAGACCACCT GCAACCCCTG CCCCCTGGGT TACAAGGAAG AAAATAACAC | 8220 |
| AGGTGAATGT TGTGGGAGAT GTTTGCCTAC GGCTTGCACC ATTCAGCTAA GAGGAGGACA | 8280 |
| GATCATGACA CTGAAGCGTG ATGAGACGCT CCAGGATGGC TGTGATACTC ACTTCTGCAA | 8340 |
| GGTCAATGAG AGAGGAGAGT ACTTCTGGGA GAAGAGGGTC ACAGGCTGCC CACCCTTTGA | 8400 |
| TGAACACAAG TGTCTGGCTG AGGGAGGTAA AATTATGAAA ATTCCAGGCA CCTGCTGTGA | 8460 |
| CACATGTGAG GAGCCTGAGT GCAACGACAT CACTGCCAGG CTGCAGTATG TCAAGGTGGG | 8520 |
| AAGCTGTAAG TCTGAAGTAG AGGTGGATAT CCACTACTGC CAGGGCAAAT GTGCCAGCAA | 8580 |
| AGCCATGTAC TCCATTGACA TCAACGATGT GCAGGACCAG TGCTCCTGCT GCTCTCCGAC | 8640 |
| ACGGACGGAG CCCATGCAGG TGGCCCTGCA CTGCACCAAT GGCTCTGTTG TGTACCATGA | 8700 |
| GGTTCTCAAT GCCATGGAGT GCAAATGCTC CCCCAGGAAG TGCAGCAAGT GAGGCTGCTG | 8760 |
| CAGCTGCATG GGTGCCTGCT GCTGCCTGCC TTGGCCTGAT GGCCAGGCCA GAGTGCTGCC | 8820 |
| AGTCCTCTGC ATGTTCTGCT CTTGTGCCCT TCTGAGCCCA CAATAAAGGC TGAGCTCTTA | 8880 |
| TCTTGCTGCA TGTTCTGCTC TTGTGCCCTT CTGAGCCCAC AAT | |

AAC7 DNA sequence
Gene name: KIAA1294 protein
Probeset Accession #: AA432248
Nucleic Acid Accession #: AB037715
Coding sequence: 370-3489 (predicted start/stop codons underlined)

| | |
|---|---|
| GAACGCTCAC AGAACAGGCA GTGCAATTCC ATGTTCCTCT TAAGTATGTT AGCCCTACCG | 60 |
| GGAGCTGAGC TGGCCAGTCT ACTTGGAGAG GAAAAGTAGA TCTGGGGAAG GTGGAAGGGT | 120 |
| CAGTTCCTAA GTGACTTCCT CCTCGGGGAT GGTAAGGGCA TTTGCTGATC TCCAGTGACT | 180 |
| GCCTGGTGCC TCATGGTCAG ACTCGGCTGT CTCACTCCCA GATATCTGAT TTTGCAAAAA | 240 |
| GGGACACACC TATCTGCAGC AAAGAAGACA CTGACCAGAT TGCGAGCGGT GCTTTTGGAT | 300 |
| GCTCTGTAGC CACCCGGGGC CCAGGAGGAC TGACTCGGCA GCAGGATTCG TGCATGGGAA | 360 |
| TCGGAGACCA TGGCAGTGCA GCTGGTGCCC GACTCAGCTC TCGGCCTGCT GATGATGACG | 420 |
| GAGGGCCGCC GATGTCAAGT ACATCTTCTT GATGACAGGA AGCTGGAACT CCTAGTACAG | 480 |
| CCCAAGCTGT TGGCCAAGGA GCTTCTTGAC CTTGTGGCTT CTCACTTCAA TCTGAAGGAA | 540 |
| AAGGAGTACT TTGGAATAGC ATTCACAGAT GAAACGGGAC ACTTAAACTG GCTTCAGCTA | 600 |
| GATCGAAGAG TATTGGAACA TGACTTCCCT AAAAAGTCAG GACCCGTGGT TTTATACTTT | 660 |
| TGTGTCAGGT TCTATATAGA AAGCATTTCA TACCTGAAGG ATAATGCTAC CATTGAGCTT | 720 |
| TTCTTTCTGA ACGCGAAGTC CTGCATCTAC AAGGAGCTTA TTGACGTTGA CAGCGAAGTG | 780 |
| GTGTTTGAAT TAGCTTCCTA TATTTTACAG GAGGCAAAGG GAGATTTTTC TAGCAATGAA | 840 |
| GTTGTGAGGA GTGACTTGAA GAAGCTGCCA GCCCTTCCCA CCCAAGCCCT GAAGGAGCAC | 900 |
| CCTTCCCTGG CCTACTGTGA AGACAGAGTC ATTGAGCACT ACAAGAAACT GAACGGTCAG | 960 |
| ACAAGAGGTC AAGCAATCGT AAACTACATG AGCATCGTGG AGTCTCTCCC AACCTACGGG | 1020 |
| GTTCACTATT ATGCAGTGAA GGACAAGCAG GGCATACCAT GGTGGCTGGG CCTGAGCTAC | 1080 |
| AAAGGGATCT TCCAGTATGA CTACCATGAT AAAGTGAAGC CAAGAAAGAT ATTCCAATGG | 1140 |
| AGACAGTTGG AAAACCTGTA CTTCAGAGAA AAGAAGTTTT CCGTGGAAGT TCATGACCCA | 1200 |
| CGCAGGGCTT CAGTGACAAG GAGGACGTTT GGGCACAGCG GCATTGCAGT GCACACGTGG | 1260 |
| TATGCATGTC CGGCATTGAT CAAGTCCATC TGGGCTATGG CCATAAGCCA ACACCAGTTC | 1320 |
| TATCTGGACA GAAAGCAGAG TAAGTCCAAA ATCCATGCAG CACGCAGCCT GAGTGAGATC | 1380 |
| GCCATCGACC TGACCGAGAC GGGGACGCTG AAGACCTCGA AGCTGGCCAA CATGGGTAGC | 1440 |
| AAGGGGAAGA TCATCAGCGG CAGCAGCGGC AGCCTGCTGT CTTCAGGTTC TCAGGAATCA | 1500 |
| GATAGCTCGC AGTCGGCCAA GAAGGACATG CTGGCTGCCT TGAAGTCCAG GCAGGAAGCT | 1560 |
| CTGGAGGAAA CCCTGCGTCA GAGGCTGGAG GAACTGAAGA AGCTGTGTCT CCGAGAAGCT | 1620 |
| GAGCTCACGG GCAAGCTGCC AGTAGAATAT CCCCTGGATC CAGGGGAGGA ACCACCCATT | 1680 |
| GTTCGGAGAA GAATAGGAAC AGCCTTCAAA CTGGATGAAC AGAAAATCCT GCCCAAAGGA | 1740 |
| GAGGAAGCTG AGCTGGAACG CCTGGAACGA GAGTTTGCCA TTCAGTCCCA GATTACGGAG | 1800 |
| GCCGCCCGCC GCCTAGCCAG TGACCCCAAC GTCAGCAAAA AACTGAAGAA ACAAAGGAAA | 1860 |
| ACCTCGTATC TGAATGCACT GAAGAAACTG CAGGAGATTG AAAATGCAAT CAATGAGAAC | 1920 |
| CGCATCAAGT CTGGGAAGAA ACCCACCCAG AGGGCTTCGC TGATCATAGA CGATGGAAAC | 1980 |
| ATTGCCAGTG AAGACAGCTC CCTCTCAGAT GCCCTTGTTC TTGAGGATGA AGACTCTCAG | 2040 |
| GTTACCAGCA CAATATCCCC CCTACATTCT CCTCACAAGG GACTCCCTCC TCGGCCACCG | 2100 |
| TCGCACAACA GGCCTCCTCC TCCCCAGTCC CTGGAGGGAC TCCGACAGAT GCACTATCAC | 2160 |
| CGCAACGACT ATGACAAGTC ACCCATCAAG CCCAAAATGT GGAGTGAGTC CTCTTTAGAT | 2220 |
| GAACCCTATG AGAAGGTCAA GAAGCGCTCC TCTCACAGCC ATTCCAGCAG CCACAAGCGC | 2280 |
| TTCCCCAGCA CAGGAAGCTG TGCGGAAGCC GGCGGAGGAA GCAACTCCTT GCAGAACAGC | 2340 |
| CCCATCCGCG GCCTCCCGCA CTGGAACTCC CAGTCCAGCA TGCCGTCCAC GCCAGACCTG | 2400 |
| CGGGTCCGGA GTCCCCACTA CGTCCATTCC ACGAGGTCGG TGGACATCAG CCCCACCCGA | 2460 |
| CTGCACAGCC TCGCACTGCA CTTTAGGCAC CGGAGCTCCA GCCTGGAGTC CCAGGGCAAG | 2520 |
| CTCCTGGGCT CGGAAAACGA CACCGGGAGC CCCGACTTCT ACACCCCGCG GACTCGTAGC | 2580 |
| AGCAACGGCT CAGACCCCAT GGACGACTGC TCGTCGTGCA CCAGCCACTC GAGCTCGGAG | 2640 |
| CACTACTACC CGGCGCAGAT GAACGCCAAC TACTCCACGC TGGCCGAGGA CTCGCCGTCC | 2700 |
| AAGGCGCGCC AGAGGCAGAG GCAGCGGCAG CGGGCGGCGG GCGCACTGGG CTCAGCCAGC | 2760 |
| TCGGGCAGCA TGCCCAACCT GGCGGCGCGC GGGGGTGCGG GGGGCGCGGG GGGCGCGGGG | 2820 |
| GGCGGTGTGT ACCTGCACAG CCAGAGCCAG CCCAGCTCGC AGTACCGCAT CAAGGAGTAC | 2880 |
| CCGCTGTACA TCGAGGGCGG CGCCACGCCC GTGGTGGTGC GCAGCCTGGA GAGCGACCAG | 2940 |
| GAGTGCCACT ACAGCGTCAA GGCTCAGTTC AAGACGTCCA ACTCCTACAC GGCGGGCGGC | 3000 |
| CTGTTCAAGG AGAGCTGGCG CGGCGGCGGC GGCGACGAGG GCGACACGGG CCGCCTGACG | 3060 |
| CCGTCGCGAT CGCAGATCCT GCGGACTCCG TCGCTGGGCC GCGAGGGCGC CCACGACAAG | 3120 |
| GGCGCGGGCC GTGCCGCCGT CTCAGACGAG CTGCGCCAGT GGTACCAGCG TTCCACCGCC | 3180 |
| TCGCACAAGG AGCACAGCCG CCTGTCGCAC ACCAGCTCCA CCTCCTCGGA CAGCGGCTCG | 3240 |
| CAGTACAGCA CCTCCTCCCA GAGCACCTTC GTGGCGCACA GCAGGGTCAC CAGGATGCCC | 3300 |
| CAGATGTGCA AGGCCACGTC AGCTGCCTTA CCTCAAAGCC AGAGAAGCTC GACACCGTCA | 3360 |
| AGTGAAATTG GAGCCACCCC CCCAAGCAGC CCCCACCACA TCCTAACCTG GCAGACTGGA | 3420 |
| GAAGCAACAG AAAACTCACC CATTCTGGAT GGGTCTGAGT CTCCACCTCA CCAAAGTACT | 3480 |
| GATGAATAGA GGAGCTACAA TGATAGCTGT TTCCTGGATT CCTCCCTCTA TCCAGAACTA | 3540 |
| GCTGATGTCC AGTGGTACGG GCAGGAAAAA GCCAAGCCCG GGACCCTCGT GTGAGCCAGC | 3600 |
| CCGGCCTAAT CTGACCGCCT CAACGCCATT CTGAGATCAC CTCACTGCCT CTCATTTGCC | 3660 |
| TTACCCAGAC GCACCGTCAC CCTGCACCAG CTTTGGCCCT CAGCACTTTT TTTCTCCTGT | 3720 |
| CTCCGCATTC CCTCCCCCTT GAAAACCTGA CTGAGGAGAC ATTCTGGAAG GTTCCGGTCC | 3780 |
| CACTGTGTGT CCCCTGGCGC TCTTGCCCAT AGAGAGCCAG ACACCAATCC TCAATGGCAC | 3840 |
| CTTGGTGGCT TCCCTCTGCC ATGACAGCCC CTAGGCCAGG AACCATCAGG GGGGCCAGCC | 3900 |
| GGCATCCAAT TCCTGCGGAT AAGTAGCGTT GGGAGAGAAC GGGAAAGGGG ACTTGGGTTA | 3960 |
| CAGGGTGACC CAGAAAGACG ATTCAGCTGT GTCCAGCCTG CCACCCATAC GTAGGCCAAC | 4020 |
| CAAGCACTTC ATGAAGAGGA GGCCTCGTGG CATATTCAGT TTACACCTGA AATATTCCTT | 4080 |
| GATGGGACAG CTTGTGGGGA TGGCTATGGG GGAAGGGGAG GTTGAGAAAG GAAGTTCTCG | 4140 |
| ACACCAGAAA TGCATCGGAG GACCACAATC AGTTCTATGC TGCCAAAGAT TAAAAATAAA | 4200 |
| TAAAAACATA AAAAATTAAG AGGGGCCAAG AGGAAGACAT TCTTTCTGCA AGGAAATTTC | 4260 |
| TTTTAAATTC TGAACTGCTA CTACACACAA GTGAAAGTCA ACCCTATGTA AACTGGTGTC | 4320 |
| CTCTCTCTAG CCCTCTCCCT TACTGGCCCA CTTCTCTCTC CGTAGAGAGC CTGAAAAACT | 4380 |
| GCCCCAATGC CACGGTAAAG GCGAGGAAGT CTTGGCTGGC GTTGCTGACT CACAGTCGCC | 4440 |
| ATCCATCTGG ACACAAAGAG AGACCTGTGG GAGTCATAGA GGGTACTGTT AGCCCCGGTC | 4500 |
| CATGCAGGGG GTTCAGCCGA GCCCAAGACT CAAAGCTGCT TTCCTTTCAG GATTTGTAGT | 4560 |
| AACGTAAGGT GATAATGGCC AAAAGTGGTT CTCTCTCATT AAACCAACCA GTAAAAGCGT | 4620 |
| ATCCTATTTT TTTGCATAAG GTGTTTCATT TTCGTTTTTA TGGGAAACCA AGGGAAAAGC | 4680 |
| ACATTGCGAT CCATTCAGTG TTTAACTGTC GTGGCTCATT TTCTGTTCGT TAGCACTTGT | 4740 |
| GTGACAAAAG AGCTCAGATC CGACTTCTCC TATGTGTCAC TTATTCCAAG AACCCAACTA | 4800 |
| TGCCCTTAGG TAGAAAGATT TGACTCGTGT GTCTACTAGC CAACAGGCAG AGCAGGGTTG | 4860 |
| AAAAAAATAT CAGCTCCCAA AGGGCCCATG TGTCTACATC ATCAGTTACT GTCATGCACC | 4920 |
| ACATTTGTGT GCAGATACCA AAAGAGGAGG AAAGAAGAAA AAAATTAATG TGTGGGAGCT | 4980 |
| GCACGTTTAC ATGTTTTGAG CTATGCTTCA AACACAACTG GAAAGCCATC AATCTTCAAA | 5040 |
| GGCCTCAAAA ATACTTTTAT AGTAACAAGT GCACGACTTT AGTTGGGTTA TTCAAGATGG | 5100 |
| CACAAAAAGG TTTCCGCAGA GGTGGTATGC TGTGCTTTTG GCGCAAGTGG TGGGGGGATG | 5160 |
| GGGGTGGGGG TGGAATTTTT TTCTCACTCT AATGACTTCC TATTGGAAAG GCATTGACAG | 5220 |
| CCAGGGACAG GAGCCAGGGT GGGGGTAGTT TTGTGGGAAA GCAGAACTGA AGTTAGCTTA | 5280 |
| AGCATAAAAA CAAAGAAAAA TCTTCGCTTT TCATGTATGT GGAATCCAAG AATAACCATA | 5340 |
| GGCTCTACCA GACCAGGAGG GTAAGGATGG ACACTAAAAT GAAACAAATA CCAAGGTATT | 5400 |
| CCTTCTGCTG CAGCCTGGAG ACCACCGAGA GTCGAGCTGG GGCACACACA CACCTGGCCG | 5460 |
| GGACCCGGCA GGGACAAGGC GGGCCGTGGC CTCCTCCACC AAGTCTCTCT AGACAATTCA | 5520 |
| GGGCCTGCTT TCCCCAGCTC CATGCATGGC TGGACTGGTG ATTCCAGGGT GCAGAAGGGA | 5580 |
| TTCATATTCC CAGAACGCTT TAAGTGTACA CCTGCAGGAT AAAGAGATAC CGGTTACATT | 5640 |
| ATTAAATGAT TCTAGGGATT CACTGGGGGA TATTTTTGTT GCTTTTACTT TCATGGTTAG | 5700 |
| AGCTACAAAG AACAGTGATT TTTTTTTTTT CTCCCTTCCC CATTCAGAAA CATTATACAT | 5760 |
| TGGGCCATTT TTCTTTCTCC CAAAGAAGAT TCATGGATAG TCAGACTGAA CTGTGTGCAA | 5820 |
| CAGGAAAAGT CAAAAGGGAA AAGGCAGCTG ATGAGGTTAC ATGGTTACAT GTTCTACATC | 5880 |
| ATGCAGAGTA GCTTGAAATC TAGTCTGGAG AAAACTGGAT CAAGATTCTA GCCCACTGGA | 5940 |
| GTTGCAAGGA ATGAGAGGCA AAAATTCTAA AGATTTGGGT TATATTTTCA ACTTGGGGGA | 6000 |
| CAGAGAGAAA TGGAGAGCAG GAATTACAGT TCCAACAAAC ATCATGATAG TCTGGTAGTC | 6060 |
| AAGACAGAGA TTAAGTAAAA CAGGTTTTAC TGTTTAGCTG AGTTCAGTTA ATACAAAATG | 6120 |
| TACATAAAAC GTTAGTCCTT TGAGACTGAC ATGATTAATG ATCAGTGTGG TGGGAAATGA | 6180 |
| TGTAGTTATT GTACACAAGC ACTTGCAAAC TCTTTATCCC TATTTCTTTA AAACAAAATA | 6240 |
| AGGTGAAATA CGAAGTCCTT GGTCTGATAT AAAGCCCCTA TTGGATTCTT CGGATGCGTA | 6300 |
| AAAGAAATTG CCTGTTTCAG CCAGAAGACT GGTGAAAACA CATACATCAG ACTATGTTGT | 6360 |
| GAGCCAGGTT GATTTTTTAT TTTATTATAT GCAGGTGAGT GTTGAAACTG TTAAAATTCC | 6420 |
| AATTTGTTTT CATTCAGTAT TAGTTTAGTT CTAAATATAG CAAACCCCAT CCAGGTGCTA | 6480 |
| TCAGATGACC AGTTACTGCT TAGTTAACTA GGTGTAAAGT TTTACATATA CATTAATTTC | 6540 |
| AATAGTTTAT TACAAGTTGT GTAAAATGGA CTCTAGTTTA ATAATGGGGG AAAAAAGATT | 6600 |
| AGGTTGGTCC TGAAACTGAC TGTAGAGCAT GTAAAATGAT TTTACTGGAT TCTGTTCAAC | 6660 |
| TGTAATGAAT GAAAAAGATG TACGTTGTAG ACAAAGTTGC AGAATTAAAA AAAGAAATCT | 6720 |
| GCTTTTAATT TATTCTTTTT GTATTAAGAA TTTGTATAGT ATCTTTACAT TTTGCAAAAC | 6780 |
| AGTGTTGTCA ACACTTATTA AAGCATTTTC AAAATG | |

ACG8 DNA sequence
Gene name: ubiquitin E3 ligase SMURF2
Unigene number: Hs.21806 (3′UTR only)
Probeset Accession #: AA398243
Nucleic Acid Accession #: AF301463 cluster
Coding sequence: 9-2255 (predicted start/stop codons underlined)

| | |
|---|---|
| CCGGGGACAT GTCTAACCCC GGAGGCCGGA GGAACGGGCC CGTCAAGCTG CGCCTGACAG | 60 |
| TACTCTGTGC AAAAAACCTG GTGAAAAAGG ATTTTTTCCG ACTTCCTGAT CCATTTGCTA | 120 |
| AGGTGGTGGT TGATGGATCT GGGCAATGCC ATTCTACAGA TACTGTGAAG AATACGCTTG | 180 |
| ATCCAAAGTG GAATCAGCAT TATGACCTGT ATATTGGAAA GTCTGATTCA GTTACGATCA | 240 |
| GTGTATGGAA TCACAAGAAG ATCCATAAGA AACAAGGTGC TGGATTTCTC GGTTGTGTTC | 300 |
| GTCTTCTTTC CAATGCCATC AACCGCCTCA AAGACACTGG TTATCAGAGG TTGGATTTAT | 360 |
| GCAAACTCGG GCCAAATGAC AATGATACAG TTAGAGGACA GATAGTAGTA AGTCTTCAGT | 420 |
| CCAGAGACCG AATAGGCACA GGAGGACAAG TTGTGGACTG CAGTCGTTTA TTTGATAACG | 480 |
| ATTTACCAGA CGGCTGGGAA GAAAGGAGAA CCGCCTCTGG AAGAATCCAG TATCTAAACC | 540 |
| ATATAACAAG AACTACGCAA TGGGAGCGCC CAACACGACC GGCATCCGAA TATTCTAGCC | 600 |
| CTGGCAGACC TCTTAGCTGC TTTGTTGATG AGAACACTCC AATTAGTGGA ACAAATGGTG | 660 |
| CAACATGTGG ACAGTCTTCA GATCCCAGGC TGGCAGAGAG GAGAGTCAGG TCACAACGAC | 720 |
| ATAGAAATTA CATGAGCAGA ACACATTTAC ATACTCCTCC AGACCTACCA GAAGGCTATG | 780 |
| AACAGAGGAC AACGCAACAA GGCCAGGTGT ATTTCTTACA TACACAGACT GGTGTGAGCA | 840 |
| CATGGCATGA TCCAAGAGTG CCCAGGGATC TTAGCAACAT CAATTGTGAA GAGCTTGGTC | 900 |
| CGTTGCCTCC TGGATGGGAG ATCCGTAATA CGGCAACAGG CAGAGTTTAT TTCGTTGACC | 960 |
| ATAACAACAG AACAACACAA TTTACAGATC CTCGGCTGTC TGCTAACTTG CATTTAGTTT | 1020 |
| TAAATCGGCA GAACCAATTG AAAGACCAAC AGCAACAGCA AGTGGTATCG TTATGTCCTG | 1080 |
| ATGACACAGA ATGCCTGACA GTCCCAAGGT ACAAGCGAGA CCTGGTTCAG AAACTAAAAA | 1140 |
| TTTTGCGGCA AGAACTTTCC CAACAACAGC CTCAGGCAGG TCATTGCCGC ATTGAGGTTT | 1200 |
| CCAGGGAAGA GATTTTTGAG GAATCATATC GACAGGTCAT GAAAATGAGA CCAAAAGATC | 1260 |
| TCTGGAAGCG ATTAATGATA AAATTTCGTG GAGAAGAAGG CCTTGACTAT GGAGGCGTTG | 1320 |
| CCAGGGAATG GTTGTATCTC TTGTCACATG AAATGTTGAA TCCATACTAT GGCCTCTTCC | 1380 |
| AGTATTCAAG AGATGATATT TATACATTGC AGATCAATCC TGATTCTGCA GTTAATCCGG | 1440 |
| AACATTTATC CTATTTCCAC TTTGTTGGAC GAATAATGGG AATGGCTGTG TTTCATGGAC | 1500 |
| ATTATATTGA TGGTGGTTTC ACATTGCCTT TTTATAAGCA ATTGCTTGGG AAGTCAATTA | 1560 |
| CCTTGGATGA CATGGAGTTA GTAGATCCGG ATCTTCACAA CAGTTTAGTG TGGATACTTG | 1620 |
| AGAATGATAT TACAGGTGTT TTGGACCATA CCTTCTGTGT TGAACATAAT GCATATGGTG | 1680 |
| AAATTATTCA GCATGAACTT AAACCAAATG GCAAAAGTAT CCCTGTTAAT GAAGAAAATA | 1740 |
| AAAAAGAATA TGTCAGGCTC TATGTGAACT GGAGATTTTT ACGAGGCATT GAGGCTCAAT | 1800 |
| TCTTGGCTCT GCAGAAAGGA TTTAATGAAG TAATTCCACA ACATCTGCTG AAGACATTTG | 1860 |
| ATGAGAAGGA GTTAGAGCTC ATTATTTGTG GACTTGGAAA GATAGATGTT AATGACTGGA | 1920 |
| AGGTAAACAC CCGGTTAAAA CACTGTACAC CAGACAGCAA CATTGTCAAA TGGTTCTGGA | 1980 |
| AAGCTGTGGA GTTTTTTGAT GAAGAGCGAC GAGCAAGATT GCTTCAGTTT GTGACAGGAT | 2040 |
| CCTCTCGAGT GCCTCTGCAG GGCTTCAAAG CATTGCAAGG TGCTGCAGGC CCGAGACTCT | 2100 |
| TTACCATACA CCAGATTGAT GCCTGCACTA ACAACCTGCC GAAAGCCCAC ACTTGCTTCA | 2160 |
| ATCGAATAGA CATTCCACCC TATGAAAGCT ATGAAAAGCT ATATGAAAAG CTGCTAACAG | 2220 |
| CCATTGAAGA AACATGTGGA TTTGCTGTGG AATGACAAGC TTCAAGGATT TACCCAGGAC | |

ACH1 DNA sequence
Gene name: EST
Unigene number: Hs.30089
Probeset Accession #: AA410480
CAT cluster#: 96816_1
Coding sequence: Partial sequence, possible frameshift. Predicted stop codon
underlined.

| | |
|---|---|
| CTCCACTATG GACAGAGCCT CCACTGAGCT GCTGCCTGCC CGCCACATAC CCAGCTGACA | 60 |
| GGGGCCCCGC AGAGCCATGC AGCTGTGCTG GGGTGATCCT GGGCTTCCTC CTGTTCCGAG | 120 |
| GCCACAACTC CCAGCCCACA ATGACCCAGA CCTCTAGCTC TCAGGGAGGC CTTGGCGGTC | 180 |
| TAAGTCTGAC CACAGAGCCA GTTTCTTCCA ACCCAGGATA CATCCCTTCC TCAGAGGCTA | 240 |
| ACAGGCCAAG CCATCTGTCC AGCACTGGTA CCCCAGGCGC AGGTGTCCCC AGCAGTGGAA | 300 |
| GAGACGGAGG CACAAGCAGA GACACATTTC AAACTGTTCC CCCCAATTCA ACCACCATGA | 360 |
| GCCTGAGCAT GAGGGAAGAT GCGACCATCC TGCCCAGCCC CACGTCAGAG ACTGTGCTCA | 420 |
| CTGTGGCTGC ATTTGGTGTT ATCAGCTTCA TTGTCATCCT GGTGGTTGTG GTGATCATCC | 480 |
| TAGTTGGTGT GGTCAGCCTG AGGTTCAAGT GTCGGAAGAG CAAGGAGTCT GGAGATCCCC | 540 |
| AGAAACCTGG AGAGCGGGAG GAGAAGCTGG GACATAGGAG GGAACCCTAC CCCTGGAATT | 600 |
| GACTTGGACT CTGGGTCTGG AAACGCAAGT TCAAATCTCA CCCATTTGTT CCAGGAGGTT | 660 |
| CTGGCTGATG AGGAAGACCC TTGTGGGAGG GGGGCCCCTG CCCTCCAGTT AGCTCTTCTT | 720 |
| GGCTGTGCTG GGTTCCATGT TCTCATGCAG GGATGGAGTC GGGTGGAGAG CCCACTCTGG | 780 |
| CTAGGGGGCG GCAGGCTGAG AGCTCACCTG TTCAGCAGAG AAGTGGAACT CACTTTGCTC | 840 |
| CTGGAGCCTC CCTACACAGT ACTTATCTGG GAAGGGAATG CCGGACTCTT GTTGGCCCCT | 900 |
| TTGTCCCCCC GACTGGCCCC CTTCGCCG | |

ACJ2 DNA sequence
Gene name: Complement component C1q receptor
Unigene number: Hs.97199
Probeset Accession #: AA487558
Nucleic Acid Accession #: NM_012072
Coding sequence: 149-2107. predicted start/stop codons underlined

| | |
|---|---|
| AAAGCCCTCA GCCTTTGTGT CCTTCTCTGC GCCGGAGTGG CTGCAGCTCA CCCCTCAGCT | 60 |
| CCCCTTGGGG CCCAGCTGGG AGCCGAGATA GAAGCTCCTG TCGCCGCTGG GCTTCTCGCC | 120 |
| TCCCGCAGAG GGCCACACAG AGACCGGGAT GGCCACCTCC ATGGGCCTGC TGCTGCTGCT | 180 |
| GCTGCTGCTC CTGACCCAGC CCGGGGCGGG GACGGGAGCT GACACGGAGG CGGTGGTCTG | 240 |
| CGTGGGGACC GCCTGCTACA CGGCCCACTC GGGCAAGCTG AGCGCTGCCG AGGCCCAGAA | 300 |
| CCACTGCAAC CAGAACGGGG GCAACCTGGC CACTGTGAAG AGCAAGGAGG AGGCCCAGCA | 360 |
| CGTCCAGCGA GTACTGGCCC AGCTCCTGAG GCGGGAGGCA GCCCTGACGG CGAGGATGAG | 420 |
| CAAGTTCTGG ATTGGGCTCC AGCGAGAGAA GGGCAAGTGC CTGGACCCTA GTCTGCCGCT | 480 |
| GAAGGGCTTC AGCTGGGTGG GCGGGGGGGA GGACACGCCT TACTCTAACT GGCACAAGGA | 540 |
| GCTCCGGAAC TCGTGCATCT CCAAGCGCTG TGTGTCTCTG CTGCTGGACC TGTCCCAGCC | 600 |
| GCTCCTTCCC AACCGCCTGC CCAAGTGGTC TGAGGGCCCC TGTGGGAGCC CAGGCTCCCC | 660 |
| CGGAAGTAAC ATTGAGGGCT TCGTGTGCAA GTTCAGCTTC AAAGGCATGT GCCGGCCTCT | 720 |
| GGCCCTGGGG GGCCCAGGTC AGGTGACCTA CACCACCCCC TTCCAGACCA CCAGTTCCTC | 780 |
| CTTGGAGGCT GTGCCCTTTG CCTCTGCGGC CAATGTAGCC TGTGGGGAAG GTGACAAGGA | 840 |
| CGAGACTCAG AGTCATTATT TCCTGTGCAA GGAGAAGGCC CCCGATGTGT TCGACTGGGG | 900 |
| CAGCTCGGGC CCCCTCTGTG TCAGCCCCAA GTATGGCTGC AACTTCAACA ATGGGGGCTG | 960 |
| CCACCAGGAC TGCTTTGAAG GGGGGGATGG CTCCTTCCTC TGCGGCTGCC GACCAGGATT | 1020 |
| CCGGCTGCTG GATGACCTGG TGACCTGTGC CTCTCGAAAC CCTTGCAGCT CCAGCCCATG | 1080 |
| TCGTGGGGGG GCCACGTGCG TCCTGGGACC CCATGGGAAA AACTACACGT GCCGCTGCCC | 1140 |
| CCAAGGGTAC CAGCTGGACT CGAGTCAGCT GGACTGTGTG GACGTGGATG AATGCCAGGA | 1200 |
| CTCCCCCTGT GCCCAGGAGT GTGTCAACAC CCCTGGGGGC TTCCGCTGCG AATGCTGGGT | 1260 |
| TGGCTATGAG CCGGGCGGTC CTGGAGAGGG GGCCTGTCAG GATGTGGATG AGTGTGCTCT | 1320 |
| GGGTCGCTCG CCTTGCGCCC AGGGCTGCAC CAACACAGAT GGCTCATTTC ACTGCTCCTG | 1380 |
| TGAGGAGGGC TACGTCCTGG CCGGGGAGGA CGGGACTCAG TGCCAGGACG TGGATGAGTG | 1440 |
| TGTGGGCCCG GGGGGCCCCC TCTGCGACAG CTTGTGCTTC AACACACAAG GGTCCTTCCA | 1500 |
| CTGTGGCTGC CTGCCAGGCT GGGTGCTGGC CCCAAATGGG GTCTCTTGCA CCATGGGGCC | 1560 |
| TGTGTCTCTG GGACCACCAT CTGGGCCCCC CGATGAGGAG GACAAAGGAG AGAAAGAAGG | 1620 |
| GAGCACCGTG CCCCGCGCTG CAACAGCCAG TCCCACAAGG GGCCCCGAGG GCACCCCCAA | 1680 |
| GGCTACACCC ACCACAAGTA GACCTTCGCT GTCATCTGAC GCCCCCATCA CATCTGCCCC | 1740 |
| ACTCAAGATG CTGGCCCCCA GTGGGTCCTC AGGCGTCTGG AGGGAGCCCA GCATCCATCA | 1800 |
| CGCCACAGCT GCCTCTGGCC CCCAGGAGCC TGCAGGTGGG GACTCCTCCG TGGCCACACA | 1860 |
| AAACAACGAT GGCACTGACG GGCAAAAGCT GCTTTTATTC TACATCCTAG GCACCGTGGT | 1920 |
| GGCCATCCTA CTCCTGCTGG CCCTGGCTCT GGGGCTACTG GTCTATCGCA AGCGGAGAGC | 1980 |
| GAAGAGGGAG GAGAAGAAGG AGAAGAAGCC CCAGAATGCG GCAGACAGTT ACTCCTGGGT | 2040 |
| TCCAGAGCGA GCTGAGAGCA GGGCCATGGA GAACCAGTAC AGTCCGACAC CTGGGACAGA | 2100 |
| CTGCTGAAAG TGAGGTGGCC CTAGAGACAC TAGAGTCACC AGCCACCATC CTCAGAGCTT | 2160 |
| TGAACTCCCC ATTCCAAAGG GGCACCGACA TTTTTTTGAA AGACTGGACT GGAATCTTAG | 2220 |
| CAAACAATTG TAAGTCTCCT CCTTAAAGGC CCCTTGGAAC ATGCAGGTAT TTTCTACGGG | 2280 |
| TGTTTGATGT TCCTGAAGTG GAAGCTGTGT GTTGGCGTGC CACGGTGGGG ATTTCGTGAC | 2340 |
| TCTATAATGA TTGTTACTCC CCCTCCCTTT TCAAATTCCA ATGTGACCAA TTCCGGATCA | 2400 |
| GGGTGTGAGG AGGCTGGGGC TAAGGGGCTC CCCTGAATAT CTTCTCTGCT CACTTCCACC | 2460 |
| ATCTAAGAGG AAAAGGTGAG TTGCTCATGC TGATTAGGAT TGAAATGATT TGTTTCTCTT | 2520 |
| CCTAGGATGA AAACTAAATC AATTAATTAT TCAATTAGGT AAGAAGATCT GGTTTTTTGG | 2580 |
| TCAAAGGGAA CATGTTCGGA CTGGAAACAT TTCTTTACAT TTGCATTCCT CCATTTCGCC | 2640 |
| AGCACAAGTC TTGCTAAATG TGATACTGTT GACATCCTCC AGAATGGCCA GAAGTGCAAT | 2700 |
| TAACCTCTTA GGTGGCAAGG AGGCAGGAAG TGCCTCTTTA GTTCTTACAT TTCTAATAGC | 2760 |
| CTTGGGTTTA TTTGCAAAGG AAGCTTGAAA AATATGAGAA AAGTTGCTTG AAGTGCATTA | 2820 |
| CAGGTGTTTG TGAAGTCACA TAATCTACGG GGCTAGGGCG AGAGAGGCCA GGGATTTGTT | 2880 |
| CACAGATACT TGAATTAATT CATCCAAATG TACTGAGGTT ACCACACACT TGACTACGGA | 2940 |
| TGTGATCAAC ACTAACAAGG AAACAAATTC AAGGACAACC TGTCTTTGAG CCAGGGCAGG | 3000 |
| CCTCAGACAC CCTGCCTGTG GCCCCGCCTC CACTTCATCC TGCCCGGAAT GCCAGTGCTC | 3060 |
| CGAGCTCAGA CAGAGGAAGC CCTGCAGAAA GTTCCATCAG GCTGTTTGCT AAAGGATGTG | 3120 |
| TGAACGGGAG ATGATGCACT GTGTTTTGAA AGTTGTCATT TTAAAGCATT TTAGCACAGT | 3180 |
| TCATAGTCCA CAGTTGATGC AGCATCCTGA GATTTTAAAT CCTGAAGTGT GGGTGGCGCA | 3240 |
| CACACCAAGT AGGGAGCTAG TCAGGCAGTT TGCTTAAGGA ACTTTTGTTC TCTGTCTCTT | 3300 |
| TTCCTTAAAA TTGGGGGTAA GGAGGGAAGG AAGAGGGAAA GAGATGACTA ACTAAAATCA | 3360 |
| TTTTTACAGC AAAAACTGCT CAAAGCCATT TAAATTATAT CCTCATTTTA AAAGTTACAT | 3420 |
| TTGCAAATAT TTCTCCCTAT GATAATGCAG TCGATAGTGT GCACTCTTTC TCTCTCTCTC | 3480 |
| TCTCTCTCAC ACACACACAC ACACACACAC ACACACACAC AGAGACACGG CACCATTCTG | 3540 |
| CCTGGGGCAC TGGAACACAT TCCTGGGGGT CACCGATGGT CAGAGTCACT AGAAGTTACC | 3600 |
| TGAGTATCTC TGGGAGGCCT CATGTCTCCT GTGGGCTTTT TACCACCACT GTGCAGGAGA | 3660 |
| ACAGACAGAG GAAATGTGTC TCCCTCCAAG GCCCCAAAGC CTCAGAGAAA GGGTGTTTCT | 3720 |
| GGTTTTGCCT TAGCAATGCA TCGGTCTCTG AGGTGACACT CTGGAGTGGT TGAAGGGCCA | 3780 |
| CAAGGTGCAG GGTTAATACT CTTGCCAGTT TTGAAATATA GATGCTATGG TTCAGATTGT | 3840 |
| TTTTAATAGA AAACTAAAGG GGCAGGGGAA GTGAAAGGAA AGATGGAGGT TTTGTGCGGC | 3900 |
| TCGATGGGGC ATTTGGAACT TCTTTTTAAA GTCATCTCAT GGTCTCCAGT TTTCAGTTGG | 3960 |
| AACTCTGGTG TTTAACACTT AAGGGAGACA AAGGCTGTGT CCATTTGGCA AAACTTCCTT | 4020 |
| GGCCACGAGA CTCTAGGTGA TGTGTGAAGC TGGGCAGTCT GTGGTGTGGA GAGCAGCCAT | 4080 |
| CTGTCTGGCC ATTCAGAGGA TTCTAAAGAC ATGGCTGGAT GCGCTGCTGA CCAACATCAG | 4140 |
| CACTTAAATA AATGCAAATG CAACATTTCT CCCTCTGGGC CTTGAAAATC CTTGCCCTTA | 4200 |
| TCATTTGGGG TGAAGGAGAC ATTTCTGTCC TTGGCTTCCC ACAGCCCCAA CGCAGTCTGT | 4260 |
| GTATGATTCC TGGGATCCAA CGAGCCCTCC TATTTTCACA GTGTTCTGAT TGCTCTCACA | 4320 |
| GCCCAGGCCC ATCGTCTGTT CTCTGAATGC AGCCCTGTTC TCAACAACAG GGAGGTCATG | 4380 |
| GAACCCCTCT GTGGAACCCA CAAGGGGAGA AATGGGTGAT AAAGAATCCA GTTCCTCAAA | 4440 |
| ACCTTCCCTG GCAGGCTGGG TCCCTCTCCT GCTGGGTGGT GCTTTCTCTT GGACACCACT | 4500 |
| CCCACCACGG GGGGAGAGCC AGCAACCCAA CCAGACAGCT CAGGTTGTGC ATCTGATGGA | 4560 |
| AACCACTGGG CTCAAACACG TGCTTTATTC TCCTGTTTAT TTTTGCTGTT ACTTTGAAGC | 4620 |
| ATGGAAATTC TTGTTTGGGG GATCTTGGGG CTACAGTAGT GGGTAAACAA ATGCCCACCG | 4680 |
| GCCAAGAGGC CATTAACAAA TCGTCCTTGT CCTGAGGGGC CCCAGCTTGC TCGGGCGTGG | 4740 |
| CACAGTGGGG AATCCAAGGG TCACAGTATG GGGAGAGGTG CACCCTGCCA CCTGCTAACT | 4800 |
| TCTCGCTAGA CACAGTGTTT CTGCCCAGGT GACCTGTTCA GCAGCAGAAC AAGCCAGGGC | 4860 |
| CATGGGGACG GGGGAAGTTT TCACTTGGAG ATGGACACCA AGACAATGAA GATTTGTTGT | 4920 |
| CCAAATAGGT CAATAATTCT GGGAGACTCT TGGAAAAAAC TGAATATATT CAGGACCAAC | 4980 |
| TCTCTCCCTC CCCTCATCCC ACATCTCAAA GCAGACAATG TAAAGAGAGA ACATCTCACA | 5040 |
| CACCCAGCTC GCCATGCCTA CTCATTCCTG AATTTCAGGT GCCATCACTG CTCTTTCTTT | 5100 |
| CTTCTTTGTC ATTTGAGAAA GGATGCAGGA GGACAATTCC CACAGATAAT CTGAGGAATG | 5160 |
| CAGAAAAACC AGGGCAGGAC AGTTATCGAC AATGCATTAG AACTTGGTGA GCATCCTCTG | 5220 |
| TAGAGGGACT CCACCCCTGC TCAACAGCTT GGCTTCCAGG CAAGACCAAC CACATCTGGT | 5280 |
| CTCTGCCTTC GGTGGCCCAC ACACCTAAGC GTCATCGTCA TTGCCATAGC ATCATGATGC | 5340 |
| AACACATCTA CGTGTAGCAC TACGACGTTA TGTTTGGGTA ATGTGGGGAT GAACTGCATG | 5400 |
| AGGCTCTGAT TAAGGATGTG GGGAAGTGGG CTGCGGTCAC TGTCGGCCTT GCAAGGCCAC | 5460 |
| CTGGAGGCCT GTCTGTTAGC CAGTGGTGGA GGAGCAAGGC TTCAGGAAGG GCCAGCCACA | 5520 |
| TGCCATCTTC CCTGCGATCA GGCAAAAAAG TGGAATTAAA AAGTCAAACC TTTATATGCA | 5580 |
| TGTGTTATGT CCATTTTGCA GGATGAACTG AGTTTAAAAG AATTTTTTTT TCTCTTCAAG | 5640 |
| TTGCTTTGTC TTTTCCATCC TCATCACAAG CCCTTGTTTG AGTGTCTTAT CCCTGAGCAA | 5700 |
| TCTTTCGATG GATGGAGATG ATCATTAGGT ACTTTTGTTT CAACCTTTAT TCCTGTAAAT | 5760 |
| ATTTCTGTGA AAACTAGGAG AACAGAGATG AGATTTGACA AAAAAAAATT GAATTAAAAA | 5820 |
| TAACACAGTC TTTTTAAAAC TAACATAGGA AAGCCTTTCC TATTATTTCT CTTCTTAGCT | 5880 |
| TCTCCATTGT CTAAATCAGG AAAACAGGAA AACACAGCTT TCTAGCAGCT GCAAAATGGT | 5940 |
| TTAATGCCCC CTACATATTT CCATCACCTT GAACAATAGC TTTAGCTTGG GAATCTGAGA | 6000 |
| TATGATCCCA GAAAACATCT GTCTCTACTT CGGCTGCAAA ACCCATGGTT TAAATCTATA | 6060 |
| TGGTTTGTGC ATTTTCTCAA CTAAAAATAG AGATGATAAT CCGAATTCTC CATATATTCA | 6120 |
| CTAATCAAAG ACACTATTTT CATACTAGAT TCCTGAGACA AATACTCACT GAAGGGCTTG | 6180 |
| TTTAAAAATA AATTGTGTTT TGGTCTGTTC TTGTAGATAA TGCCCTTCTA TTTTAGGTAG | 6240 |
| AAGCTCTGGA ATCCCTTTAT TGTGCTGTTG CTCTTATCTG CAAGGTGGCA AGCAGTTCTT | 6300 |
| TTCAGCAGAT TTTGCCCACT ATTCCTCTGA GCTGAAGTTC TTTGCATAGA TTTGGCTTAA | 6360 |
| GCTTGAATTA GATCCCTGCA AAGGCTTGCT CTGTGATGTC AGATGTAATT GTAAATGTCA | 6420 |
| GTAATCACTT CATGAATGCT AAATGAGAAT GTAAGTATTT TTAAATGTGT GTATTTCAAA | 6480 |
| TTTGTTTGAC TAATTCTGGA ATTACAAGAT TTCTATGCAG GATTTACCTT CATCCTGTGC | 6540 |
| ATGTTTCCCA AACTGTGAGG AGGGAAGGCT CAGAGATCGA GCTTCTCCTC TGAGTTCTAA | 6600 |
| CAAAATGGTG CTTTGAGGGT CAGCCTTTAG GAAGGTGCAG CTTTGTTGTC CTTTGAGCTT | 6660 |
| TCTGTTATGT GCCTATCCTA ATAAACTCTT AAACACATT | |

ACJ3 DNA sequence
Gene name: FLT1/vascular endothelial growth factor receptor
Unigene number: Hs.138671
Probeset Accession #: AA047437
Nucleic Acid Accession #: NM_002019
Coding sequence: 250-4266 (predicted start/stop codons underlined)

| | |
|---|---|
| GCGGACACTC CTCTCGGCTC CTCCCCGGCA GCGGCGGCGG CTCGGAGCGG GCTCCGGGGC | 60 |
| TCGGGTGCAG CGGCCAGCGG GCCTGGCGGC GAGGATTACC CGGGGAAGTG GTTGTCTCCT | 120 |
| GGCTGGAGCC GCGAGACGGG CGCTCAGGGC GCGGGGCCGG CGGCGGCGAA CGAGAGGACG | 180 |
| GACTCTGGCG GCCGGGTCGT TGGCCGGGGG AGCGCGGGCA CCGGGCGAGC AGGCCGCGTC | 240 |
| GCGCTCACCA TGGTCAGCTA CTGGGACACC GGGGTCCTGC TGTGCGCGCT GCTCAGCTGT | 300 |
| CTGCTTCTCA CAGGATCTAG TTCAGGTTCA AAATTAAAAG ATCCTGAACT GAGTTTAAAA | 360 |
| GGCACCCAGC ACATCATGCA AGCAGGCCAG ACACTGCATC TCCAATGCAG GGGGGAAGCA | 420 |
| GCCCATAAAT GGTCTTTGCC TGAAATGGTG AGTAAGGAAA GCGAAAGGCT GAGCATAACT | 480 |
| AAATCTGCCT GTGGAAGAAA TGGCAAACAA TTCTGCAGTA CTTTAACCTT GAACACAGCT | 540 |
| CAAGCAAACC ACACTGGCTT CTACAGCTGC AAATATCTAG CTGTACCTAC TTCAAAGAAG | 600 |
| AAGGAAACAG AATCTGCAAT CTATATATTT ATTAGTGATA CAGGTAGACC TTTCGTAGAG | 660 |
| ATGTACAGTG AAATCCCCGA AATTATACAC ATGACTGAAG GAAGGGAGCT CGTCATTCCC | 720 |
| TGCCGGGTTA CGTCACCTAA CATCACTGTT ACTTTAAAAA AGTTTCCACT TGACACTTTG | 780 |
| ATCCCTGATG GAAAACGCAT AATCTGGGAC AGTAGAAAGG GCTTCATCAT ATCAAATGCA | 840 |
| ACGTACAAAG AAATAGGGCT TCTGACCTGT GAAGCAACAG TCAATGGGCA TTTGTATAAG | 900 |
| ACAAACTATC TCACACATCG ACAAACCAAT ACAATCATAG ATGTCCAAAT AAGCACACCA | 960 |
| CGCCCAGTCA AATTACTTAG AGGCCATACT CTTGTCCTCA ATTGTACTGC TACCACTCCC | 1020 |
| TTGAACACGA GAGTTCAAAT GACCTGGAGT TACCCTGATG AAAAAAATAA GAGAGCTTCC | 1080 |
| GTAAGGCGAC GAATTGACCA AAGCAATTCC CATGCCAACA TATTCTACAG TGTTCTTACT | 1140 |
| ATTGACAAAA TGCAGAACAA AGACAAAGGA CTTTATACTT GTCGTGTAAG GAGTGGACCA | 1200 |
| TCATTCAAAT CTGTTAACAC CTCAGTGCAT ATATATGATA AAGCATTCAT CACTGTGAAA | 1260 |
| CATCGAAAAC AGCAGGTGCT TGAAACCGTA GCTGGCAAGC GGTCTTACCG GCTCTCTATG | 1320 |
| AAAGTGAAGG CATTTCCCTC GCCGGAAGTT GTATGGTTAA AAGATGGGTT ACCTGCGACT | 1380 |
| GAGAAATCTG CTCGCTATTT GACTCGTGGC TACTCGTTAA TTATCAAGGA CGTAACTGAA | 1440 |
| GAGGATGCAG GGAATTATAC AATCTTGCTG AGCATAAAAC AGTCAAATGT GTTTAAAAAC | 1500 |
| CTCACTGCCA CTCTAATTGT CAATGTGAAA CCCCAGATTT ACGAAAAGGC CGTGTCATCG | 1560 |
| TTTCCAGACC CGGCTCTCTA CCCACTGGGC AGCAGACAAA TCCTGACTTG TACCGCATAT | 1620 |
| GGTATCCCTC AACCTACAAT CAAGTGGTTC TGGCACCCCT GTAACCATAA TCATTCCGAA | 1680 |
| GCAAGGTGTG ACTTTTGTTC CAATAATGAA GAGTCCTTTA TCCTGGATGC TGACAGCAAC | 1740 |
| ATGGGAAACA GAATTGAGAG CATCACTCAG CGCATGGCAA TAATAGAAGG AAAGAATAAG | 1800 |
| ATGGCTAGCA CCTTGGTTGT GGCTGACTCT AGAATTTCTG GAATCTACAT TTGCATAGCT | 1860 |
| TCCAATAAAG TTGGGACTGT GGGAAGAAAC ATAAGCTTTT ATATCACAGA TGTGCCAAAT | 1920 |
| GGGTTTCATG TTAACTTGGA AAAAATGCCG ACGGAAGGAG AGGACCTGAA ACTGTCTTGC | 1980 |
| ACAGTTAACA AGTTCTTATA CAGAGACGTT ACTTGGATTT TACTGCGGAC AGTTAATAAC | 2040 |
| AGAACAATGC ACTACAGTAT TAGCAAGCAA AAAATGGCCA TCACTAAGGA GCACTCCATC | 2100 |
| ACTCTTAATC TTACCATCAT GAATGTTTCC CTGCAAGATT CAGGCACCTA TGCCTGCAGA | 2160 |
| GCCAGGAATG TATACACAGG GGAAGAAATC CTCCAGAAGA AAGAAATTAC AATCAGAGAT | 2220 |
| CAGGAAGCAC CATACCTCCT GCGAAACCTC AGTGATCACA CAGTGGCCAT CAGCAGTTCC | 2280 |
| ACCACTTTAG ACTGTCATGC TAATGGTGTC CCCGAGCCTC AGATCACTTG GTTTAAAAAC | 2340 |
| AACCACAAAA TACAACAAGA GCCTGGAATT ATTTTAGGAC CAGGAAGCAG CACGCTGTTT | 2400 |
| ATTGAAAGAG TCACAGAAGA GGATGAAGGT GTCTATCACT GCAAAGCCAC CAACCAGAAG | 2460 |
| GGCTCTGTGG AAAGTTCAGC ATACCTCACT GTTCAAGGAA CCTCGGACAA GTCTAATCTG | 2520 |
| GAGCTGATCA CTCTAACATG CACCTGTGTG GCTGCGACTC TCTTCTGGCT CCTATTAACC | 2580 |
| CTCCTTATCC GAAAAATGAA AAGGTCTTCT TCTGAAATAA AGACTGACTA CCTATCAATT | 2640 |
| ATAATGGACC CAGATGAAGT TCCTTTGGAT GAGCAGTGTG AGCGGCTCCC TTATGATGCC | 2700 |
| AGCAAGTGGG AGTTTGCCCG GGAGAGACTT AAACTGGGCA AATCACTTGG AAGAGGGGCT | 2760 |
| TTTGGAAAAG TGGTTCAAGC ATCAGCATTT GGCATTAAGA AATCACCTAC GTGCCGGACT | 2820 |
| GTGGCTGTGA AAATGCTGAA AGAGGGGGCC ACGGCCAGCG AGTACAAAGC TCTGATGACT | 2880 |
| GAGCTAAAAA TCTTGACCCA CATTGGCCAC CATCTGAACG TGGTTAACCT GCTGGGAGCC | 2940 |
| TGCACCAAGC AAGGAGGGCC TCTGATGGTG ATTGTTGAAT ACTGCAAATA TGGAAATCTC | 3000 |
| TCCAACTACC TCAAGAGCAA ACGTGACTTA TTTTTTCTCA ACAAGGATGC AGCACTACAC | 3060 |
| ATGGAGCCTA AGAAAGAAAA AATGGAGCCA GGCCTGGAAC AAGGCAAGAA ACCAAGACTA | 3120 |
| GATAGCGTCA CCAGCAGCGA AAGCTTTGCG AGCTCCGGCT TTCAGGAAGA TAAAAGTCTG | 3180 |
| AGTGATGTTG AGGAAGAGGA GGATTCTGAC GGTTTCTACA AGGAGCCCAT CACTATGGAA | 3240 |
| GATCTGATTT CTTACAGTTT TCAAGTGGCC AGAGGCATGG AGTTCCTGTC TTCCAGAAAG | 3300 |
| TGCATTCATC GGGACCTGGC AGCGAGAAAC ATTCTTTTAT CTGAGAACAA CGTGGTGAAG | 3360 |
| ATTTGTGATT TTGGCCTTGC CCGGGATATT TATAAGAACC CCGATTATGT GAGAAAAGGA | 3420 |
| GATACTCGAC TTCCTCTGAA ATGGATGGCT CCCGAATCTA TCTTTGACAA AATCTACAGC | 3480 |
| ACCAAGAGCG ACGTGTGGTC TTACGGAGTA TTGCTGTGGG AAATCTTCTC CTTAGGTGGG | 3540 |
| TCTCCATACC CAGGAGTACA AATGGATGAG GACTTTTGCA GTCGCCTGAG GGAAGGCATG | 3600 |
| AGGATGAGAG CTCCTGAGTA CTCTACTCCT GAAATCTATC AGATCATGCT GGACTGCTGG | 3660 |
| CACAGAGACC CAAAAGAAAG GCCAAGATTT GCAGAACTTG TGGAAAAACT AGGTGATTTG | 3720 |
| CTTCAAGCAA ATGTACAACA GGATGGTAAA GACTACATCC CAATCAATGC CATACTGACA | 3780 |
| GGAAATAGTG GGTTTACATA CTCAACTCCT GCCTTCTCTG AGGACTTCTT CAAGGAAAGT | 3840 |
| ATTTCAGCTC CGAAGTTTAA TTCAGGAAGC TCTGATGATG TCAGATATGT AAATGCTTTC | 3900 |
| AAGTTCATGA GCCTGGAAAG AATCAAAACC TTTGAAGAAC TTTTACCGAA TGCCACCTCC | 3960 |
| ATGTTTGATG ACTACCAGGG CGACAGCAGC ACTCTGTTGG CCTCTCCCAT GCTGAAGCGC | 4020 |
| TTCACCTGGA CTGACAGCAA ACCCAAGGCC TCGCTCAAGA TTGACTTGAG AGTAACCAGT | 4080 |
| AAAAGTAAGG AGTCGGGGCT GTCTGATGTC AGCAGGCCCA GTTTCTGCCA TTCCAGCTGT | 4140 |
| GGGCACGTCA GCGAAGGCAA GCGCAGGTTC ACCTACGACC ACGCTGAGCT GGAAAGGAAA | 4200 |
| ATCGCGTGCT GCTCCCCGCC CCCAGACTAC AACTCGGTGG TCCTGTACTC CACCCCACCC | 4260 |
| ATCTAGAGTT TGACACGAAG CCTTATTTCT AGAAGCACAT GTGTATTTAT ACCCCCAGGA | 4320 |
| AACTAGCTTT TGCCAGTATT ATGCATATAT AAGTTTACAC CTTTATCTTT CCATGGGAGC | 4380 |
| CAGCTGCTTT TTGTGATTTT TTTAATAGTG CTTTTTTTTT TTGACTAACA AGAATGTAAC | 4440 |
| TCCAGATAGA GAAATAGTGA CAAGTGAAGA ACACTACTGC TAAATCCTCA TGTTACTCAG | 4500 |
| TGTTAGAGAA ATCCTTCCTA AACCCAATGA CTTCCCTGCT CCAACCCCCG CCACCTCAGG | 4560 |
| GCACGCAGGA CCAGTTTGAT TGAGGAGCTG CACTGATCAC CCAATGCATC ACGTACCCCA | 4620 |
| CTGGGCCAGC CCTGCAGCCC AAAACCCAGG GCAACAAGCC CGTTAGCCCC AGGGGATCAC | 4680 |
| TGGCTGGCCT GAGCAACATC TCGGGAGTCC TCTAGCAGGC CTAAGACATG TGAGGAGGAA | 4740 |
| AAGGAAAAAA AGCAAAAAGC AAGGGAGAAA AGAGAAACCG GGAGAAGGCA TGAGAAAGAA | 4800 |
| TTTGAGACGC ACCATGTGGG CACGGAGGGG GACGGGGCTC AGCAATGCCA TTTCAGTGGC | 4860 |
| TTCCCAGCTC TGACCCTTCT ACATTTGAGG GCCCAGCCAG GAGCAGATGG ACAGCGATGA | 4920 |
| GGGGACATTT TCTGGATTCT GGGAGGCAAG AAAAGGACAA ATATCTTTTT TGGAACTAAA | 4980 |
| GCAAATTTTA GACCTTTACC TATGGAAGTG GTTCTATGTC CATTCTCATT CGTGGCATGT | 5040 |
| TTTGATTTGT AGCACTGAGG GTGGCACTCA ACTCTGAGCC CATACTTTTG GCTCCTCTAG | 5100 |
| TAAGATGCAC TGAAAACTTA GCCAGAGTTA GGTTGTCTCC AGGCCATGAT GGCCTTACAC | 5160 |
| TGAAAATGTC ACATTCTATT TTGGGTATTA ATATATAGTC CAGACACTTA ACTCAATTTC | 5220 |
| TTGGTATTAT TCTGTTTTGC ACAGTTAGTT GTGAAAGAAA GCTGAGAAGA ATGAAAATGC | 5280 |
| AGTCCTGAGG AGAGTTTTCT CCATATCAAA ACGAGGGCTG ATGGAGGAAA AAGGTCAATA | 5340 |
| AGGTCAAGGG AAGACCCCGT CTCTATACCA ACCAAACCAA TTCACCAACA CAGTTGGGAC | 5400 |
| CCAAAACACA GGAAGTCAGT CACGTTTCCT TTTCATTTAA TGGGGATTCC ACTATCTCAC | 5460 |
| ACTAATCTGA AAGGATGTGG AAGAGCATTA GCTGGCGCAT ATTAAGCACT TTAAGCTCCT | 5520 |
| TGAGTAAAAA GGTGGTATGT AATTTATGCA AGGTATTTCT CCAGTTGGGA CTCAGGATAT | 5580 |
| TAGTTAATGA GCCATCACTA GAAGAAAAGC CCATTTTCAA CTGCTTTGAA ACTTGCCTGG | 5640 |
| GGTCTGAGCA TGATGGGAAT AGGGAGACAG GGTAGGAAAG GGCGCCTACT CTTCAGGGTC | 5700 |
| TAAAGATCAA GTGGGCCTTG GATCGCTAAG CTGGCTCTGT TTGATGCTAT TTATGCAAGT | 5760 |
| TAGGGTCTAT GTATTTAGGA TGCGCCTACT CTTCAGGGTC TAAAGATCAA GTGGGCCTTG | 5820 |
| GATCGCTAAG CTGGCTCTGT TTGATGCTAT TTATGCAAGT TAGGGTCTAT GTATTTAGGA | 5880 |
| TGTCTGCACC TTCTGCAGCC AGTCAGAAGC TGGAGAGGCA ACAGTGGATT GCTGCTTCTT | 5940 |
| GGGGAGAAGA GTATGCTTCC TTTTATCCAT GTAATTTAAC TGTAGAACCT GAGCTCTAAG | 6000 |
| TAACCGAAGA ATGTATGCCT CTGTTCTTAT GTGCCACATC CTTGTTTAAA GGCTCTCTGT | 6060 |
| ATGAAGAGAT GGGACCGTCA TCAGCACATT CCCTAGTGAG CCTACTGGCT CCTGGCAGCG | 6120 |
| GCTTTTGTGG AAGACTCACT AGCCAGAAGA GAGGAGTGGG ACAGTCCTCT CCACCAAGAT | 6180 |
| CTAAATCCAA ACAAAAGCAG GCTAGAGCCA GAAGAGAGGA CAAATCTTTG TTGTTCCTCT | 6240 |
| TCTTTACACA TACGCAAACC ACCTGTGACA GCTGGCAATT TTATAAATCA GGTAACTGGA | 6300 |
| AGGAGGTTAA ACTCAGAAAA AAGAAGACCT CAGTCAATTC TCTACTTTTT TTTTTTTTTT | 6360 |
| TCCAAATCAG ATAATAGCCC AGCAAATAGT GATAACAAAT AAAACCTTAG CTGTTCATGT | 6420 |
| CTTGATTTCA ATAATTAATT CTTAATCATT AAGAGACCAT AATAAATACT CCTTTTCAAG | 6480 |
| AGAAAAGCAA AACCATTAGA ATTGTTACTC AGCTCCTTCA AACTCAGGTT TGTAGCATAC | 6540 |
| ATGAGTCCAT CCATCAGTCA AAGAATGGTT CCATCTGGAG TCTTAATGTA GAAAGAAAAA | 6600 |
| TGGAGACTTG TAATAATGAG CTAGTTACAA AGTGCTTGTT CATTAAAATA GCACTGAAAA | 6660 |
| TTGAAACATG AATTAACTGA TAATATTCCA ATCATTTGCC ATTTATGACA AAAATGGTTG | 6720 |
| GCACTAACAA AGAACGAGCA CTTCCTTTCA GAGTTTCTGA GATAATGTAC GTGGAACAGT | 6780 |
| CTGGGTGGAA TGGGGCTGAA ACCATGTGCA AGTCTGTGTC TTGTCAGTCC AAGAAGTGAC | 6840 |
| ACCGAGATGT TAATTTTAGG GACCCGTGCC TTGTTTCCTA GCCCACAAGA ATGCAAACAT | 6900 |
| CAAACAGATA CTCGCTAGCC TCATTTAAAT TGATTAAAGG AGGAGTGCAT CTTTGGCCGA | 6960 |
| CAGTGGTGTA ACTGTGTGTG TGTGTGTGTG TGTGTGTGTG TGTGTGTGTG TGTGGGTGTG | 7020 |
| GGTGTATGTG TGTTTTGTGC ATAACTATTT AAGGAAACTG GAATTTTAAA GTTACTTTTA | 7080 |
| TACAAACCAA GAATATATGC TACAGATATA AGACAGACAT GGTTTGGTCC TATATTTCTA | 7140 |
| GTCATGATGA ATGTATTTTG TATACCATCT TCATATAATA TACTTAAAAA TATTTCTTAA | 7200 |
| TTGGGATTTG TAATCGTACC AACTTAATTG ATAAACTTGG CAACTGCTTT TATGTTCTGT | 7260 |
| CTCCTTCCAT AAATTTTTCA AAATACTAAT TCAACAAAGA AAAAGCTCTT TTTTTTCCTA | 7320 |
| AAATAAACTC AAATTTATCC TTGTTTAGAG CAGAGAAAAA TTAAGAAAAA CTTTGAAATG | 7380 |
| GTCTCAAAAA ATTGCTAAAT ATTTTCAATG GAAAACTAAA TGTTAGTTTA GCTGATTGTA | 7440 |
| TGGGGTTTTC GAACCTTTCA CTTTTTGTTT GTTTTACCTA TTTCACAACT GTGTAAATTG | 7500 |
| CCAATAATTC CTGTCCATGA AAATGCAAAT TATCCAGTGT AGATATATTT GACCATCACC | 7560 |
| CTATGGATAT TGGCTAGTTT TGCCTTTATT AAGCAAATTC ATTTCAGCCT GAATGTCTGC | 7620 |
| CTATATATTC TCTGCTCTTT GTATTCTCCT TTGAACCCGT TAAAACATCC TGTGGCACTC | |

ACJ9 DNA sequence
Gene name: Purine nucleoside phosphorylase
Unigene number: Hs.75514
Probeset Accession #: K02574
Nucleic acid Accession #: X00737 cluster
Coding sequence: 110-979 (predicted start/stop codons underlined)

| | |
|---|---|
| AACTGTGCGA ACCAGACCCG GCAGCCTTGC TCAGTTCAGC ATAGCGGAGC GGATCCGATC | 60 |
| GGATCGGAGC ACACCGGAGC AGGCTCATCG AGAAGGCGTC TGCGAGACCA TGGAGAACGG | 120 |
| ATACACCTAT GAAGATTATA AGAACACTGC AGAATGGCTT CTGTCTCATA CTAAGCACCG | 180 |
| ACCTCAAGTT GCAATAATCT GTGGTTCTGG ATTAGGAGGT CTGACTGATA AATTAACTCA | 240 |
| GGCCCAGATC TTTGACTACA GTGAAATCCC CAACTTTCCT CGAAGTACAG TGCCAGGTCA | 300 |
| TGCTGGCCGA CTGGTGTTTG GGTTCCTGAA TGGCAGGGCC TGTGTGATGA TGCAGGGCAG | 360 |
| GTTCCACATG TATGAAGGGT ACCCACTCTG GAAGGTGACA TTCCCAGTGA GGGTTTTCCA | 420 |
| CCTTCTGGGT GTGGACACCC TGGTAGTCAC CAATGCAGCA GGAGGGCTGA ACCCCAAGTT | 480 |
| TGAGGTTGGA GATATCATGC TGATCCGTGA CCATATCAAC CTACCTGGTT TCAGTGGTCA | 540 |
| GAACCCTCTC AGAGGGCCCA ATGATGAAAG GTTTGGAGAT CGTTTCCCTG CCATGTCTGA | 600 |
| TGCCTACGAC CGGACTATGA GGCAGAGGGC TCTCAGTACC TGGAAACAAA TGGGGGAGCA | 660 |
| ACGTGAGCTA CAGGAAGGCA CCTATGTGAT GGTGGCAGGC CCCAGCTTTG AGACTGTGGC | 720 |
| AGAATGTCGT GTGCTGCAGA AGCTGGGAGC AGACGCTGTT GGCATGAGTA CAGTACCAGA | 780 |
| AGTTATCGTT GCACGGCACT GTGGACTTCG AGTCTTTGGC TTCTCACTCA TCACTAACAA | 840 |
| GGTCATCATG GATTATGAAA GCCTGGAGAA GGCCAACCAT GAAGAAGTCT TAGCAGCTGG | 900 |
| CAAACAAGCT GCACAGAAAT TGGAACAGTT TGTCTCCATT CTTATGGCCA GCATTCCACT | 960 |
| CCCTGACAAA GCCAGTTGAC CTGCCTTGGA GTCGTCTGGC ATCTCCCACA CAAGACCCAA | 1020 |
| GTAGCTGCTA CCTTCTTTGG CCCCTTGCTG GAGTCATGTG CCTCTGTCCT TAGGTTGTAG | 1080 |
| CAGAAAGGAA AAGATTCCTG TCCTTCACCT TTCCCACTTT CTTCTACCAG ACCCTTCTGG | 1140 |
| TGCCAGATCC TCTTCTCAAA GCTGGGATTA CAGGTGTGAG CATAGTGAGA CCTTGGCGCT | 1200 |
| ACAAAATAAA GCTGTTCTCA TTCCTGTTCT TTCTTACACA AGAGCTGGAG CCCGTGCCCT | 1260 |
| ACCACACATC TGTGGAGATG CCCAGGATTT GACTCGGGCC TTAGAACTTT GCATAGCAGC | 1320 |
| TGCTACTAGC TCTTTGAGAT AATACATTCC GAGGGGCTCA GTTCTGCCTT ATCTAAATCA | 1380 |
| CCAGAGACCA AACAAGGACT AATCCAATAC CTCTTGGA | |

ACK4 DNA sequence
Gene name: EST
Unigene number: Hs.265499
Probeset Accession #: R68763
CAT cluster#: Cluster 46668_2
Sequence: Both the EST corresponding to the probeset accession and exon
prediction; number and the CAT cluster align with the Homo sapiens BAC clone
AC009414 RP11-490M8. Using FGENESH, 2 exons predicted on this BAC clone upstream
of the probeset.
predicted exon 1: bases 5808-5837 of BAC clone AC009414

| | |
|---|---|
| AAAGTCTCGC CCAAACTTTG TTCGGCACAA CCAGCGCCGA GGGGGCGGCG CAGGCCAGGT | 60 |
| GGGAGGGGGC CCGCAGCGGG CGGCCGTACC TTCGCAAACG CCCGCTTCGT ACTCGGTGAG | 120 |
| GGAGTCGCCA TTGAGCGGGG GGCGGATGAC ACAACGCAGC CCCCGGTCGC AGGTTCCGTA | 180 |
| AATCCCGAAG GTGCCGCCGC AGCTCTCGTT CCTCTGGCTG GCGCACGTGT AGCAGCAGCC | 240 |
| GCAGACGCCC TGCACGATGC TCCCCGGGCA GTTCCTGGGC TCCTCGCACT TGGACTCGTC | 300 |
| ACAGGGCAGG CAGACCAGCG CCCGGGTGCC GGAGCGCGCC AGCAGCAGCA GCAGCCCCAG | 360 |
| CAGCGAGACC AGGAGGTGCC CGCAGCCGGC CAACCCCCTG TCCCCCGCCA CCAAGTACAT | 420 |
| CCTCCTGCGC CGCCGCCGCC TCCTCCTCGC AGCCGGGCCG GGAGCGGGGC GGGCGCCCTC | 480 |
| CCCTGCGCGG GGCACACGCG CCGCCGCCGC CGCACCAGCA GCCCGCGGTC CTCACCGCCC | 540 |
| CTCTCGGGGC CCCCGGGGCG CGCCTCCCCT CGCGGGGCGA GGCCCCCGCC CCTTCTGCGG | 600 |
| GCCGCGCCGA CCCCGAGCCC ACGAGCCTTG GCGCCGGCGG CAGCTTCCCC TCCTCCTCCT | 660 |
| CCTCCTCCTC CCGGGAGGGA GGGGGAAAAA AGAAAAAAGT TTCCTCCCGG CAGCTCCGGT | 720 |
| TCAACCCAAA CTTCTGGCGC GGCGGCGGCG GTGGCTGCTG CGCTCGGCTC CAGCCCGGGC | 780 |
| CGGCGGCGCC TCCTCCCTCT CCTCCTCCGA GTCGGCCGGC CCCGCAGCGG CGCAGCCTCC | 840 |
| GGGCCGGTCC CCGCCTCCCG AGCTGCCGAG TGGGCGCGGT GGCGCAGCAC AAGATCCGCG | 900 |
| GCGTCCGCTC CGCGCGCCCC GCTCGCCTCA CTCCTGCGCC GCTCCTCCGG GCGCTTGTTT | 960 |
| ATGGCTGGAG CCTCAGCCGC TCGGGCTGCG CCCTCCCCCA TCCTACCTCC TCCCCCAGAC | 1020 |
| CTTCCCCCCA CCCCCACGCG CCGCGCGCCG CTCATTGGCT GCCCCCCCTC CCCGGCCCGG | 1080 |
| CCGGCCCCCT CCGCCTCCCC CTCCCCCTCT CGGGCGGCCG GGCCCTTCCT CCCTCCCTCA | 1140 |
| CACGCCTCCA CCTCTTCCCG ATCTCCTCCT CCCCGAGCCC GGCGCACCGA GCCGGCCGTG | 1200 |
| CCACCGAGCT GCGGCTCTGG CCCCGGCGCC GCGGGTGCGC TGCGGATGGG CTTGGGGCGC | 1260 |
| ACCCAGCGAG CAGCGAGAGT CGCGGTGTCC CGGGCGCTCG CTGGCACCGT GGCCGCAGCG | 1320 |
| GCCGGCCTGG GAGCCAGGAG GGCGAGGCGG CTGCACCTTC GGGGCCAGAT TGGAGTTCGA | 1380 |
| AGAGTGGCGG GTACCCCAGA AGCTCGGGGC CGGGGCGATG GCTGCAGCCT CGGGAGGGTA | 1440 |
| TCGCCGGATC GAACTCCGGG AAAGGGAAGC AAAGGCATGG AACCTCCGCA CACTGGATGA | |
| predicted ACK4 gene seq (predicted start/stop codons underlined) | |
| ATGCCCCCGG AACAGCATCA TCAGCCCAAC AAAGTCTCGC CCAAACTTTG TTGGGCACAA | 60 |
| CCAGCGCCGA GGGGGCGGCG CAGGCCAGGT GGGAGGGGGC CCGCAGCGGG CGGCCGTACC | 120 |
| TTCGCAAACG CCCGCTTCGT ACTCGGTGAG GGAGTCGCCA TTGAGCGGGG GGCGGATGAC | 180 |
| ACAACGCAGC CCCCGGTCGC AGGTTCCGTA AATCCCGAAG GTGCCGCCGC AGCTCTCGTT | 240 |
| CCTCTGGCTG GCGCACGTGT AGCAGCAGCC GCAGACGCCC TGCACGATGC TCCCCGGGCA | 300 |
| GTTCCTGGGC TCCTCGCACT TGGACTCGTC ACAGGGCAGG CAGACCAGCG CCCGGGTGCC | 360 |
| GGAGCGCGCC AGCAGCAGCA GCAGCCCCAG CAGCGAGACC AGGAGGTGCC CGCAGCCGGC | 420 |
| CAACCCCCTG TCCCCCGCCA CCAAGTACAT CCTCCTGCGC CGCCGCCGCC TCCTCCTCGC | 480 |
| AGCCGGGCCG GGAGCGGGGC GGGCGCCCTC CCCTGCGCGG GGCACACGCG CCGCCGCCGC | 540 |
| CGCACCAGCA GCCCGCGGTC CTCACCGCCC CTCTCGGGGC CCCCGGGGCG CGCCTCCCCT | 600 |
| CGCGGGGCGA GGCCCCCGCC CCTTCTGCGG GCCGCGCCGA CCCCGAGCCC ACGAGCCTTG | 660 |
| GCGCCGGCGG CAGCTTCCCC TCCTCCTCCT CCTCCTCCTC CCGGGAGGGA GGGGGAAAAA | 720 |
| AGAAAAAAGT TTCCTCCCGG CAGCTCCGGT TCAACCCAAA CTTCTGGCGC GGCGGCGGCG | 780 |
| GTGGCTGCTG CGCTCGGCTC CAGCCCGGGC CGGCGGCGCC TCCTCCCTCT CCTCCTCCGA | 840 |
| GTCGGCCGGC CCCGCAGCGG CGCAGCCTCC GGGCCGGTCC CCGCCTCCCG AGCTGCCGAG | 900 |
| TGGGCGCGGT GGCGCAGCAC AAGATCCGCG GCGTCCGCTC CGCGCGCCCC GCTCGCCTCA | 960 |
| CTCCTGCGCC GCTCCTCCGG GCGCTTGTTT ATGGCTGGAG CCTCAGCCGC TCGGGCTGCG | 1020 |
| CCCTCCCCCA TCCTACCTCC TCCCCCAGAC CTTCCCCCCA CCCCCACGCG CCGCGCGCCG | 1080 |
| CTCATTGGCT GCCCCCCCTC CCCGGCCCGG CCGGCCCCCT CCGCCTCCCC CTCCCCCTCT | 1140 |
| CGGGCGGCCG GGCCCTTCCT CCCTCCCTCA CACGCCTCCA CCTCTTCCCG ATCTCCTCCT | 1200 |
| CCCCGAGCCC GGCGCACCGA GCCGGCCGTG CCACCGAGCT GCGGCTCTGG CCCCGGCGCC | 1260 |
| GCGGGTGCGC TGCGGATGGG CTTGGGGCGC ACCCAGCGAG CAGCGAGAGT CGCGGTGTCC | 1320 |
| CGGGCGCTCG CTGGCACCGT GGCCGCAGCG GCCGGCCTGG GAGCCAGGAG GGCGAGGCGG | 1380 |
| CTGCACCTTC GGGGCCAGAT TGGAGTTCGA AGAGTGGCGG GTACCCCAGA AGCTCGGGGC | 1440 |
| CGGGGCGATG GCTGCAGCCT CGGGAGGGTA TCGCCGGATC GAACTCCGGG AAAGGGAAGC | 1500 |
| AAAGGCATGG AACCTCCGCA CACTGGATGA | |

AAA8 DNA sequence
Gene name: ETL protein, with extended open reading frame
Unigene number: Hs.57958
Probeset Accession #: D58024
Nucleotide Accession #: AF192403
Coding sequence: 151-2136. Underlined sequences correspond to extended sequence
not included in AF192403.

| | |
|---|---|
| ATGAAAACAG CCGCACTCAC TCCGCCGCGC TCTCCGCCAC CGCCACCACT GCGGCCACCG | 60 |
| CCAATGAAAC GCCTCCCGCT CCTAGTGGTT TTTTCCACTT TGTTGAATTG TTCCTATACT | 120 |
| CAAAATTGCA CCAAGACACC TTGTCTCCCA AATGCAAAAT GTGAAATACG CAATGGAATT | 180 |
| GAAGCCTGCT ATTGCAACAT GGGATTTTCA GGAAATGGTG TCACAATTTG TGAAGATGAT | 240 |
| AATGAATGTG GAAATTTAAC TCAGTCCTGT GGCGAAAATG CTAATTGCAC TAACACAGAA | 300 |
| GGAAGTTATT ATTGTATGTG TGTACCTGGC TTCAGATCCA GCAGTAACCA AGACAGGTTT | 360 |
| ATCACTAATG ATGGAACCGT CTGTATAGAA AATGTGAATG CAAACTGCCA TTTAGATAAT | 420 |
| GTCTGTATAG CTGCAAATAT TAATAAAACT TTAACAAAAA TCAGATCCAT AAAAGAACCT | 480 |
| GTGGCTTTGC TACAAGAAGT CTATAGAAAT TCTGTGACAG ATCTTTCACC AACAGATATA | 540 |
| ATTACATATA TAGAAATATT AGCTGAATCA TCTTCATTAC TAGGTTACAA GAACAACACT | 600 |
| ATCTCAGCCA AGGACACCCT TTCTAACTCA ACTCTTACTG AATTTGTAAA AACCGTGAAT | 660 |
| AATTTTGTTC AAAGGGATAC ATTTGTAGTT TGGGACAAGT TATCTGTGAA TCATAGGAGA | 720 |
| ACACATCTTA CAAAACTCAT GCACACTGTT GAACAAGCTA CTTTAAGGAT ATCCCAGAGC | 780 |
| TTCCAAAAGA CCACAGAGTT TGATACAAAT TCAACGGATA TAGCTCTCAA AGTTTTCTTT | 840 |
| TTTGATTCAT ATAACATGAA ACATATTCAT CCTCATATGA ATATGGATGG AGACTACATA | 900 |
| AATATATTTC CAAAGAGAAA AGCTGCATAT GATTCAAATG GCAATGTTGC AGTTGCATTT | 960 |
| TTATATTATA AGAGTATTGG TCCTTTGCTT TCATCATCTG ACAACTTCTT ATTGAAACCT | 1020 |
| CAAAATTATG ATAATTCTGA AGAGGAGGAA AGAGTCATAT CTTCAGTAAT TTCAGTCTCA | 1080 |
| ATGAGCTCAA ACCCACCCAC ATTATATGAA CTTGAAAAAA TAACATTTAC ATTAAGTCAT | 1140 |
| CGAAAGGTCA CAGATAGGTA TAGGAGTCTA TGTGCATTTT GGAATTACTC ACCTGATACC | 1200 |
| ATGAATGGCA GCTGGTCTTC AGAGGGCTGT GAGCTGACAT ACTCAAATGA GACCCACACC | 1260 |
| TCATGCCGCT GTAATCACCT GACACATTTT GCAATTTTGA TGTCCTCTGG TCCTTCCATT | 1320 |
| GGTATTAAAG ATTATAATAT TCTTACAAGG ATCACTCAAC TAGGAATAAT TATTTCACTG | 1380 |
| ATTTGTCTTG CCATATGCAT TTTTACCTTC TGGTTCTTCA GTGAAATTCA AAGCACCAGG | 1440 |
| ACAACAATTC ACAAAAATCT TTGCTGTAGC CTATTTCTTG CTGAACTTGT TTTTCTTGTT | 1500 |
| GGGATCAATA CAAATACTAA TAAGCTCNTT TCTGTTTCAA TCATTGCCGG ACTGCTACAC | 1560 |
| TACTTCTTTT TAGCTGCTTT TGCATGGATG TGCATTGAAG GCATACATCT CTATCTCATT | 1620 |
| GTTGTGGGTG TCATCTACAA CAAGGGATTT TTGCACAAGA ATTTTTATAT CTTTGGCTAT | 1680 |
| CTAAGCCCAG CCGTGGTAGT TGGATTTTCG GCAGCACTAG GATACAGATA TTATGGCACA | 1740 |
| ACAAAAGTAT GTTGGCTTAG CACCGAAACA CACTTTATTT GGAGTTTTAT AGGACCAGCA | 1800 |
| TGCCTAATCA TTCTTGTTAA TCTCTTGGCT TTTGGAGTCA TCATATACAA AGTTTTTCGT | 1860 |
| CACACTGCAG GGTTGAAACC AGAAGTTAGT TGCTTTGAGA ACATAAGGTC TTGTGCAAGA | 1920 |
| GGAGCCCTCG CTCTTCTGTT CCTTCTCGGC ACCACCTGGA TCTTTGGGGT TCTCCATGTT | 1980 |
| GTGCACGCAT CAGTGGTTAC AGCTTACCTC TTCACAGTCA GCAATGCTTT CCAGGGGATG | 2040 |
| TTCATTTTTT TATTCCTGTG TGTTTTATCT AGAAAGATTC AAGAAGAATA TTACAGATTG | 2100 |
| TTCAAAAATG TCCCCTGTTG TTTTGGATGT TTAAGGTAAA CATAGAGAAT GGTGGATAAT | 2160 |
| TACAACTGCA CTAAAAATAA AAATTCCAAG CTGTGGATGA CCAATGTATA AAAATGACTC | 2220 |
| ATCAAATTAT CCAATTATTA ACTACTAGAC AAAAAGTATT TTAAATCAGT TTTTCTGTTT | 2280 |
| ATGCTATAGG AACTGTAGAT AATAAGGTAA AATTATGTAT CATATAGATA TACTATGTTT | 2340 |
| TTCTATGTGA AATAGTTCTG TCAAAAATAG TATTGCAGAT ATTTGGAAAG TAATTGGTTT | 2400 |
| CTCAGGAGTG ATATCACTGC ACCCAAGGAA AGATTTTCTT TCTAACACGA GAAGTATATG | 2460 |
| AATGTCCTGA AGGAAACCAC TGGCTTGATA TTTCTGTGAC TCGTGTTGCC TTTGAAACTA | 2520 |
| GTCCCCTACC ACCTCGGTAA TGAGCTCCAT TACAGAAAGT GGAACATAAG AGAATGAAGG | 2580 |
| GGCAGAATAT CAAACAGTGA AAAGGGAATG ATAAGATGTA TTTTGAATGA ACTGTTTTTT | 2640 |
| CTGTAGACTA GCTGAGAAAT TGTTGACATA AAATAAAGAA TTGAAGAAAC ACATTTTACC | 2700 |
| ATTTTGTGAA TTGTTCTGAA CTTAAATGTC CACTAAAACA ACTTAGACTT CTGTTTGCTA | 2760 |
| AATCTGTTTC TTTTTCTAAT ATTCTAAAAA AAAAAAAAAG GTTTMCCYCC CAAATTGAAA | 2820 |
| AAAAAAGGGA AAAAAAAATC TGTTTCTAAG GTTAGACTGA GATATATACT ATTTCCTTAC | 2880 |
| TTATTTCACA GATTGTGACT TTGGATAGTT AATCAGTAAA ATATAAATGT GTCGA | |

AAC6 DNA sequence
Gene name: Homo sapiens cDNA FLJ13465 fis, clone PLACE1003493, weakly similar to
endothelial cell multimerin precursor
Unigene number: Hs.134797
Probeset Accession #: AA025351
Nucleotide Accession #: AK023527
Coding sequence: predicted 75-2921
Extended sequence: 729-3465 (underlined sequence)

| | |
|---|---|
| AAGACAACGT CACTAGCAGT TTCTGGAGCT ACTTGCCAAG GCTGAGTGTG AGCTGAGCCT | 60 |
| GCCCCACCAC CAAGATGATC CTGAGCTTGC TGTTCAGCCT TGGGGGCCCC CTGGGCTGGG | 120 |
| GGCTGCTGGG GGCATGGGCC CAGGCTTCCA GTACTAGCCT CTCTGATCTG CAGAGCTCCA | 180 |
| GGACACCTGG GGTCTGGAAG GCAGAGGCTG AGGACACCAG CAAGGACCCC GTTGGACGTA | 240 |
| ACTGGTGCCC CTACCCAATG TCCAAGCTGG TCACCTTACT AGCTCTTTGC AAAACAGAGA | 300 |
| AATTCCTCAT CCACTCGCAG CAGCCGTGTC CGCAGGGAGC TCCAGACTGC CAGAAAGTCA | 360 |
| AAGTCATGTA CCGCATGGCC CACAAGCCAG TGTACCAGGT CAAGCAGAAG GTGCTGACCT | 420 |
| CTTTGGCCTG GAGGTGCTGC CCTGGCTACA CGGGCCCCAA CTGCGAGCAC CACGATTCCA | 480 |
| TGGCAATCCC TGAGCCTGCA GATCCTGGTG ACAGCCACCA GGAACCTCAG GATGGACCAG | 540 |
| TCAGCTTCAA ACCTGGCCAC CTTGCTGCAG TGATCAATGA GGTTGAGGTG CAACAGGAAC | 600 |
| AGCAGGAACA TCTGCTGGGA GATCTCCAGA ATGATGTGCA CCGGGTGGCA GACAGCCTGC | 660 |
| CAGGCCTGTG GAAAGCCCTG CCTGGTAACC TCACAGCTGC AGTGATGGAA GCAAATCAAA | 720 |
| CAGGGCACGA GTTCCCTGAT AGATCCTTGG AGCAGGTGCT GCTACCCCAC GTGGACACCT | 780 |
| TCCTACAAGT GCATTTCAGC CCCATCTGGA GGAGCTTTAA CCAAAGCCTG CACAGCCTTA | 840 |
| CCCAGGCCAT AAGAAACCTG TCTCTTGACG TGGAGGCCAA CCGCCAGGCC ATCTCCAGAG | 900 |
| TCCAGGACAG TGCCGTGGCC AGGGCTGACT TCCAGGAGCT TGGTGCCAAA TTTGAGGCCA | 960 |
| AGGTCCAGGA GAACACTCAG AGAGTGGGTC AGCTGCGACA GGACGTGGAG GACCGCCTGC | 1020 |
| ACGCCCAGCA CTTTACCCTG CACCGCTCGA TCTCAGAGCT CCAAGCCGAT GTGGACACCA | 1080 |
| AATTGAAGAG GCTGCACAAG GCTCAGGAGG CCCCAGGGAC CAATGGCAGT CTGGTGTTGG | 1140 |
| CAACGCCTGG GGCTGGGGCA AGGCCTGAGC CGGACAGCCT GCAGGCCAGG CTGGGCCAGC | 1200 |
| TGCAGAGGAA CCTCTCAGAG CTGCACATGA CCACGGCCCG CAGGGAGGAG GAGTTGCAGT | 1260 |
| ACACCCTGGA GGACATGAGG GCCACCCTGA CCCGGCACGT GGATGAGATC AAGGAACTGT | 1320 |
| ACTCCGAATC GGACGAGACT TTCGATCAGA TTAGCAAGGT GGAGCGGCAG GTGGAGGAGC | 1380 |
| TGCAGGTGAA CCACACGGCG CTCCGTGAGC TGCGCGTGAT CCTGATGGAG AAGTCTCTGA | 1440 |
| TCATGGAGGA GAACAAGGAG GAGGTGGAGC GGCAGCTCCT GGAGCTCAAC CTCACGCTGC | 1500 |
| AGCACCTGCA GGGTGGCCAT GCCGACCTCA TCAAGTACGT GAAGGACTGC AATTGCCAGA | 1560 |
| AGCTCTATTT AGACCTGGAC GTCATCCGGG AGGGCCAGAG GGACGCCACG CGTGCCCTGG | 1620 |
| AGGAGACCCA GGTGAGCCTG GACGAGCGGC GGCAGCTGGA CGGCTCCTCC CTGCAGGCCC | 1680 |
| TGCAGAACGC CGTGGACGCC GTGTCGCTGG CCGTGGACGC GCACAAAGCG GAGGGCGAGC | 1740 |
| GGGCGCGGGC GGCCACGTCG CGGCTCCGGA GCCAAGTGCA GGCGCTGGAT GACGAGGTGG | 1800 |
| GCGCGCTGAA GGCGGCCGCG GCCGAGGCCC GCCACGAGGT GCGCCAGCTG CACAGCGCCT | 1860 |
| TCGCCGCCCT GCTGGAGGAC GCGCTGCGGC ACGAGGCGGT GCTGGCCGCG CTCTTCGGGG | 1920 |
| AGGAGGTGCT GGAGGAGATG TCTGAGCAGA CGCCGGGACC GCTGCCCCTG AGCTACGAGC | 1980 |
| AGATCCGCGT GGCCCTGCAG GACGCCGCTA GCGGGCTGCA GGAGCAGGCG CTCGGCTGGG | 2040 |
| ACGAGCTGGC CGCCCGAGTG ACGGCCCTGG AGCAGGCCTC GGAGCCCCCG CGGCCGGCAG | 2100 |
| AGCACCTGGA GCCCAGCCAC GACGCGGGCC GCGAGGAGGC CGCCACCACC GCCCTGGCCG | 2160 |
| GGCTGGCGCG GGAGCTCCAG AGCCTGAGCA ACGACGTCAA GAATGTCGGG CGGTGCTGCG | 2220 |
| AGGCYGAGGC CGGGGCCGGG GCCGCCTCCC TCAACGCCTC CCTTGACGGC CTCCACAACG | 2280 |
| CACTCTTCGC CACTCAGCGC AGCTTGGAGC AGCACCAGCG GCTCTTCCAC AGCCTCTTTG | 2340 |
| GGAACTTCCA AGGGCTCATG GAAGCCAACG TCAGCCTGGA CCTGGGGAAG CTGCAGACCA | 2400 |
| TGCTGAGCAG GAAAGGGAAA AAGCAGCAGA AAGACCTGGA AGCTCCCCGG AAGAGGGACA | 2460 |
| AGAAGGAAGC GGAGCCTTTC GTGGACATAC GGGTCACAGG GCCTGTGCCA GGTGCCTTGG | 2520 |
| GCGCGGCGCT CTGGGAGGCA GRWTCCCCTG TGGCCTTCTA TGCCAGCTTT TCAGAAGGGA | 2580 |
| CGGCTGCCCT GCAGACAGTG AAGTTCAACA CCACATACAT CAACATTGGC AGCAGCTACT | 2640 |
| TCCCTGAACA TGGCTACTTC CGAGCCCCTG AGCGTGGTGT CTACCTGTTT GCAGTGAGCG | 2700 |
| TTGAATTTGG CCCAGGGCCA GGCACCGGGC AGCTGGTGTT TGGAGGTCAC CATCGGACTC | 2760 |
| CAGTCTGTAC CACTGGGCAG GGGAGTGGAA GCACAGCAAC GGTCTTTGCC ATGGCTGAGC | 2820 |
| TGCAGAAGGG TGAGCGAGTA TGGTTTGAGT TAACCCAGGG ATCAATAACA AAGAGAAGCC | 2880 |
| TGTCGGGCAC TGCATTTGGG GGCTTCCTGA TGTTTAAGAC CTGAACCCCA GCCCCAATCT | 2940 |
| GATCAGACAT CATGGACTCG CCCAGCTCTC CTCGGCCTGG GGCTCTGGCC AAGGATGGGC | 3000 |
| TGGAGGTCAT TCAGTTGGTC TGTCTCTTCC CTGGAAACCT TCTGCAAAGA TGGTGTGGTG | 3060 |
| TACGTGGCTT CCCTGTAACC ACATGGGGCT TGGCCATTTC TCCATGATGA GAAGGACTGG | 3120 |
| AATGCTTCTC CGGGCAGGAC ATGGTCCTAG GAAGCCTGAA CCTTGGCTTG GCATGCCTTC | 3180 |
| TCAGACAGCA CGGCCTGGGC TCCAACTCTT CACCACACCC TGTATTCTAC AACTTCTTTG | 3240 |
| GTGTTTTGCT CCTCCTGTGG TTGGAAACTT CTGTACAACA CTTTAAACTT TTCTCTTGCT | 3300 |
| TCCTCTTCTC TTCTCCCTTA TCGTATGATA GAAAGACATT CTTCCCCAGG AGGAATGTTT | 3360 |
| AAAATGGAGG CAACATTTTG GCCAACATTG GAAAGCACTA GAGGGCAATG GGATTAAACC | 3420 |
| AACCTGCTTG GTCTCTATTA GTCAGTAATG AAGACGACAG CCTGGCCAAC CAAGGGAAAC | 3480 |
| TCTGATGATT TTATAAGTTT GATAGTTCCT CCTGTGTTCA TTCTCCTTCC TGCCACCTTG | 3720 |
| TGAAGATGCC TTGGTTCCTC TTCACTGTCT GCCATGATTG TAAGTTTCCT GAGGCCTCCC | 3780 |
| CAGCCATGTG GAACAGTGAG TCAATTAAAC CTCTTTCCTT TATAAATT | |

| | |
|---|---|
| ACH7 DNA sequence | |
| Gene name: ESTs | |
| Unigene number: Hs.3807 | |
| Probeset Accession #: AA292694 | |
| BAC Accession #: | ALI161751 |
| FGENESH predicted exons: FGENESH predicts 2 exons on the minus strand of AL161751 | |
| upstream of the ACH7 probeset. | |
| FGENESH predicted exon 1: | |

| | |
|---|---|
| ATGGGCAAAG ACTTCATGAC TAAAACACCA AAAGCATTTG CAACAAAAGC CAAAATTGAC | 60 |
| AAATGGGATC TAATTAAACT AAAGAGCTTC TGCACAGCAA AAGAAACTAT CATCAGAGTG | 120 |
| AACAGTCAAC CTACAGACTG GCAGAAAACT TTTGCAATCT ATCCATCTGA CAAAGGGGTA | 180 |
| ATAGCCAGAA TCTACAAGGA GCTTGAACAA ATTTATAAGA AAAAAAAACC AACAAAAA | |
| FGENESH predicted exon 2: | |
| CGCTCCGCAC ACATTTCCTG TCGCGGCCTA AGGGAAACTG TTGGCCGCTG GGCCCGCGGG | 60 |
| GGGATTCTTG GCAGTTGGGG GGTCCGTCGG GAGCGAGGGC GGAGGGGAAG GGAGGGGGAA | 120 |
| CCGGGTTGGG GAAGCCAGCT GTAGAGGGCG GTGACCGCGC TCCAGACACA GCTCTGCGTC | 180 |
| CTCGAGCGGG ACAGATCCAA GTTGGGAGCA GCTCTGCGTG CGGGGCCTCA GAGAATGAGG | 240 |
| CCGGCGTTCG CCCTGTGCCT CCTCTGGCAG GCGCTCTGGC CCGGGCCGGG CGGCGGCGAA | 300 |
| CACCCCACTG CCGACCGTGC TGGCTGCTCG GCCTCGGGGG CCTGCTACAG CCTGCACCAC | 360 |
| GCTACCATGA AGCGGCAGGC GGCCGAGGAG GCCTGCATCC TGCGAGGTGG GGCGCTCAGC | 420 |
| ACCGTGCGTG CGGGCGCCGA GCTGCGCGCT GTGCTCGCGC TCCTGCGGGC AGGCCCAGGG | 480 |
| CCCGGAGGGG GCTCCAAAGA CCTGCTGTTC TGGGTCGCAC TGGAGCGCAG GCGTTCCCAC | 540 |
| TGCACCCTGG AGAACGAGCC TTTGCGGGGT TTCTCCTGGC TGTCCTCCGA CCCCGGCGGT | 600 |
| CTCGAAAGCG ACACGCTGCA GTGGGTGGAG GAGCCCCAAC GCTCCTGCAC CGCGCGGAGA | 660 |
| TGCGCGGTAC TCCAGGCCAC CGGTGGGGTC GAGCCCGCAG CTGGAAGGAG ATGCGATGCC | 720 |
| ACCTGCGCGC CAACGGCTAC CTGTGCAAGT ACCAGTTTGA GGTCTTGTGT CCTGCGCCGC | 780 |
| GCCCCGGGGC CGCCTCTAAC TTGAGCTATC GCGCGCCCTT CCAGCTGCAC AGCGCCGCTC | 840 |
| TGGACTTGAG TCCACCTGGG ACCGAGGTGA GTGCGCTCTG CCGGGGACAG CTCCCGATCT | 900 |
| CAGTTACTTG CATCGCGGAC GAAATCGGCG CTCGCTGGGA CAAACTCTCG GGCGATGTGT | 960 |
| TGTGTCCCTG CCCCGGGAGG TACCTCCGTG CTGGCAAATG CGCAGAGCTC CCTAACTGCC | 1020 |
| TAGACGACTT GGGAGGCTTT GCCTGCGAAT GTGCTACGGG CTTCGAGCTG GGGAAGGACG | 1080 |
| GCCGCTCTTG TGTGACCAGT GGGGAAGGAC AGCCGACCCT TGGGGGGACC GGGGTGCCCA | 1140 |
| CCAGGCGCCC GCCGGCCACT GCAACCAGCC CCGTGCCGCA GAGAACATGG CCAATCAGGG | 1200 |
| TCGACGAGAA GCTGGGAGAG ACACCACTTG TCCCTGAACA AGACAATTCA GTAACATCTA | 1260 |
| TTCCTGAGAT TCCTCGATGG GGATCACAGA GCACGATGTC TACCCTTCAA ATGTCCCTTC | 1320 |
| AAGCCGAGTC AAAGGCCACT ATCACCCCAT CAGGGAGCGT GATTTCCAAG TTTAATTCTA | 1380 |
| CGACTTCCTC TGCCACTCCT CAGGCTTTCG ACTCCTCCTC TGCCGTGGTC TTCATATTTG | 1440 |
| TGAGCACAGC AGTAGTAGTG TTGGTGATCT TGACCATGAC AGTACTGGGG CTTGTCAAGC | 1500 |
| TCTGCTTTCA CGAAAGCCCC TCTTCCCAGC CAAGGAAGGA GTCTATGGGC CCGCCGGGCC | 1560 |
| TGGAGAGTGA TCCTGAGCCC GCTGCTTTGG GCTCCAGTTC TGCACATTGC ACAAACAATG | 1620 |
| GGGTGAAAGT CGGGGACTGT GATCTGCGGG ACAGAGCAGA AGGTGCCTTG CTGGCGGAGT | 1680 |
| CCCCTCTTGG CTCTAGTGAT GCATAG | |

ACH7 predicted coding seq (predicted start/stop codons underlined)

| | |
|---|---|
| ATGGGCAAAG ACTTCATGAC TAAAACACCA AAAGCATTTG CAACAAAAGC CAAAATTGAC | 60 |
| AAATGGGATC TAATTAAACT AAAGAGCTTC TGCACAGCAA AAGAAACTAT CATCAGAGTG | 120 |
| AACAGTCAAC CTACAGACTG GCAGAAAACT TTTGCAATCT ATCCATCTGA CAAAGGGGTA | 180 |
| ATAGCCAGAA TCTACAAGGA GCTTGAACAA ATTTATAAGA AAAAAAAACC AACAAAAACG | 240 |
| CTCCGCACAC ATTTCCTGTC GCGGCCTAAG GGAAACTGTT GGCCGCTGGG CCCGCGGGGG | 300 |
| GATTCTTGGC AGTTGGGGGG TCCGTCGGGA GCGAGGGCGG AGGGGAAGGG AGGGGGAACC | 360 |
| GGGTTGGGGA AGCCAGCTGT AGAGGGCGGT GACCGCGCTC CAGACACAGC TCTGCGTCCT | 420 |
| CGAGCGGGAC AGATCCAAGT TGGGAGCAGC TCTGCGTGCG GGGCCTCAGA GAATGAGGCC | 480 |
| GGCGTTCGCC CTGTGCCTCC TCTGGCAGGC GCTCTGGCCC GGGCCGGGCG GCGGCGAACA | 540 |
| CCCCACTGCC GACCGTGCTG GCTGCTCGGC CTCGGGGGCC TGCTACAGCC TGCACCACGC | 600 |
| TACCATGAAG CGGCAGGCGG CCGAGGAGGC CTGCATCCTG CGAGGTGGGG CGCTCAGCAC | 660 |
| CGTGCGTGCG GGCGCCGAGC TGCGCGCTGT GCTCGCGCTC CTGCGGGCAG GCCCAGGGCC | 720 |
| CGGAGGGGGC TCCAAAGACC TGCTGTTCTG GGTCGCACTG GAGCGCAGGC GTTCCCACTG | 780 |
| CACCCTGGAG AACGAGCCTT TGCGGGGTTT CTCCTGGCTG TCCTCCGACC CCGGCGGTCT | 840 |
| CGAAAGCGAC ACGCTGCAGT GGGTGGAGGA GCCCCAACGC TCCTGCACCG CGCGGAGATG | 900 |
| CGCGGTACTC CAGGCCACCG GTGGGGTCGA GCCCGCAGCT GGAAGGAGAT GCGATGCCAC | 960 |
| CTGCGCGCCA ACGGCTACCT GTGCAAGTAC CAGTTTGAGG TCTTGTGTCC TGCGCCGCGC | 1020 |
| CCCGGGGCCG CCTCTAACTT GAGCTATCGC GCGCCCTTCC AGCTGCACAG CGCCGCTCTG | 1080 |
| GACTTCAGTC CACCTGGGAC CGAGGTGAGT GCGCTCTGCC GGGGACAGCT CCCGATCTCA | 1140 |
| GTTACTTGCA TCGCGGACGA AATCGGCGCT CGCTGGGACA AACTCTCGGG CGATGTGTTG | 1200 |
| TGTCCCTGCC CCGGGAGGTA CCTCCGTGCT GGCAAATGCG CAGAGCTCCC TAACTGCCTA | 1260 |
| GACGACTTGG GAGGCTTTGC CTGCGAATGT GCTACGGGCT TCGAGCTGGG GAAGGACGGC | 1320 |
| CGCTCTTGTG TGACCAGTGG GGAAGGACAG CCGACCCTTG GGGGGACCGG GGTGCCCACC | 1380 |
| AGGCGCCCGC CGGCCACTGC AACCAGCCCC GTGCCGCAGA GAACATGGCC AATCAGGGTC | 1440 |
| GACGAGAAGC TGGGAGAGAC ACCACTTGTC CCTGAACAAG ACAATTCAGT AACATCTATT | 1500 |
| CCTGAGATTC CTCGATGGGG ATCACAGAGC ACGATGTCTA CCCTTCAAAT GTCCCTTCAA | 1560 |
| GCCGAGTCAA AGGCCACTAT CACCCCATCA GGGAGCGTGA TTTCCAAGTT TAATTCTACG | 1620 |
| ACTTCCTCTG CCACTCCTCA GGCTTTCGAC TCCTCCTCTG CCGTGGTCTT CATATTTGTG | 1680 |
| AGCACAGCAG TAGTAGTGTT GGTGATCTTG ACCATGACAG TACTGGGGCT TGTCAAGCTC | 1740 |
| TGCTTTCACG AAAGCCCCTC TTCCCAGCCA AGGAAGGAGT CTATGGGCCC GCCGGGCCTG | 1800 |
| GAGAGTGATC CTGAGCCCGC TGCTTTGGGC TCCAGTTCTG CACATTGCAC AAACAATGGG | 1860 |
| GTGAAAGTCG GGGACTGTGA TCTGCGGGAC AGAGCAGAGG GTGCCTTGCT GGCGGAGTCC | 1920 |
| CCTCTTGGCT CTAGTGATGC ATAG | |

AAD3 DNA sequence
Gene name: ESTs
Unigene number: Hs.17404
Probeset Accession #: N39584
Nucleic Acid Accession #: N39584
Coding sequence: no identified ORF; possible frameshifts

| | |
|---|---|
| AAATGGGATT GAGTTAAAAC TATTTTATTT TAAATATACA TTTTAAAGCA GTTCTTTTTT | 60 |
| TTTTTTTTTT TTTTATTATA CACACACTTC AAGAGAATAT GCACAGTCTA GGCCGGGCAC | 120 |
| GGTGGCTCAC GCCTGTAATC CCAGCACTTT GGGAGGCCGA GGCATGTGGA TCACCTGAGG | 180 |
| TCAGGAGTTT GAGACCAGCC TAGACAACAT GGTGAAACCT TGTCTCTATG AAAAATACAA | 240 |
| AATTTGCTGG GAGTGGTGGT GCATGCCTGT AATCCCAGCT ACTTGGAAGG CTGAGGCAGG | 300 |
| AGAATGTCTT GAACCTAGGA GGTGGAGGTT GCAGTGAGCT GAGATTGCAC CATTGCACTC | 360 |
| CAGCCTGTGC AACAAAAGTG AAACTCCATT TCAAGAAAAA AAAAAAAAAA AGAATATGCA | 420 |
| CAGTCTGAAT GTATACCAGG AGTGTGAGAG ACACATGCCC ACTTCATGCA ACTCCTAAAC | 480 |
| TCAAAGTCTA AATCAGATAT TTTTATTAAC AATGACAACT TGTTGCCAAC TCCCTGTTTC | 540 |
| TAATCACCAA AGACCCAGGG TACCTAAAAG GACTTTGCAA CCAAGCAAAG TCACTGTCTT | 600 |
| CAAATCTGGA TACACACTTT CCCCTCTGTA GATTCAAAAG GTGCTTCCTT CCCGGCTGTC | 660 |
| TCCAGCTTCC TTACTCTCTT TTCTGGGATT TCTTTTTCTT CTTTCTTTCT GGCTCTTCCT | 720 |
| CCACTGGCTG AACTGGGTCC CCTAACTGAA ACAGCCCCTG ACTTAGCCCA AGCATGCTTC | 780 |
| CTTTAGCTGC TGTGAGAATT TTGTCTTCCT CACCAGCCAG GTCCTCAAGG CAAAGTCCTC | 840 |
| AGCCAGTGCT TTAAGAGCAA CTTCCCGCAA ATCAGAAACT CACTGTGATT CCAAAAATGT | 900 |
| TTCTGAGCCC TGGACCCCTG CCCCCAAAAT ATTTTCATCT TTCCCCCAAA CCTCCTTTAA | 960 |
| AGGAGCATGC ATAACAGTGT GCTGAAAGAC AGTTGTTGGT TTTTTGATTT TAGCATATTA | 1020 |
| TTTCCTGTAT GAAATATGTT TTATATAATC TCCTATTATT TTTATCTTAT GTTTTGTATT | 1080 |
| GTTGATAAAT CCCTTTTTGT CCTTCTAAGA TGTTCTATTG TAAAATCACT TATAAGGTAT | 1140 |
| GATTACTCTT TATGCTATTA CTTTATATGC CATTTGGGTA ATAAATAGTA AATGGTTGAT | 1200 |
| GATATGATTG ACTGATGCGC AGTCCAGAGC ATGTATGAAT AATCTCATAA AACAGTATCA | 1260 |
| CAGACATTAA GCTAAACTGT TTCGTTTTTT TGAAAGAACA ACTCATACTT TGGAACAGTT | 1320 |
| GTCAATATTA ATTTGTTGCA AATATTTAAT TTAAATAAAC ATTTTTGTAC CATGAAAAAA | 1380 |

AAD4 DNA sequence
Gene name: ERG
Unigene number: Hs.279477 / Hs.45514
Probeset Accession #: R32894
Nucleic Acid Accession #: M17254
Coding sequence: 257-1645 (predicted start/stop codons underlined)

| | |
|---|---|
| GTCCGCGCGT GTCCGCGCCC GCGTGTGCCA GCGCGCGTGC CTTGGCCGTG CGCGCCGAGC | 60 |
| CGGGTCGCAC TAACTCCCTC GGCGCCGACG GCGGCGCTAA CCTCTCGGTT ATTCCAGGAT | 120 |
| CTTTGGAGAC CCGAGGAAAG CCGTGTTGAC CAAAAGCAAG ACAAATGACT CACAGAGAAA | 180 |
| AAAGATGGCA GAACCAAGGG CAACTAAAGC CGTCAGGTTC TGAACAGCTG GTAGATGGGC | 240 |
| TGGCTTACTG AAGGACATGA TTCAGACTGT CCCGGACCCA GCAGCTCATA TCAAGGAAGC | 300 |
| CTTATCAGTT GTGAGTGAGG ACCAGTCGTT GTTTGAGTGT GCCTACGGAA CGCCACACCT | 360 |
| GGCTAAGACA GAGATGACCG CGTCCTCCTC CAGCGACTAT GGACAGACTT CCAAGATGAG | 420 |
| CCCACGCGTC CCTCAGCAGG ATTGGCTGTC TCAACCCCCA GCCAGGGTCA CCATCAAAAT | 480 |
| GGAATGTAAC CCTAGCCAGG TGAATGGCTC AAGGAACTCT CCTGATGAAT GCAGTGTGGC | 540 |
| CAAAGGCGGG AAGATGGTGG GCAGCCCAGA CACCGTTGGG ATGAACTACG GCAGCTACAT | 600 |
| GGAGGAGAAG CACATGCCAC CCCCAAACAT GACCACGAAC GAGCGCAGAG TTATCGTGCC | 660 |
| AGCAGATCCT ACGCTATGGA GTACAGACCA TGTGCGGCAG TGGCTGGAGT GGGCGGTGAA | 720 |
| AGAATATGGC CTTCCAGACG TCAACATCTT GTTATTCCAG AACATCGATG GGAAGGAACT | 780 |
| GTGCAAGATG ACCAAGGACG ACTTCCAGAG GCTCACCCCC AGCTACAACG CCGACATCCT | 840 |
| TCTCTCACAT CTCCACTACC TCAGAGAGAC TCCTCTTCCA CATTTGACTT CAGATGATGT | 900 |
| TGATAAAGCC TTACAAAACT CTCCACGGTT AATGCATGCT AGAAACACAG ATTTACCATA | 960 |
| TGAGCCCCCC AGGAGATCAG CCTGGACCGG TCACGGCCAC CCCACGCCCC AGTCGAAAGC | 1020 |
| TGCTCAACCA TCTCCTTCCA CAGTGCCCAA AACTGAAGAC CAGCGTCCTC AGTTAGATCC | 1080 |
| TTATCAGATT CTTGGACCAA CAAGTAGCCG CCTTGCAAAT CCAGGCAGTG GCCAGATCCA | 1140 |
| GCTTTGGCAG TTCCTCCTGG AGCTCCTGTC GGACAGCTCC AACTCCAGCT GCATCACCTG | 1200 |
| GGAAGGCACC AACGGGGAGT TCAAGATGAC GGATCCCGAC GAGGTGGCCC GGCGCTGGGG | 1260 |
| AGAGCGGAAG AGCAAACCCA ACATGAACTA CGATAAGCTC AGCCGCGCCC TCCGTTACTA | 1320 |
| CTATGACAAG AACATCATGA CCAAGGTCCA TGGGAAGCGC TACGCCTACA AGTTCGACTT | 1380 |
| CCACGGGATC GCCCAGGCCC TCCAGCCCCA CCCCCCGGAG TCATCTCTGT ACAAGTACCC | 1440 |
| CTCAGACCTC CCGTACATGG GCTCCTATCA CGCCCACCCA CAGAAGATGA ACTTTGTGGC | 1500 |
| GCCCCACCCT CCAGCCCTCC CCGTGACATC TTCCAGTTTT TTTGCTGCCC CAAACCCATA | 1560 |
| CTGGAATTCA CCAACTGGGG GTATATACCC CAACACTAGG CTCCCCACCA GCCATATGCC | 1620 |
| TTCTCATCTG GGCACTTACT ACTAAAGACC TGGCGGAGGC TTTTCCCATC AGCGTGCATT | 1680 |
| CACCAGCCCA TCGCCACAAA CTCTATCGGA GAACATGAAT CAAAAGTGCC TCAAGAGGAA | 1740 |
| TGAAAAAAGC TTTACTGGGG CTGGGGAAGG AAGCCGGGGA AGAGATCCAA AGACTCTTGG | 1800 |
| GAGGGAGTTA CTGAAGTCTT ACTACAGAAA TGAGGAGGAT GCTAAAAATG TCACGAATAT | 1860 |
| GGACATATCA TCTGTGGACT GACCTTGTAA AAGACAGTGT ATGTAGAAGC ATGAAGTCTT | 1920 |
| AAGGACAAAG TGCCAAAGAA AGTGGTCTTA AGAAATGTAT AAACTTTAGA GTAGAGTTTG | 1980 |
| AATCCCACTA ATGCAAACTG GGATGAAACT AAAGCAATAG AAACAACACA GTTTTGACCT | 2040 |
| AACATACCGT TTATAATGCC ATTTTAAGGA AAACTACCTG TATTTAAAAA TAGTTTCATA | 2100 |
| TCAAAAACAA GAGAAAAGAC ACGAGAGAGA CTGTGGCCCA TCAACAGACG TTGATATGCA | 2160 |
| ACTGCATGGC ATGTGCTGTT TTGGTTGAAA TCAAATACAT TCCGTTTGAT GGACAGCTGT | 2220 |
| CAGCTTTCTC AAACTGTGAA GATGACCCAA AGTTTCCAAC TCCTTTACAG TATTACCGGG | 2280 |
| ACTATGAACT AAAAGGTGGG ACTGAGGATG TGTATAGAGT GAGCGTGTGA TTGTAGACAG | 2340 |
| AGGGGTGAAG AAGGAGGAGG AAGAGGCAGA GAAGGAGGAG ACCAGGCTGG GAAAGAAACT | 2400 |
| TCTCAAGCAA TGAAGACTGG ACTGAGGACA TTTGGGGACT GTGTACAATG AGTTATGGAG | 2460 |
| ACTCGAGGGT TCATGCAGTC AGTGTTATAC CAAACCCAGT GTTAGGAGAA AGGACACAGC | 2520 |
| GTAATGGAGA AAGGGAAGTA GTAGAATTCA GAAACAAAAA TGCGCATCTC TTTCTTTGTT | 2580 |
| TGTCAAATGA AAATTTTAAC TGGAATTGTC TGATATTTAA GAGAAACATT CAGGACCTCA | 2640 |
| TCATTATGTG GGGGCTTTGT TCTCCACAGG GTCAGGTAAG AGATGGCCTT CTTGGCTGCC | 2700 |
| ACAATCAGAA ATCACGCAGG CATTTTGGGT AGGCGGCCTC CAGTTTTCCT TTGAGTCGCG | 2760 |
| AACGCTGTGC GTTTGTCAGA ATGAAGTATA CAAGTCAATG TTTTTCCCCC TTTTTATATA | 2820 |
| ATAATTATAT AACTTATGCA TTTATACACT ACGAGTTGAT CTCGGCCAGC CAAAGACACA | 2880 |
| CGACAAAAGA GACAATCGAT ATAATGTGGC CTTGAATTTT AACTCTGTAT GCTTAATGTT | 2940 |
| TACAATATGA AGTTATTAGT TCTTAGAATG GAGAATGTAT GTAATAAAAT AAGCTTGGCC | 3000 |
| TAGCATGGCA AATCAGATTT ATACAGGAGT CTGCATTTGC ACTTTTTTTA GTGACTAAAG | 3060 |
| TTGCTTAATG AAAACATGTG CTGAATGTTG TGGATTTTGT GTTATAATTT ACTTTGTCCA | 3120 |
| GGAACTTGTG CAAGGGAGAG CCAAGGAAAT AGGATGTTTG GCACCC | |

AAD5 DNA sequence
Gene name: activin A receptor type II-like 1 (ALK-1)
Unigene number: Hs.8881 / Hs.172670
Prabeset Accession #: T57112
Nucleic Acid Accession #: NM_000020
Coding sequence: 283-1794 (predicted start/stop codons underlined)

| | |
|---|---|
| AGGAAACGGT TTATTAGGAG GGAGTGGTGG AGCTGGGCCA GGCAGGAAGA CGCTGGAATA | 60 |
| AGAAACATTT TTGCTCCAGC CCCCATCCCA GTCCCGGGAG GCTGCCGCGC CAGCTGCGCC | 120 |
| GAGCGAGCCC CTCCCCGGCT CCAGCCCGGT CCGGGGCCGC GCCGGACCCC AGCCCGCCGT | 180 |
| CCAGCGCTGG CGGTGCAACT GCGGCCGCGC GGTGGAGGGG AGGTGGCCCC GGTCCGCCGA | 240 |
| AGGCTAGCGC CCCGCCACCC GCAGAGCGGG CCCAGAGGGA CCATGACCTT GGGCTCCCCC | 300 |
| AGGAAAGGCC TTCTGATGCT GCTGATGGCC TTGGTGACCC AGGGAGACCC TGTGAAGCCG | 360 |
| TCTCGGGGCC CGCTGGTGAC CTGCACGTGT GAGAGCCCAC ATTGCAAGGG GCCTACCTGC | 420 |
| CGGGGGGCCT GGTGCACAGT AGTGCTGGTG CGGGAGGAGG GGAGGCACCC CCAGGAACAT | 480 |
| CGGGGCTGCG GGAACTTGCA CAGGGAGCTC TGCAGGGGGC GCCCCACCGA GTTCGTCAAC | 540 |
| CACTACTGCT GCGACAGCCA CCTCTGCAAC CACAACGTGT CCCTGGTGCT GGAGGCCACC | 600 |
| CAACCTCCTT CGGAGCAGCC GGGAACAGAT GGCCAGCTGG CCCTGATCCT GGGCCCCGTG | 660 |
| CTGGCCTTGC TGGCCCTGGT GGCCCTGGGT GTCCTGGGCC TGTGGCATGT CCGACGGAGG | 720 |
| CAGGAGAAGC AGCGTGGCCT GCACAGCGAG CTGGGAGAGT CCAGTCTCAT CCTGAAAGCA | 780 |
| TCTGAGCAGG GCGACACGAT GTTGGGGGAC CTCCTGGACA GTGACTGCAC CACAGGGAGT | 840 |
| GGCTCAGGGC TCCCCTTCCT GGTGCAGAGG ACAGTGGCAC GGCAGGTTGC CTTGGTGGAG | 900 |
| TGTGTGGGAA AAGGCCGCTA TGGCGAAGTG TGGCGGGGCT TGTGGCACGG TGAGAGTGTG | 960 |
| GCCGTCAAGA TCTTCTCCTC GAGGGATGAA CAGTCCTGGT TCCGGGAGAC TGAGATCTAT | 1020 |
| AACACAGTAT TGCTCAGACA CGACAACATC CTAGGCTTCA TCGCCTCAGA CATGACCTCC | 1080 |
| CGCAACTCGA GCACGCAGCT GTGGCTCATC ACGCACTACC ACGAGCACGG CTCCCTCTAC | 1140 |
| GACTTTCTGC AGAGACAGAC GCTGGAGCCC CATCTGGCTC TGAGGCTAGC TGTGTCCGCG | 1200 |
| GCATGCGGCC TGGCGCACCT GCACGTGGAG ATCTTCGGTA CACAGGGCAA ACCAGCCATT | 1260 |
| GCCCACCGCG ACTTCAAGAG CCGCAATGTG CTGGTCAAGA GCAACCTGCA GTGTTGCATC | 1320 |
| GCCGACCTGG GCCTGGCTGT GATGCACTCA CAGGGCAGCG ATTACCTGGA CATCGGCAAC | 1380 |
| AACCCGAGAG TGGGCACCAA GCGGTACATG GCACCCGAGG TGCTGGACGA GCAGATCCGC | 1440 |
| ACGGACTGCT TTGAGTCCTA CAAGTGGACT GACATCTGGG CCTTTGGCCT GGTGCTGTGG | 1500 |
| GAGATTGCCC GCCGGACCAT CGTGAATGGC ATCGTGGAGG ACTATAGACC ACCCTTCTAT | 1560 |
| GATGTGGTGC CCAATGACCC CAGCTTTGAG GACATGAAGA AGGTGGTGTG TGTGGATCAG | 1620 |
| CAGACCCCCA CCATCCCTAA CCGGCTGGCT GCAGACCCGG TCCTCTCAGG CCTAGCTCAG | 1680 |
| ATGATGCGGG AGTGCTGGTA CCCAAACCCC TCTGCCCGAC TCACCGCGCT GCGGATCAAG | 1740 |
| AAGACACTAC AAAAAATTAG CAACAGTCCA GAGAAGCCTA AAGTGATTCA ATAGCCCAGG | 1800 |
| AGCACCTGAT TCCTTTCTGC CTGCAGGGGG CTGGGGGGGT GGGGGGCAGT GGATGGTGCC | 1860 |
| CTATCTGGGT AGAGGTAGTG TGAGTGTGGT GTGTGCTGGG GATGGGCAGC TGCGCCTGCC | 1920 |
| TGCTCGGCCC CCAGCCCACC CAGCCAAAAA TACAGCTGGG CTGAAACCTG ATCCCCTGCT | 1980 |
| GTCTGGCCTG CTCAAAGCGG CAGGCTCCCT GACGCCTGGC TCTCTCCCCA CCCCTATGGC | 2040 |
| CAGCATGGTG CACCCCCTAC CACTCCCGGG ACAGGATGCA AAAGAGGCTC CAGAGTCAGA | 2100 |
| GTGCCAAGCC AGGGAATCCC AGTCCCAGAC TCAGAGCCCG GGCCTGCACT TTGCCCCCTG | 2160 |
| CCCTTGATCA ACCCCACTGC CCCACCAGAG CTGCCAGGGT GGCACAGGGC CCTGTCCAGC | 2220 |
| CCCTGGCACA CACTTCCCTG CCAGGCCTCA GCCTCTAGCA TAAGCTCCAG AGAGCCAGGG | 2280 |
| CCCATCAGTT TCTCTCTGTG GATTTGTATC TCAGCTCCAT GATGCCTTGG GCTTTCTGTC | 2340 |
| TCCTCAACAA GAGTGCAGCT TGCTGAATGT CAGCTGCCTG AGAGAGCTGG GGCCTGACTT | 2400 |
| ACTAGGGCAT TAAATCCTAA GAGGTCCTAC TGAGGTGTGG CAGGATCACA GGCCAGTGGA | 2460 |
| AAAAGGGCAG GTCAGATGGG CAAGGCCCAG GACTTTCAGA TTAACTGAGA GGATATCGAG | 2520 |
| GCCAAGCATG GCAGGGGGAA GGTCAGTGGG TGTCAAGAGA CCCAGGTCTG ACCCCGGATG | 2580 |
| TTTGCTCCAT GTGACAAAAG CAGGCCTGTC TCAGGACCTT TTCTTTTCTT TTTTCCTTCT | 2640 |
| TTTTTTTTTT GACACGGAGT TTCGCTCTTG TTGTCCAGGC TAGAGTGCAA TGGCATGATC | 2700 |
| CCAGCTCACC GCAACGTCTA CCTCCCAGGT TCAAATCATT CTCTTGCCTC AGACTCCCGA | 2760 |
| GTAGCTGGGA TTACAGGCAC ATGCCACCAT GCCTGGCTAA TTTTGTATAT TTAGTAGAAA | 2820 |
| CAGGGTTTCA CCATGCTGGC CATGCTGGTT CTCGAACTCC TGACCTCAGG TGTTCCACCT | 2880 |
| ACCTCAGCCT CCCAAAGTGC TGGGGTTACA GGTGTGAGCC ATCGCGCCTG GCCAGGACCT | 2940 |
| TTGTTTCTTA TCTACATATT GGAAGATTTG GTCCTGATGT CCTTTGAGGC TTCTTTAGCT | 3000 |
| CTAGTTCTCT GACACTTCAG CCTATATCAC AGCTAACTTC YTCAGTCTCA TCTATTCCTT | 3060 |
| ATGCTCCAGC CCCTGGCAAT TTGCCTCAAG ATGGGGGTTT GAAAATAACT TTACCTGACT | 3120 |
| CAAGGAGTGT CTGGAGCACC TCCTAGTCTA AGTCTGCAAG CTCCAGTTCT TGCCTAAAAC | 3180 |
| CATGCCAGTG GCCACCCTTG GGCTCAGACA GCTCTGGGCC TTTTGACCAC AAGCCAGCCC | 3240 |
| CTCGCCCTCT CTGTGGCATA GTCTTCTCTG CCCCAGGACT GCAGGGCGGC TTCCTCCAAG | 3300 |
| GCTTCCAAGG CTCAAAAGAA ATTTGGCTCC ATCCAAGAAG GCTCCAGCTC CCCTACTGGC | 3360 |
| CCCTGGCTTC AGGCCCACAC CCCTGGGCCA GGSCCAGAGA GTGTGTCTCA GGAGAATTCA | 3420 |
| ATGGGCTCTA GAGAGACACA CAGAAAGTTT GGGCATTTGG GAAATTTTCA AGGRTGTATG | 3480 |
| TATGGYTCAC GTATGGWGCA GGTTGTCCTG GTCCYKGGGT GCAGGGAAGT GGGCTGCAGG | 3540 |
| GAAGTGGATT GGAGGGGAGC TTGAGGAATA TAAGGAGCGG GGGTGGAGAC TCAGGCTATG | 3600 |
| GACAAGGACA GCCCCAAGGT TGGGAAGACC TGGCCTTAGT CGTCCTCAGC CTAGGGCAGG | 3660 |
| GCAGTGAAGA AAGCTCTCCC CGCTCCTGCT GTAATGACCC AGAGTAGCCT CCCCAGGCCG | 3720 |
| GCATCTTATG TGTGTCTTCC ACCATCCTCA TGGTGGCACT TTTCTAGGCC TGTCTCCCAG | 3780 |
| CATTGTGCAA GGCTCGGAAG AGAACCACCA AGTGAAACTG GGTGAAAACA GAAAGCTCAA | 3840 |
| TGGATGGGCT AGGTTCCCAG ATCATTAGGG CAGAGTTTGC ACGTCCTCTG GTTCACTGGG | 3900 |
| AATCCACCCA GCCCACGAAT CATCTCCCTC TTTGAAGGAT TTTWATTTCT ACTGGGTTTT | 3960 |
| GGAACAAACT CCTGCTGAGA CCCCACAGCC AGAAACTGAA AGCAGCAGCT CCCCAAAGCC | 4020 |
| TGGAAAATCC CTAAGAGAAG GCCTGGGGGA MAGGAAKTGG AGTGACAGGG GACAGGTAGA | 4080 |
| GAGAAGGGGG CCCAATGGCC AGGGAGTGAA GGAGGTGGCG TTGCTGAGAG CAGTCTGCAC | 4140 |
| ATGCTTCTGT CTGAGTGCAG GAAGGTGTTC CAGGGTCGAA ATTACACTTC TCGTACCTGG | 4200 |
| AGACGCTGTT TGTGGGAGCA CTGGGCTCAT GCCTGGCACA CAATAGGTCT GCAATAAACC | 4260 |
| ATGGTTAAAT CCTGAAAAAA AAAAAAAAA | |

AAD8 DNA sequence
Gene name: ESTs
Unigene number: Hs.144953
Probeset Accession #: AA404418
Nucleic Acid Accession 4: n/a
Coding sequence: no ORF identified; possible frameshifts

| | |
|---|---|
| TATGTCCACC AAAGACACCT CGTTGGTCAT GTTCTATCAC CTCTTCGTCA AATTGACATC | 60 |
| AGGTCCTAAC AGGTCACTTT CAAGATACAG AAGAGGCAAA TTTTGTTTTG AGACTTGGCC | 120 |
| ATTCCTAGGG TCAGCAAAGT GTATTCCTGG CAGCCAGACC TTCAGTCACT TATCAGGAAA | 180 |
| TGCTTGACCT AAAGACAGAC AATTCTTTCC CCAAACTTTG CTGTTTCTTT TTTGAGTCTT | 240 |
| TGTTGAAAGA TTTCTTTTAA AAGGCGTTCG TGTGAGAAGA TCACAGCAAC AAATCTGGCT | 300 |
| TGTTCTGTTT TAGACTTACT TTCTTAACTC TTGGGCAGAA GAAAATGAAT GAGATTTGAA | 360 |
| GACCTTTGAT ACCTTGGGTA GACAAAGCTT GCCTTGAAAC TAGAAATAAG ACGAAACTAG | 420 |
| ATTTTAAGGG GAAAAAATTT GCTAGTGGTA ATATAATTGG TTTTGTTTCA TTTTTTTATG | 480 |
| AGTCTGAGGA GTTGACATTA AACGTTGGGA TGTTGCTTTG TTAATGAAGT CATTTCAATT | 540 |
| TTTGCAACTC TTAACATCTG CATGCTTCCA TAAACAGTGG GTTGGAACAA AAGAAAATGT | 600 |
| GACTAAGGGA TATTCCTTAA ATTCTTTTTT ATGTTATGAG AGAGAATATT GGAATATAAA | 660 |
| GAATGTTACT TTATCTGGTA AACCATCTCA TAGGCCAGAA GCACTAACAG TTTGAATGGT | 720 |
| TGGCTTAAAA AAAAACGGGA GTCTTTGAAT TTAAGCTTAT GTAAAATTAC TATGCAAATA | 780 |
| TAGGTTATTA TTTATTTTTA CAGTGAAAAT AAAACACTAT TGAAGTATAA ATGGAAAGAA | 840 |
| AATAAAAGCA AAGCCTGTTT AATATAGAGA CATTAATGTT GATATCACTG TACGAACAGT | 900 |
| CATAGCTTGC TGCTCACTGC CGTTAAAGGG TTGACATACA AACATTGTGG AAGAGATTTC | 960 |
| AGTTTGAGGG CTAGTGTCTG AATTATGGAC TCCTTACCCT ACTCCACCAC TTAAAACATT | 1020 |
| TTAGAGACTT TTGTGAAATT AACAGGTCAT ATAATTAATA ATTGTTGTTT TATGTACATT | 1080 |
| TATTGAAAGG CCATATTGAG GCTCCATTGA TTTTTTTTCC TGCATATTTA TCAGTATCGA | 1140 |
| ATTAGAAAAT TGAACCTTCA GTGTTACTAG ATGGAAATCT ACCAAAAAGT AGCAAGGTTT | 1200 |
| ACGAATGGTG GGATTTATTG GTGATTAAAC ATTTTTTTCC TGTATTTTAT AAGTTTCACA | 1260 |
| TTACATTTAC AATGAGAAAA AAATGTAAAT GTAGAATTAA AGTCTTGTTA ATATCGTAAT | 1320 |
| TTGCCTATTG CTGTACTAAA AGAAGCTTCT ATAAAATGTA TCATTCTCAT CCTTAGATTC | 1380 |
| AGGCCAGAAA GTAACTTTCA GTGTTAGGTA TTTGAAATAA TGCAGCCTGT CATATGTACT | 1440 |
| CTGGTTACCA GAATGAAAAA ACAAAAAGAG ATACATACAT AGTAAGGAAA CATGAAATTG | 1500 |
| GAGGAATTGA TCCCCATGTG TATTGCAGCT TCATATACCA GTAGTCTCTA ATAAGTCATT | 1560 |
| GCTTTAATAA AAAAAAAAAT AGAAAATTTA AA | |

ACA2 DNA sequence
Gene name: EST
Unigene number: Hs.16450
Probeset Accession #: AA478778
Nucleic Acid Accession #: AA478778
Coding sequence: no ORF identified; possible frameshifts

| | |
|---|---|
| TATTTTTGTA CGTAAAATGA TTCTATTATG ACTGCCTTTG CATGTAGTAA TATGACAAAG | 60 |
| TGATCCTTCA TTATCACGGT ACACTATTGT TTACTTTTCA TCTGTAAATG TTTTATTGTT | 120 |
| ACTTTTTTAA AATGAATTTT TTTAAAACAA TCTAGCCATC ATCAAGGTGC TATAAGAGTT | 180 |
| GTATAAAAGA TATTTTTGGC ATTTCTAGGC AAGTATCAGC CAATAAGTAT GTTAGTGATA | 240 |
| TCACAGATTG TACCAACTAT TAACTATGTT AAATAAGTAT TCAGTTTCAT GTGATCTCTG | 300 |
| GGAAAAAAAT ATGCTGCCTT GGTGCTAATA TTGTATGTAT TTAAATGATC ATCTGACTCA | 360 |
| GAAATATAAA CACTTTTAAT GAAAGGGAGG AACGGAAGGA CAATTTCCAG TGCACAGAAT | 420 |
| CACTTGGATG AAATAAGACC AGCTCTTTAC CCTTATTTTT GGATATGCCT TTTTTGGAAG | 480 |
| AGACTTAGAC TTTATCCTTA TTGTTGTTAG TGTTGTTAAT ATTCGTTGCT TCAGCCCACG | 540 |
| GTGCCTTGGT CTCTCCACAA TCAAATGGAG GATCCCCCAA GCAGCTTCAT TACAGAGTGA | 600 |
| TATTGGGAAA GTGAGATCCT CTCACCATTT TGCCAAGATA CTCTAAAATG ACATCCAAGT | 660 |
| TTACCAGTAG AAAGACACAG GATGCACAGA ATGGGCATGA CCTTCAGCTC ACGAGCACAC | 720 |
| CTGGAGAAAT TCAGAACCAG GTTCTGAATC ATCACGATTG CCTTTTGCAT GAAAACATCG | 780 |
| GCTGGTGATG TGACTTCTCT TCAGGCCATG AGCCTAACAY CCTGCCGGTT TTCATGCCCG | 840 |
| CTGCAGTAAT GGACGTTTGT GTGAAGAAAT GAACTGTGGA GTACAAAA CTTTGAGTCT | 900 |
| TTCCGATTGC TCATTAATTC ACTTTTTTGT TACTTCTTTC CAAAATGGAA GTGCTGAAGC | 960 |
| CATGGTCTTT CTGCCCCTCC AAGCTGATGA AGGGAAGCCT TTGCCAATGG CCCATGGAAG | 1020 |
| ACACTTGGTT TGAGAAACCC TGCCCACTTC CAAAGACCAA AGAGATTAGG AAAAGCCTGG | 1080 |
| CAGTATTCTC CAACTCCAAA CAAGCTCTAG AGTGCTCCAG GAAAAGTTAT ATTCAGTATA | 1140 |
| TGAATAAGTG TTATTCTCCA TTATTAATGT GTTCTGAAAA TATATTATGA ATAAATACAT | 1200 |
| CACCACACCC AAAAAAAAAA AAAAAAAAAA AAAA | |

ACA4 DNA sequence
Gene name: alpha satellite junction DNA sequence
Unigene number: Hs.247946
Probeset Accession #: M21305
Nucleic Acid Accession #: M21305
Coding sequence: 1-165 (predicted start/stop codons underlined)

| | |
|---|---|
| ATGGAATGGA ATGGAATGGC ATGGAATCGT ATAAAGTGGA ATGGAATCAA CTCGAGTGGA | 60 |
| ATGGAATGGA ATGGAATGGA ATGGAATGCA GTACAATGCA ATAGAATGGA ATGGAATGAA | 120 |
| CTCGAGTTGA CTGGAATGGA ATGGAATGGA ATGCATTTGA ATTGA | |

ACG6 DNA sequence
Gene name: intercellular adhesion molecule 2 (ICAM2)
Unigene number: Hs.83733
Probeset Accession #: M32334
Nucleic Acid Accession #: NM_000873
Coding sequence: 63-890 (predicted start/stop codons underlined)

| | |
|---|---|
| CTAAAGATCT CCCTCCAGGC AGCCCTTGGC TGGTCCCTGC GAGCCCGTGG AGACTGCCAG | 60 |
| AGATGTCCTC TTTCGGTTAC AGGACCCTGA CTGTGGCCCT CTTCACCCTG ATCTGCTGTC | 120 |
| CAGGATCGGA TGAGAAGGTA TTCGAGGTAC ACGTGAGGCC AAAGAAGCTG GCGGTTGAGC | 180 |
| CCAAAGGGTC CCTCGAGGTC AACTGCAGCA CCACCTGTAA CCAGCCTGAA GTGGGTGGTC | 240 |
| TGGAGACCTC TCTAAATAAG ATTCTGCTGG ACGAACAGGC TCAGTGGAAA CATTACTTGG | 300 |
| TCTCAAACAT CTCCCATGAC ACGCTCCTCC AATGCCACTT CACCTGCTCC GGGAAGCAGG | 360 |
| AGTCAATGAA TTCCAACGTC AGCGTGTACC AGCCTCCAAG GCAGGTCATC CTGACACTGC | 420 |
| AACCCACTTT GGTGGCTGTG GGCAAGTCCT TCACCATTGA GTGCAGGGTG CCCACCGTGG | 480 |
| AGCCCCTGGA CAGCCTCACC CTCTTCCTGT TCCGTGGCAA TGAGACTCTG CACTATGAGA | 540 |
| CCTTCGGGAA GGCAGCCCCT GCTCCGCAGG AGGCCACAGC CACATTCAAC AGCACGGCTG | 600 |
| ACAGAGAGGA TGGCCACCGC AACTTCTCCT GCCTGGCTGT GCTGGACTTG ATGTCTCGCG | 660 |
| GTGGCAACAT CTTTCACAAA CACTCAGCCC CGAAGATGTT GGAGATCTAT GAGCCTGTGT | 720 |
| CGGACAGCCA GATGGTCATC ATAGTCACGG TGGTGTCGGT GTTGCTGTCC CTGTTCGTGA | 780 |
| CATCTGTCCT GCTCTGCTTC ATCTTCGGCC AGCACTTGCG CCAGCAGCGG ATGGGCACCT | 840 |
| ACGGGGTGCG AGCGGCTTGG AGGAGGCTGC CCCAGGCCTT CCGGCCATAG CAACCATGAG | 900 |
| TGGCATGGCC ACCACCACGG TGGTCACTGG AACTCAGTGT GACTCCTCAG GGTTGAGGTC | 960 |
| CAGCCCTGGC TGAAGGACTG TGACAGGCAG CAGAGACTTG GGACATTGCC TTTTCTAGCC | 1020 |
| CGAATACAAA CACCTGGACT T | |

ACG7 DNA sequence
Gene name: Cadherin 5, VE-cadherin (CDH5)
Unigene number: Hs.76206
Probeset Accession #: X79981
Nucleic Acid Accession #: NM_001795
Coding sequence: 25-2379 (predicted start/stop codons underlined)

| | |
|---|---|
| GCACGATCTG TTCCTCCTGG GAAGATGCAG AGGCTCATGA TGCTCCTCGC CACATCGGGC | 60 |
| GCCTGCCTGG GCCTGCTGGC AGXGGCAGCA GTGGCAGCAG CAGGTGCTAA CCCTGCCCAA | 120 |
| CGGGACACCC ACAGCCTGCT GCCCACCCAC CGGCGCCAAA AGAGAGATTG GATTTGGAAC | 180 |
| CAGATGCACA TTGATGAAGA GAAAAACACC TCACTTCCCC ATCATGTAGG CAAGATCAAG | 240 |
| TCAAGCGTGA GTCGCAAGAA TGCCAAGTAC CTGCTCAAAG GAGAATATGT GGGCAAGGTC | 300 |
| TTCCGGGTCG ATGCAGAGAC AGGAGACGTG TTCGCCATTG AGAGGCTGGA CCGGGAGAAT | 360 |
| ATCTCAGAGT ACCACCTCAC TGCTGTCATT GTGGACAAGG ACACTGGTGA AAACCTGGAG | 420 |
| ACTCCTTCCA GCTTCACCAT CAAAGTTCAT GACGTGAACG ACAACTGGCC TGTGTTCACG | 480 |
| CATCGGTTGT TCAATGCGTC CGTGCCTGAG TCGTCGGCTG TGGGGACCTC AGTCATCTCT | 540 |
| GTGACAGCAG TGGATGCAGA CGACCCCACT GTGGGAGACC ACGCCTCTGT CATGTACCAA | 600 |
| ATCCTGAAGG GGAAAGAGTA TTTTGCCATC GATAATTCTG GACGTATTAT CACAATAACG | 660 |
| AAAAGCTTGG ACCGAGAGAA GCAGGCCAGG TATGAGATCG TGGTGGAAGC GCGAGATGCC | 720 |
| CAGGGCCTCC GGGGGGACTC GGGCACGGCC ACCGTGCTGG TCACTCTGCA AGACATCAAT | 780 |
| GACAACTTCC CCTTCTTCAC CCAGACCAAG TACACATTTG TCGTGCCTGA AGACACCCGT | 840 |
| GTGGGCACCT CTGTGGGCTC TCTGTTTGTT GAGGACCCAG ATGAGCCCCA GAACCGGATG | 900 |
| ACCAAGTACA GCATCTTGCG GGGCGACTAC CAGGACGCTT TCACCATTGA GACAAACCCC | 960 |
| GCCCACAACG AGGGCATCAT CAAGCCCATG AAGCCTCTGG ATTATGAATA CATCCAGCAA | 1020 |
| TACAGCTTCA TCGTCGAGGC CACAGACCCC ACCATCGACC TCCGATACAT GAGCCCTCCC | 1080 |
| GCGGGAAACA GAGCCCAGGT CATTATCAAC ATCACAGATG TGGACGAGCC CCCCATTTTC | 1140 |
| CAGCAGCCTT TCTACCACTT CCAGCTGAAG GAAAACCAGA AGAAGCCTCT GATTGGCACA | 1200 |
| GTGCTGGCCA TGGACCCTGA TGCGGCTAGG CATAGCATTG GATACTCCAT CCGCAGGACC | 1260 |
| AGTGACAAGG GCCAGTTCTT CCGAGTCACA AAAAAGGGGG ACATTTACAA TGAGAAAGAA | 1320 |
| CTGGACAGAG AAGTCTACCC CTGGTATAAC CTGACTGTGG AGGCCAAAGA ACTGGATTCC | 1380 |
| ACTGGAACCC CCACAGGAAA AGAATCCATT GTGCAAGTCC ACATTGAAGT TTTGGATGAG | 1440 |
| AATGACAATG CCCCGGAGTT TGCCAAGCCC TACCAGCCCA AAGTGTGTGA GAACGCTGTC | 1500 |
| CATGGCCAGC TGGTCCTGCA GATCTCCGCA ATAGACAAGG ACATAACACC ACGAAACGTG | 1560 |
| AAGTTCAAAT TCACCTTGAA TACTGAGAAC AACTTTACCC TCACGGATAA TCACGATAAC | 1620 |
| ACGGCCAACA TCACAGTCAA GTATGGGCAG TTTGACCGGG AGCATACCAA GGTCCACTTC | 1680 |
| CTACCCGTGG TCATCTCAGA CAATGGGATG CCAAGTCGCA CGGGCACCAG CACGCTGACC | 1740 |
| GTGGCCGTGT GCAAGTGCAA CGAGCAGGGC GAGTTCACCT TCTGCGAGGA TATGGCCGCC | 1800 |
| CAGGTGGGCG TGAGCATCCA GGCAGTGGTA GCCATCTTAC TCTGCATCCT CACCATCACA | 1860 |
| GTGATCACCC TGCTCATCTT CCTGCGGCGG CGGCTCCGGA AGCAGGCCCG CGCGCACGGC | 1920 |
| AAGAGCGTGC CGGAGATCCA CGAGCAGCTG GTCACCTACG ACGAGGAGGG CGGCGGCGAG | 1980 |
| ATGGACACCA CCAGCTACGA TGTGTCGGTG CTCAACTCGG TGCGCCGCGG CGGGGCCAAG | 2040 |
| CCCCCGCGGC CCGCGCTGGA CGCCCGGCCT TCCCTCTATG CGCAGGTGCA GAAGCCACCG | 2100 |
| AGGCACGCGC CTGGGGCACA CGGAGGGCCC GGGGAGATGG CAGCCATGAT CGAGGTGAAG | 2160 |
| AAGGACGAGG CGGACCACGA CGGCGACGGC CCCCCCTACG ACACGCTGCA CATCTACGGC | 2220 |
| TACGAGGGCT CCGAGTCCAT AGCCGAGTCC CTCAGCTCCC TGGGCACCGA CTCATCCGAC | 2280 |
| TCTGACGTGG ATTACGACTT CCTTAACGAC TGGGGACCCA GGTTTAAGAT GCTGGCTGAG | 2340 |
| CTGTACGGCT CGGACCCCCG GGAGGAGCTG CTGTATTAGG CGGCCGAGGT CACTCTGGGC | 2400 |
| CTGGGGACCC AAACCCCCTG CAGCCCAGGC CAGTCAGACT CCAGGCACCA CAGCCTCCAA | 2460 |
| AAATGGCAGT GACTCCCCAG CCCAGCACCC CTTCCTCGTG GGTCCCAGAG ACCTCATCAG | 2520 |
| CCTTGGGATA GCAAACTCCA GGTTCCTGAA ATATCCAGGA ATATATGTCA GTGATGACTA | 2580 |
| TTCTCAAATG CTGGCAAATC CAGGCTGGTG TTCTGTCTGG GCTCAGACAT CCACATAACC | 2640 |
| CTGTCACCCA CAGACCGCCG TCTAACTCAA AGACTTCCTC TGGCTCCCCA AGGCTGCAAA | 2700 |
| GCAAAACAGA CTGTGTTTAA CTGCTGCAGG GTCTTTTTCT AGGGTCCCTG AACGCCCTGG | 2760 |
| TAAGGCTGGT GAGGTCCTGG TGCCTATCTG CCTGGAGGCA AAGGCCTGGA CAGCTTGACT | 2820 |
| TGTGGGGCAG GATTCTCTGC AGCCCATTCC CAAGGGAGAC TGACCATCAT GCCCTCTCTC | 2880 |
| GGGAGCCCTA GCCCTGCTCC AACTCCATAC TCCACTCCAA GTGCCCCACC ACTCCCCAAC | 2940 |
| CCCTCTCCAG GCCTGTCAAG AGGGAGGAAG GGGCCCCATG GCAGCTCCTG ACCTTGGGTC | 3000 |
| CTGAAGTGAC CTCACTGGCC TGCCATGCCA GTAACTGTGC TGTACTGAGC ACTGAACCAC | 3060 |
| ATTCAGGGAA ATGCTTATTA AACCTTGAAG CAACTGTGAA TTCATTCTGG AGGGGCAGTG | 3120 |
| GAGATCAGGA GTGACAGATC ACAGGGTGAG GGCCACCTCC ACACCCACCC CCTCTGGAGA | 3180 |
| AGGCCTGGAA GAGCTGAGAC CTTGCTTTGA GACTCCTCAG CACCCCTCCA GTTTTGCCTG | 3240 |
| AGAAGGGGCA GATGTTCCCG GAGATCAGAA GACGTCTCCC CTTCTCTGCC TCACCTGGTC | 3300 |
| GCCAATCCAT GCTCTCTTTC TTTTCTCTGT CTACTCCTTA TCCCTTGGTT TAGAGGAACC | 3360 |
| CAAGATGTGG CCTTTAGCAA AACTGACAAT GTCCAAACCC ACTCATGACT GCATGACGGA | 3420 |
| GCCGAGCATG TGTCTTTACA CCTCGCTGTT GTCACATCTC AGGGAACTGA CCCTCAGGCA | 3480 |
| CACCTTGCAG AAGGAAGGCC CTGCCCTGCC CAACCTCTGT GGTCACCCAT GCATCATTCC | 3540 |
| ACTGGAACGT TTCACTGCAA ACACACCTTG GAGAAGTGGC ATCAGTCAAC AGAGAGGGGC | 3600 |
| AGGGAAGGAG ACACCAAGCT CACCCTTCGT CATGGACCGA GGTTCCCACT CTGGCAAAGC | 3660 |
| CCCTCACACT GCAAGGGATT GTAGATAACA CTGACTTGTT TGTTTTAACC AATAACTAGC | 3720 |
| TTCTTATAAT GATTTTTTTA CTAATGATAC TTACAAGTTT CTAGCTCTCA CAGACATATA | 3780 |
| GAATAAGGGT TTTTGCATAA TAAGCAGGTT GTTATTTAGG TTAACAATAT TAATTCAGGT | 3840 |
| TTTTTAGTTG GAAAAACAAT TCCTGTAACC TTCTATTTTC TATAATTGTA GTAATTGCTC | 3900 |
| TACAGATAAT GTCTATATAT TGGCCAAACT GGTGCATGAC AAGTACTGTA TTTTTTTATA | 3960 |
| CCTAAATAAA GAAAAATCTT TAGCCTGGGC AACAAAAAAA | |

ACG9 DNA sequence
Gene name: lysyl oxidase-like 2 (LOXL2)
Unigene number: Hs.83354
Probeset Accession #: U89942
Nucleic Acid Accession #: NM_002318 cluster
Coding sequence: 248-2572 (predicted start/stop codons underlined)

| | |
|---|---|
| ACTCCAGCGC GCGGCTACCT ACGCTTGGTG CTTGCTTTCT CCAGCCATCG GAGACCAGAG | 60 |
| CCGCCCCCTC TGCTCGAGAA AGGGGCTCAG CGGCGGCGGA AGCGGAGGGG GACCACCGTG | 120 |
| GAGAGCGCGG TCCCAGCCCG GCCACTGCGG ATCCCTGAAA CCAAAAAGCT CCTGCTGCTT | 180 |
| CTGTACCCCG CCTGTCCCTC CCAGCTGCGC AGGGCCCCTT CGTGGGATCA TCAGCCCGAA | 240 |
| GACAGGGATG GAGAGGCCTC TGTGCTCCCA CCTCTGCAGC TGCCTGGCTA TGCTGGCCCT | 300 |
| CCTGTCCCCC CTGAGGCTGG CACAGTATGA CAGCTGGCCC CATTACCCCG AGTACTTCCA | 360 |
| GCAACCGGCT CCTGAGGATC ACCAGCCCCA GGCCCCCGCC AACGTGGCCA AGATTCAGCT | 420 |
| GCGCCTGGCT GGGCAGAAGA GGAAGCACAG CGAGGGCCGG GTGGAGGTGT ACTATGATGG | 480 |
| CCAGTGGGGC ACCGTGTGCG ATGACGACTT CTCCATCCAC GCTGCCCACG TCGTCTGCCG | 540 |
| GGAGCTGGGC TATGTGGAGG CCAAGTCCTG GACTGCCAGC TCCTCCTACG GCAAGGGAGA | 600 |
| AGGGCCCATC TGGTTAGACA ATCTCCACTG TACTGGCAAC GAGGCGACCC TTGCAGCATG | 660 |
| CACCTCCAAT GGCTGGGGCG TCACTGACTG CAAGCACACG GAGGATGTCG GTGTGGTGTG | 720 |
| CAGCGACAAA AGGATTCCTG GGTTCAAATT TGACAATTCG TTGATCAACC AGATAGAGAA | 780 |
| CCTGAATATC CAGGTGGAGG ACATTCGGAT TCGAGCCATC CTCTCAACCT ACCGCAAGCG | 840 |
| CACCCCAGTG ATGGAGGGCT ACGTGGAGGT GAAGGAGGGC AAGACCTGGA AGCAGATCTG | 900 |
| TGACAAGCAC TGGACGGCCA AGAATTCCCG CGTGGTCTGC GGCATGTTTG GCTTCCCTGG | 960 |
| GGAGAGGACA TACAATACCA AAGTGTACAA AATGTTTGCC TCACGGAGGA AGCAGCGCTA | 1020 |
| CTGGCCATTC TCCATGGACT GCACCGGCAC AGAGGCCCAC ATCTCCAGCT GCAAGCTGGG | 1080 |
| CCCCCAGGTG TCACTGGACC CCATGAAGAA TGTCACCTGC GAGAATGGGC TGCCGGCCGT | 1140 |
| GGTGAGTTGT GTGCCTGGGC AGGTCTTCAG CCCTGACGGA CCCTCGAGAT TCCGGAAAGC | 1200 |
| ATACAAGCCA GAGCAACCCC TGGTGCGACT GAGAGGCGGT GCCTACATCG GGGAGGGCCG | 1260 |
| CGTGGAGGTG CTCAAAAATG GAGAATGGGG GACCGTCTGC GACGACAAGT GGGACCTGGT | 1320 |
| GTCGGCCAGT GTGGTCTGCA GAGAGCTGGG CTTTGGGAGT GCCAAAGAGG CAGTCACTGG | 1380 |
| CTCCCGACTG GGGCAAGGGA TCGGACCCAT CCACCTCAAC GAGATCCAGT GCACAGGCAA | 1440 |
| TGAGAAGTCC ATTATAGACT GCAAGTTCAA TGCCGAGTCT CAGGGCTGCA ACCACGAGGA | 1500 |
| GGATGCTGGT GTGAGATGCA ACACCCCTGC CATGGGCTTG CAGAAGAAGC TGCGCCTGAA | 1560 |
| CGGCGGCCGC AATCCCTACG AGGGCCGAGT GGAGGTGCTG GTGGAGAGAA ACGGGTCCCT | 1620 |
| TGTGTGGGGG ATGGTGTGTG GCCAAAACTG GGGCATCGTG GAGGCCATGG TGGTCTGCCG | 1680 |
| CCAGCTGGGC CTGGGATTCG CCAGCAACGC CTTCCAGGAG ACCTGGTATT GGCACGGAGA | 1740 |
| TGTCAACAGC AACAAAGTGG TCATGAGTGG AGTGAAGTGC TCGGGAACGG AGCTGTCCCT | 1800 |
| GGCGCACTGC CGCCACGACG GGGAGGACGT GGCCTGCCCC CAGGGCGGAG TGCAGTACGG | 1860 |
| GGCCGGAGTT GCCTGCTCAG AAACCGCCCC TGACCTGGTC CTCAATGCGG AGATGGTGCA | 1920 |
| GCAGACCACC TACCTGGAGG ACCGGCCCAT GTTCATGCTG CAGTGTGCCA TGGAGGAGAA | 1980 |
| CTGCCTCTCG GCCTCAGCCG CGCAGACCGA CCCCACCACG GGCTACCGCC GGCTCCTGCG | 2040 |
| CTTCTCCTCC CAGATCCAGA ACAATGGCCA GTCCGACTTC CGGCCCAAGA AGGGCGGCCA | 2100 |
| CGCGTGGATC TGGCACGACT GTCACAGGCA CTACCACAGC ATGGAGGTGT TCACCCACTA | 2160 |
| TGACCTGCTG AACCTCAATG GCACCAAGGT GGCAGAGGGC CACAAGGCCA GCTTCTGCTT | 2220 |
| GGAGGACACA GAATGTGAAG GAGACATCCA GAAGAATTAC GAGTGTGCCA ACTTCGGCGA | 2280 |
| TCAGGGCATC ACCATGGGCT GCTGGGACAT GTACCGCCAT GACATCGACT GCCAGTGGGT | 2340 |
| TGACATCACT GACGTGCCCC CTGGAGACTA CCTGTTCCAG GTTGTTATTA ACCCCAACTT | 2400 |
| CGAGGTTGCA GAATCCGATT ACTCCAACAA CATCATGAAA TGCAGGAGCC GCTATGACGG | 2460 |
| CCACCGCATC TGGATGTACA ACTGCCACAT AGGTGGTTCC TTCAGCGAAG AGACGGAAAA | 2520 |
| AAAGTTTGAG CACTTCAGCG GGCTCTTAAA CAACCAGCTG TCCCCGCAGT AAAGAAGCCT | 2580 |
| GCGTGGTCAA CTCCTGTCTT CAGGCCACAC CACATCTTCC ATGGGACTTC CCCCCAACAA | 2640 |
| CTGAGTCTGA ACGAATGCCA CGTGCCCTCA CCCAGCCCGG CCCCCACCCT GTCCAGACCC | 2700 |
| CTACAGCTGT GTCTAAGCTC AGGAGGAAAG GGACCCTCCC ATCATTCATG GGGGGCTGCT | 2760 |
| ACCTGACCCT TGGGGCCTGA GAAGGCCTTG GGGGGGTGGG GTTTGTCCAC AGAGCTGCTG | 2820 |
| GAGCAGCACC AAGAGCCAGT CTTGACCGGG ATGAGGCCCA CAGACAGGTT GTCATCAGCT | 2880 |
| TGTCCCATTC AAGCCACCGA GCTCACCACA GACACAGTGG AGCCGCGCTC TTCTCCAGTG | 2940 |
| ACACGTGGAC AAATGCGGGC TCATCAGCCC CCCCAGAGAG GGTCAGGCCG AACCCCATTT | 3000 |
| CTCCTCCTCT TAGGTCATTT TCAGCAAACT TGAATATCTA GACCTCTCTT CCAATGAAAC | 3060 |
| CCTCCAGTCT ATTATAGTCA CATAGATAAT GGTGCCACGT GTTTTCTGAT TTGGTGAGCT | 3120 |
| CAGACTTGGT GCTTCCCTCT CCACAACCCC CACCCCTTGT TTTTCAAGAT ACTATTATTA | 3180 |
| TATTTTCACA GACTTTTGAA GCACAAATTT ATTGGCATTT AATATTGGAC ATCTGGGCCC | 3240 |
| TTGGAAGTAC AAATCTAAGG AAAAACCAAC CCACTGTGTA AGTGACTCAT CTTCCTGTTG | 3300 |
| TTCCAATTCT GTGGGTTTTT GATTCAACGG TGCTATAACC AGGGTCCTGG GTGACAGGGC | 3360 |
| GCTCACTGAG CACCATGTGT CATCACAGAC ACTTACACAT ACTTGAAACT TGGAATAAAA | 3420 |
| GAAAGATTTA TG | |

ACH2 DNA sequence
Gene name:TIE tyrosine-protein kinase
Unigene number: Hs.78824
Probeset Accession #: X60957
Nucleic Acid Accession #: NM_005424 cluster
Coding sequence: 37-3452 (predicted start/stop codons underlined)

| | |
|---|---|
| CGCTCGTCCT GGCTGGCCTG GGTCGGCCTC TGGAGTATGG TCTGGCGGGT GCCCCCTTTC | 60 |
| TTGCTCCCCA TCCTCTTCTT GGCTTCTCAT GTGGGCGCGG CGGTGGACCT GACGCTGCTG | 120 |
| GCCAACCTGC GGCTCACGGA CCCCCAGCGC TTCTTCCTGA CTTGCGTGTC TGGGGAGGCC | 180 |
| GGGGCGGGGA GGGGCTCGGA CGCCTGGGGC CCGCCCCTGC TGCTGGAGAA GGACGACCGT | 240 |
| ATCGTGCGCA CCCCGCCCGG GCCACCCCTG CGCCTGGCGC GCAACGGTTC GCACCAGGTC | 300 |
| ACGCTTCGCG GCTTCTCCAA GCCCTCGGAC CTCGTGGGCG TCTTCTCCTG CGTGGGCGGT | 360 |
| GCTGGGGCGC GGCGCACGCG CGTCATCTAC GTGCACACA GCCCTGGAGC CCACCTGCTT | 420 |
| CCAGACAAGG TCACACACAC TGTGAACAAA GGTGAGACCG CTGTACTTTC TGCACGTGTG | 480 |
| CACAAGGAGA AGCAGACAGA CGTGATCTGG AAGAGCAACG GATCCTACTT CTACACCCTG | 540 |
| GACTGGCATG AAGCCCAGGA TGGGCGGTTC CTGCTGCAGC TCCCAAATGT GCAGCCACCA | 600 |
| TCGAGCGGCA TCTACAGTGC CACTTACCTG GAAGCCAGCC CCCTGGGCAG CGCCTTCTTT | 660 |
| CGGCTCATCG TGCGGGGTTG TGGGGCTGGG CGCTGGGGGC CAGGCTGTAC CAAGGAGTGC | 720 |
| CCAGGTTGCC TACATGGAGG TGTCTGCCAC GACCATGACG GCGAATGTGT ATGCCCCCCT | 780 |
| GGCTTCACTG GCACCCGCTG TGAACAGGCC TGCAGAGAGG GCCGTTTTGG GCAGAGCTGC | 840 |
| CAGGAGCAGT GCCCAGGCAT ATCAGGCTGC CGGGGCCTCA CCTTCTGCCT CCCAGACCCC | 900 |
| TATGGCTGCT CTTGTGGATC TGGCTGGAGA GGAAGCCAGT GCCAAGAAGC TTGTGCCCCT | 960 |
| GGTCATTTTG GGGCTGATTG CCGACTCCAG TGCCAGTGTC AGAATGGTGG CACTTGTGAC | 1020 |
| CGGTTCAGTG GTTGTGTCTG CCCCTCTGGG TGGCATGGAG TGCACTGTGA GAAGTCAGAC | 1080 |
| CGGATCCCCC AGATCCTCAA CATGGCCTCA GAACTGGAGT TCAACTTAGA GACGATGCCC | 1140 |
| CGGATCAACT GTGCAGCTGC AGGGAACCCC TTCCCCGTGC GGGGCAGCAT AGAGCTACGC | 1200 |
| AAGCCAGACG GCACTGTGCT CCTGTCCACC AAGGCCATTG TGGAGCCAGA GAAGACCACA | 1260 |
| GCTGAGTTCG AGGTGCCCCG CTTGGTTCTT GCGGACAGTG GGTTCTGGGA GTGCCGTGTG | 1320 |
| TCCACATCTG GCGGCCAAGA CAGCCGGCGC TTCAAGGTCA ATGTGAAAGT GCCCCCCGTG | 1380 |
| CCCCTGGCTG CACCTCGGCT CCTGACCAAG CAGAGCCGCC AGCTTGTGGT CTCCCCGCTG | 1440 |
| GTCTCGTTCT CTGGGGATGG ACCCATCTCC ACTGTCCGCC TGCACTACCG GCCCCAGGAC | 1500 |
| AGTACCATGG ACTGGTCGAC CATTGTGGTG GACCCCAGTG AGAACGTGAC GTTAATGAAC | 1560 |
| CTGAGGCCAA AGACAGGATA CAGTGTTCGT GTGCAGCTGA GCCGGCCAGG GGAAGGAGGA | 1620 |
| GAGGGGGCCT GGGGGCCTCC CACCCTCATG ACCACAGACT GTCCTGAGCC TTTGTTGCAG | 1680 |
| CCGTGGTTGG AGGGCTGGCA TGTGGAAGGC ACTGACCGGC TGCGAGTGAG CTGGTCCTTG | 1740 |
| CCCTTGGTGC CCGGGCCACT GGTGGGCGAC GGTTTCCTGC TGCGCCTGTG GGACGGGACA | 1800 |
| CGGGGGCAGG AGCGGCGGGA GAACGTCTCA TCCCCCCAGG CCCGCACTGC CCTCCTGACG | 1860 |
| GGACTCACGC CTGGCACCCA CTACCAGCTG GATGTGCAGC TCTACCACTG CACCCTCCTG | 1920 |
| GGCCCGGCCT CGCCCCCTGC ACACGTGCTT CTGCCCCCCA GTGGGCCTCC AGCCCCCCGA | 1980 |
| CACCTCCACG CCCAGGCCCT CTCAGACTCC GAGATCCAGC TGACATGGAA GCACCCGGAG | 2040 |
| GCTCTGCCTG GGCCAATATC CAAGTACGTT GTGGAGGTGC AGGTGGCTGG GGGTGCAGGA | 2100 |
| GACCCACTGT GGATAGACGT GGACAGGCCT GAGGAGACAA GCACCATCAT CCGTGGCCTC | 2160 |
| AACGCCAGCA CGCGCTACCT CTTCCGCATG CGGGCCAGCA TTCAGGGGCT CGGGGACTGG | 2220 |
| AGCAACACAG TAGAAGAGTC CACCCTGGGC AACGGGCTGC AGGCTGAGGG CCCAGTCCAA | 2280 |
| GAGAGCCGGG CAGCTGAAGA GGGCCTGGAT CAGCAGCTGA TCCTGGCGGT GGTGGGCTCC | 2340 |
| GTGTCTGCCA CCTGCCTCAC CATCCTGGCC GCCCTTTTAA CCCTGGTGTG CATCCGCAGA | 2400 |
| AGCTGCCTGC ATCGGAGACG CACCTTCACC TACCAGTCAG GCTCGGGCGA GGAGACCATC | 2460 |
| CTGCAGTTCA GCTCAGGGAC CTTGACACTT ACCCGGCGGC CAAAACTGCA GCCCGAGCCC | 2520 |
| CTGAGCTACC CAGTGCTAGA GTGGGAGGAC ATCACCTTTG AGGACCTCAT CGGGGAGGGG | 2580 |
| AACTTCGGCC AGGTCATCCG GGCCATGATC AAGAAGGACG GGCTGAAGAT GAACGCAGCC | 2640 |
| ATCAAAATGC TGAAAGAGTA TGCCTCTGAA AATGACCATC GTGACTTTGC GGGAGAACTG | 2700 |
| GAAGTTCTGT GCAAATTGGG GCATCACCCC AACATCATCA ACCTCCTGGG GGCCTGTAAG | 2760 |
| AACCGAGGTT ACTTGTATAT CGCTATTGAA TATGCCCCCT ACGGGAACCT GCTAGATTTT | 2820 |
| CTGCGGAAAA GCCGGGTCCT AGAGACTGAC CCAGCTTTTG CTCGAGAGCA TGGGACAGCC | 2880 |
| TCTACCCTTA GCTCCCGGCA GCTGCTGCGT TTCGCCAGTG ATGCGGCCAA TGGCATGCAG | 2940 |
| TACCTGAGTG AGAAGCAGTT CATCCACAGG GACCTGGCTG CCCGGAATGT GCTGGTCGGA | 3000 |
| GAGAACCTAG CCTCCAAGAT TGCAGACTTC GGCCTTTCTC GGGGAGAGGA GGTTTATGTG | 3060 |
| AAGAAGACGA TGGGGCGTCT CCCTGTGCGC TGGATGGCCA TTGAGTCCCT GAACTACAGT | 3120 |
| GTCTATACCA CCAAGAGTGA TGTCTGGTCC TTTGGAGTCC TTCTTTGGGA GATAGTGAGC | 3180 |
| CTTGGAGGTA CACCCTACTG TGGCATGACC TGTGCCGAGC TCTATGAAAA GCTGCCCCAG | 3240 |
| GGCTACCGCA TGGAGCAGCC TCGAAACTGT GACGATGAAG TGTACGAGCT GATGCGTCAG | 3300 |
| TGCTGGCGGG ACCGTCCCTA TGAGCGACCC CCCTTTGCCC AGATTGCGCT ACAGCTAGGC | 3360 |
| CGCATGCTGG AAGCCAGGAA GGCCTATGTG AACATGTCGC TGTTTGAGAA CTTCACTTAC | 3420 |
| GCGGGCATTG ATGCCACAGC TGAGGAGGCC TGAGCTGCCA TCCAGCCAGA ACGTGGCTCT | 3480 |
| GCTGGCCGGA GCAAACTCTG CTGTCTAACC TGTGACCAGT CTGACCCTTA CAGCCTCTGA | 3540 |
| CTTAAGCTGC CTCAAGGAAT TTTTTTAACT TAAGGGAGAA AAAAAGGGAT CTGGGGATGG | 3600 |
| GGTGGGCTTA GGGGAACTGG GTTCCCATGC TTTGTAGGTG TCTCATAGCT ATCCTGGGCA | 3660 |
| TCCTTCTTTC TAGTTCAGCT GCCCCACAGG TGTGTTTCCC ATCCCACTGC TCCCCCAACA | 3720 |
| CAAACCCCCA CTCCAGCTCC TTCGCTTAAG CCAGCACTCA CACCACTAAC ATGCCCTGTT | 3780 |
| CAGCTACTCC CACTCCCGGC CTGTCATTCA GAAAAAAATA AATGTTCTAA TAAGCTCCAA | 3840 |
| AAAAA | |

ACH3 DNA sequence
Gene name: placental growth factor (PGF; PlGF1; VEGF-related protein)
Unigene number: Hs.2894
Probeset Accession #: X54936
Nucleic Acid Accession #: NM_002632 cluster
Coding sequence: 322-768 (predicted start/stop codons underlined)

| | |
|---|---|
| GGGATTCGGG CCGCCCAGCT ACGGGAGGAC CTGGAGTGGC ACTGGGCGCC CGACGGCA | 60 |
| TCCCCGGGAC CCGCCTGCCC CTCGGCGCCC CGCCCCGCCG GGCCGCTCCC CGTCGGCTTC | 120 |
| CCCAGCCACA GCCTTACCTA CGGGCTCCTG ACTCCGCAAG GCTTCCAGAA GATGCTCGAA | 180 |
| CCACCGGCCG GGGCCTCGGG GCAGCAGTGA GGGAGGCGTC CAGCCCCCCA CTCAGCTCTT | 240 |
| CTCCTCCTGT GCCAGGGGCT CCCCGGGGGA TGAGCATGGT GGTTTTCCCT CGGAGCCCCC | 300 |
| TGGCTCGGGA CGTCTGAGAA GATGCCGGTC ATGAGGCTGT TCCCTTGCTT CCTGCAGCTC | 360 |
| CTGGCCGGGC TGGCGCTGCC TGCTGTGCCC CCCCAGCAGT GGGCCTTGTC TGCTGGGAAC | 420 |
| GGCTCGTCAG AGGTGGAAGT GGTACCCTTC CAGGAAGTGT GGGGCCGCAG CTACTGCCGG | 480 |
| GCGCTGGAGA GGCTGGTGGA CGTCGTGTCC GAGTACCCCA GCGAGGTGGA GCACATGTTC | 540 |
| AGCCCATCCT GTGTCTCCCT GCTGCGCTGC ACCGGCTGCT GCGGCGATGA GAATCTGCAC | 600 |
| TGTGTGCCGG TGGAGACGGC CAATGTCACC ATGCAGCTCC TAAAGATCCG TTCTGGGGAC | 660 |
| CGGCCCTCCT ACGTGGAGCT GACGTTCTCT CAGCACGTTC GCTGCGAATG CCGGCCTCTG | 720 |
| CGGGAGAAGA TGAAGCCGGA AAGGTGCGGC GATGCTGTTC CCCGGAGGTA ACCCACCCCT | 780 |
| TGGAGGAGAG AGACCCCGCA CCCGGCTCGT GTATTTATTA CCGTCACACT CTTCAGTGAC | 840 |
| TCCTGCTGGT ACCTGCCCTC TATTTATTAG CCAACTGTTT CCCTGCTGAA TGCCTCGCTC | 900 |
| CCTTCAAGAC GAGGGGCAGG GAAGGACAGG ACCCTCAGGA ATTCAGTGCC TTCAACAACG | 960 |
| TGAGAGAAAG AGAGAAGCCA GCCACAGACC CCTGGGAGCT TCCGCTTTGA AAGAAGCAAG | 1020 |
| ACACGTGGCC TCGTGAGGGG CAAGCTAGGC CCCAGAGGCC CTGGAGGTCT CCAGGGGCCT | 1080 |
| GCAGAAGGAA AGAAGGGGGC CCTGCTACCT GTTCTTGGGC CTCAGGCTCT GCACAGACAA | 1140 |
| GCAGCCCTTG CTTTCGGAGC TCCTGTCCAA AGTAGGGATG CGGATTCTGC TGGGGCCGCC | 1200 |
| ACGGCCTGGT GGTGGGAAGG CCGGCAGCGG GCGGAGGGGA TTCAGCCACT TCCCCCTCTT | 1260 |
| CTTCTGAAGA TCAGAACATT CAGCTCTGGA GAACAGTGGT TGCCTGGGGG CTTTTGCCAC | 1320 |
| TCCTTGTCCC CCGTGATCTC CCCTCACACT TTGCCATTTG CTTGTACTGG GACATTGTTC | 1380 |
| TTTCCGGCCG AGGTGCCACC ACCCTGCCCC CACTAAGAGA CACATACAGA GTGGGCCCCG | 1440 |
| GGCTGGAGAA AGAGCTGCCT GGATGAGAAA CAGCTCAGCC AGTGGGGATG AGGTCACCAG | 1500 |
| GGGAGGAGCC TGTGCGTCCC AGCTGAAGGC AGTGGCAGGG GAGCAGGTTC CCCAAGGGCC | 1560 |
| CTGGCACCCC CACAAGCTGT CCCTGCAGGG CCATCTGACT GCCAAGCCAG ATTCTCTTGA | 1620 |
| ATAAAGTATT CTAGTGTGGA AACGC | |

ACH4 DNA sequence
Gene name: nidogen 2 (NID2)
Unigene number: Hs.82733
Probeset Accession #: D86425
Nucleic Acid Accession #: NM_007361 cluster
Coding sequence: 1-4131 (predicted start/stop codons underlined)

| | |
|---|---|
| ATGGAGGGGG ACCGGGTGGC CGGGCGGCCG GTGCTGTCGT CGTTACCAGT GCTACTGCTG | 60 |
| CTGCAGTTGC TAATGTTGCG GGCCGCGGCG CTGCACCCAG ACGAGCTCTT CCCACACGGG | 120 |
| GAGTCGTGGT GGGACCAGCT CCTGCAGGAA GGCGACGACG TAAAGCTCAG CCGTGGTGAA | 180 |
| GCTGGCGAAT CCCCTGCACT TCTTACGAAG CCCGATTCAG CAACCTCTAC GTGGGCACCA | 240 |
| ACGGCATCAT CTCCACTCAG GACTTCCCCA GGGAAACGCA GTATGTGGAC TATGATTTCC | 300 |
| CCACCGACTT CCCGGCCATC GCCCCTTTTC TGGCGGACAT CGACACGAGC CACGGCAGAG | 360 |
| GCCGAGTCCT GTACCGAGAG GACACCTCCC CCGCAGTGCT GGGCCTGGCC GCCCGCTATG | 420 |
| TGCGCGCTGG CTTCCCGCGC TCTGCGCGCT TTTTACCCCC ACCCACGCCT TCCTGGCCAC | 480 |
| CTGGGAGCAG GTAGGCGCTT ACGAGGAGGT CAAACGCGGG CGCTGCCCTC GGGAGAGCTG | 540 |
| AACACTTTCC AGGCAGTTTT GGCATCTGAT GGGTCTGATA GCTACGCCCT CTTTCTTTAT | 600 |
| CCTGCCAACG GCCTGCAGTT CCTTGGAACC CGCCCCAAAG AGTCTTACAA TGTCCAGCTT | 660 |
| CAGCTTCCAG CTCGGGTGGG CTTCTGCCGA GGGGAGGCTG ATGATCTGAA GTCAGAAGGA | 720 |
| CCATATTTCA GCTTGACTAG CACTGAACAG TCTGTGAAAA ATCTCTATCA ACTAAGCAAC | 780 |
| CTGGGGATCC CTGGAGTGTG GGCTTTCCAT ATCGGCAGCA CTTCCCCGTT GGACAATGTC | 840 |
| AGGCCAGCTG CAGTTGGAGA CCTTTCCGCT GCCCACTCTT CTGTTCCCCT GGGACGTTCC | 900 |
| TTCAGCCATG CTACAGCCCT GGAAAGTGAC TATAATGAGG ACAATTTGGA TTACTACGAT | 960 |
| GTGAATGAGG AGGAAGCTGA ATACCTTCCG GGTGAACCAG AGGAGGCATT GAATGGCCAC | 1020 |
| AGCAGCATTG ATGTTTCCTT CCAATCCAAA GTGGATACAA AGCCTTTAGA GGAATCTTCC | 1080 |
| ACCTTGGATC CTCACACCAA AGAAGGAACA TCTCTGGGAG AGGTAGGGGG CCCAGATTTA | 1140 |
| AAAGGCCAAG TTGAGCCCTG GGATGAGAGA GAGACCAGAA GCCCAGCTCC ACCAGAGGTA | 1200 |
| GACAGAGATT CACTGGCTCC TTCCTGGGAA ACCCCACCAC CGTACCCCGA AAACGGAAGC | 1260 |
| ATCCAGCCCT ACCCAGATGG AGGGCCAGTG CCTTCGGAAA TGGATGTTCC CCCAGCTCAT | 1320 |
| CCTGAAGAAG AAATTGTTCT TCGAAGTTAC CCTGCTTCAG GTCACACTAC ACCCTTAAGT | 1380 |
| CGAGGGACGT ATGAGGTGGG ACTGGAAGAC AACATAGGTT CCAACACCGA GGTCTTCACG | 1440 |
| TATAATGCTG CCAACAAGGA AACCTGTGAA CACAACCACA GACAATGCTC CCGGCATGCC | 1500 |
| TTCTGCACGG ACTATGCCAC TGGCTTCTGC TGCCACTGCC AATCCAAGTT TTATGGAAAT | 1560 |
| GGGAAGCACT GTCTGCCTGA GGGGGCACCT CACCGAGTGA ATGGGAAAGT GAGTGGCCAC | 1620 |
| CTCCACGTGG GCCATACACC CGTGCACTTC ACTGATGTGG ACCTGCATGC GTATATCGTG | 1680 |
| GGCAATGATG GCAGAGCCTA CACGGCCATC AGCCACATCC CACAGCCAGC AGCCCAGGCC | 1740 |
| CTCCTCCCCC TCACACCAAT TGGAGGCCTG TTTGGCTGGC TCTTTGCTTT AGAAAAACCT | 1800 |
| GGCTCTGAGA ACGGCTTCAG CCTCGCAGGT GCTGCCTTTA CCCATGACAT GGAAGTTACA | 1860 |
| TTACCCGG GAGAGGAGAC GGTTCGTATC ACTCAAACTG CTGAGGGACT TGACCCAGAG | 1920 |
| AACTACCTGA GCATTAAGAC CAACATTCAA GGCCAGGTGC CTTACGTCCC AGCAAATTTC | 1980 |
| ACAGCCCACA TCTCTCCCTA CAAGGAGCTG TACCACTACT CCGACTCCAC TGTGACCTCT | 2040 |
| ACAAGTTCCA GAGACTACTC TCTGACTTTT GGTGCAATCA ACCAAACATG GTCCTACCGC | 2100 |
| ATCCACCAGA ACATCACTTA CCAGGTGTGC AGGCACGCCC CCAGACACCC GTCCTTCCCC | 2160 |
| ACCACCCAGC AGCTGAACGT GGACCGGGTC TTTGCCTTGT ATAATGATGA AGAAAGAGTG | 2220 |
| CTTAGATTTG CTGTGACCAA TCAAATTGGC CCGGTCAAAG AAGATTCAGA CCCCACTCCG | 2280 |
| GTGAATCCTT GCTATGATGG GAGCCACATG TGTGACACAA CAGCACGGTG CCATCCAGGG | 2340 |
| ACAGGTGTAG ATTACACCTG TGAGTGCGCA TCTGGGTACC AGGGAGATGG ACGGAACTGT | 2400 |
| GTGGATGAAA ATGAATGTGC AACTGGCTTT CATCGCTGTG GCCCCAACTC TGTATGTATC | 2460 |
| AACTTGCCTG GAAGCTACAG GTGTGAGTGC CGGAGTGGTT ATGAGTTTGC AGATGACCGG | 2520 |
| CATACTTGCA TCTTGATCAC CCCACCTGCC AACCCCTGTG AGGATGGCAG TCATACCTGT | 2580 |
| GCTCCTGCTG GGCAGGCCCG GTGTGTTCAC CATGGAGGCA GCACGTTCAG CTGTGCCTGC | 2640 |
| CTGCCTGGTT ATGCCGGCGA TGGGCACCAG TGCACTGATG TAGATGAATG CTCAGAAAAC | 2700 |
| AGATGTCACC CTGCAGCTAC CTGCTACAAT ACTCCTGGTT CCTTCTCCTG CCGTTGTCAA | 2760 |
| CCCGGATATT ATGGGGATGG ATTTCAGTGC ATACCTGACT CCACCTCAAG CCTGACACCC | 2820 |
| TGTGAACAAC AGCAGCGCCA TGCCCAGGCC CAGTATGCCT ACCCTGGGGC CCGGTTCCAC | 2880 |
| ATCCCCCAAT GCGACGAGCA GGGCAACTTC CTGCCCCTAC AGTGTCATGG CAGCACTGGT | 2940 |
| TTCTGCTGGT GCGTGGACCC TGATGGTCAT GAAGTTCCTG GTACCCAGAC TCCACCTGGC | 3000 |
| TCCACCCCGC CTCACTGTGG ACCATCACCA GAGCCCACCC AGAGGCCCCC GACCATCTGT | 3060 |
| GAGCGCTGGA GGGAAAACCT GCTGGAGCAC TACGGTGGCA CCCCCCGAGA TGACCAGTAC | 3120 |
| GTGCCCCAGT GCGATGACCT GGGCCACTTC ATCCCCCTGC AGTGCCACGG AAAGAGCGAC | 3180 |
| TTCTGCTGGT GTGTGGACAA AGATGGCAGA GAGGTGCAGG GCACCCGCTC CCAGCCAGGC | 3240 |
| ACCACCCCTG CGTGTATACC CACCGTCGCT CCACCCATGG TCCGGCCCAC GCCCCGGCCA | 3300 |
| GATGTGACCC CTCCATCTGT GGGCACCTTC CTGCTCTATA CTCAGGGCCA GCAGATTGGC | 3360 |
| TACTTACCCC TCAATGGCAC CAGGCTTCAG AAGGATGCAG CTAAGACCCT GCTGTCTCTG | 3420 |
| CATGGCTCCA TAATCGTGGG AATTGATTAC GACTGCCGGG AGAGGATGGT GTACTGGACA | 3480 |
| GATGTTGCTG GACGGACAAT CAGCCGTGCC GGTCTGGAAC TGGGAGCAGA GCCTGAGACG | 3540 |
| ATCGTGAATT CAGGTCTGAT AAGCCCTGAA GGACTTGCCA TAGACCACAT CCGCAGAACA | 3600 |
| ATGTACTGGA CGGACAGTGT CCTGGATAAG ATAGAGAGCG CCCTGCTGGA TGGCTCTGAG | 3660 |
| CGCAAGGTCC TCTTCTACAC AGATCTGGTG AATCCCCGTG CCATCGCTGT GGATCCAATC | 3720 |
| CGAGGCAACT TGTACTGGAC AGACTGGAAT AGAGAAGCTC CTAAAATTGA AACGTCATCT | 3780 |
| TTAGATGGAG AAAACAGAAG AATTCTGATC AATACAGACA TTGGATTGCC CAATGGCTTA | 3840 |
| ACCTTTGACC CTTTCTCTAA ACTGCTCTGC TGGGCAGATG CAGGAACCAA AAAACTGGAG | 3900 |
| TGTACACTAC CTGATGGAAC TGGACGGCGT GTCATTCAAA ACAACCTCAA GTACCCCTTC | 3960 |
| AGCATCGTAA GCTATGCAGA TCACTTCTAC CACACAGACT GGAGGAGGGA TGGTGTTGTA | 4020 |
| TCAGTAAATA AACATAGTGG CCAGTTTACT GATGAGTATC TCCCAGAACA ACGATCTCAC | 4080 |
| CTCTACGGGA TAACTGCAGT CTACCCCTAC TGCCCAACAG GAAGAAAGTA AGTACAGTAA | 4140 |
| TGTAAAGGAA GACTTGGAGT TTACAATCAG AACCTGGACC CTAAAGAACA GTGACTGCAA | 4200 |
| AGGCAAAGAA AGTAAAAAAG GAATTGGCCA TTAGACGTTC CTGAGCATCC AAGATGAACA | 4260 |
| TTTTGTAGTG CAAAAAGACT TTTGTGAAAA GCTGATACCT CAATCTTTAC TACTGTATTT | 4320 |
| TTAAAAATGA AGGTTGTTAT TGCAAGTTTA AAAAGGTAAC AGAATTTTAA CTGTTGCTTA | 4380 |
| TTAAAGCAAC TTCTTGTAAA CATTTATCAT TAATATTTAA AAGATCAAAT TCATTCAACT | 4440 |
| AAGAATTAGA GTTTAAGACT CTAAACCTGA TTTTTGCCAT GGATTCCTTC TGGCCAAGAA | 4500 |
| ATTAAAGCAC ATGTGATCAA TATAACAATA TAATCCTAAA CCTTGACAGT TGGAGAAGCC | 4560 |
| AATGCAGAAC TGATGGGAAA GGACCAATTA TTTATAGTTT CCAAACAAAA GTTCTAAGAT | 4620 |
| TTTTTACCTC TGCATCAGTG CATTTCTATT TATATCAAAA GGTGCTAAAA TGATTCAATT | 4680 |
| TGCATTTTCT GATCCTGTAG TGCCTCTATA GAAGTACCCA CAGAAAGTAA AGTATCACAT | 4740 |
| TTATAAATAC CAAAGATGTA ACAATTTTAA AATTTTCTAG ATTACTCCAA TAAAGTGTTT | 4800 |
| TAAGTTTAAA AAAAAAAAAA AAAAAAAAA | |

ACH5 DNA sequence
Gene name: SNL (singed-like; sea urchin fascin homolog-like)
Unigene number: Hs.118400
Probeset Accession #: U03057
Nucleic Acid Accession #: NM_003088
Coding sequence: 112-1593 (predicted start/stop codons underlined)

| | |
|---|---|
| GCGGAGGGTG CGTGCGGGCC GCGGCAGCCG AACAAAGGAG CAGGGGCGCC GCCGCAGGGA | 60 |
| CCCGCCACCC ACCTCCCGGG GCCGCGCAGC GGCCTCTCGT CTACTGCCAC CATGACCGCC | 120 |
| AACGGCACAG CCGAGGCGGT GCAGATCCAG TTCGGCCTCA TCAACTGCGG CAACAAGTAC | 180 |
| CTGACGGCCG AGGCGTTCGG GTTCAAGGTG AACGCGTCCG CCAGCAGCCT GAAGAAGAAG | 240 |
| CAGATCTGGA CGCTGGAGCA GCCCCCTGAC GAGGCGGGCA GCGCGGCCGT GTGCCTGCGC | 300 |
| AGCCACCTGG GCCGCTACCT GGCGGCGGAC AAGGACGGCA ACGTGACCTG CGAGCGCGAG | 360 |
| GTGCCCGGTC CCGACTGCCG TTTCCTCATC GTGGCGCACG ACGACGGTCG CTGGTCGCTG | 420 |
| CAGTCCGAGG CGCACCGGCG CTACTTCGGC GGCACCGAGG ACCGCCTGTC CTGCTTCGCG | 480 |
| CAGACGGTGT CCCCCGCCGA GAAGTGGAGC GTGCACATCG CCATGCACCC TCAGGTCAAC | 540 |
| ATCTACAGTG TCACCCGTAA GCACTACGCG CACCTGAGCG CGCGGCCGGC CGACGAGATC | 600 |
| GCCGTGGACC GCGACGTGCC CTGGGGCGTC GACTCGCTCA TCACCCTCGC CTTCCAGGAC | 660 |
| CAGCGCTACA GCGTGCAGAC CGCCGACCAC CGCTTCCTGC GCCACGACGG GCGCCTGGTG | 720 |
| GCGCGCCCCG AGCCGGCCAC TGGCTACACG CTGGAGTTCC GCTCCGGCAA GGTGGCCTTC | 780 |
| CGCGACTGCG AGGGCCGTTA CCTGGCGCCG TCGGGGCCCA GCGGCACGCT CAAGGCGGGC | 840 |
| AAGGCCACCA AGGTGGGCAA GGACGAGCTC TTTGCTCTGG AGCAGAGCTG CGCCCAGGTC | 900 |
| GTGCTGCAGG CGGCCAACGA GAGGAACGTG TCCACGCGCC AGGGTATGGA CCTGTCTGCC | 960 |
| AATCAGGACG AGGAGACCGA CCAGGAGACC TTCCAGCTGG AGATCGACCG CGACACCAAA | 1020 |
| AAGTGTGCCT TCCGTACCCA CACGGGCAAG TACTGGACGC TGACGGCCAC CGGGGGCGTG | 1080 |
| CAGTCCACCG CCTCCAGCAA GAATGCCAGC TGCTACTTTG ACATCGAGTG GCGTGACCGG | 1140 |
| CGCATCACAC TGAGGGCGTC CAATGGCAAG TTTGTGACCT CCAAGAAGAA TGGGCAGCTG | 1200 |
| GCCGCCTCGG TGGAGACAGC AGGGGACTCA GAGCTCTTCC TCATGAAGCT CATCAACCGC | 1260 |
| CCCATCATCG TGTTCCGCGG GGAGCATGGC TTCATCGGCT GCCGCAAGGT CACGGGCACC | 1320 |
| CTGGACGCCA ACCGCTCCAG CTATGACGTC TTCCAGCTGG AGTTCAACGA TGGCGCCTAC | 1380 |
| AACATCAAAG ACTCCACAGG CAAATACTGG ACGGTGGGCA GTGACTCCGC GGTCACCAGC | 1440 |
| AGCGGCGACA CTCCTGTGGA CTTCTTCTTC GAGTTCTGCG ACTATAACAA GGTGGCCATC | 1500 |
| AAGGTGGGCG GGCGCTACCT GAAGGGCGAC CACGCAGGCG TCCTGAAGGC CTCGGCGGAA | 1560 |
| ACCGTGGACC CCGCCTCGCT CTGGGAGTAC TAGGGCCGGC CCGTCCTTCC CCGCCCCTGC | 1620 |
| CCACATGGCG GCTCCTGCCA ACCCTCCCTG CTAACCCCTT CTCCGCCAGG TGGGCTCCAG | 1680 |
| GGCGGGAGGC AAGCCCCCTT GCCTTTCAAA CTGGAAACCC CAGAGAAAAC GGTGCCCCCA | 1740 |
| CCTGTCGCCC CTATGGACTC CCCACTCTCC CCTCCGCCCG GGTTCCCTAC TCCCCTCGGG | 1800 |
| TCAGCGGCTG CGGCCTGGCC CTGGGAGGGA TTTCAGATGC CCCTGCCCTC TTGTCTGCCA | 1860 |
| CGGGGCGAGT CTGGCACCTC TTTCTTCTGA CCTCAGACGG CTCTGAGCCT TATTTCTCTG | 1920 |
| GAAGCGGCTA AGGGACGGTT GGGGGCTGGG AGCCCTGGGC GTGTAGTGTA ACTGGAATCT | 1980 |
| TTTGCCTCTC CCAGCCACCT CCTCCCAGCC CCCCAGGAGA GCTGGGCACA TGTCCCAAGC | 2040 |
| CTGTCAGTGG CCCTCCCTGG TGCACTGTCC CCGAAACCCC TGCTTGGGAA GGGAAGCTGT | 2100 |
| CGGGAGGGCT AGGACTGACC CTTGTGGTGT TTTTTTGGGT GGTGGCTGGA AACAGCCCCT | 2160 |
| CTCCCACGTG GGAGAGGCTC AGCCTGGCTC CCTTCCCTGG AGCGGCAGGG CGTGACGGCC | 2220 |
| ACAGGGTCTG CCCGCTGCAC GTTCTGCCAA GGTGGTGGTG GCGGGCGGGT AGGGGTGTGG | 2280 |
| GGGCCGTCTT CCTCCTGTCT CTTTCCTTTC ACCCTAGCCT GACTGGAAGC AGAAAATGAC | 2340 |
| CAAATCAGTA TTTTTTTTAA TGAAATATTA TTGCTGGAGG CGTCCCAGGC AAGCCTGGCT | 2400 |
| GTAGTAGCGA GTGATCTGGC GGGGGGCGTC TCAGCACCCT CCCCAGGGGG TGCATCTCAG | 2460 |
| CCCCCTCTTT CCGTCCTTCC CGTCCAGCCC CAGCCCTGGG CCTGGGCTGC CGACACCTGG | 2520 |
| GCCAGAGCCC CTGCTGTGAT TGGTGCTCCC TGGGCCTCCC GGGTGGATGA AGCCAGGCGT | 2580 |
| CGCCCCCTCC GGGAGCCCTG GGGTGAGCCG CCGGGGCCCC CCTGCTGCCA GCCTCCCCCG | 2640 |
| TCCCCAACAT GCATCTCACT CTGGGTGTCT TGGTCTTTTA TTTTTTGTAA GTGTCATTTG | 2700 |
| TATAACTCTA AACGCCCATG ATAGTAGCTT CAAACTGGAA ATAGCGAAAT AAAATAACTC | 2760 |
| AGTCTGC | |

ACH6 DNA sequence
Gene name: endothelial protein C receptor (EPCR; PROCR)
Unigene number: Hs.82353
Probeset Accession #: L35545
Nucleic Acid Accession #: NM_006404
Coding sequence: 25-741 (predicted start/stop codons underlined)

| | |
|---|---|
| CAGGTCCGGA GCCTCAACTT CAGGATGTTG ACAACATTGC TGCCGATACT GCTGCTGTCT | 60 |
| GGCTGGGCCT TTTGTAGCCA AGACGCCTCA GATGGCCTCC AAAGACTTCA TATGCTCCAG | 120 |
| ATCTCCTACT TCCGCGACCC CTATCACGTG TGGTACCAGG GCAACGCGTC GCTGGGGGGA | 180 |
| CACCTAACGC ACGTGCTGGA AGGCCCAGAC ACCAACACCA CGATCATTCA GCTGCAGCCC | 240 |
| TTGCAGGAGC CCGAGAGCTG GGCGCGCACG CAGAGTGGCC TGCAGTCCTA CCTGCTCCAG | 300 |
| TTCCACGGCC TCGTGCGCCT GGTGCACCAG GAGCGGACCT TGGCCTTTCC TCTGACCATC | 360 |
| CGCTGCTTCC TGGGCTGTGA GCTGCCTCCC GAGGGCTCTA GAGCCCATGT CTTCTTCGAA | 420 |
| GTGGCTGTGA ATGGGAGCTC CTTTGTGAGT TTCCGGCCGG AGAGAGCCTT GTGGCAGGCA | 480 |
| GACACCCAGG TCACCTCCGG AGTGGTCACC TTCACCCTGC AGCAGCTCAA TGCCTACAAC | 540 |
| CGCACTCGGT ATGAACTGCG GGAATTCCTG GAGGACACCT GTGTGCAGTA TGTGCAGAAA | 600 |
| CATATTTCCG CGGAAAACAC GAAAGGGAGC CAAACAAGCC GCTCCTACAC TTCGCTGGTC | 660 |
| CTGGGCGTCC TGGTGGGCGG TTTCATCATT GCTGGTGTGG CTGTAGGCAT CTTCCTGTGC | 720 |
| ACAGGTGGAC GGCGATGTTA ATTACTCTCC AGCCCCGTCA GAAGGGGCTG GATTGATGGA | 780 |
| GGCTGGCAAG GGAAAGTTTC AGCTCACTGT GAAGCCAGAC TCCCCAACTG AAACACCAGA | 840 |
| AGGTTTGGAG TGACAGCTCC TTTCTTCTCC CACATCTGCC CACTGAAGAT TTGAGGGAGG | 900 |
| GGAGATGGAG AGGAGAGGTG GACAAAGTAC TTGGTTTGCT AAGAACCTAA GAACGTGTAT | 960 |
| GCTTTGCTGA ATTAGTCTGA TAAGTGAATG TTTATCTATC TTTGTGGAAA ACAGATAATG | 1020 |
| GAGTTGGGGC AGGAAGCCTA TGCGCCATCC TCCAAAGACA GACAGAATCA CCTGAGGCGT | 1080 |
| TCAAAAGATA TAACCAAATA AACAAGTCAT CCACAATCAA AATACAACAT TCAATACTTC | 1140 |
| CAGGTGTGTC AGACTTGGGA TGGGACGCTG ATATAATAGG GTAGAAAGAA GTAACACGAA | 1200 |
| GAAGTGGTGG AAATGTAAAA TCCAAGTCAT ATGGCAGTGA TCAATTATTA ATCAATTAAT | 1260 |
| AATATTAATA AATTTCTTAT ATTT | |

ACH8 DNA sequence
Gene name: melanoma adhesion molecule (MCAM; MUC18)
Unigene number: Hs.211579
Probeset Accession #: D51069
Nucleic Acid Accession #: NM_006500
Coding sequence: 27-1967 (predicted start and stop codons underlined)

| | |
|---|---|
| ACTTGCGTCT CGCCCTCCGG CCAAGCATGG GGCTTCCCAG GCTGGTCTGC GCCTTCTTGC | 60 |
| TCGCCGCCTG CTGCTGCTGT CCTCGCGTCG CGGGTGTGCC CGGAGAGGCT GAGCAGCCTG | 120 |
| CGCCTGAGCT GGTGGAGGTG GAAGTGGGCA GCACAGCCCT TCTGAAGTGC GGCCTCTCCC | 180 |
| AGTCCCAAGG CAACCTCAGC CATGTCGACT GGTTTTCTGT CCACAAGGAG AAGCGGACGC | 240 |
| TCATCTTCCG TGTGCGCCAG GGCCAGGGCC AGAGCGAACC TGGGGAGTAC GAGCAGCGGC | 300 |
| TCAGCCTCCA GGACAGAGGG GCTACTCTGG CCCTGACTCA AGTCACCCCC CAAGACGAGC | 360 |
| GCATCTTCTT GTGCCAGGGC AAGCGCCCTC GGTCCCAGGA GTACCGCATC CAGCTCCGCG | 420 |
| TCTACAAAGC TCCGGAGGAG CCAAACATCC AGGTCAACCC CCTGGGCATC CCTGTGAACA | 480 |
| GTAAGGAGCC TGAGGAGGTC GCTACCTGTG TAGGGAGGAA CGGGTACCCC ATTCCTCAAG | 540 |
| TCATCTGGTA CAAGAATGGC CGGCCTCTGA AGGAGGAGAA GAACCGGGTC CACATTCAGT | 600 |
| CGTCCCAGAC TGTGGAGTCG AGTGGTTTGT ACACCTTGCA GAGTATTCTG AAGGCACAGC | 660 |
| TGGTTAAAGA AGACAAAGAT GCCCAGTTTT ACTGTGAGCT CAACTACCGG CTGCCCAGTG | 720 |
| GGAACCACAT GAAGGAGTCC AGGGAAGTCA CCGTCCCTGT TTTCTACCCG ACAGAAAAAG | 780 |
| TGTGGCTGGA AGTGGAGCCC GTGGGAATGC TGAAGGAAGG GGACCGCGTG GAAATCAGGT | 840 |
| GTTTGGCTGA TGGCAACCCT CCACCACACT TCAGCATCAG CAAGCAGAAC CCCAGCACCA | 900 |
| GGGAGGCAGA GGAAGAGACA ACCAACGACA ACGGGGTCCT GGTGCTGGAG CCTGCCCGGA | 960 |
| AGGAACACAG TGGGCGCTAT GAATGTCAGG CCTGGAACTT GGACACCATG ATATCGCTGC | 1020 |
| TGAGTGAACC ACAGGAACTA CTGGTGAACT ATGTGTCTGA CGTCCGAGTG AGTCCCGCAG | 1080 |
| CCCCTGAGAG ACAGGAAGGC AGCAGCCTCA CCCTGACCTG TGAGGCAGAG AGTAGCCAGG | 1140 |
| ACCTCGAGTT CCAGTGGCTG AGAGAAGAGA CAGACCAGGT GCTGGAAAGG GGGCCTGTGC | 1200 |
| TTCAGTTGCA TGACCTGAAA CGGGAGGCAG GAGGCGGCTA TCGCTGCGTG GCGTCTGTGC | 1260 |
| CCAGCATACC CGGCCTGAAC CGCACACAGC TGGTCAAGCT GGCCATTTTT GGCCCCCCTT | 1320 |
| GGATGGCATT CAAGGAGAGG AAGGTGTGGG TGAAAGAGAA TATGGTGTTG AATCTGTCTT | 1380 |
| GTGAAGCGTC AGGGCACCCC CGGCCCACCA TCTCCTGGAA CGTCAACGGC ACGGCAAGTG | 1440 |
| AACAAGACCA AGATCCACAG CGAGTCCTGA GCACCCTGAA TGTCCTCGTG ACCCCGGAGC | 1500 |
| TGTTGGAGAC AGGTGTTGAA TGCACGGCCT CCAACGACCT GGGCAAAAAC ACCAGCATCC | 1560 |
| TCTTCCTGGA GCTGGTCAAT TTAACCACCC TCACACCAGA CTCCAACACA ACCACTGGCC | 1620 |
| TCAGCACTTC CACTGCCAGT CCTCATACCA GAGCCAACAG CACCTCCACA GAGAGAAAGC | 1680 |
| TGCCGGAGCC GGAGAGCCGG GGCGTGGTCA TCGTGGCTGT GATTGTGTGC ATCCTGGTCC | 1740 |
| TGGCGGTGCT GGGCGCTGTC CTCTATTTCC TCTATAAGAA GGGCAAGCTG CCGTGCAGGC | 1800 |
| GCTCAGGGAA GCAGGAGATC ACGCTGCCCC CGTCTCGTAA GACCGAACTT GTAGTTGAAG | 1860 |
| TTAAGTCAGA TAAGCTCCCA GAAGAGATGG GCCTCCTGCA GGGCAGCAGC GGTGACAAGA | 1920 |
| GGGCTCCGGG AGACCAGGGA GAGAAATACA TCGATCTGAG GCATTAGCCC CGAATCACTT | 1980 |
| CAGCTCCCTT CCCTGCCTGG ACCATTCCCA GCTCCCTGCT CACTCTTCTC TCAGCCAAAG | 2040 |
| CCTCCAAAGG GACTAGAGAG AAGCCTCCTG CTCCCCTCAC CTGCACACCC CCTTTCAGAG | 2100 |
| GGCCACTGGG TTAGGACCTG AGGACCTCAC TTGGCCCTGC AAGCCGCTTT TCAGGGACCA | 2160 |
| GTCCACCACC ATCTCCTCCA CGTTGAGTGA AGCTCATCCC AAGCAAGGAG CCCCAGTCTC | 2220 |
| CCGAGCGGGT AGGAGAGTTT CTTGCAGAAC GTGTTTTTTC TTTACACACA TTATGGCTGT | 2280 |
| AAATACCTGG CTCCTGCCAG CAGCTGAGCT GGGTAGCCTC TCTGAGCTGG TTTCCTGCCC | 2340 |
| CAAAGGCTGG CTTCCACCAT CCAGGTGCAC CACTGAAGTG AGGACACACC GGAGCCAGGC | 2400 |
| GCCTGCTCAT GTTGAAGTGC GCTGTTCACA CCCGCTCCGG AGAGCACCCC AGCGGCATCC | 2460 |
| AGAAGCAGCT GCAGTGTTGC TGCCACCACC CTCCTGCTCG CCTCTTCAAA GTCTCCTGTG | 2520 |
| ACATTTTTTC TTTGGTCAGA AGCCAGGAAC TGGTGTCATT CCTTAAAAGA TACGTGCCGG | 2580 |
| GGCCAGGTGT GGTGGCTCAC GCCTGTAATC CCAGCACTTT GGGAGGCCGA GGCGGGCGGA | 2640 |
| TCACAAAGTC AGGACGAGAC CATCCTGGCT AACACGGTGA AACCCTGTCT CTACTAAAAA | 2700 |
| TACAAAAAAA AATTAGCTAG GCGTAGTGGT TGGCACCTAT AGTCCCAGCT ACTCGGAAGG | 2760 |
| CTGAAGCAGG AGAATGGTAT GAATCCAGGA GGTGGAGCTT GCAGTGAGCC GAGACCGTGC | 2820 |
| CACTGCACTC CAGCCTGGGC AACACAGCGA GACTCCGTCT CGAGGAAAAA AAAAGAAAAG | 2880 |
| ACGCGTACCT GCGGTGAGGA AGCTGGGCGC TGTTTTCGAG TTCAGGTGAA TTAGCCTCAA | 2940 |
| TCCCCGTGTT CACTTGCTCC CATAGCCCTC TTGATGGATC ACGTAAAACT GAAAGGCAGC | 3000 |
| GGGGAGCAGA CAAAGATGAG GTCTACACTG TCCTTCATGG GGATTAAAGC TATGGTTATA | 3060 |
| TTAGCACCAA ACTTCTACAA ACCAAGCTCA GGGCCCCAAC CCTAGAAGGG CCCAAATGAG | 3120 |
| AGAATGGTAC TTAGGGATGG AAAACGGGGC CTGGCTAGAG CTTCGGGTGT GTGTGTCTGT | 3180 |
| CTGTGTGTAT GCATACATAT GTGTGTATAT ATGGTTTTGT CAGGTGTGTA AATTTGCAAA | 3240 |
| TTGTTTCCTT TATATATGTA TGTATATATA TATATGAAAA TATATATATA TATGAAAAAT | 3300 |
| AAAGCTTAAT TGTCCCAGAA AATCATACAT TGCTTTTTTA TTCTACATGG GTACCACAGG | 3360 |
| AACCTGGGGG CCTGTGAAAC TACAACCAAA AGGCACACAA AACCGTTTCC AGTTGGCAGC | 3420 |
| AGAGATCAGG GGTTACCTCT GCTTCTGAGC AAATGGCTCA AGCTCTACCA GAGCAGACAG | 3480 |
| CTACCCTACT TTTCAGCAGC AAAACGTCCC GTATGACGCA GCACGAAGGG CCTGGCAGGC | 3540 |
| TGTTAGCAGG AGCTATGTCC CTTCCTATCG TTTCCGTCCA CTT | |

ACH9 DNA sequence
Gene name: endothelin-1 (EDN1)
Unigene number: Hs.2271
Probeset Accession #: J05008
Nucleic Acid Accession #: NM_001955
Coding sequence: 337-975 (predicted start/stop codons underlined)

| | |
|---|---|
| GGAGCTGTTT ACCCCCACTC TAATAGGGGT TCAATATAAA AAGCCGGCAG AGAGCTGTCC | 60 |
| AAGTCAGACG CGCCTCTGCA TCTGCGCCAG GCGAACGGGT CCTGCGCCTC CTGCAGTCCC | 120 |
| AGCTCTCCAC CACCGCCGCG TGCGCCTGCA GACGCTCCGC TCGCTGCCTT CTCTCCTGGC | 180 |
| AGGCGCTGCC TTTTCTCCCC GTTAAAGGGC ACTTGGGCTG AAGGATCGCT TTGAGATCTG | 240 |
| AGGAACCCGC AGCGCTTTGA GGGACCTGAA GCTGTTTTTC TTCGTTTTCC TTTGGGTTCA | 300 |
| GTTTGAACGG GAGGTTTTTG ATCCCTTTTT TTCAGAATGG ATTATTTGCT CATGATTTTC | 360 |
| TCTCTGCTGT TTGTGGCTTG CCAAGGAGCT CCAGAAACAG CAGTCTTAGG CGCTGAGCTC | 420 |
| AGCGCGGTGG GTGAGAACGG CGGGGAGAAA CCCACTCCCA GTCCACCCTG GCGGCTCCGC | 480 |
| CGGTCCAAGC GCTGCTCCTG CTCGTCCCTG ATGGATAAAG AGTGTGTCTA CTTCTGCCAC | 540 |
| CTGGACATCA TTTGGGTCAA CACTCCCGAG CACGTTGTTC CGTATGGACT TGGAAGCCCT | 600 |
| AGGTCCAAGA GAGCCTTGGA GAATTTACTT CCCACAAAGG CAACAGACCG TGAGAATAGA | 660 |
| TGCCAATGTG CTAGCCAAAA AGACAAGAAG TGCTGGAATT TTTGCCAAGC AGGAAAAGAA | 720 |
| CTCAGGGCTG AAGACATTAT GGAGAAAGAC TGGAATAATC ATAAGAAAGG AAAAGACTGT | 780 |
| TCCAAGCTTG GGAAAAAGTG TATTTATCAG CAGTTAGTGA GAGGAAGAAA AATCAGAAGA | 840 |
| AGTTCAGAGG AACACCTAAG ACAAACCAGG TCGGAGACCA TGAGAAACAG CGTCAAATCA | 900 |
| TCTTTTCATG ATCCCAAGCT GAAAGGCAAG CCCTCCAGAG AGCGTTATGT GACCCACAAC | 960 |
| CGAGCACATT GGTGACAGAC TTCGGGGCCT GTCTGAAGCC ATAGCCTCCA CGGAGAGCCC | 1020 |
| TGTGGCCGAC TCTGCACTCT CCACCCTGGC TGGGATCAGA GCAGGAGCAT CCTCTGCTGG | 1080 |
| TTCCTGACTG GCAAAGGACC AGCGTCCTCG TTCAAAACAT TCCAAGAAAG GTTAAGGAGT | 1140 |
| TCCCCCAACC ATCTTCACTG GCTTCCATCA GTGGTAACTG CTTTGGTCTC TTCTTTCATC | 1200 |
| TGGGGATGAC AATGGACCTC TCAGCAGAAA CACACAGTCA CATTCGAATT C | |

ACJ1 DNA sequence
Gene name: BMX non-receptor tyrosine kinase
Unigene number: Hs.27372
Probeset Accession #: X83107
Nucleic Acid Accession #: NM_001721
Coding sequence: 34-2061 (predicted start/stop codons underlined)

| | |
|---|---|
| GCAAGCACGG AACAAGCTGA GACGGATGAT AATATGGATA CAAAATCTAT TCTAGAAGAA | 60 |
| CTTCTTCTCA AAAGATCACA GCAAAAGAAG AAAATGTCAC CAAATAATTA CAAAGAACGG | 120 |
| CTTTTTGTTT TGACCAAAAC AAACCTTTCC TACTATGAAT ATGACAAAAT GAAAAGGGGC | 180 |
| AGCAGAAAAG GATCCATTGA AATTAAGAAA ATCAGATGTG TGGAGAAAGT AAATCTCGAG | 240 |
| GAGCAGACGC CTGTAGAGAG ACAGTACCCA TTTCAGATTG TCTATAAAGA TGGGCTTCTC | 300 |
| TATGTCTATG CATCAAATGA AGAGAGCCGA AGTCAGTGGT TGAAAGCATT ACAAAAAGAG | 360 |
| ATAAGGGGTA ACCCCCACCT GCTGGTCAAG TACCATAGTG GGTTCTTCGT GGACGGGAAG | 420 |
| TTCCTGTGTT GCCAGCAGAG CTGTAAAGCA GCCCCAGGAT GTACCCTCTG GGAAGCATAT | 480 |
| GCTAATCTGC ATACTGCAGT CAATGAAGAG AAACACAGAG TTCCCACCTT CCCAGACAGA | 540 |
| GTGCTGAAGA TACCTCGGGC AGTTCCTGTT CTCAAAATGG ATGCACCATC TTCAAGTACC | 600 |
| ACTCTAGCCC AATATGACAA CGAATCAAAG AAAAACTATG GCTCCCAGCC ACCATCTTCA | 660 |
| AGTACCAGTC TAGCGCAATA TGACAGCAAC TCAAAGAAAA TCTATGGCTC CCAGCCAAAC | 720 |
| TTCAACATGC AGTATATTCC AAGGGAAGAC TTCCCTGACT GGTGGCAAGT AAGAAAACTG | 780 |
| AAAAGTAGCA GCAGCAGTGA AGATGTTGCA AGCAGTAACC AAAAAGAAAG AAATGTGAAT | 840 |
| CACACCACCT CAAAGATTTC ATGGGAATTC CCTGAGTCAA GTTCATCTGA AGAAGAGGAA | 900 |
| AACCTGGATG ATTATGACTG GTTTGCTGGT AACATCTCCA GATCACAATC TGAACAGTTA | 960 |
| CTCAGACAAA AGGGAAAAGA AGGAGCATTT ATGGTTAGAA ATTCGAGCCA AGTGGGAATG | 1020 |
| TACACAGTGT CCTTATTTAG TAAGGCTGTG AATGATAAAA AAGGAACTGT CAAACATTAC | 1080 |
| CACGTGCATA CAAATGCTGA GAACAAATTA TACCTGGCAG AAAACTACTG TTTTGATTCC | 1140 |
| ATTCCAAAGC TTATTCATTA TCATCAACAC AATTCAGCAG GCATGATCAC ACGGCTCCGC | 1200 |
| CACCCTGTGT CAACAAAGGC CAACAAGGTC CCCGACTCTG TGTCCCTGGG AAATGGAATC | 1260 |
| TGGGAACTGA AAAGAGAAGA GATTACCTTG TTGAAGGAGC TGGGAAGTGG CCAGTTTGGA | 1320 |
| GTGGTCCAGC TGGGCAAGTG GAAGGGGCAG TATGATGTTG CTGTTAAGAT GATCAAGGAG | 1380 |
| GGCTCCATGT CAGAAGATGA ATTCTTTCAG GAGGCCCAGA CTATGATGAA ACTCAGCCAT | 1440 |
| CCCAAGCTGG TTAAATTCTA TGGAGTGTGT TCAAAGGAAT ACCCCATATA CATAGTGACT | 1500 |
| GAATATATAA GCAATGGCTG CTTGCTGAAT TACCTGAGGA GTCACGGAAA AGGACTTGAA | 1560 |
| CCTTCCCAGC TCTTAGAAAT GTGCTACGAT GTCTGTGAAG GCATGGCCTT CTTGGAGAGT | 1620 |
| CACCAATTCA TACACCGGGA CTTGGCTGCT CGTAACTGCT TGGTGGACAG AGATCTCTGT | 1680 |
| GTGAAAGTAT CTGACTTTGG AATGACAAGG TATGTTCTTG ATGACCAGTA TGTCAGTTCA | 1740 |
| GTCGGAACAA AGTTTCCAGT CAAGTGGTCA GCTCCAGAGG TGTTTCATTA CTTCAAATAC | 1800 |
| AGCAGCAAGT CAGACGTATG GGCATTTGGG ATCCTGATGT GGGAGGTGTT CAGCCTGGGG | 1860 |
| AAGCAGCCCT ATGACTTGTA TGACAACTCC CAGGTGGTTC TGAAGGTCTC CCAGGGCCAC | 1920 |
| AGGCTTTACC GGCCCCACCT GGCATCGGAC ACCATCTACC AGATCATGTA CAGCTGCTGG | 1980 |
| CACGAGCTTC CAGAAAAGCG TCCCACATTT CAGCAACTCC TGTCTTCCAT TGAACCACTT | 2040 |
| CGGGAAAAAG ACAAGCATTG AAGAAGAAAT TAGGAGTGCT GATAAGAATG AATATAGATG | 2100 |
| CTGGCCAGCA TTTTCATTCA TTTTAAGGAA AGTAGGAAGG CATAAGTAAT TTTAGCTAGT | 2160 |
| TTTTAATAGT GTTCTCTGTA TTGTCTATTA TTTAGAAATG AACAAGGCAG GAAACAAAAG | 2220 |
| ATTCCCTTGA AATTTAGATC AAATTAGTAA TTTTGTTTTA TGCTGCTCCT GATATAACAC | 2280 |
| TTTCCAGCCT ATAGCAGAAG CACATTTTCA GACTGCAATA TAGAGACTGT GTTCATGTGT | 2340 |
| AAAGACTGAG CAGAACTGAA AAATTACTTA TTGGATATTC ATTCTTTTCT TTATATTGTC | 2400 |
| ATTGTCACAA CAATTAAATA TACTACCAAG TACAGAAATG TGGAAAAAAA AAACCG | |

ACJ4 DNA sequence
Gene name: prostaglandin G/H synthase 2 (COX-2; PGHS-2)
Unigene number: Hs.196384
Probeset Accession #: D28235
Nucleic Acid Accession #: NM_000963
Coding sequence: 135-1949 (predicted start/stop codons underlined)

| | |
|---|---|
| CAATTGTCAT ACGACTTGCA GTGAGCGTCA GGAGCACGTC CAGGAACTCC TCAGCAGCGC | 60 |
| CTCCTTCAGC TCCACAGCCA GACGCCCTCA GACAGCAAAG CCTACCCCCG CGCCGCGCCC | 120 |
| TGCCCGCCGC TCGGATGCTC GCCCGCGCCC TGCTGCTGTG CGCGGTCCTG GCGCTCAGCC | 180 |
| ATACAGCAAA TCCTTGCTGT TCCCACCCAT GTCAAAACCG AGGTGTATGT ATGAGTGTGG | 240 |
| GATTTGACCA GTATAAGTGC GATTGTACCC GGACAGGATT CTATGGAGAA AACTGCTCAA | 300 |
| CACCGGAATT TTTGACAAGA ATAAAATTAT TTCTGAAACC CACTCCAAAC ACAGTGCACT | 360 |
| ACATACTTAC CCACTTCAAG GGATTTTGGA ACGTTGTGAA TAACATTCCC TTCCTTCGAA | 420 |
| ATGCAATTAT GAGTTATGTC TTGACATCCA GATCACATTT GATTGACAGT CCACCAACTT | 480 |
| ACAATGCTGA CTATGGCTAC AAAAGCTGGG AAGCCTTCTC TAACCTCTCC TATTATACTA | 540 |
| GAGCCCTTCC TCCTGTGCCT GATGATTGCC CGACTCCCTT GGGTGTCAAA GGTAAAAAGC | 600 |
| AGCTTCCTGA TTCAAATGAG ATTGTGGAAA AATTGCTTCT AAGAAGAAAG TTCATCCCTG | 660 |
| ATCCCCAGGG CTCAAACATG ATGTTTGCAT TCTTTGCCCA GCACTTCACG CATCAGTTTT | 720 |
| TCAAGACAGA TCATAAGCGA GGGCCAGCTT TCACCAACGG GCTGGGCCAT GGGGTGGACT | 780 |
| TAAATCATAT TTACGGTGAA ACTCTGGCTA GACAGCGTAA ACTGCGCCTT TTCAAGGATG | 840 |
| GAAAAATGAA ATATCAGATA ATTGATGGAG AGATGTATCC TCCCACAGTC AAAGATACTC | 900 |
| AGGCAGAGAT GATCTACCCT CCTCAAGTCC CTGAGCATCT ACGGTTTGCT GTGGGGCAGG | 960 |
| AGGTCTTTGG TCTGGTGCCT GGTCTGATGA TGTATGCCAC AATCTGGCTG CGGGAACACA | 1020 |
| ACAGAGTATG CGATGTGCTT AAACAGGAGC ATCCTGAATG GGGTGATGAG CAGTTGTTCC | 1080 |
| AGACAAGCAG GCTAATACTG ATAGGAGAGA CTATTAAGAT TGTGATTGAA GATTATGTGC | 1140 |
| AACACTTGAG TGGCTATCAC TTCAAACTGA AATTTGACCC AGAACTACTT TTCAACAAAC | 1200 |
| AATTCCAGTA CCAAAATCGT ATTGCTGCTG AATTTAACAC CCTCTATCAC TGGCATCCCC | 1260 |
| TTCTGCCTGA CACCTTTCAA ATTCATGACC AGAAATACAA CTATCAACAG TTTATCTACA | 1320 |
| ACAACTCTAT ATTGCTGGAA CATGGAATTA CCCAGTTTGT TGAATCATTC ACCAGGCAAA | 1380 |
| TTGCTGGCAG GGTTGCTGGT GGTAGGAATG TTCCACCCGC AGTACAGAAA GTATCACAGG | 1440 |
| CTTCCATTGA CCAGAGCAGG CAGATGAAAT ACCAGTCTTT TAATGAGTAC CGCAAACGCT | 1500 |
| TTATGCTGAA GCCCTATGAA TCATTTGAAG AACTTACAGG AGAAAAGGAA ATGTCTGCAG | 1560 |
| AGTTGGAAGC ACTCTATGGT GACATCGATG CTGTGGAGCT GTATCCTGCC CTTCTGGTAG | 1620 |
| AAAAGCCTCG GCCAGATGCC ATCTTTGGTG AAACCATGGT AGAAGTTGGA GCACCATTCT | 1680 |
| CCTTGAAAGG ACTTATGGGT AATGTTATAT GTTCTCCTGC CTACTGGAAG CCAAGCACTT | 1740 |
| TTGGTGGAGA AGTGGGTTTT CAAATCATCA ACACTGCCTC AATTCAGTCT CTCATCTGCA | 1800 |
| ATAACGTGAA GGGCTGTCCC TTTACTTCAT TCAGTGTTCC AGATCCAGAG CTCATTAAAA | 1860 |
| CAGTCACCAT CAATGCAAGT TCTTCCCGCT CCGGACTAGA TGATATCAAT CCCACAGTAC | 1920 |
| TACTAAAAGA ACGTTCGACT GAACTGTAGA AGTCTAATGA TCATATTTAT TTATTTATAT | 1980 |
| GAACCATGTC TATTAATTTA ATTATTTAAT AATATTTATA TTAAACTCCT TATGTTACTT | 2040 |
| AACATCTTCT GTAACAGAAG TCAGTACTCC TGTTGCGGAG AAAGGAGTCA TACTTGTGAA | 2100 |
| GACTTTTATG TCACTACTCT AAAGATTTTG CTGTTGCTGT TAAGTTTGGA AAACAGTTTT | 2160 |
| TATTCTGTTT TATAAACCAG AGAGAAATGA GTTTTGACGT CTTTTTACTT GAATTTCAAC | 2220 |
| TTATATTATA AGAACGAAAG TAAAGATGTT TGAATACTTA AACACTATCA CAAGATGGCA | 2280 |
| AAATGCTGAA AGTTTTTACA CTGTCGATGT TTCCAATGCA TCTTCCATGA TGCATTAGAA | 2340 |
| GTAACTAATG TTTGAAATTT TAAAGTACTT TTGGTTATTT TTCTGTCATC AAACAAAAAC | 2400 |
| AGGTATCAGT GCATTATTAA ATGAATATTT AAATTAGACA TTACCAGTAA TTTCATGTCT | 2460 |
| ACTTTTTAAA ATCAGCAATG AAACAATAAT TTGAAATTTC TAAATTCATA GGGTAGAATC | 2520 |
| ACCTGTAAAA GCTTGTTTGA TTTCTTAAAG TTATTAAACT TGTACATATA CCAAAAAGAA | 2580 |
| GCTGTCTTGG ATTTAAATCT GTAAAATCAG ATGAAATTTT ACTACAATTG CTTGTTAAAA | 2640 |
| TATTTTAAAA GTGATGTTCC TTTTTCACCA AGAGTATAAA CCTTTTTAGT GTGACTGTTA | 2700 |
| AAACTTCCTT TTAAATCAAA ATGCCAAATT TATTAAGGTG GTGGAGCCAC TGCAGTGTTA | 2760 |
| TCTCAAAATA AGAATATTTT GTTGAGATAT TCCAGAATTT GTTTATATGG CTGGTAACAT | 2820 |
| GTAAAATCTA TATCAGCAAA AGGGTCTACC TTTAAAATAA GCAATAACAA AGAAGAAAAC | 2880 |
| CAAATTATTG TTCAAATTTA GGTTTAAACT TTTGAAGCAA ACTTTTTTTT ATCCTTGTGC | 2940 |
| ACTGCAGGCC TGGTACTCAG ATTTTGCTAT GAGGTTAATG AAGTACCAAG CTGTGCTTGA | 3000 |
| ATAACGATAT GTTTTCTCAG ATTTTCTGTT GTACAGTTTA ATTTAGCAGT CCATATCACA | 3060 |
| TTGCAAAAGT AGCAATGACC TCATAAAATA CCTCTTCAAA ATGCTTAAAT TCATTTCACA | 3120 |
| CATTAATTTT ATCTCAGTCT TGAAGCCAAT TCAGTAGGTG CATTGGAATC AAGCCTGGCT | 3180 |
| ACCTGCATGC TGTTCCTTTT CTTTTCTTCT TTTAGCCATT TTGCTAAGAG ACACAGTCTT | 3240 |
| CTCATCACTT CGTTTCTCCT ATTTTGTTTT ACTAGTTTTA AGATCAGAGT TCACTTTCTT | 3300 |
| TGGACTCTGC CTATATTTTC TTACCTGAAC TTTTGCAAGT TTTCAGGTAA ACCTCAGCTC | 3360 |
| AGGACTGCTA TTTAGCTCCT CTTAAGAAGA TTAAAAGAGA AAAAAAAAGG CCCTTTTAAA | 3420 |
| AATAGTATAC ACTTATTTTA AGTGAAAAGC AGAGAATTTT ATTTATAGCT AATTTTAGCT | 3480 |
| ATCTGTAACC AAGATGGATG CAAAGAGGCT AGTGCCTCAG AGAGAACTGT ACGGGGTTTG | 3540 |
| TGACTGGAAA AAGTTACGTT CCCATTCTAA TTAATGCCCT TTCTTATTTA AAAACAAAAC | 3600 |
| CAAATGATAT CTAAGTAGTT CTCAGCAATA ATAATAATGA CGATAATACT TCTTTTCCAC | 3660 |
| ATCTCATTGT CACTGACATT TAATGGTACT GTATATTACT TAATTTATTG AAGATTATTA | 3720 |
| TTTATGTCTT ATTAGGACAC TATGGTTATA AACTGTGTTT AAGCCTACAA TCATTGATTT | 3780 |
| TTTTTTGTTA TGTCACAATC AGTATATTTT CTTTGGGGTT ACCTCTCTGA ATATTATGTA | 3840 |
| AACAATCCAA AGAAATGATT GTATTAAGAT TTGTGAATAA ATTTTTAGAA ATCTGATTGG | 3900 |
| CATATTGAGA TATTTAAGGT TGAATGTTTG TCCTTAGGAT AGGCCTATGT GCTAGCCCAC | 3960 |
| AAAGAATATT GTCTCATTAG CCTGAATGTG CCATAAGACT GACCTTTTAA AATGTTTTGA | 4020 |
| GGGATCTGTG GATGCTTCGT TAATTTGTTC AGCCACAATT TATTGAGAAA ATATTCTGTG | 4080 |
| TCAAGCACTG TGGGTTTTAA TATTTTTAAA TCAAACGCTG ATTACAGATA ATAGTATTTA | 4140 |
| TATAAATAAT TGAAAAAAAT TTTCTTTTGG GAAGAGGGAG AAAATGAAAT AAATATCATT | 4200 |
| AAAGATAACT CAGGAGAATC TTCTTTACAA TTTTACGTTT AGAATGTTTA AGGTTAAGAA | 4260 |
| AGAAATAGTC AATATGCTTG TATAAAACAC TGTTCACTGT TTTTTTTAAA AAAAAAACTT | 4320 |
| GATTTGTTAT TAACATTGAT CTGCTGACAA AACCTGGGAA TTTGGGTTGT GTATGCGAAT | 4380 |
| GTTTCAGTGC CTCAGACAAA TGTGTATTTA ACTTATGTAA AAGATAAGTC TGGAAATAAA | 4440 |
| TGTCTGTTTA TTTTTGTACT ATTTA | |

ACJ6 DNA sequence
Gene name: SEC14-like-1
Unigene number: Hs.75232
Probeset Accession #: D67029
Nucleic Acid Accession #: NM_003003
Coding sequence: 304-2451 (predicted start/stop codons underlined

| | |
|---|---|
| CAAGTGCCGT CGCCGCGCCC CTTCCCCCTC CCGCCTCCCC GGCCCCCTCC CCGGAACCGG | 60 |
| CGGTCGAGCT ACGGTCGCGG ACGAGTGGAA CCGAGACTGC CCCGCGGAGC CGCCGGTATG | 120 |
| AGCGCCCCTC GCCACCCCGT GTCCCAGGCC CGGCCTTTCT GACAAGAGCT AGACTTCGGG | 180 |
| CTCCTTGAGG ATATTCAGTT TTGTATGTTT GAATATCCTC TCACCATGTT CAGCATAAAG | 240 |
| TACCATTCTT AATGATTATC CTCAACAAGA CAGGTGTGAG AGGGTTGCTG TTGCATTGCA | 300 |
| ATCATGGTGC AAAAATACCA GTCCCCAGTG AGAGTGTACA AATACCCCTT TGAATTAATT | 360 |
| ATGGCTGCCT ATGAAAGGAG GTTCCCTACA TGTCCTTTGA TTCCGATGTT CGTGGGCAGT | 420 |
| GACACTGTGA GTGAATTCAA GAGCGAAGAT GGGGCTATTC ATGTCATTGA AAGGCGCTGC | 480 |
| AAGCTGGATG TAGATGCACC GAGACTGCTG AAGAAGATTG CAGGAGTTGA TTATGTTTAT | 540 |
| TTTGTCCAGA AAAACTCACT GAATTCTCGG GAACGTACTT TGCACATTGA GGCTTATAAT | 600 |
| GAAACGTTTT CCAATCGGGT CATCATTAAT GAGCATTGCT GCTACACCGT TCACCCTGAA | 660 |
| AATGAAGATT GGACCTGTTT TGAACAGTCT GCAAGTTTAG ATATTAAATC TTTCTTTGGT | 720 |
| TTTGAAAGTA CAGTGGAAAA AATTGCAATG AAACAATATA CCAGCAACAT TAAAAAAGGA | 780 |
| AAGGAAATCA TCGAATACTA CCTTCGCCAA TTAGAAGAAG AAGGCATAAC CTTTGTGCCC | 840 |
| CGTTGGAGTC CGCCTTCCAT CACGCCCTCT TCAGAGACAT CTTCATCATC CTCCAAGAAA | 900 |
| CAAGCAGCGT CCATGGCCGT CGTCATCCCA GAAGCTGCCC TCAAGGAGGG GCTGAGTGGT | 960 |
| GATGCCCTCA GCAGCCCCAG TGCACCTGAG CCCGTGGTGG GCACCCCTGA CGACAAACTA | 1020 |
| GATGCCGACC ACATCAAGAG ATACCTGGGC GATTTGACTC CGCTGCAGGA GAGCTGCCTC | 1080 |
| ATTAGACTTC GCCAGTGGCT CCAGGAGACC CACAAGGGCA AAATTCCAAA AGATGAGCAT | 1140 |
| ATTCTTCGGT TCCTCCGTGC ACGGGATTTT AATATTGACA AAGCCAGAGA GATCATGTGT | 1200 |
| CAGTCTTTGA CGTGGAGAAA GCAGCATCAG GTAGACTACA TTCTTGAAAC CTGGACCCCT | 1260 |
| CCTCAGGTCC TTCAGGATTA CTACGCGGGA GGCTGGCATC ATCACGACAA AGATGGGCGG | 1320 |
| CCCCTCTACG TGCTCAGGCT GGGGCAGATG GACACCAAAG GCTTGGTGAG AGCGCTCGGG | 1380 |
| GAGGAAGCCC TGCTGAGATA CGTTCTCTCC GTAAATGAAG AACGGCTAAG GCGATGCGAA | 1440 |
| GAGAATACAA AAGTCTTTGG TCGGCCTATC AGCTCATGGA CCTGCCTGGT GGACTTGGAA | 1500 |
| GGGCTGAACA TGCGCCACTT GTGGAGACCT GGTGTGAAAG CGCTGCTGCG GATCATCGAG | 1560 |
| GTGGTGGAGG CCAACTACCC TGAGACACTG GGCCGCCTTC TCATCCTGCG GGCGCCCAGG | 1620 |
| GTATTTCCTG TGCTCTGGAC GCTGGTTAGT CCGTTCATTG ATGACAACAC CAGAAGGAAG | 1680 |
| TTCCTCATTT ATGCAGGAAA TGACTACAG GGTCCTGGAG GCCTGCTGGA TTACATCGAC | 1740 |
| AAAGAGATTA TTCCAGATTT CCTGAGTGG GAGTGCATGT GCGAAGTGCC AGAGGGTGGA | 1800 |
| CTGGTCCCCA AATCTCTGTA CCGGACTGCA GAGGAGCTGG AGAACGAAGA CCTGAAGCTC | 1860 |
| TGGACTGAGA CCATCTACCA GTCTGCAAGC GTCTTCAAAG GAGCCCCACA TGAGATTCTC | 1920 |
| ATTCAGATTG TGGATGCCTC GTCAGTCATC ACTTGGGATT TCGACGTGTG CAAAGGGGAC | 1980 |
| ATTGTGTTTA ACATCTATCA CTCCAAGAGG TCGCCACAAC CACCCAAAAA GGACTCCCTG | 2040 |
| GGAGCCCACA GCATCACCTC TCCGGGTGGG AACAATGTGC AGCTCATAGA CAAAGTCTGG | 2100 |
| CAGCTGGGCC GCGACTACAG CATGGTGGAG TCGCCTCTGA TCTGCAAAGA AGGAGAAAGC | 2160 |
| GTGCAGGGTT CCCATGTGAC CAGGTGGCCG GGCTTCTACA TCCTGCAGTG GAAATTCCAC | 2220 |
| AGCATGCCTG CGTGCGCCGC CAGCAGCCTT CCCCGGGTGG ACGACGTGCT TGCGTCCCTG | 2280 |
| CAGGTCTCTT CGCACAAGTG TAAAGTGATG TACTACACCG AGGTGATCGG CTCGGAGGAT | 2340 |
| TTCAGAGGTT CCATGACGAG CCTGGAGTCC AGCCACAGCG GCTTCTCCCA GCTGAGTGCC | 2400 |
| GCCACCACCT CCTCCAGCCA GTCCCACTCC AGCTCCATGA TCTCCAGGTA GTGCCGCGCT | 2460 |
| GCCTGCACCT AGTGTGCAGA GGGGACGGCC GCCCCTCCTC GGACAGCAGC TGCACCCGCC | 2520 |
| CACCCAGCGG CGACATTGTA CAGACTCCTC TCACCTCTAG ATAGCAAATA GCTCTCAGAT | 2580 |
| GGTAAACGTA GTCGTTTGAT CCCAAAACTA CCTTGGCAGG TAGTTTTAAC TCTGATCCTA | 2640 |
| ACTTAACTCA ATAGCCATAG ATTTTGTATA CGTTGTGCAC AAAATCCAAC CAGAGCGCAA | 2700 |
| GGGCTCTCTT GAAAGAAAAG TAGTTTCTGT ACCAATTAAA GGATTGACGT GGTCTCAGAT | 2760 |
| ATTGATGCAA AAAATTTTTC CAACGAACTC CGCATTGTCC ATTAGTGAAT GAATTCCTGT | 2820 |
| GACATCCTCC AGAGATGGCC CCTCCTCACC TGGGACGGAA GCTGCCAGCT CGCTTCCCCC | 2880 |
| AAGCTGCCTC ATGGCCCGCA CGCCGCCTCA CGGCCCCCAT GCTTCCCGCC AGTCAAGATG | 2940 |
| GTCTGTGGAC TTAGGGCCAG CCCTTGAGGT CCTTATCCTC TGAGGATTCA GAGGTTGCCT | 3000 |
| GCGGAGTACC TTGTCCCAGG GCCAGACACA CCCACACCAC CCACTGTCTG CAGTGGGGCC | 3060 |
| GGGGGCTCAG GAGGGGCTCT CAGGGACTCC TGGTGACTCC AGGAAAATGC TGCCATCGTT | 3120 |
| AAACATTACT TTCTCTTTCC TCCTTTTCAA ATCTTTTTGA TACTTTTTAG AGCAGGATTT | 3180 |
| TTCTGTATGT GAACTTGGGT GGGGGGGTTC TTCCCGTTTC CTTCCGTGCG TCGCCCCTCT | 3240 |
| CACCTGCAGT CAGCTCCCAG CCCAGTGTAG GCCATCTCCT CTGTGCCCTC TGGAGGCTCA | 3300 |
| TTGTCTCAGA GCCCAGACAG TTCCAGCCAC TAGGAGGCCG TCTTGGAACC AGCAAGTCGC | 3360 |
| ATTTGCCACT TGACACTGTC CATGGGGTTT TATTAGTAGC TAAGCAGCAG CTCTCGCATC | 3420 |
| CACTTCAGGG TGGCGTGTGG CATGTAGGAG TCCTGCTTCT TTGTACATGG GAATTGTGGA | 3480 |
| CTCATGCGTG TGTGTGTGTG CATGTGCTGT GTGTGTGCAT GTGTGCATGA CGGTGGGGGT | 3540 |
| GCTGGGGGGA CGGGGTGAGT GGAAACTTAG TTTGAGTAAT GAAGGAATCT TCACAGAAGC | 3600 |
| AAATCAGAAT ATGGGATTTG TTTGCCTTTT ACATTTTGTT TAATTCCTGA TTTTAAAGCC | 3660 |
| TGCTCTATCT GGTACAGGCC CTTATTTTTT CAGCTTTTTA TGGGAAAAGC AGGTTATTTG | 3720 |
| AGAATCTGTC CAGAAGTTGC ATAGGGGATG GCCTCCACGA TAAGGACATG CAACACGTGT | 3780 |
| TTCTGTGTGC AGCAGAGGCC GTGTTTTTCA TGCCAAACCC CACGCGGCTG TCAACTGTGT | 3840 |
| GCGTGGTAGG CATGGAGATC CTGGTTGTGC CGTCTCAGCT CCGCTCTGAA GGCACTGTGT | 3900 |
| GGGTGCTGCG TGACTGGAGA GCTGTGTGGA GGCCATGTGT GCCCCGTGCA GGGATCAGGA | 3960 |
| GGGCGGGGGA GGGACCGAGC AGCCCTCTTG CCCGGTCGGG TCAGCCCTAG TGGCTGCCTG | 4020 |
| CACACTGTAG ACGTCCCAGG GCCTGTGCTG TGATCACCTG CCTTTGGACC ACATTTGTGT | 4080 |
| TTGCTCTTAG AGATCGAGCT CCTCAGTGGT ACCTGAAGCC TTTGCTTCCG GAAAGCGCGG | 4140 |
| TAGGGTTCGT AGGTAGGGCT AGTAGGTAGG GTTAGTAGGT AGGGCTAGTA GGTAGGGCTA | 4200 |
| GTAGGTAGGG TTAGTAGGTA GGGTTCGTAG GTAGGGCTGG TAGGTAGGGT TAGTAGGTAG | 4260 |
| GGCTAGTAGG TAGGGTTCGT AGGTAGGGCT AGTAGGTAGG GTTAGTAGGT AGGGCTAGTA | 4320 |
| GGTAGGGCTA GTAGGTAGGG TTAGTAGGTA GGGTTCGTAG GTAGGGCTGG TAGGTAGGGT | 4380 |
| TAGTAGGTAG GGCTAGTAGG TAGGGTTCGT AGGTAGGGCT AGTAGGTAGG GTTAGTAGGT | 4440 |
| AGGGCTAGTA GGTAGGGCTA GTAGGTAGGG TTAGTAGGTA GGGTTCGTAG GTAGGGCTGG | 4500 |
| TAGGTAGGGT TAGTAGGTAG GGCTAGTAGG TAGGGCTAGT AGGTAGGGCT AGTAGGTAGG | 4560 |
| GTTAGTAGGT AGGGCTAGTA GGTAGGGCTA GTAGGTAGGG TTAGTAGGTA GGGTTCGTAG | 4620 |
| GTAGGGCTGG TAGGTAGGGT TAGTAGGTAG GGCTAGTAGG TAGGGCTAGT AGGTAGGGCT | 4680 |
| AGTAGGTAGG GCTAGTAGGT AGGGCTAGTA GGTAGGGCTA GTAGGTAGGG CTAGTAGGTA | 4740 |
| GGGTTCGTAG GTAGGGTTCG TAGGTAGGGT TCGTAGGTAG GGTTAGTAGC GCGTCTGTGC | 4800 |
| TGCTTCCACC TGGTGCTTCC TGTTCCCAAA TCACAAGGGC CTGAAGGTGG TCCCTGCTTT | 4860 |
| CTCTTTCTCT TTCTCTGTGT CTCAGATGGC GATTTTGCTG ACAGCTGCCA AGAAAATGCT | 4920 |
| TCACTCAACA GTCCTCATGT GCCCAGAGAT GTTTATAGAA CTGTTTGAAT TGCAGCCATC | 4980 |
| CCCTGCCCCC TCCCAGGCTG AAGATCTGTT CTTTTTAAGT TGATTCGGGA GTGGCATTCT | 5040 |
| TTTATACCCA AAGACTGTAG TGCATCTTGA AGAGCTCAAA GCACATGACC GCACAAATGC | 5100 |
| TTACAGGGTT TCCTCCCGAG TAATCCAATC TCACTCCCCT TGTAAGGGAA TTCTGGGGCA | 5160 |
| GCTATGGTTT GAGTATGCAG TTTGCATCGT GTTTCTACCT TTAGTACCTT GCCACTCTTT | 5220 |
| TAAAACGCTG CTGTCATTTC CCATTTCTTA GTACTAATGA TTCTTTGATT CTCCCTCTAT | 5280 |
| TATGTCTTAA TTCACTTTCC TTCCTAAATT TGTTATTTGC ATATCAAATT CTGTAAATGT | 5340 |
| TTTGTAAAGA TATTACCTCA CTTGGTAATA CAATACTGAT AGTCTTTAAA AGATTTTTTT | 5400 |
| ATTGTTATCA ATAATAAATG TGAACTATTT AAAG | |

ACJ8 DNA sequence
Gene name: intercellular adhesion molecule 1 (ICAM1; CD54)
Unigene number: Hs.168383
Probeset Accession #: M24283
Nucleic Acid Accession #: NM_000201
Coding sequence: 58-1656 (predicted start/stop codons underlined)

| | |
|---|---|
| GCGCCCCAGT CGACGCTGAG CTCCTCTGCT ACTCAGAGTT GCAACCTCAG CCTCGCTATG | 60 |
| GCTCCCAGCA GCCCCCGGCC CGCGCTGCCC GCACTCCTGG TCCTGCTCGG GGCTCTGTTC | 120 |
| CCAGGACCTG GCAATGCCCA GACATCTGTG TCCCCCTCAA AAGTCATCCT GCCCCGGGGA | 180 |
| GGCTCCGTGC TGGTGACATG CAGCACCTCC TGTGACCAGC CCAAGTTGTT GGGCATAGAG | 240 |
| ACCCCGTTGC CTAAAAAGGA GTTGCTCCTG CCTGGGAACA ACCGGAAGGT GTATGAACTG | 300 |
| AGCAATGTGC AAGAAGATAG CCAACCAATG TGCTATTCAA ACTGCCCTGA TGGGCAGTCA | 360 |
| ACAGCTAAAA CCTTCCTCAC CGTGTACTGG ACTCCAGAAC GGGTGGAACT GGCACCCCTC | 420 |
| CCCTCTTGGC AGCCAGTGGG CAAGAACCTT ACCCTACGCT GCCAGGTGGA GGGTGGGGCA | 480 |
| CCCCGGGCCA ACCTCACCGT GGTGCTGCTC CGTGGGGAGA AGGAGCTGAA ACGGGAGCCA | 540 |
| GCTGTGGGGG AGCCCGCTGA GGTCACGACC ACGGTGCTGG TGAGGAGAGA TCACCATGGA | 600 |
| GCCAATTTCT CGTGCCGCAC TGAACTGGAC CTGCGGCCCC AAGGGCTGGA GCTGTTTGAG | 660 |
| AACACCTCGG CCCCCTACCA GCTCCAGACC TTTGTCCTGC CAGCGACTCC CCCACAACTT | 720 |
| GTCAGCCCCC GGGTCCTAGA GGTGGACACG CAGGGGACCG TGGTCTGTTC CCTGGACGGG | 780 |
| CTGTTCCCAG TCTCGGAGGC CCAGGTCCAC CTGGCACTGG GGGACCAGAG GTTGAACCCC | 840 |
| ACAGTGACCT ATGGCAACGA CTCCTTCTCG GCCAAGGCCT CAGTCAGTGT GACCGCAGAG | 900 |
| GACGAGGGCA CCCAGCGGCT GACGTGTGCA GTAATACTGG GGAACCAGAG CCAGGAGACA | 960 |
| CTGCAGACAG TGACCATCTA CAGCTTTCCG GCGCCCAACG TGATTCTGAC GAAGCCAGAG | 1020 |
| GTCTCAGAAG GGACCGAGGT GACAGTGAAG TGTGAGGCCC ACCCTAGAGC CAAGGTGACG | 1080 |
| CTGAATGGGG TTCCAGCCCA GCCACTGGGC CCGAGGGCCC AGCTCCTGCT GAAGGCCACC | 1140 |
| CCAGAGGACA ACGGGCGCAG CTTCTCCTGC TCTGCAACCC TGGAGGTGGC CGGCCAGCTT | 1200 |
| ATACACAAGA ACCAGACCCG GGAGCTTCGT GTCCTGTATG GCCCCCGACT GGACGAGAGG | 1260 |
| GATTGTCCGG GAAACTGGAC GTGGCCAGAA AATTCCCAGC AGACTCCAAT GTGCCAGGCT | 1320 |
| TGGGGGAACC CATTGCCCGA GCTCAAGTGT CTAAAGGATG GCACTTTCCC ACTGCCCATC | 1380 |
| GGGGAATCAG TGACTGTCAC TCGAGATCTT GAGGGCACCT ACCTCTGTCG GGCCAGGAGC | 1440 |
| ACTCAAGGGG AGGTCACCCG CGAGGTGACC GTGAATGTGC TCTCCCCCCG GTATGAGATT | 1500 |
| GTCATCATCA CTGTGGTAGC AGCCGCAGTC ATAATGGGCA CTGCAGGCCT CAGCACGTAC | 1560 |
| CTCTATAACC GCCAGCGGAA GATCAAGAAA TACAGACTAC AACAGGCCCA AAAAGGGACC | 1620 |
| CCCATGAAAC CGAACACACA AGCCACGCCT CCCTGAACCT ATCCCGGGAC AGGGCCTCTT | 1680 |
| CCTCGGCCTT CCCATATTGG TGGCAGTGGT GCCACACTGA ACAGAGTGGA AGACATATGC | 1740 |
| CATGCAGCTA CACCTACCGG CCCTGGGACG CCGGAGGACA GGGCATTGTC CTCAGTCAGA | 1800 |
| TACAACAGCA TTTGGGGCCA TGGTACCTGC ACACCTAAAA CACTAGGCCA CGCATCTGAT | 1860 |
| CTGTAGTCAC ATGACTAAGC CAAGAGGAAG GAGCAAGACT CAAGACATGA TTGATGGATG | 1920 |
| TTAAAGTCTA GCCTGATGAG AGGGGAAGTG GTGGGGGAGA CATAGCCCCA CCATGAGGAC | 1980 |
| ATACAACTGG GAAATACTGA AACTTGCTGC CTATTGGGTA TGCTGAGGCC CACAGACTTA | 2040 |
| CAGAAGAAGT GGCCCTCCAT AGACATGTGT AGCATCAAAA CACAAAGGCC CACACTTCCT | 2100 |
| GACGGATGCC AGCTTGGGCA CTGCTGTCTA CTGACCCCAA CCCTTGATGA TATGTATTTA | 2160 |
| TTCATTTGTT ATTTTACCAG CTATTTATTG AGTGTCTTTT ATGTAGGCTA AATGAACATA | 2220 |
| GGTCTCTGGC CTCACGGAGC TCCCAGTCCA TGTCACATTC AAGGTCACCA GGTACAGTTG | 2280 |
| TACAGGTTGT ACACTGCAGG AGAGTGCCTG GCAAAAAGAT CAAATGGGGC TGGGACTTCT | 2340 |
| CATTGGCCAA CCTGCCTTTC CCCAGAAGGA GTGATTTTTC TATCGGCACA AAAGCACTAT | 2400 |
| ATGGACTGGT AATGGTTCAC AGGTTCAGAG ATTACCCAGT GAGGCCTTAT TCCTCCCTTC | 2460 |
| CCCCCAAAAC TGACACCTTT GTTAGCCACC TCCCCACCCA CATACATTTC TGCCAGTGTT | 2520 |
| CACAATGACA CTCAGCGGTC ATGTCTGGAC ATGAGTGCCC AGGGAATATG CCCAAGCTAT | 2580 |
| GCCTTGTCCT CTTGTCCTGT TTGCATTTCA CTGGGAGCTT GCACTATTGC AGCTCCAGTT | 2640 |
| TCCTGCAGTG ATCAGGGTCC TGCAAGCAGT GGGGAAGGGG GCCAAGGTAT TGGAGGACTC | 2700 |
| CCTCCCAGCT TTGGAAGGGT CATCCGCGTG TGTGTGTGTG TGTATGTGTA GACAAGCTCT | 2760 |
| CGCTCTGTCA CCCAGGCTGG AGTGCAGTGG TGCAATCATG GTTCACTGCA GTCTTGACCT | 2820 |
| TTTGGGCTCA AGTGATCCTC CCACCTCAGC CTCCTGAGTA GCTGGGACCA TAGGCTCACA | 2880 |
| ACACCACACC TGGCAAATTT GATTTTTTTT TTTTTTTTCA GAGACGGGGT CTCGCAACAT | 2940 |
| TGCCCAGACT TCCTTTGTGT TAGTTAATAA AGCTTTCTCA ACTGCC | |

ACK3 DNA sequence
Gene name: angiopoietin 1 receptor (TIE-2; TEK)
Unigene number: Hs.89640
Probeset Accession #: L06139
Nucleic Acid Accession #: NM_000459
Coding sequence: 149-3523 (predicted start/stop codons underlined)

| | |
|---|---|
| CTTCTGTGCT GTTCCTTCTT GCCTCTAACT TGTAAACAAG ACGTACTAGG ACGATGCTAA | 60 |
| TGGAAAGTCA CAAACCGCTG GGTTTTTGAA AGGATCCTTG GGACCTCATG CACATTTGTG | 120 |
| GAAACTGGAT GGAGAGATTT GGGGAAGCAT GGACTCTTTA GCCAGCTTAG TTCTCTGTGG | 180 |
| AGTCAGCTTG CTCCTTTCTG GAACTGTGGA AGGTGCCATG GACTTGATCT TGATCAATTC | 240 |
| CCTACCTCTT GTATCTGATG CTGAAACATC TCTCACCTGC ATTGCCTCTG GGTGGCGCCC | 300 |
| CCATGAGCCC ATCACCATAG GAAGGGACTT TGAAGCCTTA ATGAACCAGC ACCAGGATCC | 360 |
| GCTGGAAGTT ACTCAAGATG TGACCAGAGA ATGGGCTAAA AAAGTTGTTT GGAAGAGAGA | 420 |
| AAAGGCTAGT AAGATCAATG GTGCTTATTT CTGTGAAGGG CGAGTTCGAG GAGAGGCAAT | 480 |
| CAGGATACGA ACCATGAAGA TGCGTCAACA AGCTTCCTTC CTACCAGCTA CTTTAACTAT | 540 |
| GACTGTGGAC AAGGGAGATA ACGTGAACAT ATCTTTCAAA AAGGTATTGA TTAAAGAAGA | 600 |
| AGATGCAGTG ATTTACAAAA ATGGTTCCTT CATCCATTCA GTGCCCCGGC ATGAAGTACC | 660 |
| TGATATTCTA GAAGTACACC TGCCTCATGC TCAGCCCCAG GATGCTGGAG TGTACTCGGC | 720 |
| CAGGTATATA GGAGGAAACC TCTTCACCTC GGCCTTCACC AGGCTGATAG TCCGGAGATG | 780 |
| TGAAGCCCAG AAGTGGGGAC CTGAATGCAA CCATCTCTGT ACTGCTTGTA TGAACAATGG | 840 |
| TGTCTGCCAT GAAGATACTG GAGAATGCAT TTGCCCTCCT GGGTTTATGG GAAGGACGTG | 900 |
| TGAGAAGGCT TGTGAACTGC ACACGTTTGG CAGAACTTGT AAAGAAAGGT GCAGTGGACA | 960 |
| AGAGGGATGC AAGTCTTATG TGTTCTGTCT CCCTGACCCC TATGGGTGTT CCTGTGCCAC | 1020 |
| AGGCTGGAAG GGTCTGCAGT GCAATGAAGC ATGCCACCCT GGTTTTTACG GGCCAGATTG | 1080 |
| TAAGCTTAGG TGCAGCTGCA ACAATGGGGA GATGTGTGAT CGCTTCCAAG GATGTCTCTG | 1140 |
| CTCTCCAGGA TGGCAGGGGC TCCAGTGTGA GAGAGAAGGC ATACCGAGGA TGACCCCAAA | 1200 |
| GATAGTGGAT TTGCCAGATC ATATAGAAGT AAACAGTGGT AAATTTAATC CCATTTGCAA | 1260 |
| AGCTTCTGGC TGGCCGCTAC CTACTAATGA AGAAATGACC CTGGTGAAGC CGGATGGGAC | 1320 |
| AGTGCTCCAT CCAAAAGACT TTAACCATAC GGATCATTTC TCAGTAGCCA TATTCACCAT | 1380 |
| CCACCGGATC CTCCCCCCTG ACTCAGGAGT TTGGGTCTGC AGTGTGAACA CAGTGGCTGG | 1440 |
| GATGGTGGAA AAGCCCTTCA ACATTTCTGT TAAAGTTCTT CCAAAGCCCC TGAATGCCCC | 1500 |
| AAACGTGATT GACACTGGAC ATAACTTTGC TGTCATCAAC ATCAGCTCTG AGCCTTACTT | 1560 |
| TGGGGATGGA CCAATCAAAT CCAAGAAGCT TCTATACAAA CCCGTTAATC ACTATGAGGC | 1620 |
| TTGGCAACAT ATTCAAGTGA CAAATGAGAT TGTTACACTC AACTATTTGG AACCTCGGAC | 1680 |
| AGAATATGAA CTCTGTGTGC AACTGGTCCG TCGTGGAGAG GGTGGGGAAG GGCATCCTGG | 1740 |
| ACCTGTGAGA CGCTTCACAA CAGCTTCTAT CGGACTCCCT CCTCCAAGAG GTCTAAATCT | 1800 |
| CCTGCCTAAA AGTCAGACCA CTCTAAATTT GACCTGGCAA CCAATATTTC CAAGCTCGGA | 1860 |
| AGATGACTTT TATGTTGAAG TGGAGAGAAG GTCTGTGCAA AAAAGTGATC AGCAGAATAT | 1920 |
| TAAAGTTCCA GGCAACTTGA CTTCGGTGCT ACTTAACAAC TTACATCCCA GGGAGCAGTA | 1980 |
| CGTGGTCCGA GCTAGAGTCA ACACCAAGGC CCAGGGGGAA TGGAGTGAAG ATCTCACTGC | 2040 |
| TTGGACCCTT AGTGACATTC TTCCTCCTCA ACCAGAAAAC ATCAAGATTT CCAACATTAC | 2100 |
| ACACTCCTCG GCTGTGATTT CTTGGACAAT ATTGGATGGC TATTCTATTT CTTCTATTAC | 2160 |
| TATCCGTTAC AAGGTTCAAG GCAAGAATGA AGACCAGCAC GTTGATGTGA AGATAAAGAA | 2220 |
| TGCCACCATC ATTCAGTATC AGCTCAAGGG CCTAGAGCCT GAAACAGCAT ACCAGGTGGA | 2280 |
| CATTTTTGCA GAGAACAACA TAGGGTCAAG CAACCCAGCC TTTTCTCATG AACTGGTGAC | 2340 |
| CCTCCCAGAA TCTCAAGCAC CAGCGGACCT CGGAGGGGGG AAGATGCTGC TTATAGCCAT | 2400 |
| CCTTGGCTCT GCTGGAATGA CCTGCCTGAC TGTGCTGTTG GCCTTTCTGA TCATATTGCA | 2460 |
| ATTGAAGAGG GCAAATGTGC AAAGGAGAAT GGCCCAAGCC TTCCAAAACG TGAGGGAAGA | 2520 |
| ACCAGCTGTG CAGTTCAACT CAGGGACTCT GGCCCTAAAC AGGAAGGTCA AAAACAACCC | 2580 |
| AGATCCTACA ATTTATCCAG TGCTTGACTG GAATGACATC AAATTTCAAG ATGTGATTGG | 2640 |
| GGAGGGCAAT TTTGGCCAAG TTCTTAAGGC GCGCATCAAG AAGGATGGGT TACGGATGGA | 2700 |
| TGCTGCCATC AAAAGAATGA AAGAATATGC CTCCAAAGAT GATCACAGGG ACTTTGCAGG | 2760 |
| AGAACTGGAA GTTCTTTGTA AACTTGGACA CCATCCAAAC ATCATCAATC TCTTAGGAGC | 2820 |
| ATGTGAACAT CGAGGCTACT TGTACCTGGC CATTGAGTAC GCGCCCCATG GAAACCTTCT | 2880 |
| GGACTTCCTT CGCAAGAGCC GTGTGCTGGA GACGGACCCA GCATTTGCCA TTGCCAATAG | 2940 |
| CACCGCGTCC ACACTGTCCT CCCAGCAGCT CCTTCACTTC GCTGCCGACG TGGCCCGGGG | 3000 |
| CATGGACTAC TTGAGCCAAA AACAGTTTAT CCACAGGGAT CTGGCTGCCA GAAACATTTT | 3060 |
| AGTTGGTGAA AACTATGTGG CAAAAATAGC AGATTTTGGA TTGTCCCGAG GTCAAGAGGT | 3120 |
| GTACGTGAAA AAGACAATGG GAAGGCTCCC AGTGCGCTGG ATGGCCATCG AGTCACTGAA | 3180 |
| TTACAGTGTG TACACAACCA ACAGTGATGT ATGGTCCTAT GGTGTGTTAC TATGGGAGAT | 3240 |
| TGTTAGCTTA GGAGGCACAC CCTACTGCGG GATGACTTGT GCAGAACTCT ACGAGAAGCT | 3300 |
| GCCCCAGGGC TACAGACTGG AGAAGCCCCT GAACTGTGAT GATGAGGTGT ATGATCTAAT | 3360 |
| GAGACAATGC TGGCGGGAGA AGCCTTATGA GAGGCCATCA TTTGCCCAGA TATTGGTGTC | 3420 |
| CTTAAACAGA ATGTTAGAGG AGCGAAAGAC CTACGTGAAT ACCACGCTTT ATGAGAAGTT | 3480 |
| TACTTATGCA GGAATTGACT GTTCTGCTGA AGAAGCGGCC TAGGACAGAA CATCTGTATA | 3540 |
| CCCTCTGTTT CCCTTTCACT GGCATGGGAG ACCCTTGACA ACTGCTGAGA AAACATGCCT | 3600 |
| CTGCCAAAGG ATGTGATATA TAAGTGTACA TATGTGCTGG AATTCTAACA AGTCATAGGT | 3660 |
| TAATATTTAA GACACTGAAA AATCTAAGTG ATATAAATCA GATTCTTCTC TCTCATTTTA | 3720 |
| TCCCTCACCT GTAGCATGCC AGTCCCGTTT CATTTAGTCA TGTGACCACT CTGTCTTGTG | 3780 |
| TTTCCACAGC CTGCAAGTTC AGTCCAGGAT GCTAACATCT AAAAATAGAC TTAAATCTCA | 3840 |
| TTGCTTACAA GCCTAAGAAT CTTTAGAGAA GTATACATAA GTTTAGGATA AAATAATGGG | 3900 |
| ATTTTCTTTT CTTTTCTCTG GTAATATTGA CTTGTATATT TTAAGAAATA ACAGAAAGCC | 3960 |
| TGGGTGACAT TTGGGAGACA TGTGACATTT ATATATTGAA TTAATATCCC TACATGTATT | 4020 |
| GCACATTGTA AAAAGTTTTA GTTTTGATGA GTTGTGAGTT TACCTTGTAT ACTGTAGGCA | 4080 |
| CACTTTGCAC TGATATATCA TGAGTGAATA AATGTCTTGC CTACTCAAAA AAAAAAAA | |

PZA6 DNA sequence
Gene name: prostate differentiation factor (PLAB; MIC-1)
Unigene number: Hs.116577
Probeset Accession #: AB000584
Nucleic Acid Accession #: NM_004864
Coding sequence: 26-952 (predicted start/stop codons underlined)

| | |
|---|---|
| CGGAACGAGG GCAACCTGCA CAGCCATGCC CGGGCAAGAA CTCAGGACGG TGAATGGCTC | 60 |
| TCAGATGCTC CTGGTGTTGC TGGTGCTCTC GTGGCTGCCG CATGGGGGCG CCCTGTCTCT | 120 |
| GGCCGAGGCG AGCCGCGCAA GTTTCCCGGG ACCCTCAGAG TTGCACTCCG AAGACTCCAG | 180 |
| ATTCCGAGAG TTGCGGAAAC GCTACGAGGA CCTGCTAACC AGGCTGCGGG CCAACCAGAG | 240 |
| CTGGGAAGAT TCGAACACCG ACCTCGTCCC GGCCCCTGCA GTCCGGATAC TCACGCCAGA | 300 |
| AGTGCGGCTG GGATCCGGCG GCCACCTGCA CCTGCGTATC TCTCGGGCCG CCCTTCCCGA | 360 |
| GGGGCTCCCC GAGGCCTCCC GCCTTCACCG GGCTCTGTTC CGGCTGTCCC CGACGGCGTC | 420 |
| AAGGTCGTGG GACGTGACAC GACCGCTGCG GCGTCAGCTC AGCCTTGCAA GACCCCAAGC | 480 |
| GCCCGCGCTG CACCTGCGAC TGTCGCCGCC GCCGTCGCAG TCGGACCAAC TGCTGGCAGA | 540 |
| ATCTTCGTCC GCACGGCCCC AGCTGGAGTT GCACTTGCGG CCGCAAGCCG CCAGGGGGCG | 600 |
| CCGCAGAGCG CGTGCGCGCA ACGGGGACGA CTGTCCGCTC GGGCCCGGGC GTTGCTGCCG | 660 |
| TCTGCACACG GTCCGCGCGT CGCTGGAAGA CCTGGGCTGG GCCGATTGGG TGCTGTCGCC | 720 |
| ACGGGAGGTG CAAGTGACCA TGTGCATCGG CGCGTGCCCG AGCCAGTTCC GGGCGGCAAA | 780 |
| CATGCACGCG CAGATCAAGA CGAGCCTGCA CCGCCTGAAG CCCGACACGG AGCCAGCGCC | 840 |
| CTGCTGCGTG CCCGCCAGCT ACAATCCCAT GGTGCTCATT CAAAAGACCG ACACCGGGGT | 900 |
| GTCGCTCCAG ACCTATGATG ACTTGTTAGC CAAAGACTGC CACTGCATAT GAGCAGTCCT | 960 |
| GGTCCTTCCA CTGTGCACCT GCGCGGGGGA GGCGACCTCA GTTGTCCTGC CCTGTGGAAT | 1020 |
| GGGCTGAAGG TTCCTGAGAC ACCCGATTCC TGCCCAAACA GCTGTATTTA TATAAGTCTG | 1080 |
| TTATTTATTA TTAATTTATT GGGGTGACCT TCTTGGGGAC TCGGGGGCTG GTCTGATGGA | 1140 |
| ACTGTGTATT TATTTAAAAC TCTGGTGATA AAAATAAAGC TGTCTGAACT GTTAAAAAAA | 1200 |

AAC8 DNA sequence
Gene name: none
Unigene number: Hs.6682
Probeset Accession #: AA227926
Nucleic Acid Accession #: none
Coding sequence: no ORF identified, possible frameshifts

| | |
|---|---|
| AAGCTGCAGT TAGCCAAGAT CGCATCATTG CACTCCAGCC TAGGGGACAA GAGCGCGAGA | 60 |
| CTTCATCTCA AAGATTTTTA AATAATAGCT AAAGGTATGC TCTCTAGGTC ATCCTTAGTT | 120 |
| TATTAGTACT GTACTTAAAA ATTATTTTTT TAATAGTCAA TTTTGGGAGA TAATTATTTC | 180 |
| TTTCCTTATA TTTTCCAATT AGTTGGTGTC TAAAAATAAA TGTTTTGTCT AATTTTAGAT | 240 |
| CAGGTATACA TTCACAAAAG CATAAATCAT AGTCTCACAG GAAATTCACC AATTTTCCAT | 300 |
| ATGTCGTGAG ATAACTGTCC TTTCTACAAC CTCATAACAA TGAATTTATA TAATTACCTA | 360 |
| GATTTTCTTA GTGTGAATCT ACCCATTAGT TTTATTTTCT TGGTAGTTAT TTTTTTCCCT | 420 |
| CCTCTCTGTT ACTATTGGGC TTAAAATACA CAGGAGGACG GTTACAGTGT CCTAATAGCT | 480 |
| GTTACATGTG TGTGTTTCAG CGTACTTGAA TCAAGTGTAC ATTTATAGTA CCAATAACCG | 540 |
| CCTTTACAGC TTTACAGTTA ACAATTCTCT CACAAAACTG TAGAGCATTA GGCATCTGAG | 600 |
| AGCCATAGAG GGCCAACTTT GTTCCAGAGT GAACATGCTT TTTTTCCTCA ACATATACAC | 660 |
| TACTGATTTT TTTTAAAAGT ATGACTTTCA AGTGAATTAA TGTATTGGTT AGGAGAACTG | 720 |
| CTTGCTAAGT CCTTATTACC TCTTGTTAAA GCCTCAGAAG GCCGTGCTGA AAGCCAGAGG | 780 |
| GGAAAAAAAG AGTAATGCAC AGGTATCTCT TTTGCAGTGG TGACTGTATT TTGAGTACCT | 840 |
| TGTGTGACAG GGTATTATTA CAGCATCTTG TGGGAAAACC TATTAGGCCT TTGCATGTTA | 900 |
| AAGCTGTATA ATTTGTTGGG TTGTGAGTGG TCTGACTTAA ATGTGTATTA TAAAATTTAG | 960 |
| ACATCAAATT TTCCTACTAA CTAACTTTAT TAGATGCATA CTTGGAAGCA CAGTCATATC | 1020 |
| ACACTGGGAG GCAATGCAAT GTGGTTACCT GGTCCTAGGT TTGAACTGTC TTATTTCAAA | 1080 |
| AGATTTCTGA ATTAATTTTT CCCTAGAATT TCTCCTTCAT TCCAAAGTAC AAACATACTT | 1140 |
| TGAAGAATGA AACAGATTGT TCCCATGAAT GTATGCTCAT ACTCGACTAG AAACGATCTA | 1200 |
| TGTTAAATGA CTGTGTATAT GAATTATTTC AAGTACTACC CCAAATAACT TTCTTATTGC | 1260 |
| TCTGAAAGAA GAAAAGCAAT GTAAATCACT ATGATTATTG CACAAACAAC CAGAATTCTC | 1320 |
| CAACAATTTT AAGTAATCTG ATCCTCTTCT TGGAGAAAAT TGTTACCTAA TAGTTTTTCC | 1380 |
| TTATGAATGT TATTACTACT GGTATAAATC AAATTTCTAT AAATTTCCTA CTTAAAGTCT | 1440 |
| TAARAACTGG GTTCTTCCTT TGATGTTATT CATGTTCAGA AAGGGAAACA ACACTTTACT | 1500 |
| TTTTTAGGGA CAATTTCTAG AATCTATAGT AGTATCAGGA TATATTTTGC TTTAAAATAT | 1560 |
| ATTTTGGTTA TTTTGAATAC AGACATTGGC TCCAAATTTT CATCTTTGCA CAATAGTATG | 1620 |
| ACTTTTCACT AGAACTTCTC AACATTTGGG AACTTTGCAA ATATGAGCAT CATATGTGTT | 1680 |
| AAGGCTGTAT CATTTAATGC TATGAGATAC ATTGTTTTCT CCCTATGCCA AACAGGTGAA | 1740 |
| CAAACGTAGT TGTTTTTTAC TGATACTAAA TGTTGGCTAC CTGTGATTTT ATAGTATGCA | 1800 |
| CATGTCAGAA AAAGGCAAGA CAAATGGCCT CTTGTACTGA ATACTTCGGC AAACTTATTG | 1860 |
| GGGTCTTCAT TTTCTGACAG ACAGGATTTG ACTCAATATT TGTAGAGCTT GCGTAGGAAT | 1920 |
| GGGATTACAT GGGTAGTGAT GCACTGGTAG GAAATGGTTT TTAGTTATTG ACTCAGGAAT | 1980 |
| TCATCTGAGG ATGAATCTTT TATGTCTTTT TATTGTAAGG CATATCTGGA ATTTACTTTA | 2040 |
| TAAAGGAGGG GTTTAGGAAA GCTTTGTCCT AAAAATTGGG CCCCGGGGAT GGGAACTTCA | 2100 |
| TTTTCAGTTG CCAAGGGGTA GAAAAATAAT ATGTGTGTTG TTATGTTTAT GTTAACATAT | 2160 |
| TATTAGGTAC TATCTATGAA TGTATTTAAA TATTTTTCAT ATTCTGTGAC AAGCATTTAT | 2220 |
| AATTTGCAAC AAGTGGAGTC CATTTAGCCC AGTGGGAAAG TCTTGGAACT CAGGTTACCC | 2280 |
| TTGAAGGATA TGCTGGCAGC CATCTCTTTG ATCTGTGCTT AAACTGTAAT TTATAGACCA | 2340 |
| GCTAAATCCC TAACTTGGAT CTGGAATGCA TTAGTTATGA CCTTGTACCA TTCCCAGAAT | 2400 |
| TTCAGGGGCA TCGTGGGTTT GGTCTAGTGA TTGAAAACAC AAGAACAGAG AGATCCAGCT | 2460 |
| GAAAAAGAGT GATCCTCAAT ATCCTAACTA ACTGGTCCTC AACTCAAGCA GAGTTTCTTC | 2520 |
| ACTCTGGCAC TGTGATCATG AAACTTAGTA GAGGGGATTG TGTGTATTTT ATACAAATTT | 2580 |
| AATACAATGT CTTACATTGA TAAAATTCTT AAAGAGCAAA ACTGCATTTT ATTTCTGCAT | 2640 |
| CCACATTCCA ATCATATTAG AACTAAGATA TTTATCTATG AAGATATAAA TGGTGCAGAG | 2700 |
| AGACTTTCAT CTGTGGATTG CGTTGTTTCT CTAGGGTTCC TCAGCCACTG ATGCCTCGCC | 2760 |
| ACAAGCCATG TGATATGTGA AATAAAAAGG GATTCTTCCT ATAGCCTAAA TGAAGTTCCC | 2820 |
| TCTGGGGAGA GTTCTGGTAC TGCAATCACA ATGCCAGATG GTGTTTATGG GCTATTTGTG | 2880 |
| TAAGTAAGTG GTAAGATGCT ATGAAGTAAG TGTGTTTGTT TTCATCTTAT GGAAACTCTT | 2940 |
| GATGCATGTG CTTTTGTATG GAATAAATTT TGGTGCAATA TGATGTCATT CAACTTTGCA | 3000 |
| TTGAATTGAA TTTTGGTTGT ATTTATATGT ATTATACCTG TCACGCTTCT AGTTGCTTCA | 3060 |
| ACCATTTTAT AACCATTTTT GTACATATTT TACTTGAAAA TATTTTAAAT GGAAATTTAA | 3120 |
| ATAAACATTT GATAGTTTAC ATAAAAAAAA AAAAAAAAAA A | |

AAD2 DNA sequence
Gene name: Thrombospondin-1
Unigene number: Hs.87409
Probeset Accession #: AA232645
Nucleic Acid Accession #: NM_003246
Coding sequence: 112-3624 (predicted start/stop codons underlined)

| | |
|---|---|
| GGACGCACAG GCATTCCCCG CGCCCCTCCA GCCCTCGCCG CCCTCGCCAC CGCTCCCGGC | 60 |
| CGCCGCGCTC CGGTACACAC AGGATCCCTG CTGGGCACCA ACAGCTCCAC CATGGGGCTG | 120 |
| GCCTGGGGAC TAGGCGTCCT GTTCCTGATG CATGTGTGTG GCACCAACCG CATTCCAGAG | 180 |
| TCTGGCGGAG ACAACAGCGT GTTTGACATC TTTGAACTCA CCGGGGCCGC CCGCAAGGGG | 240 |
| TCTGGGCGCC GACTGGTGAA GGGCCCCGAC CCTTCCAGCC CAGCTTTCCG CATCGAGGAT | 300 |
| GCCAACCTGA TCCCCCCTGT GCCTGATGAC AAGTTCCAAG ACCTGGTGGA TGCTGTGCGG | 360 |
| GCAGAAAAGG GTTTCCTCCT TCTGGCATCC CTGAGGCAGA TGAAGAAGAC CCGGGGCACG | 420 |
| CTGCTGGCCC TGGAGCGGAA AGACCACTCT GGCCAGGTCT TCAGCGTGGT GTCCAATGGC | 480 |
| AAGGCGGGCA CCCTGGACCT CAGCCTGACC GTCCAAGGAA AGCAGCACGT GGTGTCTGTG | 540 |
| GAAGAAGCTC TCCTGGCAAC CGGCCAGTGG AAGAGCATCA CCCTGTTTGT GCAGGAAGAC | 600 |
| AGGGCCCAGC TGTACATCGA CTGTGAAAAG ATGGAGAATG CTGAGTTGGA CGTCCCCATC | 660 |
| CAAAGCGTCT TCACCAGAGA CCTGGCCAGC ATCGCCAGAC TCCGCATCGC AAAGGGGGGC | 720 |
| GTCAATGACA ATTTCCAGGG GGTGCTGCAG AATGTGAGGT TTGTCTTTGG AACCACACCA | 780 |
| GAAGACATCC TCAGGAACAA AGGCTGCTCC AGCTCTACCA GTGTCCTCCT CACCCTTGAC | 840 |
| AACAACGTGG TGAATGGTTC CAGCCCTGCC ATCCGCACTA ACTACATTGG CCACAAGACA | 900 |
| AAGGACTTGC AAGCCATCTG CGGCATCTCC TGTGATGAGC TGTCCAGCAT GGTCCTGGAA | 960 |
| CTCAGGGGCC TGCGCACCAT TGTGACCACG CTGCAGGACA GCATCCGCAA AGTGACTGAA | 1020 |
| GAGAACAAAG AGTTGGCCAA TGAGCTGAGG CGGCCTCCCC TATGCTATCA CAACGGAGTT | 1080 |
| CAGTACAGAA ATAACGAGGA ATGGACTGTT GATAGCTGCA CTGAGTGTCA CTGTCAGAAC | 1140 |
| TCAGTTACCA TCTGCAAAAA GGTGTCCTGC CCCATCATGC CCTGCTCCAA TGCCACAGTT | 1200 |
| CCTGATGGAG AATGCTGTCC TCGCTGTTGG CCCAGCGACT CTGCGGACGA TGGCTGGTCT | 1260 |
| CCATGGTCCG AGTGGACCTC CTGTTCTACG AGCTGTGGCA ATGGAATTCA GCAGCGCGGC | 1320 |
| CGCTCCTGCG ATAGCCTCAA CAACCGATGT GAGGGCTCCT CGGTCCAGAC ACGGACCTGC | 1380 |
| CACATTCAGG AGTGTGACAA AAGATTTAAA CAGGATGGTG GCTGGAGCCA CTGGTCCCCG | 1440 |
| TGGTCATCTT GTTCTGTGAC ATGTGGTGAT GGTGTGATCA CAAGGATCCG GCTCTGCAAC | 1500 |
| TCTCCCAGCC CCCAGATGAA TGGGAAACCC TGTGAAGGCG AAGCGCGGGA GACCAAAGCC | 1560 |
| TGCAAGAAAG ACGCCTGCCC CATCAATGGA GGCTGGGGTC CTTGGTCACC ATGGGACATC | 1620 |
| TGTTCTGTCA CCTGTGGAGG AGGGGTACAG AAACGTAGTC GTCTCTGCAA CAACCCCGCA | 1680 |
| CCCCAGTTTG GAGGCAAGGA CTGCGTTGGT GATGTAACAG AAAACCAGAT CTGCAACAAG | 1740 |
| CAGGACTGTC CAATTGATGG ATGCCTGTCC AATCCCTGCT TTGCCGGCGT GAAGTGTACT | 1800 |
| AGCTACCCTG ATGGCAGCTG GAAATGTGGT GCTTGTCCCC CTGGTTACAG TGGAAATGGC | 1860 |
| ATCCAGTGCA CAGATGTTGA TGAGTGCAAA GAAGTGCCTG ATGCCTGCTT CAACCACAAT | 1920 |
| GGAGAGCACC GGTGTGAGAA CACGGACCCC GGCTACAACT GCCTGCCCTG CCCCCCACGC | 1980 |
| TTCACCGGCT CACAGCCCTT CGGCCAGGGT GTCGAACATG CCACGGCCAA CAAACAGGTG | 2040 |
| TGCAAGCCCC GTAACCCCTG CACGGATGGG ACCCACGACT GCAACAAGAA CGCCAAGTGC | 2100 |
| AACTACCTGG GCCACTATAG CGACCCCATG TACCGCTGCG AGTGCAAGCC TGGCTACGCT | 2160 |
| GGCAATGGCA TCATCTGCGG GGAGGACACA GACCTGGATG GCTGGCCCAA TGAGAACCTG | 2220 |
| GTGTGCGTGG CCAATGCGAC TTACCACTGC AAAAAGGATA ATTGCCCCAA CCTTCCCAAC | 2280 |
| TCAGGGCAGG AAGACTATGA CAAGGATGGA ATTGGTGATG CCTGTGATGA TGACGATGAC | 2340 |
| AATGATAAAA TTCCAGATGA CAGGGACAAC TGTCCATTCC ATTACAACCC AGCTCAGTAT | 2400 |
| GACTATGACA GAGATGATGT GGGAGACCGC TGTGACAACT GTCCCTACAA CCACAACCCA | 2460 |
| GATCAGGCAG ACACAGACAA CAATGGGCAA GGAGACGCCT GTGCTGCAGA CATTGATGGA | 2520 |
| GACGGTATCC TCAATGAACG GGACAACTGC CAGTACGTCT ACAATGTGGA CCAGAGAGAC | 2580 |
| ACTGATATGG ATGGGGTTGG AGATCAGTGT GACAATTGCC CCTTGGAACA CAATCCGGAT | 2640 |
| CAGCTGGACT CTGACTCAGA CCGCATTGGA GATACCTGTG ACAACAATCA GGATATTGAT | 2700 |
| GAAGATGGCC ACCAGAACAA TCTGGACAAC TGTCCCTATG TGCCCAATGC CAACCAGGCT | 2760 |
| GACCATGACA AAGATGGCAA GGGAGATGCC TGTGACCACG ATGATGACAA CGATGGCATT | 2820 |
| CCTGATGACA AGGACAACTG CAGACTCGTG CCCAATCCCG ACCAGAAGGA CTCTGACGGC | 2880 |
| GATGGTCGAG GTGATGCCTG CAAAGATGAT TTTGACCATG ACAGTGTGCC AGACATCGAT | 2940 |
| GACATCTGTC CTGAGAATGT TGACATCAGT GAGACCGATT TCCGCCGATT CCAGATGATT | 3000 |
| CCTCTGGACC CCAAAGGGAC ATCCCAAAAT GACCCTAACT GGGTTGTACG CCATCAGGGT | 3060 |
| AAAGAACTCG TCCAGACTGT CAACTGTGAT CCTGGACTCG CTGTAGGTTA TGATGAGTTT | 3120 |
| AATGCTGTGG ACTTCAGTGG CACCTTCTTC ATCAACACCG AAAGGGACGA TGACTATGCT | 3180 |
| GGATTTGTCT TTGGCTACCA GTCCAGCAGC CGCTTTTATG TTGTGATGTG GAAGCAAGTC | 3240 |
| ACCCAGTCCT ACTGGGACAC CAACCCCACG AGGGCTCAGG GATACTCGGG CCTTTCTGTG | 3300 |
| AAAGTTGTAA ACTCCACCAC AGGGCCTGGC GAGCACCTGC GGAACGCCCT GTGGCACACA | 3360 |
| GGAAACACCC CTGGCCAGGT GCGCACCCTG TGGCATGACC CTCGTCACAT AGGCTGGAAA | 3420 |
| GATTTCACCG CCTACAGATG GCGTCTCAGC CACAGGCCAA AGACGGGTTT CATTAGAGTG | 3480 |
| GTGATGTATG AAGGGAAGAA AATCATGGCT GACTCAGGAC CCATCTATGA TAAAACCTAT | 3540 |
| GCTGGTGGTA GACTAGGGTT GTTTGTCTTC TCTCAAGAAA TGGTGTTCTT CTCTGACCTG | 3600 |
| AAATACGAAT GTAGAGATCC CTAATCATCA AATTGTTGAT TGAAAGACTG ATCATAAACC | 3660 |
| AATGCTGGTA TTGCACCTTC TGGAACTATG GGCTTGAGAA AACCCCCAGG ATCACTTCTC | 3720 |
| CTTGGCTTCC TTCTTTTCTG TGCTTGCATC AGTGTGGACT CCTAGAACGT GCGACCTGCC | 3780 |
| TCAAGAAAAT GCAGTTTTCA AAAACAGACT CATCAGCATT CAGCCTCCAA TGAATAAGAC | 3840 |
| ATCTTCCAAG CATATAAACA ATTGCTTTGG TTTCCTTTTG AAAAAGCATC TACTTGCTTC | 3900 |
| AGTTGGGAAG GTGCCCATTC CACTCTGCCT TTGTCACAGA GCAGGGTGCT ATTGTGAGGC | 3960 |
| CATCTCTGAG CAGTGGACTC AAAAGCATTT TCAGGCATGT CAGAGAAGGG AGGACTCACT | 4020 |
| AGAATTAGCA AACAAAACCA CCCTGACATC CTCCTTCAGG AACACGGGGA GCAGAGGCCA | 4080 |
| AAGCACTAAG GGGAGGGCGC ATACCCGAGA CGATTGTATG AAGAAAATAT GGAGGAACTG | 4140 |
| TTACATGTTC GGTACTAAGT CATTTTCAGG GGATTGAAAG ACTATTGCTG GATTTCATGA | 4200 |
| TGCTGACTGG CGTTAGCTGA TTAACCCATG TAAATAGGCA CTTAAATAGA AGCAGGAAAG | 4260 |
| GGAGACAAAG ACTGGCTTCT GGACTTCCTC CCTGATCCCC ACCCTTACTC ATCACCTTGC | 4320 |
| AGTGGCCAGA ATTAGGGAAT CAGAATCAAA CCAGTGTAAG GCAGTGCTGG CTGCCATTGC | 4380 |
| CTGGTCACAT TGAAATTGGT GGCTTCATTC TAGATGTAGC TTGTGCAGAT GTAGCAGGAA | 4440 |
| AATAGGAAAA CCTACCATCT CAGTGAGCAC CAGCTGCCTC CCAAAGGAGG GGCAGCCGTG | 4500 |
| CTTATATTTT TATGGTTACA ATGGCACAAA ATTATTATCA ACCTAACTAA AACATTCCTT | 4560 |
| TTCTCTTTTT TCCGTAATTA CTAGGTAGTT TTCTAATTCT CTCTTTTGGA AGTATGATTT | 4620 |
| TTTTAAAGTC TTTACGATGT AAAATATTTA TTTTTTACTT ATTCTGGAAG ATCTGGCTGA | 4680 |
| AGGATTATTC ATGGAACAGG AAGAAGCGTA AAGACTATCC ATGTCATCTT TGTTGAGAGT | 4740 |
| CTTCGTGACT GTAAGATTGT AAATACAGAT TATTTATTAA CTCTGTTCTG CCTGGAAATT | 4800 |
| TAGGCTTCAT ACGGAAAGTG TTTGAGAGCA AGTAGTTGAC ATTTATCAGC AAATCTCTTG | 4860 |
| CAAGAACAGC ACAAGGAAAA TCAGTCTAAT AAGCTGCTCT GCCCCTTGTG CTCAGAGTGG | 4920 |
| ATGTTATGGG ATTCCTTTTT TCTCTGTTTT ATCTTTTCAA GTGGAATTAG TTGGTTATCC | 4980 |
| ATTTGCAAAT GTTTTAAATT GCAAAGAAAG CCATGAGGTC TTCAATACTG TTTTACCCCA | 5040 |
| TCCCTTGTGC ATATTTCCAG GGAGAAGGAA AGGATATACA CTTTTTTCTT TCATTTTTCC | 5100 |
| AAAAGAGAAA AAAATGACAA AAGGTGAAAC TTACATACAA ATATTACCTC ATTTGTTGTG | 5160 |
| TGACTGAGTA AAGAATTTTT GGATCAAGCG GAAAGAGTTT AAGTGTCTAA CAAACTTAAA | 5220 |
| GCTACTGTAG TACCTAAAAA GTCAGTGTTG TACATAGCAT AAAAACTCTG CAGAGAAGTA | 5280 |
| TTCCCAATAA GGAAATAGCA TTGAAATGTT AAATACAATT TCTGAAAGTT ATGTTTTTTT | 5340 |
| TCTATCATCT GGTATACCAT TGCTTTATTT TTATAAATTA TTTTCTCATT GCCATTGGAA | 5400 |
| TAGAATATTC AGATTGTGTA GATATGCTAT TTAAATAATT TATCAGGAAA TACTGCCTGT | 5460 |
| AGAGTTAGTA TTTCTATTTT TATATAATGT TTGCACACTG AATTGAAGAA TTGTTGGTTT | 5520 |
| TTTCTTTTTT TTGTTTTTTT TTTTTTTTTT TTTTTTTTTG CTTTTGACCT CCCATTTTTA | 5580 |
| CTATTTGCCA ATACCTTTTT CTAGGAATGT GCTTTTTTTT GTACACATTT TTATCCATTT | 5640 |
| TACATTCTAA AGCAGTGTAA GTTGTATATT ACTGTTTCTT ATGTACAAGG AACAACAATA | 5700 |
| AATCATATGG AAATTTATAT TT | |

AAD9 DNA sequence
Gene name: LIM homeobox protein cofactor (CLIM-1)
Unigene number: Hs.4980
Probeset Accession #: F13782
Nucleic Acid Accession #: AF047337
Coding sequence: 110-1231 (predicted start/stop codons underlined)

| | |
|---|---|
| GTGAGCGTGT GTGCGTGCGT CTACTTTGTA CTGGGAAGAA CACAGCCCAT GTGCTCTGCA | 60 |
| TGGACGTTAC TGATACTCTG TTTAGCTTGA TTTTCGAAAA GCAGGCAAGA TGTCCAGCAC | 120 |
| ACCACATGAC CCCTTCTATT CTTCTCCTTT CGGCCCATTT TATAGGAGGC ATACACCATA | 180 |
| CATGGTACAG CCAGAGTACC GAATCTATGA GATGAACAAG AGACTGCAGT CTCGCACAGA | 240 |
| GGATAGTGAC AACCTCTGGT GGGACGCCTT TGCCACTGAA TTTTTTGAAG ATGACGCCAC | 300 |
| ATTAACCCTT TCATTTTGTT TGGAAGATGG ACCAAAGCGA TACACTATCG GCAGGACCCT | 360 |
| CATCCCCCGT TACTTTAGCA CTGTGTTTGA AGGAGGGGTG ACCGACCTGT ATTACATTCT | 420 |
| CAAACACTCG AAAGAGTCAT ACCACAACTC ATCCATCACG GTGGACTGCG ACCAGTGTAC | 480 |
| CATGGTCACC CAGCACGGGA AGCCCATGTT TACCAAGGTA TGTACAGAAG GCAGACTGAT | 540 |
| CTTGGAGTTC ACCTTTGATG ATCTCATGAG AATCAAAACA TGGCACTTTA CCATTAGACA | 600 |
| ATACCGAGAG TTAGTCCCGA GAAGCATCCT AGCCATGCAT GCACAAGATC CTCAGGTCCT | 660 |
| GGATCAGCTG TCCAAAAACA TCACCAGGAT GGGGCTAACA AACTTCACCC TCAACTACCT | 720 |
| CAGGTTGTGT GTAATATTGG AGCCAATGCA GGAACTGATG TCGAGACATA AAACTTACAA | 780 |
| CCTCAGTCCC CGAGACTGCC TGAAGACCTG CTTGTTTCAG AAGTGGCAGA GGATGGTGGC | 840 |
| TCCGCCAGCA GAACCCACAA GGCAACCAAC AACCAAACGG AGAAAAAGGA AAAATTCCAC | 900 |
| CAGCAGCACT TCCAACAGCA GCGCTGGGAA CAATGCAAAC AGCACTGGCA GCAAGAAGAA | 960 |
| GACCACAGCT GCAAACCTGA GTCTGTCCAG TCAGGTACCT GATGTGATGG TGGTAGGAGA | 1020 |
| GCCAACTCTG ATGGGAGGTG AGTTTGGGGA CGAGGACGAA AGGCTAATCA CTAGATTAGA | 1080 |
| AAACACGCAA TATGATGCGG CCAACGGCAT GGACGACGAG GAGGACTTCA ACAATTCACC | 1140 |
| CGCGCTGGGG AACAACAGCC CGTGGAACAG TAAACCTCCC GCCACTCAAG AGACCAAATC | 1200 |
| AGAAAACCCC CCACCCCAGG CTTCCCAATA AGATGATCGG CACCAGAATC CACTGTCAAT | 1260 |
| AGGCCCGTGG GTGATCATTA CAATTGCAAA TCTTTACTTA CAGGAGAGGA AACAGAAGAG | 1320 |
| ATAAAAACTT TTCCATGCAA ATATCTATTT CTAAACCACA ATGATCTGAT TTTCTTTCTT | 1380 |
| CTTTCTTTTT TTCTAATTGA GAGGATTATT CCCAGTAAGC TTCCATGACC CTTTCTTGGA | 1440 |
| GGCCTTCACA GGTAATACAG ATACTGGCAC TGATTGTAAT TAAAATGAGA GAAAACTCTA | 1500 |
| GCGCATCTTC TGGCACGGTT TTAACAACGT GTTTGTGTTG AATTTCCTTT TTATGCATCA | 1560 |
| AACGAAGGCC ATATTGTCCA TAAATGCTCA GTGCTCAGGA TCTCATTAAT ATGCCGAACC | 1620 |
| TAACTACAGA TGACTTTTTA ATATTGTAAA ATATTTTCTG CTTTTTGACT TGCATCTGAG | 1680 |
| AGTTTCTTGT TTCAGTAAAA AAAGAAAAGA CAAAAAAATC AGCTTTGGAA AGTAATTTAA | 1740 |
| ATGTACCTTA TTTTTTTTTT CTTTATGTTT TCTTTCATTG GGCAACAGCT AAGAGGGCCC | 1800 |
| AGCAAGGTAA TTTATGGTTG AGCTGATGTC AATTGGTTCT TGTCTTGAGT CGACTCAATT | 1860 |
| TAGCCCAAGT GCTGAAACAA GAAATGTCAT TTTTTTCATC AAAGACACCA GGGCAGATTT | 1920 |
| TTAAGTAAAG AAAGACAATT GGACCCTTAA GAATTTATGC ATTTGTAAAG TTGCTGTTGA | 1980 |
| TCCAAATATT TTCAAGCCAT GTAATCCATT GGTTTTGTGG GCAGTTTAAT AAACCTGAAC | 2040 |
| CTTTGTGTGT TTTCTAATTG TACCTGAGTT GACCATCCTT TCTTTTTATA GTATATTTCT | 2100 |
| TGTATGATAT TTTGTAAAGC TCTCACCTGG TTCTTTTATG GGGACTTTTC GTTTTTGGGC | 2160 |
| AACTCCAGTG TATTTATGTG AAACTTTATA AGAGAATTAA TTTTTCCATT TGCATATTAA | 2220 |
| TATGTTCCTC CACACATGTA AAGGCACAGT GGCTCCGTGT GTTAAAAAAC AGCTGTATTT | 2280 |
| TATGTATGCT TTACTGATAA GTGTGCCAAT AATAAACTGT GTTAATGACC | |

AAE1 DNA sequence
Gene name: guanine nucleotide binding protein 11
Unigene number: Hs.83381
Probeset Accession #: U31384
Nucleic Acid Accession #: NM_004126.1
Coding sequence: 108-329 (predicted start/stop codons underlined)

| | |
|---|---|
| GGCACGAGCT CGTGCCGGCC TTCAGTTGTT TCGGGACGCG CCGAGCTTCG CCGCTCTTCC | 60 |
| AGCGGCTCCG CTGCCAGAGC TAGCCCGAGC CCGGTTCTGG GGCGAAAATG CCTGCCCTTC | 120 |
| ACATCGAAGA TTTGCCAGAG AAGGAAAAAC TGAAAATGGA AGTTGAGCAG CTTCGCAAAG | 180 |
| AAGTGAAGTT GCAGAGACAA CAAGTGTCTA AATGTTCTGA AGAAATAAAG AACTATATTG | 240 |
| AAGAACGTTC TGGAGAGGAT CCTCTAGTAA AGGGAATTCC AGAAGACAAG AACCCCTTTA | 300 |
| AAGAAAAAGG CAGCTGTGTT ATTTCATAAA TAACTTGGGA GAAACTGCAT CCTAAGTGGA | 360 |
| AGAACTAGTT TGTTTTAGTT TTCCCAGATA AAACCAACAT GCTTTTTAAG GAAGGAAGAA | 420 |
| TGAAATTAAA AGGAGACTTT CTTAAGCACC ATATAGATAG GGTTATGTAT AAAAGCATAT | 480 |
| GTGCTACTCA TCTTTGCTCA CTATGCAGTC TTTTTTAAGA GAGCAGAGAG TATCAGATGT | 540 |
| ACAATTATGG AAATAAGAAC ATTACTTGAG CATGACACTT CTTTCAGTAT ATTGCTTGAT | 600 |
| GCTTCAAATA AAGTTTTGTC TT | |

AAE2 DNA sequence
Gene name: Transcription factor 4 (immunoglobulin transcription factor 2) (ITF-2)
(SL3-3 Enhancer factor 2) (SEF-2)
Unigene number: Hs.289068
Probeset Accession #: M74719
Nucleic Acid Accession #: NM_003199.1
coding sequence: 200-2203 (predicted start/stop codons underlined)

| | |
|---|---|
| CGGGGGGATC TTGGCTGTGT GTCTGCGGAT CTGTAGTGGC GGCGGCGGCG GCGGCGGCGG | 60 |
| GGAGGCAGCA GGCGCGGGAG CGGGCGCAGG AGCAGGCGGC GGCGGTGGCG GCGGCGGTTA | 120 |
| GACATGAACG CCGCCTCGGC GCCGGCGGTG CACGGAGAGC CCCTTCTCGC GCGCGGGCGG | 180 |
| TTTGTGTGAT TTTGCTAAAA TGCATCACCA ACAGCGAATG GCTGCCTTAG GGACGGACAA | 240 |
| AGAGCTGAGT GATTTACTGG ATTTCAGTGC GATGTTTTCA CCTCCTGTGA GCAGTGGGAA | 300 |
| AAATGGACCA ACTTCTTTGG CAAGTGGACA TTTTACTGGC TCAAATGTAG AAGACAGAAG | 360 |
| TAGCTCAGGG TCCTGGGGGA ATGGAGGACA TCCAAGCCCG TCCAGGAACT ATGGAGATGG | 420 |
| GACTCCCTAT GACCACATGA CCAGCAGGGA CCTTGGGTCA CATGACAATC TCTCTCCACC | 480 |
| TTTTGTCAAT TCCAGAATAC AAAGTAAAAC AGAAAGGGGC TCATACTCAT CTTATGGGAG | 540 |
| AGAATCAAAC TTACAGGGTT GCCACCAGCA GAGTCTCCTT GGAGGTGACA TGGATATGGG | 600 |
| CAACCCAGGA ACCCTTTCGC CCACCAAACC TGGTTCCCAG TACTATCAGT ATTCTAGCAA | 660 |
| TAATCCCCGA AGGAGGCCTC TTCACAGTAG TGCCATGGAG GTACAGACAA AGAAAGTTCG | 720 |
| AAAAGTTCCT CCAGGTTTGC CATCTTCAGT CTATGCTCCA TCAGCAAGCA CTGCCGACTA | 780 |
| CAATAGGGAC TCGCCAGGCT ATCCTTCCTC CAAACCAGCA ACCAGCACTT TCCCTAGCTC | 840 |
| CTTCTTCATG CAAGATGGCC ATCACAGCAG TGACCCTTGG AGCTCCTCCA GTGGGATGAA | 900 |
| TCAGCCTGGC TATGCAGGAA TGTTGGGCAA CTCTTCTCAT ATTCCACAGT CCAGCAGCTA | 960 |
| CTGTAGCCTG CATCCACATG AACGTTTGAG CTATCCATCA CACTCCTCAG CAGACATCAA | 1020 |
| TTCCAGTCTT CCTCCGATGT CCACTTTCCA TCGTAGTGGT ACAAACCATT ACAGCACCTC | 1080 |
| TTCCTGTACG CCTCCTGCCA ACGGGACAGA CAGTATAATG GCAAATAGAG GAAGCGGGGC | 1140 |
| AGCCGGCAGC TCCCAGACTG GAGATGCTCT GGGGAAAGCA CTTGCTTCGA TCTATTCTCC | 1200 |
| AGATCACACT AACAACAGCT TTTCATCAAA CCCTTCAACT CCTGTTGGCT CTCCTCCATC | 1260 |
| TCTCTCAGCA GGCACAGCTG TTTGGTCTAG AAATGGAGGA CAGGCCTCAT CGTCTCCTAA | 1320 |
| TTATGAAGGA CCCTTACACT CTTTGCAAAG CCGAATTGAA GATCGTTTAG AAAGACTGGA | 1380 |
| TGATGCTATT CATGTTCTCC GGAACCATGC AGTGGGCCCA TCCACAGCTA TGCCTGGTGG | 1440 |
| TCATGGGGAC ATGCATGGAA TCATTGGACC TTCTCATAAT GGAGCCATGG GTGGTCTGGG | 1500 |
| CTCAGGGTAT GGAACCGGCC TTCTTTCAGC CAACAGACAT TCACTCATGG TGGGGACCCA | 1560 |
| TCGTGAAGAT GGCGTGGCCC TGAGAGGCAG CCATTCTCTT CTGCCAAACC AGGTTCCGGT | 1620 |
| TCCACAGCTT CCTGTCCAGT CTGCGACTTC CCCTGACCTG AACCCACCCC AGGACCCTTA | 1680 |
| CAGAGGCATG CCACCAGGAC TACAGGGGCA GAGTGTCTCC TCTGGCAGCT CTGAGATCAA | 1740 |
| ATCCGATGAC GAGGGTGATG AGAACCTGCA AGACACGAAA TCTTCGGAGG ACAAGAAATT | 1800 |
| AGATGACGAC AAGAAGGATA TCAAATCAAT TACTAGCAAT AATGACGATG AGGACCTGAC | 1860 |
| ACCAGAGCAG AAGGCAGAGC GTGAGAAGGA GCGGAGGATG GCCAACAATG CCCGAGAGCG | 1920 |
| TCTGCGGGTC CGTGACATCA ACGAGGCTTT CAAAGAGCTC GGCCGCATGG TGCAGCTCCA | 1980 |
| CCTCAAGAGT GACAAGCCCC AGACCAAGCT CCTGATCCTC CACCAGGCGG TGGCCGTCAT | 2040 |
| CCTCAGTCTG GAGCAGCAAG TCCGAGAAAG GAATCTGAAT CCGAAAGCTG CGTGTCTGAA | 2100 |
| AAGAAGGGAG GAAGAGAAGG TGTCCTCGGA GCCTCCCCCT CTCTCCTTGG CCGGCCCACA | 2160 |
| CCCTGGAATG GGAGACGCAT CGAATCACAT GGGACAGATG TAAAAGGGTC CAAGTTGCCA | 2220 |
| CATTGCTTCA TTAAAACAAG AGACCACTTC CTTAACAGCT GTATTATCTT AAACCCACAT | 2280 |
| AAACACTTCT CCTTAACCCC CATTTTTGTA ATATAAGACA AGTCTGAGTA GTTATGAATC | 2340 |
| GCAGACGCAA GAGGTTTCAG CATTCCCAAT TATCAAAAAA CAGAAAAACA AAAAAAAGAA | 2400 |
| AGAAAAAAGT GCAACTTGAG GGACGACTTT CTTTAACATA TCATTCAGAA TGTGCAAAGC | 2460 |
| AGTATGTACA GGCTGAGACA CAGCCCAGAG ACTGAACGGC | |

AAE4 DNA sequence
Gene name: phosphatidylcholine 2-acylhydrolase
Unigene number: Hs.211587
Probeset Accession #: M68874
Nucleic Acid Accession #: M68874
Coding sequence: 139-2388 (predicted start/stop codons underlined)

| | |
|---|---|
| GAATTCTCCG GAGCTGAAAA AGGATCCTGA CTGAAAGCTA GAGGCATTGA GGAGCCTGAA | 60 |
| GATTCTCAGG TTTTAAAGAC GCTAGAGTGC CAAAGAAGAC TTTGAAGTGT GAAAACATTT | 120 |
| CCTGTAATTG AAACCAAAAT GTCATTTATA GATCCTTACC AGCACATTAT AGTGGAGCAC | 180 |
| CAGTATTCCC ACAAGTTTAC GGTAGTGGTG TTACGTGCCA CCAAAGTGAC AAAGGGGGCC | 240 |
| TTTGGTGACA TGCTTGATAC TCCAGATCCC TATGTGGAAC TTTTTATCTC TACAACCCCT | 300 |
| GACAGCAGGA AGAGAACAAG ACATTTCAAT AATGACATAA ACCCTGTGTG GAATGAGACC | 360 |
| TTTGAATTTA TTTTGGATCC TAATCAGGAA AATGTTTTGG AGATTACGTT AATGGATGCC | 420 |
| AATTATGTCA TGGATGAAAC TCTAGGGACA GCAACATTTA CTGTATCTTC TATGAAGGTG | 480 |
| GGAGAAAAGA AAGAAGTTCC TTTTATTTTC AACCAAGTCA CTGAAATGGT TCTAGAAATG | 540 |
| TCTCTTGAAG TTTGCTCATG CCCAGACCTA CGATTTAGTA TGGCTCTGTG TGATCAGGAG | 600 |
| AAGACTTTCA GACAACAGAG AAAAGAACAC ATAAGGGAGA GCATGAAGAA ACTCTTGGGT | 660 |
| CCAAAGAATA GTGAAGGATT GCATTCTGCA CGTGATGTGC CTGTGGTAGC CATATTGGGT | 720 |
| TCAGGTGGGG GTTTCCGAGC CATGGTGGGA TTCTCTGGTG TGATGAAGGC ATTATACGAA | 780 |
| TCAGGAATTC TGGATTGTGC TACCTACGTT GCTGGTCTTT CTGGCTCCAC CTGGTATATG | 840 |
| TCAACCTTGT ATTCTCACCC TGATTTTCCA GAGAAAGGGC CAGAGGAGAT TAATGAAGAA | 900 |
| CTAATGAAAA ATGTTAGCCA CAATCCCCTT TTACTTCTCA CACCACAGAA AGTTAAAAGA | 960 |
| TATGTTGAGT CTTTATGGAA GAAGAAAAGC TCTGGACAAC CTGTCACCTT TACTGACATC | 1020 |
| TTTGGGATGT TAATAGGAGA AACACTAATT CATAATAGAA TGAATACTAC TCTGAGCAGT | 1080 |
| TTGAAGGAAA AAGTTAATAC TGCACAATGC CCTTTACCTC TTTTCACCTG TCTTCATGTC | 1140 |
| AAACCTGACG TTTCAGAGCT GATGTTTGCA GATTGGGTTG AATTTAGTCC ATACGAAATT | 1200 |
| GGCATGGCTA AATACGGTAC TTTTATGGCT CCCGACTTAT TTGGAAGCAA ATTTTTTATG | 1260 |
| GGAACAGTCG TTAAGAAGTA TGAAGAAAAC CCCTTGCATT TCTTAATGGG TGTCTGGGGC | 1320 |
| AGTGCCTTTT CCATATTGTT CAACAGAGTT TTGGGCGTTT CTGGTTCACA AAGCAGAGGC | 1380 |
| TCCACAATGG AGGAAGAATT AGAAAATATT ACCACAAAGC ATATTGTGAG TAATGATAGC | 1440 |
| TCGGACAGTG ATGATGAATC ACACGAACCC AAAGGCACTG AAAATGAAGA TGCTGGAAGT | 1500 |
| GACTATCAAA GTGATAATCA AGCAAGTTGG ATTCATCGTA TGATAATGGC CTTGGTGAGT | 1560 |
| GATTCAGCTT TATTCAATAC CAGAGAAGGA CGTGCTGGGA AGGTACACAA CTTCATGCTG | 1620 |
| GGCTTGAATC TCAATACATC TTATCCACTG TCTCCTTTGA GTGACTTTGC CACACAGGAC | 1680 |
| TCCTTTGATG ATGATGAACT GGATGCAGCT GTAGCAGATC CTGATGAATT TGAGCGAATA | 1740 |
| TATGAGCCTC TGGATGTCAA AAGTAAAAAG ATTCATGTAG TGGACAGTGG GCTCACATTT | 1800 |
| AACCTGCCGT ATCCCTTGAT ACTGAGACCT CAGAGAGGGG TTGATCTCAT AATCTCCTTT | 1860 |
| GACTTTTCTG CAAGGCCAAG TGACTCTAGT CCTCCGTTCA AGGAACTTCT ACTTGCAGAA | 1920 |
| AAGTGGGCTA AAATGAACAA GCTCCCCTTT CCAAAGATTG ATCCTTATGT GTTTGATCGG | 1980 |
| GAAGGGCTGA AGGAGTGCTA TGTCTTTAAA CCCAAGAATC CTGATATGGA GAAAGATTGC | 2040 |
| CCAACCATCA TCCACTTTGT TCTGGCCAAC ATCAACTTCA GAAAGTACAA GGCTCCAGGT | 2100 |
| GTTCCAAGGG AAACTGAGGA AGAGAAAGAA ATCGCTGACT TTGATATTTT TGATGACCCA | 2160 |
| GAATCACCAT TTTCAACCTT CAATTTTCAA TATCCAAATC AAGCATTCAA AAGACTACAT | 2220 |
| GATCTTATGC ACTTCAATAC TCTGAACAAC ATTGATGTGA TAAAAGAAGC CATGGTTGAA | 2280 |
| AGCATTGAAT ATAGAAGACA GAATCCATCT CGTTGCTCTG TTTCCCTTAG TAATGTTGAG | 2340 |
| GCAAGAAGAT TTTTCAACAA GGAGTTTCTA AGTAAACCCA AAGCATAGTT CATGTACTGG | 2400 |
| AAATGGCAGC AGTTTCTGAT GCTGAGGCAG TTTGCAATCC CATGACAACT GGATTTAAAA | 2460 |
| GTACAGTACA GATAGTCGTA CTGATCATGA GAGACTGGCT GATACTCAAA GTTGCAGTTA | 2520 |
| CTTAGCTGCA TGAGAATAAT ACTATTATAA GTTAGGTGAC AAATGATGTT GATTATGTAA | 2580 |
| GGATATACTT AGCTACATTT TCAGTCAGTA TGAACTTCCT GATACAAATG TAGGGATATA | 2640 |
| TACTGTATTT TTAAACATTT CTCACCAACT TTCTTATGTG TGTTCTTTTT AAAAATTTTT | 2700 |
| TTTCTTTTAA AATATTTAAC AGTTCAATCT CAATAAGACC TCGCATTATG TATGAATGTT | 2760 |
| ATTCACTGAC TAGATTTATT CATACCATGA GACAACACTA TTTTTATTTA TATATGCATA | 2820 |
| TATATACATA CATGAAATAA ATACATCAAT ATAAAAATAA AAAAAAACGG AATTC | |

ACA1 DNA sequence
Gene name: tissue factor pathway inhibitor 2 TFPI2, placental protein 5 (PP5)
Unigene number: Hs.78045
Probeset Accession #: D29992
Nucleic Acid Accession #: D29992.1
Coding sequence: 57-764 (predicted start/stop codons underlined)

| | |
|---|---|
| GCCGCCAGCG GCTTTCTCGG ACGCCTTGCC CAGCGGGCCG CCCGACCCCC TGCACCATGG | 60 |
| ACCCCGCTCG CCCCCTGGGG CTGTCGATTC TGCTGCTTTT CCTGACGGAG GCTGCACTGG | 120 |
| GCGATGCTGC TCAGGAGCCA ACAGGAAATA ACGCGGAGAT CTGTCTCCTG CCCCTAGACT | 180 |
| ACGGACCCTG CCGGGCCCTA CTTCTCCGTT ACTACTACGA CAGGTACACG CAGAGCTGCC | 240 |
| GCCAGTTCCT GTACGGGGGC TGCGAGGGCA ACGCCAACAA TTTCTACACC TGGGAGGCTT | 300 |
| GCGACGATGC TTGCTGGAGG ATAGAAAAAG TTCCCAAAGT TTGCCGGCTG CAAGTGAGTG | 360 |
| TGGACGACCA GTGTGAGGGG TCCACAGAAA AGTATTTCTT TAATCTAAGT TCCATGACAT | 420 |
| GTGAAAAATT CTTTTCCGGT GGGTGTCACC GGAACCGGAT TGAGAACAGG TTTCCAGATG | 480 |
| AAGCTACTTG TATGGGCTTC TGCGCACCAA AGAAAATTCC ATCATTTTGC TACAGTCCAA | 540 |
| AAGATGAGGG ACTGTGCTCT GCCAATGTGA CTCGCTATTA TTTTAATCCA AGATACAGAA | 600 |
| CCTGTGATGC TTTCACCTAT ACTGGCTGTG GAGGGAATGA CAATAACTTT GTTAGCAGGG | 660 |
| AGGATTGCAA ACGTGCATGT GCAAAAGCTT TGAAAAAGAA AAAGAAGATG CCAAAGCTTC | 720 |
| GCTTTGCCAG TAGAATCCGG AAAATTCGGA AGAAGCAATT TTAAACATTC TTAATATGTC | 780 |
| ATCTTGTTTG TCTTTATGGC TTATTTGCCT TTATGGTTGT ATCTGAAGAA TAATATGACA | 840 |
| GCATGAGGAA ACAAATCATT GGTGATTTAT TCACCAGTTT TTATTAATAC AAGTCACTTT | 900 |
| TTCAAAAATT TGGATTTTTT TATATATAAC TAGCTGCTAT TCAAATGTGA GTCTACCATT | 960 |
| TTTAATTTAT GGTTCAACTG TTTGTGAGAC GAATTCTTGC AATGCATAAG ATATAAAAGC | 1020 |
| AAATATGACT CACTCATTTC TTGGGGTCGT ATTCCTGATT TCAGAAGAGG ATCATAACTG | 1080 |
| AAACAACATA AGACAATATA ATCATGTGCT TTTAACATAT TTGAGAATAA AAAGGACTAG | 1140 |
| CC | |

ACB8 DNA sequence
Gene name: myosin X
Unigene number: Hs.61638
Probeset Accession #: N77151
Nucleic Acid Accession #: NM_012334
Coding sequence: 223-6399 (predicted start/stop codons underlined)

| | |
|---|---|
| GAGACAAAGG CTGCCGTCGG GACGGGCGAG TTAGGGACTT GGGTTTGGGC GAACAAAAGG | 60 |
| TGAGAAGGAC AAGAAGGGAC CGGGCGATGG CAGCAGGGGA GCCCCGCGGG CGCGCGTCCT | 120 |
| CGGGAGTGGC GCCGTGACAC GCATGGTTTC CCCCGACCCG CGGCGGCGCT GACTTCCGCG | 180 |
| AGTCGGAGCG GCACTCGGCG AGTCCGGGAC TGCGCTGGAA CAATGGATAA CTTCTTCACC | 240 |
| GAGGGAACAC GGGTCTGGCT GAGAGAAAAT GGCCAGCATT TTCCAAGTAC TGTAAATTCC | 300 |
| TGTGCAGAAG GCATCGTCGT CTTCCGGACA GACTATGGTC AGGTATTCAC TTACAAGCAG | 360 |
| AGCACAATTA CCCACCAGAA GGTGACTGCT ATGCACCCCA CGAACGAGGA GGGCGTGGAT | 420 |
| GACATGGCGT CCTTGACAGA GCTCCATGGC GGCTCCATCA TGTATAACTT ATTCCAGCGG | 480 |
| TATAAGAGAA ATCAAATATA TACCTACATC GGCTCCATCC TGGCCTCCGT GAACCCCTAC | 540 |
| CAGCCCATCG CCGGGCTGTA CGAGCCTGCC ACCATGGAGC AGTACAGCCG GCGCCACCTG | 600 |
| GGCGAGCTGC CCCCGCACAT CTTCGCCATC GCCAACGAGT GCTACCGCTG CCTGTGGAAG | 660 |
| CGCTACGACA ACCAGTGCAT CCTCATCAGT GGTGAAAGTG GGGCAGGTAA AACCGAAAGC | 720 |
| ACTAAATTGA TCCTCAAGTT TCTGTCAGTC ATCAGTCAAC AGTCTTTGGA ATTGTCCTTA | 780 |
| AAGGAGAAGA CATCCTGTGT TGAACGAGCT ATTCTTGAAA GCAGCCCCAT CATGGAAGCT | 840 |
| TTCGGCAATG CGAAGACCGT GTACAACAAC AACTCTAGTC GCTTTGGGAA GTTTGTTCAG | 900 |
| CTGAACATCT GTCAGAAAGG AAATATTCAG GGCGGGAGAA TTGTAGATTA TTTATTAGAA | 960 |
| AAAAACCGAG TAGTAAGGCA AAATCCCGGG GAAAGGAATT ATCACATATT TTATGCACTG | 1020 |
| CTGGCAGGGC TGGAACATGA AGAAAGAGAA GAATTTTATT TATCTACGCC AGAAAACTAC | 1080 |
| CACTACTTGA ATCAGTCTGG ATGTGTAGAA GACAAGACAA TCAGTGACCA GGAATCCTTT | 1140 |
| AGGGAAGTTA TTACGGCAAT GGACGTGATG CAGTTCAGCA AGGAGGAAGT TCGGGAAGTG | 1200 |
| TCGAGGCTGC TTGCTGGTAT ACTGCATCTT GGGAACATAG AATTTATCAC TGCTGGTGGG | 1260 |
| GCACAGGTTT CCTTCAAAAC AGCTTTGGGC AGATCTGCGG AGTTACTTGG GCTGGACCCA | 1320 |
| ACACAGCTCA CAGATGCTTT GACCCAGAGA TCAATGTTCC TCAGGGGAGA AGAGATCCTC | 1380 |
| ACGCCTCTCA ATGTTCAACA GGCAGTAGAC AGCAGGGACT CCCTGGCCAT GGCTCTGTAT | 1440 |
| GCGTGCTGCT TTGAGTGGGT AATCAAGAAG ATCAACAGCA GGATCAAAGG CAATGAGGAC | 1500 |
| TTCAAGTCTA TTGGCATCCT CGACATCTTT GGATTTGAAA ACTTTGAGGT TAATCACTTT | 1560 |
| GAACAGTTCA ATATAAACTA TGCAAACGAG AAACTTCAGG AGTACTTCAA CAAGCATATT | 1620 |
| TTTTCTTTAG AACAACTAGA ATATAGCCGG GAAGGATTAG TGTGGGAAGA TATTGACTGG | 1680 |
| ATAGACAATG GAGAATGCCT GGACTTGATT GAGAAGAAAC TTGGCCTCCT AGCCCTTATC | 1740 |
| AATGAAGAAA GCCATTTTCC TCAAGCCACA GACAGCACCT TATTGGAGAA GCTACACAGT | 1800 |
| CAGCATGCGA ATAACCACTT TTATGTGAAG CCCAGAGTTG CAGTTAACAA TTTTGGAGTG | 1860 |
| AAGCACTATG CTGGAGAGGT GCAATATGAT GTCCGAGGTA TCTTGGAGAA GAACAGAGAT | 1920 |
| ACATTTCGAG ATGACCTTCT CAATTTGCTA AGAGAAAGCC GATTTGACTT TATCTACGAT | 1980 |
| CTTTTTGAAC ATGTTTCAAG CCGCAACAAC CAGGATACCT TGAAATGTGG AAGCAAACAT | 2040 |
| CGGCGGCCTA CAGTCAGCTC ACAGTTCAAG GACTCACTGC ATTCCTTAAT GGCAACGCTA | 2100 |
| AGCTCCTCTA ATCCTTTCTT TGTTCGCTGT ATCAAGCCAA ACATGCAGAA GATGCCAGAC | 2160 |
| CAGTTTGACC AGGCGGTTGT GCTGAACCAG CTGCGGTACT CAGGGATGCT GGAGACTGTG | 2220 |
| AGAATCCGCA AAGCTGGGTA TGCGGTCCGA AGACCCTTTC AGGACTTTTA CAAAAGGTAT | 2280 |
| AAAGTGCTGA TGAGGAATCT GGCTCTGCCT GAGGACGTCC GAGGGAAGTG CACGAGCCTG | 2340 |
| CTGCAGCTCT ATGATGCCTC CAACAGCGAG TGGCAGCTGG GGAAGACCAA GGTCTTTCTT | 2400 |
| CGAGAATCCT TGGAACAGAA ACTGGAGAAG CGGAGGGAAG AGGAAGTGAG CCACGCGGCC | 2460 |
| ATGGTGATTC GGGCCCATGT CTTGGGCTTC TTAGCACGAA AACAATACAG AAAGGTCCTT | 2520 |
| TATTGTGTGG TGATAATACA GAAGAATTAC AGAGCATTCC TTCTGAGGAG GAGATTTTTG | 2580 |
| CACCTGAAAA AGGCAGCCAT AGTTTTCCAG AAGCAACTCA GAGGTCAGAT TGCTCGGAGA | 2640 |
| GTTTACAGAC AATTGCTGGC AGAGAAAAGG GAGCAAGAAG AAAAGAAGAA ACAGGAAGAG | 2700 |
| GAAGAAAAGA AGAAACGGGA GGAAGAAGAA AGAGAAAGAG AGAGAGAGCG AAGAGAAGCC | 2760 |
| GAGCTCCGCG CCCAGCAGGA AGAAGAAACG AGGAAGCAGC AAGAACTCGA AGCCTTGCAG | 2820 |
| AAGAGCCAGA AGGAAGCTGA ACTGACCCGT GAACTGGAGA AACAGAAGGA AAATAAGCAG | 2880 |
| GTGGAAGAGA TCCTCCGTCT GGAGAAAGAA ATCGAGGACC TGCAGCGCAT GAAGGAGCAG | 2940 |
| CAGGAGCTGT CGCTGACCGA GGCTTCCCTG CAGAAGCTGC AGGAGCGGCG GGACCAGGAG | 3000 |
| CTCCGCAGGC TGGAGGAGGA AGCGTGCAGG GCGGCCCAGG AGTTCCTCGA GTCCCTCAAT | 3060 |
| TTCGACGAGA TCGACGAGTG TGTCCGGAAT ATCGAGCGGT CCCTGTCGGT GGGAAGCGAA | 3120 |
| TTTTCCAGCG AGCTGGCTGA GAGCGCATGC GAGGAGAAGC CCAACTTCAA CTTCAGCCAG | 3180 |
| CCCTACCCAG AGGAGGAGGT CGATGAGGGC TTCGAAGCCG ACGACGACGC CTTCAAGGAC | 3240 |
| TCCCCCAACC CCAGCGAGCA CGGCCACTCA GACCAGCGAA CAAGTGGCAT CCGGACCAGC | 3300 |
| GATGACTCTT CAGAGGAGGA CCCATACATG AACGACACGG TGGTGCCCAC CAGCCCCAGT | 3360 |
| GCGGACAGCA CGGTGCTGCT CGCCCCATCA GTGCAGGACT CCGGGAGCCT ACACAACTCC | 3420 |
| TCCAGCGGCG AGTCCACCTA CTGCATGCCC CAGAACGCTG GGGACTTGCC CTCCCCAGAC | 3480 |
| GGCGACTACG ACTACGACCA GGATGACTAT GAGGACGGTG CCATCACTTC CGGCAGCAGC | 3540 |
| GTGACCTTCT CCAACTCCTA CGGCAGCCAG TGGTCCCCCG ACTACCGCTG CTCTGTGGGG | 3600 |
| ACCTACAACA GCTCGGGTGC CTACCGGTTC AGCTCTGAGG GGGCGCAGTC CTCGTTTGAA | 3660 |
| GATAGTGAAG AGGACTTTGA TTCCAGGTTT GATACAGATG ATGAGCTTTC ATACCGGCGT | 3720 |
| GACTCTGTGT ACAGCTGTGT CACTCTGCCG TATTTCCACA GCTTTCTGTA CATGAAAGGT | 3780 |
| GGCCTGATGA ACTCTTGGAA ACGCCGCTGG TGCGTCCTCA AGGATGAAAC CTTCTTGTGG | 3840 |
| TTCCGCTCCA AGCAGGAGGC CCTCAAGCAA GGCTGGCTCC ACAAAAAAGG GGGGGGCTCC | 3900 |
| TCCACGCTGT CCAGGAGAAA TTGGAAGAAG CGCTGGTTTG TCCTCCGCCA GTCCAAGCTG | 3960 |
| ATGTACTTTG AAAACGACAG CGAGGAGAAG CTCAAGGGCA CCGTAGAAGT GCGAACGGCA | 4020 |
| AAAGAGATCA TAGATAACAC CACCAAGGAG AATGGGATCG ACATCATTAT GGCCGATAGG | 4080 |
| ACTTTCCACC TGATTGCAGA GTCCCCAGAA GATGCCAGCC AGTGGTTCAG CGTGCTGAGT | 4140 |
| CAGGTCCACG CGTCCACGGA CCAGGAGATC CAGGAGATGC ATGATGAGCA GGGAAACCCA | 4200 |
| CAGAATGCTG TGGGCACCTT GGATGTGGGG CTGATTGATT CTGTGTGTGC CTCGACAGC | 4260 |
| CCTGATAGAC CCAACTCGTT TGTGATCATC ACGGCCAACC GGGTGCTGCA CTGCAACGCC | 4320 |
| GACACGCCGG AGGAGATGCA CCACTGGATA ACCCTGCTGC AGAGGTCCAA AGGGGACACC | 4380 |
| AGAGTGGAGG GCCAGGAATT CATCGTGAGA GGATGGTTGC ACAAAGAGGT GAAGAACAGT | 4440 |
| CCGAAGATGT CTTCACTGAA ACTGAAGAAA CGGTGGTTTG TACTCACCCA CAATTCCCTG | 4500 |
| GATTACTACA AGAGTTCAGA GAAGAACGCG CTCAAACTGG GGACCCTGGT CCTCAACAGC | 4560 |
| CTCTGCTCTG TCGTCCCCCC AGATGAGAAG ATATTCAAAG AGACAGGCTA CTGGAACGTC | 4620 |
| ACCGTGTACG GGCGCAAGCA CTGTTACCGG CTCTACACCA AGCTGCTCAA CGAGGCCACC | 4680 |
| CGGTGGTCCA GTGCCATTCA AAACGTGACT GACACCAAGG CCCCGATCGA CACCCCCACC | 4740 |
| CAGCAGCTGA TTCAAGATAT CAAGGAGAAC TGCCTGAACT CGGATGTGGT GGAACAGATT | 4800 |
| TACAAGCGGA ACCCGATCCT TCGATACACC CATGACCCCT TGCACTCCCC GCTCCTGCCC | 4860 |
| CTTCCGTATG GGGACATAAA TCTCAACTTG CTCAAAGACA AAGGCTATAC CACCCTTCAG | 4920 |
| GATGAGGCCA TCAAGATATT CAATTCCCTG CAGCAACTGG AGTCCATGTC TGACCCAATT | 4980 |
| CCAATAATCC AGGGCATCCT ACAGACAGGG CATGACCTGC GACCTCTGCG GGACGAGCTG | 5040 |
| TACTGCCAGC TTATCAAACA GACCAACAAA GTGCCCCACC CCGGCAGTGT GGGCAACCTG | 5100 |
| TACAGCTGGC AGATCCTGAC ATGCCTGAGC TGCACCTTCC TGCCGAGTCG AGGGATTCTC | 5160 |
| AAGTATCTCA AGTTCCATCT GAAAAGGATA CGGGAACAGT TTCCAGGAAC CGAGATGGAA | 5220 |
| AAATACGCTC TCTTCACTTA CGAATCTCTT AAGAAAACCA AATGCCGAGA GTTTGTGCCT | 5280 |
| TCCCGAGATG AAATAGAAGC TCTGATCCAC AGGCAGGAAA TGACATCCAC GGTCTATTGC | 5340 |
| CATGGCGGCG GCTCCTGCAA GATCACCATC AACTCCCACA CCACTGCTGG GGAGGTGGTG | 5400 |
| GAGAAGCTGA TCCGAGGCCT GGCCATGGAG GACAGCAGGA ACATGTTTGC TTTGTTTGAA | 5460 |
| TACAACGGCC ACGTCGACAA AGCCATTGAA AGTCGAACCG TCGTAGCTGA TGTCTTAGCC | 5520 |
| AAGTTTGAAA AGCTGGCTGC CACATCCGAG GTTGGGGACC TGCCATGGAA ATTCTACTTC | 5580 |
| AAACTTTACT GCTTCCTGGA CACAGACAAC GTGCCAAAAG ACAGTGTGGA GTTTGCATTT | 5640 |
| ATGTTTGAAC AGGCCCACGA AGCGGTTATC CATGGCCACC ATCCAGCCCC GGAAGAAAAC | 5700 |
| CTCCAGGTTC TTGCTGCCCT GCGACTCCAG TATCTGCAGG GGGATTATAC TCTGCACGCT | 5760 |
| GCCATCCCAC CTCTCGAAGA GGTTTATTCC CTGCAGAGAC TCAAGGCCCG CATCAGCCAG | 5820 |
| TCAACCAAAA CCTTCACCCC TTGTGAACGG CTGGAGAAGA GGCGGACGAG CTTCCTAGAG | 5880 |
| GGGACCCTGA GGCGGAGCTT CCGGACAGGA TCCGTGGTCC GGCAGAAGGT CGAGGAGGAG | 5940 |
| CAGATGCTGG ACATGTGGAT TAAGGAAGAA GTCTCCTCTG CTCGAGCCAG TATCATTGAC | 6000 |
| AAGTGGAGGA AATTTCAGGG AATGAACCAG GAACAGGCCA TGGCCAAGTA CATGGCCTTG | 6060 |
| ATCAAGGAGT GGCCTGGCTA TGGCTCGACG CTGTTTGATG TGGAGTGCAA GGAAGGTGGC | 6120 |
| TTCCCTCAGG AACTCTGGTT GGGTGTCAGC GCGGACGCCG TCTCCGTCTA CAAGCGTGGA | 6180 |
| GAGGGAAGAC AACTGGAAGT CTTCCAGTAT GAACACATCC TCTCTTTTGG GGCACCCCTG | 6240 |
| GCGAATACGT ATAAGATCGT GGTCGATGAG AGGGAGCTGC TCTTTGAAAC CAGTGAGGTG | 6300 |
| GTGGATGTGG CCAAGCTCAT GAAAGCCTAC ATCAGCATGA TCGTGAAGAA GCGCTACAGC | 6360 |
| ACGACACGCT CCGCCAGCAG CCAGGGCAGC TCCAGGTGAA GGCGGGACAG AGCCCACCTG | 6420 |
| TCTTTGCTAC CTGAACGCAC CACCCTCTGG CCTAGGCTGG CTCCAGTGTG CCATGCCCAG | 6480 |
| CCAAAACAAA CACAGAGCTG CCCAGGCTTT CTGGAAGCTT CTGGTCTGAG GGAGGTGTCT | 6540 |
| CCGAGGATCC TTTTGCCTGC CGCCTTCATT GATCCTGTAT TAAGCTGTCA ACTTTAACAG | 6600 |
| TCTGCACAGT TTCCAAAGCT TTACTACTCT TAGAGGACAC ATGCCTTAAA AAAGGAGGGG | 6660 |
| AGGAACCACG CTGCCACCAA AGCAGCCGGA AGTGCCTTAA CTTGTGGAAC CAACACTAAT | 6720 |
| CGACCGTAAC TGTGCTACTG AAGGGAACTG CCTTTCCCCC TTCTGGGGGA GACTTAACAG | 6780 |
| AGCGTGGAAG GGGGGCATTC TCTGTCAATG ATGCACTAAC CTCCCAACCT GATTTCCCCG | 6840 |
| AATCTGAGGG AAGGTGAGGG AGTGGGAAGG GGGATGGAGA GCTCGAGGGG ACAGTGTGTT | 6900 |
| TGAGCTGGAG TGCTGCGGGC AGCCTTTCTC ATGGAATGAC ATGAATCAAC TTTTTTCTTT | 6960 |
| GTTTCATCTT TTAAGTGTAC GTGCTTGCCT GTTCGTGCAT GTGTTCATAA ACTCAACACT | 7020 |
| TTAATCATGG TTTCATGAGC ATTAAAAAGC AAAGGGAAAA AGGATGTGTA ATGGTGTACA | 7080 |
| CAGTCTGTAT ATTTTAATAA TGCAGAGCTA TAGTCTCAAT TGTTACTTTA TAAGGTGGTT | 7140 |
| TTATTAACAA ACCCAAATCC TGGATTTTCC TGTCTTTGCT GTATTTTGAA AAACACGTGT | 7200 |
| TGACTCCATT GTTTTACATG TAGCAAAGTC TGCCATCTGT GTCTGCTGTA TTATAAACAG | 7260 |
| ATAAGCAGCC TACAAGATAA CTGTATTTAT AAACCACTCT TCAACAGCTG GCTCCAGTGC | 7320 |
| TGGTTTTAGA ACAAGAATGA AGTCATTTTG GAGTCTTTCA TGTCTAAAAG ATTTAAGTTA | 7380 |
| AAAACAAAGT GTTACTTGGA AGGTTAGCTT CTATCATTCT GGATAGATTA CAGATATAAT | 7440 |
| AACCATGTTG ACTATGGGGG AGAGACGCTG CATTCCAGAA ACGTCTTAAC ACTTGAGTGA | 7500 |
| ATCTTCAAAG GACCCTGACA TTAAATGCTG AGGCTTTAAT ACACACATAT TTTATCCCAA | 7560 |
| GTTTATAATG GTGGTCTGAA CAAGGCACCT GTAAATAAAT CAGCATTTAT GACCAGAAGA | 7620 |
| AAAATAATCT GGTCTTGGAC TTTTTATTTT TATATGGAAA AGTTTTAAGG ACTTGGGCCA | 7680 |
| ACTAAGTCTA CCCACACGAA AAAAGAAATT TGCCTTGTCC CTTTGTGTAC AACCATGCAA | 7740 |
| AACTGTTTGT TGGCTCACAG AAGTTCTGAC AATAAAAGAT ACTAGCT | |

ACC3 DNA sequence
Gene name: calcitonin receptor-like (CALCRL)
Unigene number: Hs.152175
Probeset Accession #: L76380
Nucleic Acid Accession #: NM_005795
Coding sequence: 555-1940 (predicted start/stop codons underlined)

| | |
|---|---|
| GCACGAGGGA ACAACCTCTC TCTCTSCAGC AGAGAGTGTC ACCTCCTGCT TTAGGACCAT | 60 |
| CAAGCTCTGC TAACTGAATC TCATCCTAAT TGCAGGATCA CATTGCAAAG CTTTCACTCT | 120 |
| TTCCCACCTT GCTTGTGGGT AAATCTCTTC TGCGGAATCT CAGAAAGTAA AGTTCCATCC | 180 |
| TGAGAATATT TCACAAAGAA TTTCCTTAAG AGCTGGACTG GGTCTTGACC CCTGGAATTT | 240 |
| AAGAAATTCT TAAAGACAAT GTCAAATATG ATCCAAGAGA AAATGTGATT TGAGTCTGGA | 300 |
| GACAATTGTG CATATCGTCT AATAATAAAA ACCCATACTA GCCTATAGAA AACAATATTT | 360 |
| GAATAATAAA AACCCATACT AGCCTATAGA AAACAATATT TGAAAGATTG CTACCACTAA | 420 |
| AAAGAAAACT ACTACAACTT GACAAGACTG CTGCAAACTT CAATTGGTCA CCACAACTTG | 480 |
| ACAAGGTTGC TATAAAACAA GATTGCTACA ACTTCTAGTT TATGTTATAC AGCATATTTC | 540 |
| ATTTGGGCTT AATGATGGAG AAAAAGTGTA CCCTGTATTT TCTGGTTCTC TTGCCTTTTT | 600 |
| TTATGATTCT TGTTACAGCA GAATTAGAAG AGAGTCCTGA GGACTCAATT CAGTTGGGAG | 660 |
| TTACTAGAAA TAAAATCATG ACAGCTCAAT ATGAATGTTA CCAAAAGATT ATGCAAGACC | 720 |
| CCATTCAACA AGCAGAAGGC GTTTACTGCA ACAGAACCTG GGATGGATGG CTCTGCTGGA | 780 |
| ACGATGTTGC AGCAGGAACT GAATCAATGC AGCTCTGCCC TGATTACTTT CAGGACTTTG | 840 |
| ATCCATCAGA AAAAGTTACA AAGATCTGTG ACCAAGATGG AAACTGGTTT AGACATCCAG | 900 |
| CAAGCAACAG AACATGGACA AATTATACCC AGTGTAATGT TAACACCCAC GAGAAAGTGA | 960 |
| AGACTGCACT AAATTTGTTT TACCTGACCA TAATTGGACA CGGATTGTCT ATTGCATCAC | 1020 |
| TGCTTATCTC GCTTGGCATA TTCTTTTATT TCAAGAGCCT AAGTTGCCAA AGGATTACCT | 1080 |
| TACACAAAAA TCTGTTCTTC TCATTTGTTT GTAACTCTGT TGTAACAATC ATTCACCTCA | 1140 |
| CTGCAGTGGC CAACAACCAG GCCTTAGTAG CCACAAATCC TGTTAGTTGC AAAGTGTCCC | 1200 |
| AGTTCATTCA TCTTTACCTG ATGGGCTGTA ATTACTTTTG GATGCTCTGT GAAGGCATTT | 1260 |
| ACCTACACAC ACTCATTGTG GTGGCCGTGT TTGCAGAGAA GCAACATTTA ATGTGGTATT | 1320 |
| ATTTTCTTGG CTGGGGATTT CCACTGATTC CTGCTTGTAT ACATGCCATT GCTAGAAGCT | 1380 |
| TATATTACAA TGACAATTGC TGGATCAGTT CTGATACCCA TCTCCTCTAC ATTATCCATG | 1440 |
| GCCCAATTTG TGCTGCTTTA CTGGTGAATC TTTTTTTCTT GTTAAATATT GTACGCGTTC | 1500 |
| TCATCACCAA GTTAAAAGTT ACACACCAAG CGGAATCCAA TCTGTACATG AAAGCTGTGA | 1560 |
| GAGCTACTCT TATCTTGGTG CCATTGCTTG GCATTGAATT TGTGCTGATT CCATGGCGAC | 1620 |
| CTGAAGGAAA GATTGCAGAG GAGGTATATG ACTACATCAT GCACATCCTT ATGCACTTCC | 1680 |
| AGGGTCTTTT GGTCTCTACC ATTTTCTGCT TCTTTAATGG AGAGGTTCAA GCAATTCTGA | 1740 |
| GAAGAAACTG GAATCAATAC AAAATCCAAT TTGGAAACAG CTTTTCCAAC TCAGAAGCTC | 1800 |
| TTCGTAGTGC GTCTTACACA GTGTCAACAA TCAGTGATGG TCCAGGTTAT AGTCATGACT | 1860 |
| GTCCTAGTGA ACACTTAAAT GGAAAAAGCA TCCATGATAT TGAAAATGTT CTCTTAAAAC | 1920 |
| CAGAAAATTT ATATAATTGA AAATAGAAGG ATGGTTGTCT CACTGTTTGG TGCTTCTCCT | 1980 |
| AACTCAAGGA CTTGGACCCA TGACTCTGTA GCCAGAAGAC TTCAATATTA AATGACTTTG | 2040 |
| GGGAATGTCA TAAAGAAGAG CCTTCACATG AAATTAGTAG TGTGTTGATA AGAGTGTAAC | 2100 |
| ATCCAGCTCT ATGTGGGAAA AAAGAAATCC TGGTTTGTAA TGTTTGTCAG TAAATACTCC | 2160 |
| CACTATGCCT GATGTGACGC TACTAACCTG ACATCACCAA GTGTGGAATT GGAGAAAAGC | 2220 |
| ACAATCAACT TTTCTGAGCT GGTGTAAGCC AGTTCCAGCA CACCATTGAT GAATTCAAAC | 2280 |
| AAATGGCTGT AAAACTAAAC ATACATGTTG GGCATGATTC TACCCTTATT CSCCCCAAGA | 2340 |
| GACCTAGCTA AGGTCTATAA ACATGAAGGG AAAATTAGCT TTTAGTTTTA AAACTCTTTA | 2400 |
| TCCCATCTTG ATTGGGGCAG TTGACTTTTT TTTTTTCCCA GAGTGCCGTA GTCCTTTTTG | 2460 |
| TAACTACCCT CTCAAATGGA CAATACCAGA AGTGAATTAT CCCTGCTGGC TTTCTTTTCT | 2520 |
| CTATGAAAAG CAACTGAGTA CAATTGTTAT GATCTACTCA TTTGCTGACA CATCAGTTAT | 2580 |
| ATCTTGTGGC ATATCCATTG TGGAAACTGG ATGAACAGGA TGTATAATAT GCAATCTTAC | 2640 |
| TTCTATATCA TTAGGAAAAC ATCTTAGTTG ATGCTACAAA ACACCTTGTC AACCTCTTCC | 2700 |
| TGTCTTACCA AACAGTGGGA GGGAATTCCT AGCTGTAAAT ATAAATTTTG CCCTTCCATT | 2760 |
| TCTACTGTAT AAACAAATTA GCAATCATTT TATATAAAGA AAATCAATGA AGGATTTCTT | 2820 |
| ATTTTCTTGG AATTTTGTAA AAAGAAATTG TGAAAAATGA GCTTGTAAAT ACTCCATTAT | 2880 |
| TTTATTTTAT AGTCTCAAAT CAAATACATA CAACCTATGT AATTTTTAAA GCAAATATAT | 2940 |
| AATGCAACAA TGTGTGTATG TTAATATCTG ATACTGTATC TGGGCTGATT TTTTAAATAA | 3000 |
| AATAGAGTCT GGAATGCT | |

ACC4 DNA sequence
Gene name: Homo sapiens mRNA; cDNA DKFZp586E1624
Unigene number: Hs.94030
Probeset Accession #: AA452000
Nucleic Acid Accession #: AL110152.1
Coding sequence: no ORF identified, possible frameshifts

| | |
|---|---|
| ACGCGTCCGA AGACATTAAG TAAAAAATTG GAACTATGAT TTTTCTTTGT CATTTTTTAA | 60 |
| AAAAGAATTA TTTTATTAAC CTGCTGGCAT ATAATCTGGA GTTCTTTTCA CAACCTTACT | 120 |
| TTTTCTGATT TGCTTTATTG AATGATTGAA TACTCATTTC TTTCTAAAAA TATGTTGTAA | 180 |
| ATTCTCCCTT GGCAAGATTT CTCCCTATGA GGGTAGTTAT TATTTGAGTC TGCCAAGTGG | 240 |
| TTACCATGGG GCAAGGTGCC ATGATGTATT CTTGGGTGCA TTGGTTTTTT GCGCATTGTA | 300 |
| AATTTAAGAC ACTTATAGTA AGTGGACTCA TTCATAGATG AGTTTCAGAA CCTTTTACGT | 360 |
| TCTCGGTAGA GGCTTCTGTC GGACAGGCAG AAGAGTGTAT TCCTCACTTT TTTTTTTGTC | 420 |
| TTCAAATTCC AGTAAGGCAT GCCACTTTTA AGAAATTAGA ATTTTTCTAT CATCTATGCA | 480 |
| AATGATATTT ATGTTAATAT TAAATATCTT ATGTTACACT GGGAGTAATT TGAGGTGCAA | 540 |
| TTATTTTTAT TACTACTTTG AATAGAGGAC CATTATCCTT CTTTCTTCAG AAAACTAAGA | 600 |
| AGTAAGTGTA ACTTTTAAAG TAAGTATATA TCAGTGAGAG TAGGCTTGTT TTACAACTAT | 660 |
| TTCTAGCCAG TGAGTTGTGT TTTCATGTCT CATCAAAAGA CAATACCACA TTGCATCATT | 720 |
| TTACAAAATA TGTTGTCATT TTCATTTCAG TTGTAACATA GGAAAATAGA TATTTCCTAG | 780 |
| ATGATTTCTG AGTTTCTTAC TGCAAAGAAC AGTTATAAAT TGGTATACAT GTGTCTCTGT | 840 |
| AATAGGGATA ATATTGATAT ATCTGTTGCT ACATATTTAA GAATCATTCT ATCTTATGTT | 900 |
| GTCTTGAGGC CAAGATTTAC CACGTTTGCC CAGTGTATTG AATTGGTGGT AGAAGGTAGT | 960 |
| TCCATGTTCC ATTTGTAGAT CTTTAAGATT TTATCTTTGA TAACTTTAAT AGAATGTGGC | 1020 |
| TCAGTTCTGG TCCTTCAAGC CTGTATGGTT TGGATTTTCA GTAGGGGACA GTTGATGTGG | 1080 |
| AGTCAATCTC TTTGGTACAC AGGAAGCTTT ATAAAATTTC ATTCACGAAT CTCTTATTTT | 1140 |
| GGGAAGCTGT TTTGCATATG AGAAGAACAC TGTTGAAATA AGGAACTAAA GCTTTATATA | 1200 |
| TTGATCAAGG TGATTCTGAA AGTTTTAATT TTTAATGTTG TAATGTTATG TTATTGTTAA | 1260 |
| TTGTACTTTA TTATGTATTC AATAGAAAAT CATGATTTAT TAATAAAAGC TTAAATTCTC | 1320 |
| ATCTAAAAAA AAAAAAAAAA A | |

ACC5 DNA sequence
Gene name: Selectin E (endothelial adhesion molecule 1)
Unigene number: Hs.89546
Probeset Accession #: M24736
Nucleic Acid Accession #: NM_000450
Coding sequence: 117-1949 (predicted start/stop codons underlined)

| | |
|---|---|
| CCTGAGACAG AGGCAGCAGT GATACCCACC TGAGAGATCC TGTGTTTGAA CAACTGCTTC | 60 |
| CCAAAACGGA AAGTATTTCA AGCCTAAACC TTTGGGTGAA AAGAACTCTT GAAGTCATGA | 120 |
| TTGCTTCACA GTTTCTCTCA GCTCTCACTT TGGTGCTTCT CATTAAAGAG AGTGGAGCCT | 180 |
| GGTCTTACAA CACCTCCACG GAAGCTATGA CTTATGATGA GGCCAGTGCT TATTGTCAGC | 240 |
| AAAGGTACAC ACACCTGGTT GCAATTCAAA ACAAAGAAGA GATTGAGTAC CTAAACTCCA | 300 |
| TATTGAGCTA TTCACCAAGT TATTACTGGA TTGGAATCAG AAAAGTCAAC AATGTGTGGG | 360 |
| TCTGGGTAGG AACCCAGAAA CCTCTGACAG AAGAAGCCAA GAACTGGGCT CCAGGTGAAC | 420 |
| CCAACAATAG GCAAAAAGAT GAGGACTGCG TGGAGATCTA CATCAAGAGA GAAAAAGATG | 480 |
| TGGGCATGTG GAATGATGAG AGGTGCAGCA AGAAGAAGCT TGCCCTATGC TACACAGCTG | 540 |
| CCTGTACCAA TACATCCTGC AGTGGCCACG GTGAATGTGT AGAGACCATC AATAATTACA | 600 |
| CTTGCAAGTG TGACCCTGGC TTCAGTGGAC TCAAGTGTGA GCAAATTGTG AACTGTACAG | 660 |
| CCCTGGAATC CCCTGAGCAT GGAAGCCTGG TTTGCAGTCA CCCACTGGGA AACTTCAGCT | 720 |
| ACAATTCTTC CTGCTCTATC AGCTGTGATA GGGGTTACCT GCCAAGCAGC ATGGAGACCA | 780 |
| TGCAGTGTAT GTCCTCTGGA GAATGGAGTG CTCCTATTCC AGCCTGCAAT GTGGTTGAGT | 840 |
| GTGATGCTGT GACAAATCCA GCCAATGGGT TCGTGGAATG TTTCCAAAAC CCTGGAAGCT | 900 |
| TCCCATGGAA CACAACCTGT ACATTTGACT GTGAAGAAGG ATTTGAACTA ATGGGAGCCC | 960 |
| AGAGCCTTCA GTGTACCTCA TCTGGGAATT GGGACAACGA GAAGCCAACG TGTAAAGCTG | 1020 |
| TGACATGCAG GGCCGTCCGC CAGCCTCAGA ATGGCTCTGT GAGGTGCAGC CATTCCCCTG | 1080 |
| CTGGAGAGTT CACCTTCAAA TCATCCTGCA ACTTCACCTG TGAGGAAGGC TTCATGTTGC | 1140 |
| AGGGACCAGC CCAGGTTGAA TGCACCACTC AAGGGCAGTG GACACAGCAA ATCCCAGTTT | 1200 |
| GTGAAGCTTT CCAGTGCACA GCCTTGTCCA ACCCCGAGCG AGGCTACATG AATTGTCTTC | 1260 |
| CTAGTGCTTC TGGCAGTTTC CGTTATGGGT CCAGCTGTGA GTTCTCCTGT GAGCAGGGTT | 1320 |
| TTGTGTTGAA GGGATCCAAA AGGCTCCAAT GTGGCCCCAC AGGGGAGTGG GACAACGAGA | 1380 |
| AGCCCACATG TGAAGCTGTG AGATGCGATG CTGTCCACCA GCCCCCGAAG GGTTTGGTGA | 1440 |
| GGTGTGCTCA TTCCCCTATT GGAGAATTCA CCTACAAGTC CTCTTGTGCC TTCAGCTGTG | 1500 |
| AGGAGGGATT TGAATTATAT GGATCAACTC AACTTGAGTG CACATCTCAG GGACAATGGA | 1560 |
| CAGAAGAGGT TCCTTCCTGC CAAGTGGTAA AATGTTCAAG CCTGGCAGTT CCGGGAAAGA | 1620 |
| TCAACATGAG CTGCAGTGGG GAGCCCGTGT TTGGCACTGT GTGCAAGTTC GCCTGTCCTG | 1680 |
| AAGGATGGAC GCTCAATGGC TCTGCAGCTC GGACATGTGG AGCCACAGGA CACTGGTCTG | 1740 |
| GCCTGCTACC TACCTGTGAA GCTCCCACTG AGTCCAACAT TCCCTTGGTA GCTGGACTTT | 1800 |
| CTGCTGCTGG ACTCTCCCTC CTGACATTAG CACCATTTCT CCTCTGGCTT CGGAAATGCT | 1860 |
| TACGGAAAGC AAAGAAATTT GTTCCTGCCA GCAGCTGCCA AAGCCTTGAA TCAGACGGAA | 1920 |
| GCTACCAAAA GCCTTCTTAC ATCCTTTAAG TTCAAAAGAA TCAGAAACAG GTGCATCTGG | 1980 |
| GGAACTAGAG GGATACACTG AAGTTAACAG AGACAGATAA CTCTCCTCGG GTCTCTGGCC | 2040 |
| CTTCTTGCCT ACTATGCCAG ATGCCTTTAT GGCTGAAACC GCAACACCCA TCACCACTTC | 2100 |
| AATAGATCAA AGTCCAGCAG GCAAGGACGG CCTTCAACTG AAAAGACTCA GTGTTCCCTT | 2160 |
| TCCTACTCTC AGGATCAAGA AAGTGTTGGC TAATGAAGGG AAAGGATATT TTCTTCCAAG | 2220 |
| CAAAGGTGAA GAGACCAAGA CTCTGAAATC TCAGAATTCC TTTTCTAACT CTCCCTTGCT | 2280 |
| CGCTGTAAAA TCTTGGCACA GAAACACAAT ATTTTGTGGC TTTCTTTCTT TTGCCCTTCA | 2340 |
| CAGTGTTTCG ACAGCTGATT ACACAGTTGC TGTCATAAGA ATGAATAATA ATTATCCAGA | 2400 |
| GTTTAGAGGA AAAAAATGAC TAAAAATATT ATAACTTAAA AAAATGACAG ATGTTGAATG | 2460 |
| CCCACAGGCA AATGCATGGA GGGTTGTTAA TGGTGCAAAT CCTACTGAAT GCTCTGTGCG | 2520 |
| AGGGTTACTA TGCACAATTT AATCACTTTC ATCCCTATGG GATTCAGTGC TTCTTAAAGA | 2580 |
| GTTCTTAAGG ATTGTGATAT TTTTACTTGC ATTGAATATA TTATAATCTT CCATACTTCT | 2640 |
| TCATTCAATA CAAGTGTGGT AGGGACTTAA AAAACTTGTA AATGCTGTCA ACTATGATAT | 2700 |
| GGTAAAAGTT ACTTATTCTA GATTACCCCC TCATTGTTTA TTAACAAATT ATGTTACATC | 2760 |
| TGTTTTAAAT TTATTTCAAA AAGGGAAACT ATTGTCCCCT AGCAAGGCAT GATGTTAACC | 2820 |
| AGAATAAAGT TCTGAGTGTT TTTACTACAG TTGTTTTTTG AAAACATGGT AGAATTGGAG | 2880 |
| AGTAAAAACT GAATGGAAGG TTTGTATATT GTCAGATATT TTTTCAGAAA TATGTGGTTT | 2940 |
| CCACGATGAA AAACTTCCAT GAGGCCAAAC GTTTTGAACT AATAAAAGCA TAAATGCAAA | 3000 |
| CACACAAAGG TATAATTTTA TGAATGTCTT TGTTGGAAAA GAATACAGAA AGATGGATGT | 3060 |
| GCTTTGCATT CCTACAAAGA TGTTTGTCAG ATGTGATATG TAAACATAAT TCTTGTATAT | 3120 |
| TATGGAAGAT TTTAAATTCA CAATAGAAAC TCACCATGTA AAAGAGTCAT CTGGTAGATT | 3180 |
| TTTAACGAAT GAAGATGTCT AATAGTTATT CCCTATTTGT TTTCTTCTGT ATGTTAGGGT | 3240 |
| GCTCTGGAAG AGAGGAATGC CTGTGTGAGC AAGCATTTAT GTTTATTTAT AAGCAGATTT | 3300 |
| AACAATTCCA AAGGAATCTC CAGTTTTCAG TTGATCACTG GCAATGAAAA ATTCTCAGTC | 3360 |
| AGTAATTGCC AAAGCTGCTC TAGCCTTGAG GAGTGTGAGA ATCAAAACTC TCCTACACTT | 3420 |
| CCATTAACTT AGCATGTGTT GAAAAAAAAA GTTTCAGAGA AGTTCTGGCT GAACACTGGC | 3480 |
| AACGACAAAG CCAACAGTCA AAACAGAGAT GTGATAAGGA TCAGAACAGC AGAGGTTCTT | 3540 |
| TTAAAGGGGC AGAAAAACTC TGGGAAATAA GAGAGAACAA CTACTGTGAT CAGGCTATGT | 3600 |
| ATGGAATACA GTGTTATTTT CTTTGAAATT GTTTAAGTGT TGTAAATATT TATGTAAACT | 3660 |
| GCATTAGAAA TTAGCTGTGT GAAATACCAG TGTGGTTTGT GTTTGAGTTT TATTGAGAAT | 3720 |
| TTTAAATTAT AACTTAAAAT ATTTTATAAT TTTTAAAGTA TATATTTATT TAAGCTTATG | 3780 |
| TCAGACCTAT TTGACATAAC ACTATAAAGG TTGACAATAA ATGTGCTTAT GTTT | |

ACC8 DNA sequence
Gene name: Chemokine (C-X-C motif), receptor 4 (fusin)
Unigene number: Hs.89414
Probeset Accession #: L06797
Nucleic Acid Accession #: NM_003467
Coding sequence: 89-1147 (predicted start/stop codons underlined)

| | |
|---|---|
| GTTTGTTGGC TGCGGCAGCA GGTAGCAAAG TGACGCCGAG GGCCTGAGTG CTCCAGTAGC | 60 |
| CACCGCATCT GGAGAACCAG CGGTTACCAT GGAGGGGATC AGTATATACA CTTCAGATAA | 120 |
| CTACACCGAG GAAATGGGCT CAGGGGACTA TGACTCCATG AAGGAACCCT GTTTCCGTGA | 180 |
| AGAAAATGCT AATTTCAATA AAATCTTCCT GCCCACCATC TACTCCATCA TCTTCTTAAC | 240 |
| TGGCATTGTG GGCAATGGAT TGGTCATCCT GGTCATGGGT TACCAGAAGA AACTGAGAAG | 300 |
| CATGACGGAC AAGTACAGGC TGCACCTGTC AGTGGCCGAC CTCCTCTTTG TCATCACGCT | 360 |
| TCCCTTCTGG GCAGTTGATG CCGTGGCAAA CTGGTACTTT GGGAACTTCC TATGCAAGGC | 420 |
| AGTCCATGTC ATCTACACAG TCAACCTCTA CAGCAGTGTC CTCATCCTGG CCTTCATCAG | 480 |
| TCTGGACCGC TACCTGGCCA TCGTCCACGC CACCAACAGT CAGAGGCCAA GGAAGCTGTT | 540 |
| GGCTGAAAAG GTGGTCTATG TTGGCGTCTG GATCCCTGCC CTCCTGCTGA CTATTCCCGA | 600 |
| CTTCATCTTT GCCAACGTCA GTGAGGCAGA TGACAGATAT ATCTGTGACC GCTTCTACCC | 660 |
| CAATGACTTG TGGGTGGTTG TGTTCCAGTT TCAGCACATC ATGGTTGGCC TTATCCTGCC | 720 |
| TGGTATTGTC ATCCTGTCCT GCTATTGCAT TATCATCTCC AAGCTGTCAC ACTCCAAGGG | 780 |
| CCACCAGAAG CGCAAGGCCC TCAAGACCAC AGTCATCCTC ATCCTGGCTT TCTTCGCCTG | 840 |
| TTGGCTGCCT TACTACATTG GGATCAGCAT CGACTCCTTC ATCCTCCTGG AAATCATCAA | 900 |
| GCAAGGGTGT GAGTTTGAGA ACACTGTGCA CAAGTGGATT TCCATCACCG AGGCCCTAGC | 960 |
| TTTCTTCCAC TGTTGTCTGA ACCCCATCCT CTATGCTTTC CTTGGAGCCA AATTTAAAAC | 1020 |
| CTCTGCCCAG CACGCACTCA CCTCTGTGAG CAGAGGGTCC AGCCTCAAGA TCCTCTCCAA | 1080 |
| AGGAAAGCGA GGTGGACATT CATCTGTTTC CACTGAGTCT GAGTCTTCAA GTTTTCACTC | 1140 |
| CAGCTAACAC AGATGTAAAA GACTTTTTTT TATACGATAA ATAACTTTTT TTTAAGTTAC | 1200 |
| ACATTTTTCA GATATAAAAG ACTGACCAAT ATTGTACAGT TTTTATTGCT TGTTGGATTT | 1260 |
| TTGTCTTGTG TTTCTTTAGT TTTTGTGAAG TTTAATTGAC TTATTTATAT AAATTTTTTT | 1320 |
| TGTTTCATAT TGATGTGTGT CTAGGCAGGA CCTGTGGCCA AGTTCTTAGT TGCTGTATGT | 1380 |
| CTCGTGGTAG GACTGTAGAA AAGGGAACTG AACATTCCAG AGCGTGTAGT GAATCACGTA | 1440 |
| AAGCTAGAAA TGATCCCCAG CTGTTTATGC ATAGATAATC TCTCCATTCC CGTGGAACGT | 1500 |
| TTTTCCTGTT CTTAAGACGT GATTTTGCTG TAGAAGATGG CACTTATAAC CAAAGCCCAA | 1560 |
| AGTGGTATAG AAATGCTGGT TTTTCAGTTT TCAGGAGTGG GTTGATTTCA GCACCTACAG | 1620 |
| TGTACAGTCT TGTATTAAGT TGTTAATAAA AGTACATGTT AAACTTACTT AGTGTTATG | |

ACF2 DNA sequence
Gene name: Endothelial cell-specific molecule 1
Unigene number: Hs.41716
Probeset Accession #: X89426
Nucleic Acid Accession #: NM_007036
Coding sequence: 56-610 (predicted start/stop codons underlined)

| | |
|---|---|
| CTTCCCACCA GCAAAGACCA CGACTGGAGA GCCGAGCCGG AGGCAGCTGG GAAACATGAA | 60 |
| GAGCGTCTTG CTGCTGACCA CGCTCCTCGT GCCTGCACAC CTGGTGGCCG CCTGGAGCAA | 120 |
| TAATTATGCG GTGGACTGCC CTCAACACTG TGACAGCAGT GAGTGCAAAA GCAGCCCGCG | 180 |
| CTGCAAGAGG ACAGTGCTCG ACGACTGTGG CTGCTGCCGA GTGTGCGCTG CAGGGCGGGG | 240 |
| AGAAACTTGC TACCGCACAG TCTCAGGCAT GGATGGCATG AAGTGTGGCC CGGGGCTGAG | 300 |
| GTGTCAGCCT TCTAATGGGG AGGATCCTTT TGGTGAAGAG TTTGGTATCT GCAAAGACTG | 360 |
| TCCCTACGGC ACCTTCGGGA TGGATTGCAG AGAGACCTGC AACTGCCAGT CAGGCATCTG | 420 |
| TGACAGGGGG ACGGGAAAAT GCCTGAAATT CCCCTTCTTC CAATATTCAG TAACCAAGTC | 480 |
| TTCCAACAGA TTTGTTTCTC TCACGGAGCA TGACATGGCA TCTGGAGATG GCAATATTGT | 540 |
| GAGAGAAGAA GTTGTGAAAG AGAATGCTGC CGGGTCTCCC GTAATGAGGA AATGGTTAAA | 600 |
| TCCACGCTGA TCCCGGCTGT GATTTCTGAG AGAAGGCTCT ATTTTCGTGA TTGTTCAACA | 660 |
| CACAGCCAAC ATTTTAGGAA CTTTCTAGAT ATAGCATAAG TACATGTAAT TTTTGAAGAT | 720 |
| CCAAATTGTG ATGCATGGTG GATCCAGAAA ACAAAAAGTA GGATACTTAC AATCCATAAC | 780 |
| ATCCATATGA CTGAACACTT GTATGTGTTT GTTAAATATT CGAATGCATG TAGATTTGTT | 840 |
| AAATGTGTGT GTATAGTAAC ACTGAAGAAC TAAAAATGCA ATTTAGGTAA TCTTACATGG | 900 |
| AGACAGGTCA ACCAAAGAGG GAGCTAGGCA AAGCTGAAGA CCGCAGTGAG TCAAATTAGT | 960 |
| TCTTTGACTT TGATGTACAT TAATGTTGGG ATATGGAATG AAGACTTAAG AGCAGGAGAA | 1020 |
| GATGGGGAGG GGGTGGGAGT GGGAAATAAA ATATTTAGCC CTTCCTTGGT AGGTAGCTTC | 1080 |
| TCTAGAATTT AATTGTGCTT TTTTTTTTTT TTTGGCTTTG GGAAAAGTCA AAATAAAACA | 1140 |
| ACCAGAAAAC CCCTGAAGGA AGTAAGATGT TTGAAGCTTA TGGAAATTTG AGTAACAAAC | 1200 |
| AGCTTTGAAC TGAGAGCAAT TTCAAAAGGC TGCTGATGTA GTTCCCGGGT TACCTGTATC | 1260 |
| TGAAGGACGG TTCTGGGGCA TAGGAAACAC ATACACTTCC ATAAATAGCT TTAACGTATG | 1320 |
| CCACCTCAGA GATAAATCTA AGAAGTATTT TACCCACTGG TGGTTTGTGT GTGTATGAAG | 1380 |
| GTAAATATTT ATATATTTTT ATAAATAAAT GTGTTAGTGC AAGTCATCTT CCCTACCCAT | 1440 |
| ATTTATCATC CTCTTGAGGA AAGAAATCTA GTATTATTTG TTGAAAATGG TTAGAATAAA | 1500 |
| AACCTATGAC TCTATAAGGT TTTCAAACAT CTGAGGCATG ATAAATTTAT TATCCATAAT | 1560 |
| TATAGGAGTC ACTCTGGATT TCAAAAAATG TCAAAAAATG AGCAACAGAG GGACCTTATT | 1620 |
| TAAACATAAG TGCTGTGACT TCGGTGAATT TTCAATTTAA GGTATGAAAA TAAGTTTTTA | 1680 |
| GGAGGTTTGT AAAAGAAGAA TCAATTTTCA GCAGAAAACA TGTCAACTTT AAAATATAGG | 1740 |
| TGGAATTAGG AGTATATTTG AAAGAATCTT AGCACAAACA GGACTGTTGT ACTAGATGTT | 1800 |
| CTTAGGAAAT ATCTCAGAAG TATTTTATTT GAAGTGAAGA ACTTATTTAA GAATTATTTC | 1860 |
| AGTATTTACC TGTATTTTAT TCTTGAAGTT GGCCAACAGA GTTGTGAATG TGTGTGGAAG | 1920 |
| GCCTTTGAAT GTAAAGCTGC ATAAGCTGTT AGGTTTTGTT TTAAAAGGAC ATGTTTATTA | 1980 |
| TTGTTCAATA AAAAAGAACA AGATAC | |

ACF4 DNA sequence
Gene name: P53-responsive gene 2 similar to D.melanogaster peroxidasin (U11052)
Unigene number: Hs.118893
Probeset Accession #: D86983
Nucleic Acid Accession #: D86983
Coding sequence: 1-4491 (predicted stop codon underlined, sequence is open at 5′
end)

| | |
|---|---|
| AGCCGGCCGT GGTGGCTCCG TGCGTCCGAG CGTCCGTCCG CGCCGTCGGC CATGGCCAAG | 60 |
| CGCTCCAGGG GCCCCGGGCG CCGCTGCCTG TTGGCGCTCG TGCTGTTCTG CGCCTGGGGG | 120 |
| ACGCTGGCCG TGGTGGCCCA GAAGCCGGGC GCAGGGTGTC CGAGCCGCTG CCTGTGCTTC | 180 |
| CGCACCACCG TGCGCTGCAT GCATCTGCTG CTGGAGGCCG TGCCCGCCGT GGCGCCGCAG | 240 |
| ACCTCCATCC TAGATCTTCG CTTTAACAGA ATCAGAGAGA TCCAACCTGG GGCATTCAGG | 300 |
| CGGCTGAGGA ACTTGAACAC ATTGCTTCTC AATAATAATC AGATCAAGAG GATACCTAGT | 360 |
| GGAGCATTTG AAGACTTGGA AAATTTAAAA TATCTCTATC TGTACAAGAA TGAGATCCAG | 420 |
| TCAATTGACA GGCAAGCATT TAAGGGACTT GCCTCTCTAG AGCAACTATA CCTGCACTTT | 480 |
| AATCAGATAG AAACTTTGGA CCCAGATTCG TTCCAGCATC TCCCGAAGCT CGAGAGGCTA | 540 |
| TTTTTGCATA ACAACCGGAT TACACATTTA GTTCCAGGGA CATTTAATCA CTTGGAATCT | 600 |
| ATGAAGAGAT TGCGACTGGA CTCAAACACA CTTCACTGCG ACTGTGAAAT CCTGTGGTTG | 660 |
| GCGGATTTGC TGAAAACCTA CGCGGAGTCG GGGAACGCGC AGGCAGCGGC CATCTGTGAA | 720 |
| TATCCCAGAC GCATCCAGGG ACGCTCAGTG GCAACCATCA CCCCGGAAGA GCTGAACTGT | 780 |
| GAAAGGCCCC GGATCACCTC CGAGCCCCAG GACGCAGATG TGACCTCGGG GAACACCGTG | 840 |
| TACTTCACCT GCAGAGCCGA AGGCAACCCC AAGCCTGAGA TCATCTGGCT GCGAAACAAT | 900 |
| AATGAGCTGA GCATGAAGAC AGATTCCCGC CTAAACTTGC TGGACGATGG GACCCTGATG | 960 |
| ATCCAGAACA CACAGGAGAC AGACCAGGGT ATCTACCAGT GCATGGCAAA GAACGTGGCC | 1020 |
| GGAGAGGTGA AGACGCAAGA GGTGACCCTC AGGTACTTCG GGTCTCCAGC TCGACCCACT | 1080 |
| TTTGTAATCC AGCCACAGAA TACAGAGGTG CTGGTTGGGG AGAGCGTCAC GCTGGAGTGC | 1140 |
| AGCGCCACAG GCCACCCCCC GCCGCGGATC TCCTGGACGA GAGGTGACCG CACACCCTTG | 1200 |
| CCAGTTGACC CGCGGGTGAA CATCACGCCT TCTGGCGGGC TTTACATACA GAACGTCGTA | 1260 |
| CAGGGGGACA GCGGAGAGTA TGCGTGCTCT GCGACCAACA ACATTGACAG CGTCCATGCC | 1320 |
| ACCGCTTTCA TCATCGTCCA GGCTCTTCCT CAGTTCACTG TGACGCCTCA GGACAGAGTC | 1380 |
| GTTATTGAGG GCCAGACCGT GGATTTCCAG TGTGAAGCCA AGGGCAACCC GCCGCCCGTC | 1440 |
| ATCGCCTCCA CCAAGGGAGG GAGCCAGCTC TCCGTGGACC GGCGGCACCT GGTCCTGTCA | 1500 |
| TCGGGAACC TTAGAATCTC TGGTGTTGCC CTCCACGACC AGGGCCAGTA CGAATGCCAG | 1560 |
| GCTGTCAACA TCATCGGCTC CCAGAAGGTC GTGGCCCACC TGACTGTGCA GCCCAGAGTC | 1620 |
| ACCCCAGTGT TTGCCAGCAT TCCCAGCGAC ACAACAGTGG AGGTGGGCGC CAATGTGCAG | 1680 |
| CTCCCGTGCA GCTCCCAGGG CGAGCCCGAG CCAGCCATCA CCTGGAACAA GGATGGGGTT | 1740 |
| CAGGTGACAG AAAGTGGAAA ATTTCACATC AGCCCTGAAG GATTCTTGAC CATCAATGAC | 1800 |
| GTTGGCCCTG CAGACGCAGG TCGCTATGAG TGTGTGGCCC GGAACACCAT TGGGTCGGCC | 1860 |
| TCGGTGAGCA TGGTGCTCAG TGTGAACGTT CCTGACGTCA GTCGAAATGG AGATCCGTTT | 1920 |
| GTAGCTACCT CCATCGTGGA AGCGATTGCG ACTGTTGACA GAGCTATAAA CTCAACCCGA | 1980 |
| ACACATTTGT TTGACAGCCG TCCTCGTTCT CCAAATGATT TGCTGGCCTT GTTCCGGTAT | 2040 |
| CCGAGGGATC CTTACACAGT TGAACAGGCA CGGGCGGGAG AAATCTTTGA ACGGACATTG | 2100 |
| CAGCTCATTC AGGAGCATGT ACAGCATGGC TTGATGGTCG ACCTCAACGG AACAAGTTAC | 2160 |
| CACTACAACG ACCTGGTGTC TCCACAGTAC CTGAACCTCA TCGCAAACCT GTCGGGCTGT | 2220 |
| ACCGCCCACC GGCGCGTGAA CAACTGCTCG GACATGTGCT TCCACCAGAA GTACCGGACG | 2280 |
| CACGACGGCA CCTGTAACAA CCTGCAGCAC CCCATGTGGG GCGCCTCGCT GACCGCCTTC | 2340 |
| GAGCGCCTGC TGAAATCCGT GTACGAGAAT GGCTTCAACA CCCCTCGGGG CATCAACCCC | 2400 |
| CACCGACTGT ACAACGGGCA CGCCCTTCCC ATGCCGCGCC TGGTGTCCAC CACCCTGATC | 2460 |
| GGGACGGAGA CCGTCACACC CGACGAGCAG TTCACCCACA TGCTGATGCA GTGGGGCCAG | 2520 |
| TTCCTGGACC ACGACCTCGA CTCCACGGTG GTGGCCCTGA GCCAGGCACG CTTCTCCGAC | 2580 |
| GGACAGCACT GCAGCAACGT GTGCAGCAAC GACCCCCCCT GCTTCTCTGT CATGATCCCC | 2640 |
| CCCAATGACT CCCGGGCCAG GAGCGGGGCC CGCTGCATGT TCTTCGTGCG CTCCAGCCCT | 2700 |
| GTGTGCGGCA GCGGCATGAC TTCGCTGCTC ATGAACTCCG TGTACCCGCG GGAGCAGATC | 2760 |
| AACCAGCTCA CCTCCTACAT CGACGCATCC AACGTGTACG GGAGCACGGA GCATGAGGCC | 2820 |
| CGCAGCATCC GCGACCTGGC CAGCCACCGC GGCCTGCTGC GGCAGGGCAT CGTGCAGCGG | 2880 |
| TCCGGGAAGC CGCTGCTCCC CTTCGCCACC GGGCCGCCCA CGGAGTGCAT GCGGGACGAG | 2940 |
| AACGAGAGCC CCATCCCCTG CTTCCTGGCC GGGGACCACC GCGCCAACGA GCAGCTGGGC | 3000 |
| CTGACCAGCA TGCACACGCT GTGGTTCCGC GAGCACAACC GCATTGCCAC GGAGCTGCTC | 3060 |
| AAGCTGAACC CGCACTGGGA CGGCGACACC ATCTACTATG AGACCAGGAA GATCGTGGGT | 3120 |
| GCGGAGATCC AGCACATCAC CTACCAGCAC TGGCTCCCGA AGATCCTGGG GGAGGTGGGC | 3180 |
| ATGAGGACGC TGGGAGAGTA CCACGGCTAC GACCCCGGCA TCAATGCTGG CATCTTCAAC | 3240 |
| GCCTTCGCCA CCGCGGCCTT CAGGTTTGGC CACACGCTTG TCAACCCACT GCTTTACCGG | 3300 |
| CTGGACGAGA ACTTCCAGCC CATTGCACAA GATCACCTCC CCCTTCACAA AGCTTTCTTC | 3360 |
| TCTCCCTTCC GGATTGTGAA TGAGGGCGGC ATCGATCCGC TTCTCAGGGG GCTGTTCGGG | 3420 |
| GTGGCGGGGA AAATGCGTGT GCCCTCGCAG CTGCTGAACA CGGAGCTCAC GGAGCGGCTG | 3480 |
| TTCTCCATGG CACACACGGT GGCTCTGGAC CTGGCGGCCA TCAACATCCA GCGGGGCCGG | 3540 |
| GACCAGGGGA TCCCACCCTA CCACGACTAC AGGGTCTACT GCAATCTATC GGCGGCACAC | 3600 |
| ACGTTCGAGG ACCTGAAAAA TGAGATTAAA AACCCTGAGA TCCGGGAGAA ACTGAAAAGG | 3660 |
| TTGTATGGCT CGACACTCAA CATCGACCTG TTTCCGGCGC TCGTGGTGGA GGACCTGGTG | 3720 |
| CCTGGCAGCC GGCTGGGCCC CACCCTGATG TGTCTTCTCA GCACACAGTT CAAGCGCCTG | 3780 |
| CGAGATGGGG ACAGGTTGTG GTATGAGAAC CCTGGGGTGT TCTCCCCGGC CCAGCTGACT | 3840 |
| CAGATCAAGC AGACGTCGCT GGCCAGGATC CTATGCGACA ACGCGGACAA CATCACCCGG | 3900 |
| GTGCAGAGCG ACGTGTTCAG GGTGGCGGAG TTCCCTCACG GCTACGGCAG CTGTGACGAG | 3960 |
| ATCCCCAGGG TGGACCTCCG GGTGTGGCAG GACTGCTGTG AAGACTGTAG GACCAGGGGG | 4020 |
| CAGTTCAATG CCTTTTCCTA TCATTTCCGA GGCAGACGGT CTCTTGAGTT CAGCTACCAG | 4080 |
| GAGGACAAGC CGACCAAGAA AACAAGACCA CGGAAAATAC CCAGTGTTGG GAGACAGGGG | 4140 |
| GAACATCTCA GCAACAGCAC CTCAGCCTTC AGCACACGCT CAGATGCATC TGGGACAAAT | 4200 |
| GACTTCAGAG AGTTTGTTCT GGAAATGCAG AAGACCATCA CAGACCTCAG AACACAGATA | 4260 |
| AAGAAACTTG AATCACGGCT CAGTACCACA GAGTGCGTGG ATGCCGGGGG CGAATCTCAC | 4320 |
| GCCAACAACA CCAAGTGGAA AAAAGATGCA TGCACCATTT GTGAATGCAA AGACGGGCAG | 4380 |
| GTCACCTGCT TCGTGGAAGC TTGCCCCCCT GCCACCTGTG CTGTCCCCGT GAACATCCCA | 4440 |
| GGGGCCTGCT GTCCAGTCTG CTTACAGAAG AGGGCGGAGG AAAAGCCCTA GGCTCCTGGG | 4500 |
| AGGCTCCTCA GAGTTTGTCT GCTGTGCCAT CGTGAGATCG GGTGGCCGAT GGCAGGGAGC | 4560 |
| TGCGGACTGC AGACCAGGAA ACACCCAGAA CTCGTGACAT TTCATGACAA CGTCCAGCTG | 4620 |
| GTGCTGTTAC AGAAGGCAGT GCAGGAGGCT TCCAACCAGA GCATCTGCGG AGAAGGAGGC | 4680 |
| ACAGCAGGTG CCTGAAGGGA AGCAGGCAGG AGTCCTAGCT TCACGTTAGA CTTCTCAGGT | 4740 |
| TTTTATTTAA TTCTTTTAAA ATGAAAAATT GGTGCTACTA TTAAATTGCA CAGTTGAATC | 4800 |
| ATTTAGGCGC CTAAATTGGT TTTGCCTCCC AACACCATTT CTTTTTAAAT AAAGCAGGAT | 4860 |
| ACCTCTATAT GTCAGCCTTG CCTTGTTCAG ATGCCAGGAG CCGGCAGACC TGTCACCCGC | 4920 |
| AGGTGGGGTG AGTCTCGGAG CTGCCAGAGG GGCTCACCGA AATCGGGGTT CCATCACAAG | 4980 |
| CTATGTTTAA AAAGAAAATT GGTGTTTGGC AAACGGAACA GAACCTTTGA TGAGAGCGTT | 5040 |
| CACAGGGACA CTGTCTGGGG GTGCAGTGCA AGCCCCCGGC CTCTTCCCTG GGAACCTCTG | 5100 |
| AACTCCTCCT TCCTCTGGGC TCTCTGTAAC ATTTCACCAC ACGTCAGCAT CTAATCCCAA | 5160 |
| GACAAACATT CCCGCTGCTC GAAGCAGCTG TATAGCCTGT GACTCTCCGT GTGTCAGCTC | 5220 |
| CTTCCACACC TGATTAGAAC ATTCATAAGC CACATTTAGA AACAGATTTG CTTTCAGCTG | 5280 |
| TCACTTGCAC ACATACTGCC TAGTTGTGAA CCAAATGTGA AAAAACCTCC TTCATCCCAT | 5340 |
| TGTGTATCTG ATACCTGCCG AGGGCCAAGG GTGTGTGTTG ACAACGCCGC TCCCAGCCGG | 5400 |
| CCCTGGTTGC GTCCACGTCC TGAACAAGAG CCGCTTCCGG ATGGCTCTTC CCAAGGGAGG | 5460 |
| AGGAGCTCAA GTGTCGGGAA CTGTCTAACT TCAGGTTGTG TGAGTGCGTT | |

ACF5 DNA sequence
Gene name: Mitogen-activated protein kinase kinase kinase kinase 4
Unigene number: Hs.3628
Probeset Accession #: N54067
Nucleic Acid Accession #: NM_004834
Coding sequence: 80-3577 (predicted start/stop codons underlined)

| | |
|---|---|
| AATTCGAGGA TCCGGGTACC ATGGCACAGA GCGACAGAGA CATTTATTGT TATTTGTTTT | 60 |
| TTGGTGGCAA AAAGGGAAAA TGGCGAACGA CTCCCCTGCA AAAAGTCTGG TGGACATCGA | 120 |
| CCTCTCCTCC CTGCGGGATC CTGCTGGGAT TTTTGAGCTG GTGGAAGTGG TTGGAAATGG | 180 |
| CACCTATGGA CAAGTCTATA AGGGTCGACA TGTTAAAACG GGTCAGTTGG CAGCCATCAA | 240 |
| AGTTATGGAT GTCACTGAGG ATGAAGAGGA AGAAATCAAA CTGGAGATAA ATATGCTAAA | 300 |
| GAAATACTCT CATCACAGAA ACATTGCAAC ATATTATGGT GCTTTCATCA AAAAGAGCCC | 360 |
| TCCAGGACAT GATGACCAAC TCTGGCTTGT TATGGAGTTC TGTGGGGCTG GGTCCATTAC | 420 |
| AGACCTTGTG AAGAACACCA AAGGGAACAC ACTCAAAGAA GACTGGATCG CTTACATCTC | 480 |
| CAGAGAAATC CTGAGGGGAC TGGCACATCT TCACATTCAT CATGTGATTC ACCGGGATAT | 540 |
| CAAGGGCCAG AATGTGTTGC TGACTGAGAA TGCAGAGGTG AAACTTGTTG ACTTTGGTGT | 600 |
| GAGTGCTCAG CTGGACAGGA CTGTGGGGCG GAGAAATACG TTCATAGGCA CTCCCTACTG | 660 |
| GATGGCTCCT GAGGTCATCG CCTGTGATGA GAACCCAGAT GCCACCTATG ATTACAGAAG | 720 |
| TGATCTTTGG TCTTGTGGCA TTACAGCCAT TGAGATGGCA GAAGGTGCTC CCCCTCTCTG | 780 |
| TGACATGCAT CCAATGAGAG CACTGTTTCT CATTCCCAGA AACCCTCCTC CCCGGCTGAA | 840 |
| GTCAAAAAAA TGGTCGAAGA AGTTTTTTAG TTTTATAGAA GGGTGCCTGG TGAAGAATTA | 900 |
| CATGCAGCGG CCCTCTACAG AGCAGCTTTT GAAACATCCT TTTATAAGGG ATCAGCCAAA | 960 |
| TGAAAGGCAA GTTAGAATCC AGCTTAAGGA TCATATAGAT CGTACCAGGA AGAAGAGAGG | 1020 |
| CGAGAAAGAT GAAACTGAGT ATGAGTACAG TGGGAGTGAG GAAGAAGAGG AGGAAGTGCC | 1080 |
| TGAACAGGAA GGAGAGCCAA GTTCCATTGT GAACGTGCCT GGTGAGTCTA CTCTTCGCCG | 1140 |
| AGATTTCCTG AGACTGCAGC AGGAGAACAA GGAACGTTCC GAGGCTCTTC GGAGACAACA | 1200 |
| GTTACTACAG GAGCAACAGC TCCGGGAGCA GGAAGAATAT AAAAGGCAAC TGCTGGCAGA | 1260 |
| GAGACAGAAG CGGATTGAGC AGCAGAAAGA ACAGAGGCGA CGGCTAGAAG AGCAACAAAG | 1320 |
| GAGAGAGCGG GAGGCTAGAA GGCAGCAGGA ACGTGAACAG CGAAGGAGAG AACAAGAAGA | 1380 |
| AAAGAGGCGT CTAGAGGAGT TGGAGAGAAG GCGCAAAGAA GAAGAGGAGA GGAGACGGGC | 1440 |
| AGAAGAAGAA AAGAGGAGAG TTGAAAGAGA ACAGGAGTAT ATCAGGCGAC AGCTAGAAGA | 1500 |
| GGAGCAGCGG CACTTGGAAG TCCTTCAGCA GCAGCTGCTC CAGGAGCAGG CCATGTTACT | 1560 |
| GCATGACCAT AGGAGGCCGC ACCCGCAGCA CTCGCAGCAG CCGCCACCAC CGCAGCAGGA | 1620 |
| AAGGAGCAAG CCAAGCTTCC ATGCTCCCGA GCCCAAAGCC CACTACGAGC CTGCTGACCG | 1680 |
| AGCGCGAGAG GTTCCTGTGA GAACAACATC TCGCTCCCCT GTTCTGTCCC GTCGAGATTC | 1740 |
| CCCACTGCAG GGCAGTGGGC AGCAGAATAG CCAGGCAGGA CAGAGAAACT CCACCAGTAT | 1800 |
| TGAGCCCAGG CTTCTGTGGG AGAGAGTGGA GAAGCTGGTG CCCAGACCTG GCAGTGGCAG | 1860 |
| CTCCTCAGGG TCCAGCAACT CAGGATCCCA GCCCGGGTCT CACCCTGGGT CTCAGAGTGG | 1920 |
| CTCCGGGGAA CGCTTCAGAG TGAGATCATC ATCCAAGTCT GAAGGCTCTC CATCTCAGCG | 1980 |
| CCTGGAAAAT GCAGTGAAAA AACCTGAAGA TAAAAAGGAA GTTTTCAGAC CCCTCAAGCC | 2040 |
| TGCTGGCGAA GTGGATCTGA CCGCACTGGC CAAAGAGCTT CGAGCAGTGG AAGATGTACG | 2100 |
| GCCACCTCAC AAAGTAACGG ACTACTCCTC ATCCAGTGAG GAGTCGGGGA CGACGGATGA | 2160 |
| GGAGGACGAC GATGTGGAGC AGGAAGGGGC TGACGAGTCC ACCTCAGGAC CAGAGGACAC | 2220 |
| CAGAGCAGCG TCATCTCTGA ATTTGAGCAA TGGTGAAACG GAATCTGTGA AAACCATGAT | 2280 |
| TGTCCATGAT GATGTAGAAA GTGAGCCGGC CATGACCCCA TCCAAGGAGG GCACTCTAAT | 2340 |
| CGTCCGCCAG ACTCAGTCCG CTAGTAGCAC ACTCCAGAAA CACAAATCTT CCTCCTCCTT | 2400 |
| TACACCTTTT ATAGACCCCA GATTACTACA GATTTCTCCA TCTAGCGGAA CAACAGTGAC | 2460 |
| ATCTGTGGTG GGATTTTCCT GTGATGGGAT GAGACCAGAA GCCATAAGGC AAGATCCTAC | 2520 |
| CCGGAAAGGC TCAGTGGTCA ATGTGAATCC TACCAACACT AGGCCACAGA GTGACACCCC | 2580 |
| GGAGATTCGT AAATACAAGA AGAGGTTTAA CTCTGAGATT CTGTGTGCTG CCTTATGGGG | 2640 |
| AGTGAATTTG CTAGTGGGTA CAGAGAGTGG CCTGATGCTG CTGGACAGAA GTGGCCAAGG | 2700 |
| GAAGGTCTAT CCTCTTATCA ACCGAAGACG ATTTCAACAA ATGGACGTAC TTGAGGGCTT | 2760 |
| GAATGTCTTG GTGACAATAT CTGGCAAAAA GGATAAGTTA CGTGTCTACT ATTTGTCCTG | 2820 |
| GTTAAGAAAT AAAATACTTC ACAATGATCC AGAAGTTGAG AAGAAGCAGG GATGGACAAC | 2880 |
| CGTAGGGGAT TTGGAAGGAT GTGTACATTA TAAAGTTGTA AAATATGAAA GAATCAAATT | 2940 |
| TCTGGTGATT GCTTTGAAGA GTTCTGTGGA AGTCTATGCG TGGGCACCAA AGCCATATCA | 3000 |
| CAAATTTATG GCCTTTAAGT CATTTGGAGA ATTGGTACAT AAGCCATTAC TGGTGGATCT | 3060 |
| CACTGTTGAG GAAGGCCAGA GGTTGAAAGT GATCTATGGA TCCTGTGCTG GATTCCATGC | 3120 |
| TGTTGATGTG GATTCAGGAT CAGTCTATGA CATTTATCTA CCAACACATG TAAGAAAGAA | 3180 |
| CCCACACTCT ATGATCCAGT GTAGCATCAA ACCCCATGCA ATCATCATCC TCCCCAATAC | 3240 |
| AGATGGAATG GAGCTTCTGG TGTGCTATGA AGATGAGGGG GTTTATGTAA ACACATATGG | 3300 |
| AAGGATCACC AAGGATGTAG TTCTACAGTG GGGAGAGATG CCTACATCAG TAGCATATAT | 3360 |
| TCGATCCAAT CAGACAATGG GCTGGGGAGA GAAGGCCATA GAGATCCGAT CTGTGGAAAC | 3420 |
| TGGTCACTTG GATGGTGTGT TCATGCACAA AAGGGCTCAA AGACTAAAAT TCTTGTGTGA | 3480 |
| ACGCAATGAC AAGGTGTTCT TTGCCTCTGT TCGGTCTGGT GGCAGCAGTC AGGTTTATTT | 3540 |
| CATGACCTTA GGCAGGACTT CTCTTCTGAG CTGGTAGAAG CAGTGTGATC CAGGGATTAC 3600 | |
| TGGCCTCCAG AGTCTTCAAG ATCCTGAGAA CTTGGAATTC CTTGTAAC GAGCTCGGAG | 3660 |
| CTGCACCGAG GGCAACCAGG ACAGCTGTGT GTGCAGACCT CATGTGTTCG GTTCTCTCCC | 3720 |
| CTCCTTCCTG TTCCTCTTAT ATACCAGTTT ATCCCCATTC TTTTTTTTTT TCTTACTCCA | 3780 |
| AAATAAATCA AGGCTGCAAT GCAGCTGGTG CTGTTCAGAT TCCAAAAAAA AAAAAAAACC | 3840 |
| ATGGTACCCG GATCCTCGAA TTCC | |

ACF8 DNA sequence
Gene name: Phospholipase A2, group IVC (cytosolic, calcium-independent)
Unigene number: Hs.18858
Probeset Accession #: AA054087
Nucleic Acid Accession #: NM_003706
Coding sequence: 310-1935 (predicted start/stop codons underlined)

| | |
|---|---|
| CACGAGGCAG GGGCCATTTT ACCTCCAGGT TGGCCCTGCT CAGGACCAGG AGGAAACACC | 60 |
| TCCAGCCCGC GACCTCCTCC CACAGGGGGA AAAGGAAAGC AGGAGGACCA CAGAAGCTTT | 120 |
| GGCACCGAGG ATCCCCGCAG TCTTCACCCG CGGAGATTCC GGCTGAAGGA GCTGTCCAGC | 180 |
| GACTACACCG CTAAGCGCAG GGAGCCCAAG CCTCCGCACC GGATTCCGGA GCACAAGCTC | 240 |
| CACCGCGCAT GCGCACACGC CCCAGACCCA GGCTCAGGAG GACTGAGAAT TTTCTGACCG | 300 |
| CAGTGCACCA TGGGAAGCTC TGAAGTTTCC ATAATTCCTG GGCTCCAGAA AGAAGAAAAG | 360 |
| GCGGCCGTGG AGAGACGAAG ACTTCATGTG CTGAAAGCTC TGAAGAAGCT AAGGATTGAG | 420 |
| GCTGATGAGG CCCCAGTTGT TGCTGTGCTG GGCTCAGGCG GAGGACTGCG GGCTCACATT | 480 |
| GCCTGCCTTG GGGTCCTGAG TGAGATGAAA GAACAGGGCC TGTTGGATGC CGTCACGTAC | 540 |
| CTCGCAGGGG TCTCTGGATC CACTTGGGCA ATATCTTCTC TCTACACCAA TGATGGTGAC | 600 |
| ATGGAAGCTC TCGAGGCTGA CCTGAAACAT CGATTTACCC GACAGGAGTG GGACTTGGCT | 660 |
| AAGAGCCTAC AGAAAACCAT CCAAGCAGCG AGGTCTGAGA ATTACTCTCT GACCGACTTC | 720 |
| TGGGGCTACA TGGTTATCTC TAAGCAAACC AGAGAACTGC CGGAGTCTCA TTTGTCCAAT | 780 |
| ATGAAGAAGC CCGTGGAAGA AGGGACACTA CCCTACCCAA TATTTGCAGC CATTGACAAT | 840 |
| GACCTGCAAC CTTCCTGGCA GGAGGCAAGA GCACCAGAGA CCTGGTTCGA GTTCACCCCT | 900 |
| CACCACGCTG GCTTCTCTGC ACTGGGGGCC TTTGTTTCCA TAACCCACTT CGGAAGCAAA | 960 |
| TTCAAGAAGG GAAGACTGGT CAGAACTCAC CCTGAGAGAG ACCTGACTTT CCTGAGAGGT | 1020 |
| TTATGGGGAA GTGCTCTTGG TAACACTGAA GTCATTAGGG AATACATTTT TGACCAGTTA | 1080 |
| AGGAATCTGA CCCTGAAAGG TTTATGGAGA AGGGCTGTTG CTAATGCTAA AAGCATTGGA | 1140 |
| CACCTTATTT TTGCCCGATT ACTGAGGCTG CAAGAAAGTT CACAAGGGGA ACATCCTCCC | 1200 |
| CCAGAAGATG AAGGCGGTGA GCCTGAACAC ACCTGGCTGA CTGAGATGCT CGAGAATTGG | 1260 |
| ACCAGGACCT CCCTGGAAAA GCAGGAGCAG CCCCATGAGG ACCCCGAAAG GAAAGGCTCA | 1320 |
| CTCAGTAACT TGATGGATTT TGTGAAGAAA ACAGGCATTT GCGCTTCAAA GTGGGAATGG | 1380 |
| GGGACCACTC ACAACTTCCT GTACAAACAC GGTGGCATCC GGGACAAGAT AATGAGCAGC | 1440 |
| CGGAAGCACC TCCACCTGGT GGATGCTGGT TTAGCCATCA ACACTCCCTT CCCACTCGTG | 1500 |
| CTGCCCCCGA CGCGGGAGGT TCACCTCATC CTCTCCTTCG ACTTCAGTGC CGGAGATCCT | 1560 |
| TTCGAGACCA TCCGGGCTAC CACTGACTAC TGCCGCCGCC ACAAGATCCC CTTTCCCCAA | 1620 |
| GTAGAAGAGG CTGAGCTGGA TTTGTGGTCC AAGGCCCCCG CCAGCTGCTA CATCCTGAAA | 1680 |
| GGAGAAACTG GACCAGTGGT GATACATTTT CCCCTGTTCA ACATAGATGC CTGTGGAGGT | 1740 |
| GATATTGAGG CATGGAGTGA CACATACGAC ACATTCAAGC TTGCTGACAC CTACACTCTA | 1800 |
| GATGTGGTGG TGCTACTCTT GGCATTAGCC AAGAAGAATG TCAGGGAAAA CAAGAAGAAG | 1860 |
| ATCCTTAGAG AGTTGATGAA CGTGGCCGGG CTCTACTACC CGAAGGATAG TGCCCGAAGT | 1920 |
| TGCTGCTTGG CATAGATGAG CCTCAGCTTC CAGGGCACTG TGGGCCTGTT GGTCTACTAG | 1980 |
| GGCCCTGAAG TCCACCTGGC CTTCCTGTTC TTCACTCCCT TCAGCCACAC GCTTCATGGC | 2040 |
| CTTGAGTTCA CCTTGGCTGT CCTAACAGGG CCAATCACCA GTGACCAGCT AGACTGTGAT | 2100 |
| TTTGATAGCG TCATTCAGAA GAAGGTGTCC AAGGAGCTGA AGGTGGTGAA ATTTGTCCTG | 2160 |
| CAGGTCCCTC GGGAGATCCT GGAGCTGGAG CATGAGTGTC TGACAATCAG AAGCATCATG | 2220 |
| TCCAATGTCC AGATGGCCAG AATGAATGTG ATAGTTCAGA CCAATGCCTT CCACTGCTCC | 2280 |
| TTTATGACTG CACTTCTAGC CAGTAGCTCT GCACAAGTTA GCTCTGTAGA AGTAAGAACT | 2340 |
| TGGGCTTAAA TCATGGGCTA TCTCTCCACA GCCAAGTGGA GCTCTGAGAA TACAACAAGT | 2400 |
| GCTCAATAAA TGCTTGCTGA TTGACTGATG AAAAAAAAAA AAAAAAAAAA AAAAAAAAAA | 2460 |
| AAAAAAAAAA AAAAAAAAAA AAAAAAAAAA AAAA | |

ACG1 DNA sequence
Gene name: Carbohydrate (chondroitin 6/keratan) sulfotransferase 1
Unigene number: Hs.104576
Probeset Accession #: AA868063
Nucleic Acid Accession #: NM_002654
Coding sequence: 367-1602 (predicted start/stop codons underlined)

| | |
|---|---|
| GGGGAGGGCG CGGGAGGCGG AGGATGCCGC CGCGGCTGCT GCCGCCGCCG CCACCCGCGG | 60 |
| GTCCCCGGCG ACCCTACTCC AGACCCGAGG ATGGAGCCGG CGCTGGGCGC TGCAGCTGCT | 120 |
| CCCGGCGCGT CCCCGACCAG GTAGCTGGTG TCACTTCGGT GTGGTTGGAA GAAGACTTTC | 180 |
| TCCCCAGCTG CATTCCCGGA GGCGCCCTTT CGACCTGGAG GCCGGGTCTG CTGGCCACAG | 240 |
| GGCTGCCGCA CTGGCTGGGA CTGCCAGCTG GGCCTGGAGA CGCTGGTGGC TGTGGACTCC | 300 |
| CCAGCTTGGA GCAGTCCCTC TTTGACCTCA CCCCTTGGAG AAGCAGCCCC ATGAAGGTGC | 360 |
| CCAGCCATGC AATGTTCCTG GAAGGCCGTC CTCCTCCTTG CCCTGGCCTC CATTGCCATC | 420 |
| CAGTACACGG CCATCCGCAC CTTCACCGCC AAGTCCTTTC ACACCTGCCC CGGGCTGGCA | 480 |
| GAGGCCGGGC TGGCCGAGCG ACTGTGCGAG GAGAGCCCCA CCTTCGCCTA CAACCTCTCC | 540 |
| CGCAAGACCC ACATCCTCAT CCTGGCCACC ACGCGCAGCG GCTCCTCCTT CGTGGGCCAG | 600 |
| CTCTTCAACC AGCACCTGGA CGTCTTCTAC CTGTTTGAGC CCCTCTACCA CGTCCAGAAC | 660 |
| ACGCTCATCC CCCGCTTCAC CCAGGGCAAG AGCCCGGCCG ACCGGCGGGT CATGCTAGGC | 720 |
| GCCAGCCGCG ACCTCCTGCG GAGCCTCTAC GACTGCGACC TCTACTTCCT GGAGAACTAC | 780 |
| ATCAAGCCGC CGCCGGTCAA CCACACCACC GACAGGATCT TCCGCCGCGG GGCCAGCCGG | 840 |
| GTCCTCTGCT CCCGGCCTGT GTGCGACCCT CCGGGGCCAG CCGACCTGGT CCTGGAGGAG | 900 |
| GGGGACTGTG TGCGCAAGTG CGGGCTACTC AACCTGACCG TGGCGGCCGA GGCGTGCCGC | 960 |
| GAGCGCAGCC ACGTGGCCAT CAAGACGGTG CGCGTGCCCG AGGTGAACGA CCTGCGCGCC | 1020 |
| CTGGTGGAAG ACCCGCGATT AAACCTCAAG GTCATCCAGC TGGTCCGAGA CCCCCGCGGC | 1080 |
| ATTCTGGCTT CGCGCAGCGA GACCTTCCGC GACACGTACC GGCTCTGGCG GCTCTGGTAC | 1140 |
| GGCACCGGGA GGAAACCCTA CAACCTGGAC GTGACGCAGC TGACCACGGT GTGCGAGGAC | 1200 |
| TTCTCCAACT CCGTGTCCAC CGGCCTCATG CGGCCCCCGT GGCTCAAGGG CAAGTACATG | 1260 |
| TTGGTGCGCT ACGAGGACCT GGCTCGGAAC CCTATGAAGA AGACCGAGGA GATCTACGGG | 1320 |
| TTCCTGGGCA TCCCGCTGGA CAGCCACGTG GCCCGCTGGA TCCAGAACAA CACGCGGGGC | 1380 |
| GACCCCACCC TGGGCAAGCA CAAATACGGC ACCGTGCGAA ACTCGGCGGC CACGGCCGAG | 1440 |
| AAGTGGCGCT TCCGCCTCTC CTACGACATC GTGGCCTTTG CCCAGAACGC CTGCCAGCAG | 1500 |
| GTGCTGGCCC AGCTGGGCTA CAAGATCGCC GCCTCGGAGG AGGAGCTGAA GAACCCCTCG | 1560 |
| GTCAGCCTGG TGGAGGAGCG GGACTTCCGC CCCTTCTCGT GACCCGGGCG GTGCGGGTGG | 1620 |
| GGGCGGGAGG CGCAAGGTGT CGGTTTTGAT AAAATGGACC GTTTTTAACT GTTGCCTTAT | 1680 |
| TAACCCCTCC CTCTCCCACC TCATCTTCGT GTCCTTCCTG CCCCCAGCTC ACCCCACTCC | 1740 |
| CTTCTGCCCC TTTTTTGTCT CTGAAATTTG CACTACGTCT TGGACGGGAA TCACTGGGGC | 1800 |
| AGAGGGCGCC TGAAGTAGGG TCCCGCCCCC CCCACCCCAT TCAGACACAT GGATGTTGGG | 1860 |
| TCTCTGTGCG GACGGTGACA ATGTTTACAA GCACCACATT TACACATCCA CACACGCACA | 1920 |
| CGGGCACTCG CGAGGCGACT TCTCAAGCTT TTGAATGGGT GAGTGGTCGG GTATCTAGTT | 1980 |
| TTTGCACTGT CTTACTATTC AAGGTAAGAG GATACAAACA AGAGGACCAC TTGTCTCTAA | 2040 |
| TTTATGAATG GTGTCCATCC TTTCCCCATC CCTGCCTCCT GCCCCTGACG CCCATTTCCC | 2100 |
| CCCTTAGAGC AGCGAAACTG CCCCCTCCTG CCCGCCCTTG CCTGTCGGTG AGGCAGGTTT | 2160 |
| TTACTGTGAG GTGAACGTGG ACCTGTTTCT GTTTCCAGTC TGTGGTGATG CTGTCTGTCT | 2220 |
| GTCTGAGTCT CGTGGCCGCC CCTGGACCAG TGATGACTGA TGAATCTTAT GAGCTTCTGA | 2280 |
| TTGATCTCGG GGTCCATCTG TGATATTTCT TTGTGCCAAA AAGAAAAAAA AAGAGTGGAT | 2340 |
| CAGTTTGCTA AATGAACATT GAAATTGAAA TGCTTTATCT GTGTTTTCTG TAAATAAAAG | 2400 |
| AGTGCAATAA TCACC | |

ACG5 DNA sequence
Gene name: Multimerin
Unigene number: Hs.268107
Probeset Accession #: U27109
Nucleic Acid Accession #: U27109.1
Coding sequence: 72-3758 (predicted start/stop codons underlined)

| | |
|---|---|
| CTGCTATCAA AAAGGCCATA AGGATTTTGT CCCCAAATTT CACATGAGCT ACCTTGCTTC | 60 |
| AAACTACTGA GATGAAGGGG GCAAGATTAT TTGTCCTTCT TTCTAGTTTA TGGAGTGGGG | 120 |
| GCATTGGGCT TAACAACAGT AAGCATTCTT GGACTATACC TGAGGATGGG AACTCTCAGA | 180 |
| AGACTATGCC TTCTGCTTCA GTTCCTCCAA ATAAAATACA AAGTTTGCAA ATACTGCCAA | 240 |
| CCACTCGGGT CATGTCGGCG GAGATAGCTA CAACTCCAGA GGCAAGAACT TCTGAAGACA | 300 |
| GTCTTCTTAA ATCAACACTG CCTCCCTCAG AAACAAGTGC ACCTGCTGAG GGTGTGAGAA | 360 |
| ATCAAACTCT CACATCCACA GAGAAAGCAG AAGGAGTGGT CAAGTTACAG AATCTTACCC | 420 |
| TCCCAACCAA CGCTAGCATC AAGTTCAATC CTGGAGCAGA ATCAGTGGTC CTTTCCAATT | 480 |
| CTACACTGAA ATTTCTTCAG AGCTTTGCCA GAAAGTCAAA TGAACAAGCA ACTTCTCTAA | 540 |
| ACACAGTTGG AGGCACTGGA GGCATTGGAG GCGTTGGAGG CACTGGAGGC GTGGGAAATC | 600 |
| GAGCCCCACG GGAAACATAC CTCAGCCGGG GTGACAGCAG TTCCAGCCAA AGAACTGACT | 660 |
| ACCAAAAATC AAATTTCGAA ACAACTAGAG GAAAGAATTG GTGTGCTTAT GTACATACCA | 720 |
| GGTTATCTCC CACAGTGACA TTGGACAACC AGGTCACTTA TGTCCCAGGT GGGAAAGGAC | 780 |
| CTTGTGGCTG GACCGGTGGA TCCTGTCCTC AGAGATCTCA GAAGATATCC AATCCTGTCT | 840 |
| ATAGGATGCA ACATAAAATT GTCACCTCAT TGGATTGGAG GTGCTGTCCT GGATACAGTG | 900 |
| GGCCGAAATG TCAACTAAGA GCCCAGGAAC AGCAAAGTTT GATACACACC AACCAGGCTG | 960 |
| AAAGTCATAC AGCTGTTGGC AGAGGAGTAG CTGAGCAGCA GCAGCAGCAA GGCTGTGGTG | 1020 |
| ACCCAGAAGT GATGCAAAAA ATGACTGATC AGGTGAACTA CCAGGCAATG AAACTGACTC | 1080 |
| TTCTGCAGAA GAAGATTGAC AATATTTCTT TGACTGTGAA TGATGTAAGG AACACTTACT | 1140 |
| CCTCCCTAGA AGGAAAAGTC AGCGAAGATA AAAGCAGAGA ATTTCAATCT CTTCTAAAAG | 1200 |
| GTCTAAAATC CAAAAGCATT AATGTACTGA TAAGAGACAT AGTAAGAGAA CAATTTAAAA | 1260 |
| TTTTTCAAAA TGATGCAA GAGACTGTAG CACAGCTCTT CAAGACTGTA TCAAGTCTAT | 1320 |
| CAGAGGACCT CGAAAGCACC AGGCAAATAA TTCAAAAAGT TAATGAATCT GTGGTTTCAA | 1380 |
| TAGCAGCCCA GCAAAAGTTT GTTTTGGTGC AAGAGAATCG GCCCACTTTG ACTGATATAG | 1440 |
| TGGAACTAAG GAATCACATT GTGAATGTAA GGCAAGAAAT GACTCTTACA TGTGAGAAGC | 1500 |
| CTATTAAAGA ACTAGAAGTA AAGCAGACTC ATTTAGAAGG TGCTCTAGAA CAGGAACACT | 1560 |
| CAAGAAGCAT TCTGTATTAT GAATCCCTCA ATAAAACTCT TTCTAAATTG AAGGAAGTAC | 1620 |
| ATGAGCAGCT TTTATCAACT GAACAGGTAT CAGACCAGAA GAATGCTCCA GCTGCTGAGT | 1680 |
| CAGTTAGCAA TAATGTCACT GAGTACATGT CTACTTTACA TGAAAATATA AAGAAGCAGA | 1740 |
| GTTTGATGAT GCTGCAAATG TTTGAAGATT TGCACATTCA AGAAAGCAAG ATTAACAATC | 1800 |
| TCACCGTCTC TTTGGAGATG GAGAAAGAGT CTCTCAGAGG TGAATGTGAA GACATGTTAT | 1860 |
| CCAAATGCAG AAATGATTTT AAATTTCAAC TTAAGGACAC AGAAGAGAAT TTACATGTGT | 1920 |
| TAAATCAAAC ATTGGCTGAA GTTCTCTTTC CAATGGACAA TAAGATGGAC AAAATGAGTG | 1980 |
| AGCAACTAAA TGATTTGACT TATGATATGG AGATCCTTCA ACCCTTGCTT GAGCAGGGAG | 2040 |
| CATCACTCAG ACAGACAATG ACATATGAAC AACCAAAGGA AGCAATAGTG ATAAGGAAAA | 2100 |
| AGATAGAAAA TCTGACTAGT GCTGTCAATA GTCTAAATTT TATTATCAAA GAACTTACAA | 2160 |
| AAAGACACAA CTTACTTAGA AATGAAGTAC AGGGTCGTGA TGATGCCTTA GAAAGACGTA | 2220 |
| TCAATGAATA TGCCTTAGAA ATGGAAGATG GCCTCAATAA GACAATGACT ATTATAAATA | 2280 |
| ATGCTATTGA TTTCATTCAA GATAACTATG CCCTAAAAGA GACTTTAAGT ACTATTAAGG | 2340 |
| ATAATAGTGA GATCCATCAT AAATGTACCT CCGATATGGA AACTATTTTG AGATTTATTC | 2400 |
| CTCAGTTCCA CCGTCTGAAT GATTCTATTC AGACTTTGGT CAATGACAAT CAGAGATATA | 2460 |
| ACTTTGTTTT GCAAGTCGCC AAGACCCTTG CAGGTATTCC CAGAGATGAG AAACTAAATC | 2520 |
| AGTCCAACTT CCAAAAGATG TATCAAATGT TCAATGAAAC CACTTCCCAA GTGAGAAAAT | 2580 |
| ACCAGCAAAA TATGAGTCAT TTGGAAGAAA AACTACTCTT AACTACCAAG ATTTCCAAAA | 2640 |
| ATTTTGAGAC TCGGTTGCAA GACATTGAGT CTAAAGTTAC CCAGACGCTC ATACCTTATT | 2700 |
| ATATTTCAGT TAAAAAAGGC AGTGTAGTTA CAAATGAGAG AGATCAGGCT CTTCAACTGC | 2760 |
| AAGTATTAAA TTCCAGATTT AAGGCGTTGG AAGCAAAATC TATCCATCTT TCAATTAACT | 2820 |
| TCTTTTCGCT TAACAAAACT CTCCACGAAG TTTTAACAAT GTGTCACAAT GCTTCTACAA | 2880 |
| GTGTGTCAGA ACTGAATGCT ACCATCCCTA AGTGGATAAA ACATTCCCTG CCAGATATTC | 2940 |
| AACTTCTTCA GAAAGGTCTA ACAGAATTTG TGGAACCAAT AATTCAAATA AAAACTCAAG | 3000 |
| CTGCCCTATC TAATTCAACT TGTTGTATAG ATCGATCGTT GCCTGGTAGT CTGGCAAATG | 3060 |
| TTGTCAAGTC TCAGAAGCAA GTAAAATCAT TGCCAAAGAA AATTAACGCA CTTAAGAAAC | 3120 |
| CAACGGTAAA TCTTACCACA GTCCTGATAG GCCGGACTCA AAGAAACACG GACAACATAA | 3180 |
| TATATCCTGA GGAGTATTCA AGCTGTAGTC GGCATCCGTG CCAAAATGGG GGCACGTGCA | 3240 |
| TAAATGGAAG AACTAGCTTT ACCTGTGCCT GCAGACATCC TTTTACTGGT GACAACTGCA | 3300 |
| CTATCAAGCT TGTGGAAGAA AATGCTTTAG CTCCAGATTT TTCCAAAGGA TCTTACAGAT | 3360 |
| ATGCACCCAT GGTGGCATTT TTTGCATCTC ATACGTATGG AATGACTATA CCTGGTCCTA | 3420 |
| TCCTGTTTAA TAACTTGGAT GTCAATTATG GAGCTTCATA TACCCCAAGA ACTGGAAAAT | 3480 |
| TTAGAATTCC GTATCTTGGA GTATATGTTT TCAAGTACAC CATCGAGTCA TTTAGTGCTC | 3540 |
| ATATTTCTGG ATTTTTAGTG GTTGATGGAA TAGACAAGCT TGCATTTGAG TCTGAAAATA | 3600 |
| TTAACAGTGA AATACACTGT GATAGGGTTT TAACTGGGGA TGCCTTATTA GAATTAAATT | 3660 |
| ATGGGCAGGA AGTCTGGTTA CGACTTGCAA AAGGAACAAT TCCAGCCAAG TTTCCCCCTG | 3720 |
| TTACTACATT TAGTGGCTAT TTATTATATC GTACATAAGT TAGTATGAAA AACAGACTAT | 3780 |
| CACCTTTATT GAGAAACAGC CAGTGTTTTC ATTTATCTTT GCTTGCACAT CTGCTCTGTT | 3840 |
| TTGGTTTTTC TACAGGAAAT GAAAATCAAC TTGTTTTTTT AATATGAGTA AACTTGTATG | 3900 |
| TCTATTTTAT AAAATTATTT GAATATTGTT TAATGTCTGA ATATGAAAGA GTTCTTGATC | 3960 |
| CTAAAGAAAT TTAGTGGCAC AGAAAACAAA GTGAATTTGT TAGCATAATT ATTCCTATTC | 4020 |
| TTATTTCTTC ATTTTAAGTC ATTGCAATGG AAAGTAATAT TATAAAACGG TAATTACAAC | 4080 |
| ATATTATCAG TCACAGTTTT CTTTCCAATT AAACACTTAA CTTTTGTTAT TCCCTGTATA | 4140 |
| TAAATATATA ACACACATTT TCTAGATTCA CAAATTTAAA TAAATTACTC AAAAAATG | |

ACC6 DNA sequence
Gene name: Homo sapiens cDNA FLJ11502 fis, clone HEMBA1002102, weakly similar to
ANKRYIN
Unigene number: Hs.213194
Probeset Accession #: AA187101
Nucleic Acid Accession #: AK021564
Coding sequence: 1-450 (predicted stop codon underlined, 5′end sequence is open)

| | |
|---|---|
| GTCGCCGCGC GGCCGCCGGT GAGCCGCATG GAGCCCCGGG CGGCGGACGG CTGCTTCCTG | 60 |
| GGCGACGTGG GTTTCTGGGT GGAGCGGACC CCTGTGCACG AGGCAGCCCA GCGGGGTGAG | 120 |
| AGCCTGCAGC TGCAACAGCT GATCGAGAGC GGCGCCTGCG TGAACCAGGT CACCGTGGAC | 180 |
| TCCATCACGC CCCTGCACGC AGCCAGTCTG CAGGGCCAGG CGCGGTGTGT GCAGCTGCTG | 240 |
| CTGGCGGCTG GGGCCCAGGT GGATGCTCGC AACATCGACG GCAGCACCCC GCTCTGCGAT | 300 |
| GCCTGCGCCT CGGGCAGCAT CGAGTGTGTG AAGCTCTTGC TGTCCTACGG GGCCAAGGTC | 360 |
| AACCCTCCCC TGTACACAGC GTCCCCCCTG CACGAGGCCA GCTTTCCCCG CCTCCTGAGC | 420 |
| ACCCTGGCTT CGACGCCCTG GATCAACTGA GCCAGGTGGA ACTCCTGGGG GACATGGATC | 480 |
| GCAATGAATT CGACCAGTAT TTGAACACTC CTGGCCACCC AGACTCCGCC ACAGGGGCCA | 540 |
| TGGCCCTCAG TGGGCATGTT CCGGTCTCCC AGGTGACACC AACGGGTCCC ACAGAGACCA | 600 |
| GCCTCATCTC CGTCCTGGCT GATGCCACGG CCACGTACTA CAACAGCTAC AGTGTGTCAT | 660 |
| AGAGCTGGAG GCGCCCCGTC CGGTCAGCCC TCGCGCCCTC TCCTTCTTGT GCCTTGAGTG | 720 |
| GCAGAGGAGC CGTCCAGCCA CACCAGCTTT CCTCCCACCG CTCAGGGCAG GGAGGTCTGA | 780 |
| ACTGCGGCCC CAGAGCCTTT GGCCTAAGCT GGACTCTCCT TATCCGAGTG CCGCCTCTAT | 840 |
| CCCCTTCCCC ACGTTCCAGC CCCTGCAGCC CACATTTTAA GTATATTCCT TCAAGTGAGT | 900 |
| TTTCCTCCAG CCCCTGAGAG TTGCTGTCTC CCAGTGGAAT GTTCACTGAC GTCTTTTCTT | 960 |
| GGTAGCCATC ATCGAAACTA ATGGGGGGAC AGACTTGATA GCCAAGGTCC CTTCTGGTCC | 1020 |
| AGTTTTCTGA TTTAGGGTTC TCTCAAGATT AATAAAGGAA GATGGGGAAA TTTGACTCAT | 1080 |
| TAATGAGCTC GCTAACCTAC GATCTGGTGA TAATTTTGTG TGCACAGCCC AAGGACCACG | 1140 |
| AGGCTTTCTG CACTTTCTGC ACCCCCTTCC AAAGTGACCA CAAAATTTCA AAGGGACTCA | 1200 |
| TACAATTTGA GAAAAAACAG TCAACCTGAT TTGAGAAATT AACCAGTATG GCTAACTATA | 1260 |
| TCACAGAAAA TGGGATTGAG TTAAAACTAT TTTATTTTAA ATATACATTT TAAAGCAGTT | 1320 |
| CTTTTTTTTT TGTTAATTTG TTTATTATAC ACACACTTCA AGAGAATATG CACAGTCTAG | 1380 |
| GCCGGGCACG GTGGCTCACG CCTGTAATCC CAGCACTTTG GGAGGCCGAG GCATGTGGAT | 1440 |
| CACCTGAGGT CAGGAGTTTG AGACCAGCCT AGACAACATG GTGAAACCTT GTCTCTATGA | 1500 |
| AAAATACAAA ATTTGCTGGG AGTGGTGGTG CATGCCTGTA ATCCCAGCTA CTTGGAAGGC | 1560 |
| TGAGGCAGGA GAATGTCTTG AACCTAGGAG GTGGAGGTTG CAGTGAGCTG AGATTGCACC | 1620 |
| ATTGCACTCC AGCCTGTGCA ACAAGAGTGA AACTCCATTT CAAG | |

ACC7 DNA sequence
Gene name: Human RAL A gene
Unigene number: Hs.6906
Probeset Accession #: AA083572
Nucleic Acid Accession #: contig of X15014.1 and AK026850
Coding sequence: 1-621 (predicted start/stop codons underlined)

| | |
|---|---|
| ATGGCTGCAA ATAAGCCCAA GGGTCAGAAT TCTTTGGCTT TACACAAAGT CATCATGGTG | 60 |
| GGCAGTGGTG GCGTGGGCAA GTCAGCTCTG ACTCTACAGT TCATGTACGA TGAGTTTGTG | 120 |
| GAGGACTATG AGCCTACCAA AGCAGACAGC TATCGGAAGA AGGTAGTGCT AGATGGGGAG | 180 |
| GAAGTCGAGA TCGATATCTT AGATACAGCT GGGCAGGAGG ACTACGCTGC AATTAGAGAC | 240 |
| AACTACTTCC GAAGTGGGGA GGGGTTCCTC TGTGTTTTCT CTATTACAGA AATGGAATCC | 300 |
| TTTGCAGCTA CAGCTGACTT CAGGGAGCAG ATTTTAAGAG TAAAAGAAGA TGAGAATGTT | 360 |
| CCATTTCTAC TGGTTGGTAA CAAATCAGAT TTAGAAGATA AAAGACAGGT TTCTGTAGAA | 420 |
| GAGGCAAAAA ACAGAGCTGA GCAGTGGAAT GTTAACTACG TGGAAACATC TGCTAAAACA | 480 |
| CGAGCTAATG TTGACAAGGT ATTTTTTGAT TTAATGAGAG AAATTCGAGC GAGAAAGATG | 540 |
| GAAGACAGCA AAGAAAAGAA TGGAAAAAAG AAGAGGAAAA GTTTAGCCAA GAGAATCAGA | 600 |
| GAAAGATGCT GCATTTTATA ATCAAAGCCC AAACTCCTTT CTTATCTTGA CCATACTAAT | 660 |
| AAATATAATT TATAAGCATT GCCATTGAAG GCTTAATTGA CTGAAATTAC TTTAACATTT | 720 |
| TGGAAATTGT TGTATATCAC TAAAAGCATG AATTGGAACT GCAATGAAAG TCAAATTTAC | 780 |
| TTTAAAAAGA AATTAATATG GCTTCACCAA GAAGCAAAGT TCAACTTATT TCATAATTGC | 840 |
| CTACATTTAT CATGGTCCTG AATGTAGCGT GTAAGCTTGT GTTTCTTGGG CAGTCTTTCT | 900 |
| TGAAATTGAA GAGGTGAAAT GGGGGTGGGG AGTGGGAGGA AAGGTGACTT CCTCTGGTGT | 960 |
| TTATTATAAA GCTTAAATTT TATATCATTT TAAAATGTCT TGGTCTTCTA CTGCCTTGAA | 1020 |
| AAATGACAAT TGTGAACATG ATAGTTAAAC TACCACTTTT TTTAACCATT ATTATGCAAA | 1080 |
| ATTTAGAAGA AAAGTTATTG GCATGGTTGT TGCATATAGT TAAACTGAGA GTAATTCATC | 1140 |
| TGTGAATCTG CTTTAATTAC CTGGTGAGTA ACTTAGAAAA GTGGTGTAAA CTTGTACATG | 1200 |
| GAATTTTTTG AATATGCCTT AATTTAGAAA CTGAAAAATA TCCGGTTATA TGATTCTGGG | 1260 |
| TGTGTTCTTA CTGACACCAG GGGTCCGCTG CCCCATGTGT CCTGGTGAGA AAATATATGC | 1320 |
| CTGGCACAGC TTTTGTATAG AAAATTCTTG AGAAGTAACT GTCCGCTAGA AGTCTGTCCA | 1380 |
| AATTTAAAAT GTGTGCCATA TTCTGGTTCT TGAAAATAAG ATTCCAGAGC TCTTTGATCG | 1440 |
| CTTTTAATAA ACTGCAAGTT CATTTTAATT GAAGGGCCAG CATATATACT TGCAAGATAA | 1500 |
| TTTTCAGCTG CAAGGATTCA GCACCAGTTA TGTTTGAATG AACCCTCCTT TTCTCTGAGA | 1560 |
| TTCTGGTCCC TGGAAATCCC TTTCTGCTAG TGGTGAGCAT GTAAGTGTTA AGTTTTTAAT | 1620 |
| CTGGGAGCAG GGCATAGGAA GAAAATGTCA GTAGTGCTAA TGCATTTTGC ACTAGAACGC | 1680 |
| TTCGGGAAAA TATTCATGCT TGCCATCTGT TCATTTCTAA ATTTATATTC ATAAAGTTAC | 1740 |
| AGTTTGATAC AGGAATTATT AGGAGTAATT CTTTTCTGTT TCTGTTTATA ATGAAGAACA | 1800 |
| CTGTAGCTAC ATTTTCAGAA GTTAACATCA AGCCATCAAA CCTGGGTATA GTGCAGAAGA | 1860 |
| CGTGGCACAC ACTGACCACA CATTAGGCTG TGTCACCATT GTGTGGTGTA CCTGCTGGAA | 1920 |
| GAATTCTAGC ATGCTACTTG GGGACATAAT TTCAGTGGGA AATATGCCAC TGACCGATTT | 1980 |
| TTTTTTTTTT CCTCTTTGCA GTGGGGCTAG GACAGTTGAT TCAACAAAGT ATTTTTTTCT | 2040 |
| TTTTTCTCAG TCCTAATTTG GACAGGTCAA AGATGTGTTC AGGCATTCCA GGTAACAGGT | 2100 |
| GTGTATGTAA AGTTAAAAAT AGGCTTTTTA GGAACTCACT CTTTAGATAT TTACATCCAG | 2160 |
| CTTCTCATGT TAAATATTTG TCCTTAAAGG GTTTGAGATG TACATCTTTC ATTTCGTATT | 2220 |
| TCTCATAGGC TATGCCATGT GCGGAATTCA AGTTACCAAT GTAACACTGG CCAGCGGGCC | 2280 |
| CAGCAATCTC CATGTGTACT TATTACAGTC TTATTTAACC AGGGGTCCTA ACCACTAACA | 2340 |
| TTGTGACTTT GCTTTGAGAC CTTTCCTCTC CTGGGTACTG AGGTGCTATG AAGCCAACTG | 2400 |
| ACAAAGATGC ATCACGTGTC TTAGGCTGAT GCCACTACCC GATTTGTTTA TTTGCATTT | 2460 |
| GAGCCATTTA AAGACCAATA AACTTCCTTT TTTAAAAAAA AAAAAAAAAA AAAAAAAAAA | 2520 |
| A | |

ACC9 DNA sequence
Gene name: KIAA0955 protein
Unigene number: Hs.10031
Probeset Accession #: AA027168
Nucleic Acid Accession #: AB023172
Coding sequence: 314-1609 (predicted start/stop codons underlined)

| | |
|---|---|
| CTGGTTCTCA ACTTCTTTTG AAATAATGTT CATAGAGAAG GAGGGCTGTC TGAGATTCGA | 60 |
| GGGAAACAAG CTCTCAGGAC TTCCGGTCGC CATGATGGCT GTGGGCGGTA AACGCGGTTA | 120 |
| GTGCAAGCAT CTGGGCCATC TTCAATGGTA AAAAAGATAC AGTAAAGACA TAAATACCAC | 180 |
| ATTTGACAAA TGGAAAAAAA GGAGTGTCCA GAAAAGAGTA GCAGCAGTGA GGAAGAGCTG | 240 |
| CCGAGACGGG TATACAGGGA GCTACCCTGT GTTTCTGAGA CCCTTTGTGA CATCTCACAT | 300 |
| TTTTTCCAAG AAGATGATGA GACAGAGGCA GAGCCATTAT TGTTCCGTGC TGTTCCTGAG | 360 |
| TGTCAACTAT CTGGGGGGGA CATTCCCAGG AGACATTTGC TCAGAAGAGA ATCAAATAGT | 420 |
| TTCCTCTTAT GCTTCTAAAG TCTGTTTTGA GATCGAAGAA GATTATAAAA ATCGTCAGTT | 480 |
| TCTGGGGCCT GAAGGAAATG TGGATGTTGA GTTGATTGAT AAGAGCACAA ACAGATACAG | 540 |
| CGTTTGGTTC CCCACTGCTG GCTGGTATCT GTGGTCAGCC ACAGGCCTCG GCTTCCTGGT | 600 |
| AAGGGATGAG GTCACAGTGA CGATTGCGTT TGGTTCCTGG AGTCAGCACC TGGCCCTGGA | 660 |
| CCTGCAGCAC CATGAACAGT GGCTGGTGGG CGGCCCCTTG TTTGATGTCA CTGCAGAGCC | 720 |
| AGAGGAGGCT GTCGCCGAAA TCCACCTCCC CCACTTCATC TCCCTCCAAG GTGAGGTGGA | 780 |
| CGTCTCCTGG TTTCTCGTTG CCCATTTTAA GAATGAAGGG ATGGTCCTGG AGCATCCAGC | 840 |
| CCGGGTGGAG CCTTTCTATG CTGTCCTGGA AAGCCCCAGC TTCTCTCTGA TGGGCATCCT | 900 |
| GCTGCGGATC GCCAGTGGGA CTCGCCTCTC CATCCCCATC ACTTCCAACA CATTGATCTA | 960 |
| TTATCACCCC CACCCCGAAG ATATTAAGTT CCACTTGTAC CTTGTCCCCA GCGACGCCTT | 1020 |
| GCTAACAAAG GCGATAGATG ATGAGGAAGA TCGCTTCCAT GGTGTGCGCC TGCAGACTTC | 1080 |
| GCCCCCAATG GAACCCCTGA ACTTTGGTTC CAGTTATATT GTGTCTAATT CTGCTAACCT | 1140 |
| GAAAGTAATG CCCAAGGAGT TGAAATTGTC CTACAGGAGC CCTGGAGAAA TTCAGCACTT | 1200 |
| CTCAAAATTC TATGCTGGGC AGATGAAGGA ACCCATTCAA CTTGAGATTA CTGAAAAAAG | 1260 |
| ACATGGGACT TTGGTGTGGG ATACTGAGGT GAAGCCAGTG GATCTCCAGC TTGTAGCTGC | 1320 |
| ATCAGCCCCT CCTCCTTTCT CAGGTGCAGC CTTTGTGAAG GAGAACCACC GGCAACTCCA | 1380 |
| AGCCAGGATG GGGGACCTGA AAGGGGTGCT CGATGATCTC CAGGACAATG AGGTTCTTAC | 1440 |
| TGAGAATGAG AAGGAGCTGG TGGAGCAGGA AAAGACACGG CAGAGCAAGA ATGAGGCCTT | 1500 |
| GCTGAGCATG GTGGAGAAGA AAGGGGACCT GGCCCTGGAC GTGCTCTTCA GAAGCATTAG | 1560 |
| TGAAAGGGAC CCTTACCTCG TGTCCTATCT TAGACAGCAG AATTTGTAAA ATGAGTCAGT | 1620 |
| TAGGTAGTCT GGAAGAGAGA ATCCAGCGTT CTCATTGGAA ATGGATAAAC AGAAATGTGA | 1680 |
| TCATTGATTT CAGTGTTCAA GACAGAAGAA GACTGGGTAA CATCTATCAC ACAGGCTTTC | 1740 |
| AGGACAGACT TGTAACCTGG CATGTACCTA TTGACTGTAT CCTCATGCAT TTTCCTCAAG | 1800 |
| AATGTCTGAA GAAGGTAGTA ATATTCCTTT TAAATTTTTT CCAACCATTG CTTGATATAT | 1860 |
| CACTATTTTA TCCATTGACA TGATTCTTGA AGACCCAGGA TAAAGGACAT CCGGATAGGT | 1920 |
| GTGTTTATGA AGGATGGGGC CTGGAAAGGC AACTTTTCCT GATTAATGTG AAAAATAATT | 1980 |
| CCTATGGACA CTCCGTTTGA AGTATCACCT TCTCATAACT AAAAGCAGAA AAGCTAACAA | 2040 |
| AAGCTTCTCA GCTGAGGACA CTCAAGGCAT ACATGATGAC AGTCTTTTTT TTTTTTGTAT | 2100 |
| GTTAGGACTT TAACACTTTA TCTATGGCTA CTGTTATTAG AACAATGTAA ATGTATTTGC | 2160 |
| TGAAAGAGAG CACAAAAATG GGAGAAAATG CAAACATGAG CAGAAAATAT TTTCCCACTG | 2220 |
| GTGTGTAGCC TGCTACAAGG AGTTGTTGGG TTAAATGTTC ATGGTCAACT CCAAGGAATA | 2280 |
| CTGAGATGAA ATGTGGTAAA TCAACTCCAC AGAACCACCA AAAAGAAAAT GAGGGTAATT | 2340 |
| CAGCTTATTC TGAGACAGAC ATTCCTGGCA ATGTACCATA CAAAAAATAA GCCAACTCTG | 2400 |
| ACATTTGGAT TCTACCATAG ACTCTGTCAT TTTGTAGCCA TTTCAGCTGT CTTTTGATTA | 2460 |
| ATGTTTTCGT GGCACACATA TTTCCATCCT TTTATGTTTA ATCTGTTTAA AACAAGTTCC | 2520 |
| TAGTAGACAC CATCTGGTTG AGTCAGTTTT TTTTATGGTG TATTTTGAAC CCATTCTGAT | 2580 |
| AGTCTCTTTT AACTGGAAGA TTTCAATTAC TTACGTTAAT GTAATTATTA ATATGTTAGG | 2640 |
| ATTTATCCTC AGTCAGCCAG TTTGTTATGT CTTTTCTATT CTACTGTTAT CACATTTGTA | 2700 |
| CCACTTAAAG TGGAATCTAG GCACTTTATC ACCATTTAGA TCCTATTACC TTTTCTCATC | 2760 |
| TAGGATATAG TTATCTTCTA CATAATCTTT CTGTATCTTA AAACCCATCA ATAAATTATT | 2820 |
| ATATATTTTC TACTTTTAAT CACTCAGAAG ATTTAAAAAA CTCATGAGAA GAGTAATCTG | 2880 |
| TTATGTTTTT CCAGATATTT ACCATTTCTG TTGCTCTTCC TTCATTATTT TCCAAATTTC | 2940 |
| GTTCTGCAAA TTTCCACTTC TTCTGATAGA CGTTTTTTAG TTCTTTTAGA GTGGTTCTGA | 3000 |
| TAGGTACAGA TTCTCTTATT TTTTGCTTCC TCTGAGGACA TCTTTTTCTC ACCTTCATTC | 3060 |
| TCAGTGATGT TTTTTGCTTG TAGTATTTTT AGTTGACATT GTTTTCTGTT CAGCAGTTTC | 3120 |
| CTTTTAGCTT CCGTATTTCC TGATGAGAAA TCTGCAGTCA TTCAAATTGT TGTTTCCCTG | 3180 |
| TATGTAGTGT GTCATTTTTC TGTCAGATTT CAAGGTATTT ATCTTTAGTT TTTAGCCATT | 3240 |
| TCATTATGTT GGGGATGAGT TTCCTTGTTT TATTCCCTTT GGAATTTGCT CCAATTCATA | 3300 |
| AATTTGCAGT TTTATGTCTT TTACCAAACT TAGAGGTTTT CAGCCTAATT TCTAAAAATA | 3360 |
| CTTTTATTA GCCTGATTTT CATCTTTATA GGAAATAGTT TAAGTGATGA CAAGTTCCAA | 3420 |
| TAGCTTATAT GCCCAGAAGG CCTTCAAAAT AAGAATTTTG AAAGAATACA GAAAACAAAC | 3480 |
| TTTTATATCC TTCTCATGTC TTCTACTGTA AAATTCATAT GCTTTGCTAC TCTAAACCTA | 3540 |
| GTTTGAAATC AACAGTCTTG AGAATAGATG AAAATTTTGA TGAATAGTGG AATTCTTTTA | 3600 |
| AATGGAAACC TCTTACATGT GATTTTCCTT GCCATCTAGA AATAAACCAT AGTATTTATG | 3660 |
| TTGAATCAAT CAATATTATA TTTTGTTTTT TTCCTCCTCT TCTGAGACTC TTATTGTGGA | 3720 |
| AATGTTAGAC TTTTATGTTT TCCTAAATGT CCCTGATATT CTACTTATTT AGAACATCTT | 3780 |
| TTCATTTTTT CCATTATTCT GATTGGGTAA TTTTAATTTG TCTATTTTCA AATTTGCTGG | 3840 |
| AGTGTTCACC TGTTGTTGTC TGTGTCGTCC CACTGAGTGC ATTCACCACC TTTTAAATTT | 3900 |
| TGGTCACTGT ATGTATCAGT TCTAAAATTT CCATTTTGTT CTCTATATTT TAAATTTCTT | 3960 |
| GGCTTATATT CTATTTTCCT GCAAATGTGT CAGCATTTGC TTGTTTGAGC TTTTTTTTTT | 4020 |
| TCAAGACAGG GTCTCAACTC TGTTACCCAG GCTGGAGTGC AGTGGTGCGA TCTCAGCTCA | 4080 |
| CTGCAACCTC TGCCTCCTGG TTCAAGCGAT TATTGTGCCT CAGCCTCCTG AGTAGCTGGG | 4140 |
| ATTACAGGCA TGCACCACCA CAGCCCAGCT AATTTTTTGT ATTTTTAGTA GAGACAGAGT | 4200 |
| TTTGCTATGT TGGCCAGGCT GGTTTTGAAC TCCTGGCCTC AAGTGATCCA CCGACCTCAG | 4260 |
| CCTCCCAAAG TGCTGGGATT ACAGGCCACT ACACCTGGCA CATTTGAGTA TTTTTTTTTT | 4320 |
| TTTTTTTTTT TTGAGATGGA GTCTCGCTCT GTCATCTAGG CTGGAGTGCA GTGGTGTGAT | 4380 |
| CTCAGCTCAC TGCAGCCTCT GTCTCCCGGG CTCAAGCGAT TCTCTTGCCT CAGCCTCCTG | 4440 |
| AGTAGCTAGG ACTACAGGTG CATGCCAACA CGCCCGGCTA ATTTTTTTAA AAAATATTTT | 4500 |
| TAGTAGAGAC AGGGTTTCAC CATTTTGGCC AGGATGGTCT CGATCTCCTG ACCTCATGAT | 4560 |
| CCACCCGCCT CGGCCTTCCA AAGTGCTGGG ATTACAGGCA TGAGCCACCG TGCCTGGCCT | 4620 |
| CATTTGAGTA TTTTTATAAT GTCTCTTTTA AAGTCTTTGT CAGATAATTC CACTGTACAT | 4680 |
| GTTATTCAGT GTTTGGTGTC CACTGAGTTG TCATTTGCCA GACAAGTGGA GATTTTTGCA | 4740 |
| GCTCATCCTT GTATTCTCAG TAGTTCCGAT ATGTACCCTC GACATGTGAA TGTTATCTTA | 4800 |
| TGAGACTCTG TTTTATTTGT ATCCAACAGA AGATGTTTAT TATTTATTTG GCTTTCTGTG | 4860 |
| AACTGAGGTC TTAATATCAG CTCATTTTAA AAGTCTTTGC AGTGGTATTC GGATCTATCC | 4920 |
| TGTGTGTGCC TATGAGATTG GGTGCAGTGT ATCCTGTTAG CTCCATTCTC AGGGCGTTTG | 4980 |
| AATGTGAATT AGGACCAGCG CAATGAATGC TCAAGTTGGG GTTGGGCGTT AGAATTCATA | 5040 |
| AAAGTCTTTA TATGCTCAG | |

ACF6 DNA sequence
Gene name: Homo sapiens cDNA FLJ10669 fis, clone NT2RP2006275, weakly similar to
Microtubule-associated protein 1B [CONTAINS: LIGHT CHAIN LC1]
Unigene number: Hs.66048
Probeset Accession #: AA609717
Nucleic Acid Accession #: AK001531
Coding sequence: 176-2194 (predicted start/stop codons underlined)

| | |
|---|---|
| CATCTCCCCC AACCTGGGGG TCGTGTTCTT CAACGCCTGC GAGGCCGCGT CGCGGCTGGC | 60 |
| GCGCGGCGAG GATGAGGCGG AGCTGGCGCT GAGCCTCCTG GCGCAGCTGG GCATCACGCC | 120 |
| TCTGCCACTC AGCCGCGGCC CCGTGCCAGC CAAACCCACC GTGCTCTTCG AGAAGATGGG | 180 |
| CGTGGGCCGG CTGGACATGT ATGTGCTGCA CCCGCCCTCC GCCGGCGCCG AGCGCACGCT | 240 |
| GGCCTCTGTG TGCGCCCTGC TGGTGTGGCA CCCCGCCGGC CCCGGCGAGA AGGTGGTGCG | 300 |
| CGTGCTGTTC CCCGGTTGCA CCCCGCCCGC CTGCCTCCTG GACGGCCTGG TCCGCCTGCA | 360 |
| GCACTTGAGG TTCCTGCGAG AGCCCGTGGT GACGCCCCAG GACCTGGAGG GGCCGGGGCG | 420 |
| AGCCGAGAGC AAAGAGAGCG TGGGCTCCCG GGACAGCTCG AAGAGAGAGG GCCTCCTGGC | 480 |
| CACCCACCCT AGACCTGGCC AGGAGCGCCC TGGGGTGGCC CGCAAGGAGC CAGCACGGGC | 540 |
| TGAGGCCCCA CGCAAGACTG AGAAAGAAGC CAAGACCCCC CGGGAGTTGA AGAAAGACCC | 600 |
| CAAACCGAGT GTCTCCCGGA CCCAGCCGCG GGAGGTGCGC CGGGCAGCCT CTTCTGTGCC | 660 |
| CAACCTCAAG AAGACGAATG CCCAGGCGGC ACCCAAGCCC CGCAAAGCGC CCAGCACGTC | 720 |
| CCACTCTGGC TTCCCGCCGG TGGCAAATGG ACCCCGCAGC CCGCCCAGCC TCCGATGTGG | 780 |
| AGAAGCCAGC CCCCCCAGTG CAGCCTGCGG CTCTCCGGCC TCCCAGCTGG TGGCCACGCC | 840 |
| CAGCCTGGAG CTGGGGCCGA TCCCAGCCGG GGAGGAGAAG GCACTGGAGC TGCCTTTGGC | 900 |
| CGCCAGCTCA ATCCCAAGGC CACGCACACC CTCCCCTGAG TCCCACCGGA GCCCCGCAGA | 960 |
| GGGCAGCGAG CGGCTGTCGC TGAGCCCACT GCGGGGCGGG GAGGCCGGGC CAGACGCCTC | 1020 |
| ACCCACAGTG ACCACACCCA CGGTGACCAC GCCCTCACTA CCCGCAGAGG TGGGCTCCCC | 1080 |
| GCACTCGACC GAGGTGGACG AGTCCCTGTC GGTGTCCTTT GAGCAGGTGC TGCCGCCATC | 1140 |
| CGCCCCCACC AGTGAGGCTG GGCTGAGCCT CCCGCTGCGT GGCCCCCGGG CGCGGCGCTC | 1200 |
| GGCTTCCCCA CACGATGTGG ACCTGTGCCT GGTGTCACCC TGTGAATTTG AGCATCGCAA | 1260 |
| GGCGGTGCCA ATGGCACCGG CACCTGCGTC CCCCGGCAGC TCGAATGACA GCAGTGCCCG | 1320 |
| GTCACAGGAA CGGGCAGGTG GGCTGGGGGC CGAGGAGACG CCACCCACAT CGGTCAGCGA | 1380 |
| GTCCCTGCCC ACCCTGTCTG ACTCGGATCC CGTGCCCCTG GCCCCCGGTG CGGCAGACTC | 1440 |
| AGACGAAGAC ACAGAGGGCT TTGGAGTCCC TCGCCACGAC CCTTTGCCTG ACCCCCTCAA | 1500 |
| GGTCCCCCCA CCACTGCCTG ACCCATCCAG CATCTGCATG GTGGACCCCG AGATGCTGCC | 1560 |
| CCCCAAGACA GCACGGCAAA CGGAGAACGT CAGCCGCACC CGGAAGCCCC TGGCCCGCCC | 1620 |
| CAACTCACGC GCTGCCGCCC CCAAAGCCAC TCCAGTGGCT GCTGCCAAAA CCAAGGGGCT | 1680 |
| TGCTGGTGGG GACCGTGCCA GCCCACCACT CAGTGCCCGG AGTGAGCCCA GTGAGAAGGG | 1740 |
| AGGCCGGGCA CCCCTGTCCA GAAAGTCCTC AACCCCCAAG ACTGCCACTC GAGGCCCGTC | 1800 |
| GGGGTCAGCC AGCAGCCGGC CCGGGGTGTC AGCCACCCCA CCCAAGTCCC CGGTCTACCT | 1860 |
| GGACCTGGCC TACCTGCCCA GCGGGAGCAG CGCCCACCTG GTGGATGAGG AGTTCTTCCA | 1920 |
| GCGCGTGCGC GCGCTCTGCT ACGTCATCAG TGGCCAGGAC CAGCGCAAGG AGGAAGGCAT | 1980 |
| GCGGGCCGTC CTGGACGCGC TACTGGCCAG CAAGCAGCAT TGGGACCGTG ACCTGCAGGT | 2040 |
| GACCCTGATC CCCACTTTCG ACTCGGTGGC CATGCATACG TGGTACGCAG AGACGCACGC | 2100 |
| CCGGCACCAG GCGCTGGGCA TCACGGTGTT GGGCAGCAAC GGCATGGTGT CCATGCAGGA | 2160 |
| TGACGCCTTC CCGGCCTGCA AGGTGGAGTT CTAGCCCCAT CGCCGACACG CCCCCCACTC | 2220 |
| AGCCCAGCCC GCCTGTCCCT AGATTCAGCC ACATCAGAAA TAAACTGTGA CTACACTTG | |

TABLE 2

AAA4 Protein sequence:
Gene name: CGI-100 protein
tlnigene number: Hs.275253
Probeset Accession #: AA089688
Protein Accession #: NP_057124
Signal sequence: predicted 1-23 (first underlined sequence)
Transmembrane Domain: predicted 201-217 (second underlined sequence)
emp24/gp25L/p24 domain: predicted 13-227
Summary: gp25L/emp24/p24 protein family members of the cis-Golgi network
bind both COP I and II coatomer. Members of this family are implicated
in bringing cargo forward from the ER and binding to coat proteins by
their cytoplasmic domains.

| | |
|---|---|
| MGDKIWLPFP VLLLAALPPV LLPGAAGFTP SLDSDFTFTL PAGQKECFYQ PMPLKASLEI | 60 |
| EYQVLDGAGL DIDFHLASPE GKTLVFEQRK SDGVHTVETE VGDYMFCFDN TFSTISEKVI | 120 |
| FFELILDNMG EQAQEQEDWK KYITGTDILD MKLEDILESI NSIKSRLSKS GHIQTLLRAF | 180 |
| EARDRNIQES NFDRVNFWSM VNLVVMVVVS AIQVYMLKSL FEDKRKSRT | |

AAA7 Protein sequence:
Gene name: Endothelial differentiation, sphingolipid G-protein-coupled
receptor, 1
(EDG1)
Unigene number: Hs.154218
Probeset Accession #: M31210
Protein Accession #: NP_001391
7 Transmembrane Domains: predicted 50-71, 92-110, 122-140, 160-177,
201-222, 251-269, 281-301 (underlined sequences)
Summary: Endothelial differentiation, sphingolipid G-protein-coupled
receptor, 1 may regulate the differentiation of endothelial cells. It
binds the sphingolipid metabolite, sphingosine-1-phosphate, which may
function as a second messenger in cell proliferation and survival.

| | |
|---|---|
| MGPTSVPLVK AHRSSVSDYV NYDIIVRHYN YTGKLNISAD KENSIKLTSV VFILICCFII | 60 |
| LENIFVLLTI WKTKKFHRPM YYFIGNLALS DLLAGVAYTA NLLLSGATTY KLTPAQWFLR | 120 |
| EGSMFVALSA SVFSLLAIAI ERYITMLKMK LHNGSNNFRL FLLISACWVI SLILGGLPIM | 180 |
| GWNCISALSS CSTVLPLYHK HYILFCTTVF TLLLLSIVIL YCRIYSLVRT RSRRLTFRKN | 240 |
| ISKASRSSEN VALLKTVIIV LSVFIACWAP LFILLLLDVG CKVKTCDILF RAEYFLVLAV | 300 |
| LNSGTNPIIY TLTNKEMRRA FIRIMSCCKC PSGDSAGKFK RPIIAGMEFS RSKSDNSSHP | 360 |
| QKDEGDNPET IMSSGNYNSS S | |

AAB3 Protein sequence:
Gene name: Solute carrier family 20 (phosphate transporter), member 1,
Human leukaemia virus receptor 1 (GLVR1)
Unigene number: Hs.78452
Probeset Accession #: L20859
Protein Accession #: NP_005406
Transmembrane domains: predicted 24-40, 62-78, 164-180, 198-214, 232-
248, 513-529, 562-578, 604-620, 655-671
Cellular Localization: Likely a Type IIIa membrane protein (Ncyt Cexo)

| | |
|---|---|
| MATLITSTTA ATAASGPLVD YLWMLILGFI IAFVLAFSVG ANDVANSFGT AVGSGVVTLK | 60 |
| QACILASIFE TVGSVLLGAK VSETIRKGLI DVEMYNSTQG LLMAGSVSAM FGSAVWQLVA | 120 |
| SFLKLPISGT HCIVGATIGF SLVAKGQEGV KWSELIKIVM SWFVSPLLSG IMSGILFFLV | 180 |
| RAFILHKADP VPNGLRALPV FYACTVGINL FSIMYTGAPL LGFDKLPLWG TILISVGCAV | 240 |
| FCALIVWFFV CPRMKRKIER EIKCSPSESP LMEKKNSLKE DHEETKLSVG DIENKHPVSE | 300 |
| VGPATVPLQA VVEERTVSFK LGDLEEAPER ERLPSVDLKE ETSIDSTVNG AVQLPNGNLV | 360 |
| QFSQAVSNQI NSSGHSQYHT VHKDSGLYKE LLHKLHLAKV GMGDSGDK PLRRNNSYTS | 420 |
| YTMAICGMPL DSFRAKEGEQ KGEEMEKLTW PNADSKKRIR MDYTSYCNA VSDLHSASEI | 480 |
| DMSVKAAMGL GDRKGSNGSL EEWYDQDKPE VSLLFQFLQI LTACFGSFAH GGNDVSNAIG | 540 |
| PLVALYLVYD TGDVSSKVAT PIWLLLYGGV GICVGLWVWG RRVIQTMGKD LTPITPSSGF | 600 |
| SIELASALTV VIASNIGLPI STTHCKVGSV VSVGWLRSKK AVDWRLFRNI FMAWFVTVPI | 660 |
| SGVISAAIMA IFRYVILRM | |

AAB4 Protein sequence:
Gene name: Matrix metalloproteinase 10 (stromelysin 2)
Unigene number: Hs.2258
Probeset Accession #: X07820
Protein Accession #: NP_002416
Signal sequence: predicted 1-17 (underlined sequence)
Cellular Localization: predicted secreted

| | |
|---|---|
| MMHLAFLVLL CLPVCSAYPL SGAAKEEDSN KDLAQQYLEK YYNLEKDVKQ FRRKDSNLIV | 60 |
| KKIQGMQKFL GLEVTGKLDT DTLEVMRKPR CGVPDVGHFS SFPGMPKWRK THLTYRIVNY | 120 |
| TPDLPRDAVD SAIEKALKVW EEVTPLTFSR LYEGEADIMI SFAVKEHGDF YSFDGPGHSL | 180 |
| AHAYPPGPGL YGDIHFDDDE KWTEDASGTN LFLVAAHELG HSLGLFHSAN TEALMYPLYN | 240 |
| SFTELAQFRL SQDDVNGIQS LYGPPPASTE EPLVPTKSVP SGSEMPAKCD PALSFDAIST | 300 |
| LRGEYLFFKD RYFWRRSHWN PEPEFHLISA FWPSLPSYLD AAYEVNSRDT VFIFKGNEFW | 360 |
| AIRGNEVQAG YPRGIHTLGF PPTIRKIDAA VSDKEKKKTY FFAADKYWRF DENSQSMEQG | 420 |
| FPRLIADDFP GVEPKVDAVL QAFGFFYFFS GSSQFEFDPN ARMVTHILKS NSWLHC | |

AAB6 Protein sequence:
Gene name: Podocalyxin-like
Unigene number: Hs.16426
Probeset Accession #: U97519
Protein Accession #: NP_005388
Transmembrane domain: predicted 432-448 (underlined sequence)
Cellular Localization: predicted Type Ia membrane protein (Nexo)

| | |
|---|---|
| MRCALALSAL LLLLSTPPLL PSSPSPSPSP SPSQNATQTT TDSSNKTAPT PASSVTIMAT | 60 |
| DTAQQSTVPT SKANEILASV KATTLGVSSD SPGTTTLAQQ VSGPVNTTVA RGGGSGNPTT | 120 |
| TIESPKSTKS ADTTTVATST ATAKPNTTSS QNGAEDTTNS GGKSSHSVTT DLTSTKAEHL | 180 |
| TTPHPTSPLS PRQPTLTHPV ATPTSSGHDH LMKISSSSST VAIPGYTFTS PGMTTTLPSS | 240 |
| VISQRTQQTS SQMPASSTAP SSQETVQPTS PATALRTPTL PETMSSSPTA ASTTHRYPKT | 300 |
| PSPTVAHESN WAKCEDLETQ TQSEKQLVLN LTGNTLCAGG ASDEKLISLI CRAVKATFNP | 360 |
| AQDKCGIRLA SVPGSQTVVV KEITIHTKLP AKDVYERLKD KWDELKEAGV SDMKLGDQGP | 420 |
| PEEAEDRFSM PLIITIVCMA SFLLLVAALY GCCHQRLSQR KDQQRLTEEL QTVENGYHDN | 480 |
| PTLEVMETSS EMQEKKVVSL NGELGDSWIV PLDNLTKDDL DEEEDTHL | |

AAB8 Protein sequence:
Gene name: EGF-containing fibulin-like extracellular matrix protein 1
Unigene number: Hs.76224
Probeset Accession #: U03877
Protein Accession #: NP_004096 Variant 1
Signal sequence: predicted 1-17 (underlined sequence)
Summary: This gene spans approximately 18 kb of genomic DNA and consists
of 12 exons. Two transcripts with distinct 5′ UTR have been des-
cribed; the resulting proteins have distinct N-terminal amino acid se-
quences. Translation initiation from internal methionine residues was
observed with in vitro translation. A signal peptide sequence is pre-
dicted for translation initiation sites 1, 2, and 4. The protein iso-
forms contain 5 or 6 calcium-binding EGF2 domains and 5 or 6 EGF2
domains. Mutations in this gene cause the retinal disease Malattia Leven-
tinese. Transcript Variant: This variant (1) has a distinct 5′ UTR and
N-terminal protein sequence as compared to variant 2.

| | |
|---|---|
| MLKALFLTML TLALVKSQDT EETITYTQCT DGYEWDPVRQ QCKDIDECDI VPDACKGGMK | 60 |
| CVNHYGGYLC LPKTAQIIVN NEQPQQETQP AEGTSGATTG VVAASSMATS GVLPGGGFVA | 120 |
| SAAAVAGPEM QTGRNNFVIR RNPADPQRIP SNPSHRIQCA AGYEQSEHNV CQDIDECTAG | 180 |
| THNCRADQVC INLRGSFACQ CPPGYQKRGE QCVDIDECTI PPYCHQRCVN TPGSFYCQCS | 240 |
| PGFQLAANNY TCVDINECDA SNQCAQQCYN ILGSFICQCN QGYELSSDRL NCEDIDECRT | 300 |
| SSYLCQYQCV NEPGKFSCMC PQGYQVVRSR TCQDINECET TNECREDEMC WNYHGGFRCY | 360 |
| PRNPCQDPYI LTPENRCVCP VSNAMCRELP QSIVYKYMSI RSDRSVPSDI FQIQATTIYA | 420 |
| NTINTFRIKS GNENGEFYLR QTSPVSAMLV LVKSLSGPRE HIVDLEMLTV SSIGTFRTSS | 480 |
| VLRLTIIVGP FSF | |

AAB9 Protein sequence:
Gene name: Melanoma adhesion molecule, MUC 18 glycoprotein
Unigene number: Hs.211579
Probeset Accession #: M28882
Protein Accession #: NP_006491
Signal sequence: predicted 1-17 (first underlined sequence)
Transmembrane domain: predicted 559-575 (second underlined sequence)
Cellular localization: predicted Type Ia membrane protein (Nexo)

| | |
|---|---|
| MGLPRLVCAF LLAACCCCPR VAGVPGEAEQ PAPELVEVEV GSTALLKCGL SQSQGNLSBV | 60 |
| DWFSVHKEKR TLIFRVRQGQ GQSEPGEYEQ RLSLQDRGAT LALTQVTPQD ERIFLCQGKR | 120 |
| PRSQEYRIQL RVYKAPEEPN IQVNPLGIPV NSKEPEEVAT CVGRNGYPIP QVIWYKNGRP | 180 |
| LKEEKNRVHI QSSQTVESSG LYTLQSILKA QLVKEDKDAQ FYCELNYRLP SGNHMKESRE | 240 |
| VTVPVFYPTE KVWLEVEPVG MLKEGDRVEI RCLADGNPPP HFSISKQNPS TREAEEETTN | 300 |
| DNGVLVLEPA RKEHSGRYEC QAWNLDTMIS LLSEPQELLV NYVSDVRVSP AAPERQEGSS | 360 |
| LTLTCEAESS QDLEFQWLRE ETDQVLERGP VLQLHDLKRE AGGGYRCVAS VPSIPGLNRT | 420 |
| QLVKLAIFGP PWMAFKERKV NVKENMVLNL SCEASGHPRP TISWNVNGTA SEQDQDPQRV | 480 |
| LSTLNVLVTP ELLETGVECT ASNDLGKNTS ILFLELVNLT TLTPDSNTTT GLSTSTASPH | 540 |
| TRANSTSTER KLPEPESRGV VIVAVIVCIL VLAVLGAVLY FLYKKGKLPC RRSGKQEITL | 600 |
| PPSRKTELVV EVKSDKLPEE MGLLQGSSGD KRAPGDQGEK YIDLRH | |

AAC1 Protein sequence:
Gene name: Matrix metalloproteinase 1 (interstitial collagenase)
Unigene number: Hs.83169
Probeset Accession #: X54925
Protein Accession #: NP_002412
Signal sequence: predicted 1-19 (underlined sequence)
Cellular localization: predicted secreted protein

| | |
|---|---|
| MHSFPPLLLL LFWGVVSIISF PATLETQEQD VDLVQKYLEK YYNLKNDGRQ VEKRRNSGPV | 60 |
| VEKLKQMQEF FGLKVTGKPD AETLKVMKQP RCGVPDVAQF VLTEGNPRWE QTHLTYRIEN | 120 |
| YTPDLPRADV DHAIEKAFQL WSNVTPLTFT KVSEGQADIM ISFVRGDHRD NSPFDGPGGN | 180 |
| LAHAFQPGPG IGGDAHFDED ERWTNNFREY NLHRVAAHEL GHSLGLSHST DIGALMYPST | 240 |
| TFSGDVQLAQ DDIDGIQAIY GRSQNPVQPI GPQTPKACDS KLTFDAITTI RGEVMFFKDR | 300 |
| FYMRTNPFYP EVELNFISVF WPQLPNGLEA AYEFADRDEV RFFKGNKYWA VQGQNVLHGY | 360 |
| PKDIYSSFGF PRTVKHIDAA LSEENTGKTY FFVANKYWRY DEYKRSMDPG YPKMIAHDFP | 420 |
| GIGHKVDAVF MKDGFFYFFH GTRQYKFDPK TKRILTLQKA NSWFNCRKN | |

AAC3 Protein sequence:
Gene name: Branched chain aminotransferase 1, cytosolic
Unigene number: Hs.157205
Probeset Accession #: AA423987
Protein Accession #: NP_005495
Cellular Localization: cytolasmic
Summary: The lack of the cytosolic enzyme branched-chain amino acid tran-
saminase (BCT) causes cell growth inhibition. There may be at least 2 dif-
ferent clinical disorders due to a defect of branched-chain amino acid
transamination: hypervalinemia and hyperleucine-isoleucinemia. Since
there are 2 distinct BCATS, mitochondrial and cytosolic, it is possible
that one is mutant in each of these 2 conditions.

| | |
|---|---|
| MDCSNGSAEC TGEGGSKEVV GTFKAKDLIV TPATILKEKP DPNNLVFGTV FTDHMLTVEW | 60 |
| SSEFGWEKPH IKPLQNLSLH PGSSALHYAV ELFEGLKAFR GVDNKIRLFQ PNLNMDRNYR | 120 |
| SAVRATLPVF DKEELLECIQ QLVKLDQEWV PYSTSASLYI RPAFIGTEPS LGVKXPTKAL | 180 |
| LFVLLSPVGP YFSSGTFNPV SLWANPKYVR AWKGGTGDCK MGGNYGSSLF AQCEDVDNGC | 240 |
| QQVLWLYGRD HQITEVGTMN LFLYWINEDG EEELATPPLD GIILPGVTRR CILDLAHQWG | 300 |
| EFKVSERYLT MDDLTTALEG NRVREMFSSG TACVVCPVSD ILYKGETIHI PTMENGPKLA | 360 |
| SRILSKLTDI QYGREESDWT IVLS | |

ACG4 Protein sequence:
Gene name: Pentaxin-related gene, rapidly induced by IL-1 beta
Unigene number: Hs.2050
Probeset Accession #: M31166
Protein Accession #: NP_002843
Signal sequence: predicted 1-17 (underlined sequence)
Cellular localization: predicted secreted
Summary: TNF-inducible member of hyaluronate binding protein family,
related to CD44

| | |
|---|---|
| MHLLAILFCA LWSAVLAENS DDYDLMYVNL DNEIDNGLHP TEDPTPCDCG QEHSEWDKLF | 60 |
| IMLENSQMRE RMLLQATDDV LRGELQRLRE ELGRLAESLA RPCAPGAPAE ARLTSALDEL | 120 |
| LQATRDAGRR LARMEGAEAQ RPEEAGRALA AVLEELRQTR ADLHAVQGWA ARSWLPAGCE | 180 |
| TAILFPMRSK KIFGSVHPVR PMRLESFSAC IWVKATDVLN KTILFSYGTK RNPYEIQLYL | 240 |
| SYQSIVFVVG GEENKLVAEA MVSLGRWTHL CGTWNSEEGL TSLWVNGELA ATTVEMATGH | 300 |
| IVPEGGILQI GQEKNGCCVG GGFDETLAFS GRLTGFNIWD SVLSNEEIRE TGGAESCHIR | 360 |
| GNIVGWGVTE IQPHGGAQYV S | |

ACK5 Protein sequence:
Gene name: Von Willebrand factor; Coagulation factor VIII
Unigene number: Hs.110802
Probeset Accession #: M10321
Protein Accession #: NP_000543
Signal peptide: predicted 1-22 (underlined sequence)
Cellular localization: predicted secreted

| | |
|---|---|
| MIPARFAGVL LALALILPGT LCAEGTRGRS STARCSLFGS DFVNTFDGSM YSFAGYCSYL | 60 |
| LAGGCQKRSF SIIGDFQNGK RVSLSVYLGE FFDIHLFVNG TVTQGDQRVS MPYASKGLYL | 120 |
| ETEAGYYKLS GEAYGFVARI DGSGNFQVLL SDRYFNKTCG LCGNFNIFAE DDFMTQEGTL | 180 |
| TSDPYDFANS WALSSGEQWC ERASPPSSSC NISSGEMQKG LWEQCQLLKS TSVFARCHPL | 240 |
| VDPEPFVALC EKTLCECAGG LECACPALLE YARTCAQEGM VLYGWTDHSA CSPVCPAGME | 300 |
| YRQCVSPCAR TCQSLHINEM CQERCVDGCS CPEGQLLDEG LCVESTECPC VHSGKRYPPG | 360 |
| TSLSRDCNTC ICRNSQWICS NEECPGECLV TGQSHFKSFD NRYFTFSGIC QYLLARDCQD | 420 |
| HSFSIVIETV QCADDRDAVC TRSVTVRLPG LHNSLVKLKH GAGVAMDGQD IQLPLLKGDL | 480 |
| RIQHTVTASV RLSYGEDLQM DWDGRGRLLV KLSPVYAGKT CGLCGNYNGN QGDDFLTPSG | 540 |
| LAEPRVEDFG NAWKLHGDCQ DLQKQHSDPC ALNPPMTRFS EEACAVLTSP TFEACHRAVS | 600 |
| PLPYLRNCRY DVCSCSDGRE CLCGALASYA AACAGRGVRV AWREPGRCEL NCPKGQVYLQ | 660 |
| CGTPCNLTCR SLSYPDEECN EACLEGCFCP PGLYMDERGD CVPKAQCPCY YDGEIFQPED | 720 |
| IFSDHHTMCY CEDGFMHCTM SGVPGSLLPD AVLSSPLSHR SKRSLSCRPP MVKLVCPADN | 780 |
| LRAEGLECTK TCQNYDLECM SMGCVSGCLC PPGMVRHENR CVALERCPCF NQGKEYAPGE | 840 |
| TVKIGCNTCV CPDRKWNCTD HVCDATCSTI GMAHYLTFDG LKYLFPGECQ YVLVQDYCGS | 900 |
| NPGTFRILVG NKGCSHPSVK CKKRVTILVE GGEIELFDGE VNVKRPMKDE THFEVVESGR | 960 |
| YIILLLGKAL SVVWDRHLSI SVVLKQTYQE KVCGLCGNFD GIQNNDLTSS NLQVEEDPVD | 1020 |
| FGNSWKVSSQ CADTRKVPLD SSPATCHNNI MKQTMVDSSC RILTSDVFQD CNKLVDPEPY | 1080 |
| LDVCIYDTCS CESIGDCACF CDTIAAYAHV CAQHGKVVTW RTATLCPQSC EERNLRENGY | 1140 |
| ECEWRYNSCA PACQVTCQHP EPLACPVQCV EGCHAHCPPG KILDELLQTC VDPEDCPVCE | 1200 |
| VAGRRFASGK KVTLNPSDPE HCQICHCDVV NLTCEACQEP GGLVVPPTDA PVSPTTLYVE | 1260 |
| DISEPPLHDF YCSRLLDLVF LLDGSSRLSE AEFEVLKAFV VDNMERLRIS QKNVRVAVVE | 1320 |
| YHDGSHAYIG LKDRKRPSEL RRIASQVKYA GSQVASTSEV LKYTLFQIFS KIDRPEASRI | 1380 |
| ALLLMASQEP QRMSRNFVRY VQGLKKKKVI VIPVGIGPHA NLKQIRLIEK QAPENKAFVL | 1440 |
| SSVDELEQQR DEIVSYLCDL APEAPPPTLP PHMAQVTVGP GLLGVSTLGP KRNSMVLDVA | 1500 |
| FVLEGSDKIG EADFNRSKEF MEEVIQRMDV GQDSIHVTVL QYSYMVTVEY PFSEAQSKGD | 1560 |
| ILQRVREIRY QGGNRTNTGL ALRYLSDHSF LVSQGDREQA PNLVYMVTGN PASDEIKRLP | 1620 |
| GDIQVVPIGV GPNANVQELE RIGWPNAPIL IQDFETLPRE APDLVLQRCC SGEGLQIPTL | 1680 |
| SPAPDCSQPL DVILLLDGSS SFPASYFDEM KSFAKAFISK ANIGPRLTQV SVLQYGSITT | 1740 |
| IDVPWNVVPE KAHLLSLVDV MQREGGPSQI GDALGFAVRY LTSEMHGARP GASKAVVILV | 1800 |
| TDVSVDSVDA AADAAPSNRV TVFPIGIGDR YDAAQLRILA GPAGDSNVVK LQRIEDLPTM | 1860 |
| VTLGMSFLHK LCSGFVRICM DEDGNEKRPG DVWTLPDQCH TVTCQPDGQT LLKSHRVNCD | 1920 |
| RGLRPSCPNS QSPVKVEETC GCRWTCPCVC TGSSTRHIVT FDGQNFKLTG SCSYVLFQNK | 1980 |
| EQDLEVILHN GACSPGARQG CMKSIEVKHS ALSVELHSDM EVTVNGRLVS VPYVGGNMEV | 2040 |
| NVYGAIMHEV RFNHLGHIFT FTPQNNEFQL QLSPKTFASK TYGLCGICDE NGANDFMLRD | 2100 |
| GTVTTDWKTL VQEWTVQRPG QTCQPILEEQ CLVPDSSHCQ VLLLPLFAEC HKVLAPATFY | 2160 |
| AICQQDSCHQ EQVCEVIASY AHLCRTNGVC VDWRTPDFCA MSCPPSLVYN HCEHGCPRHC | 2220 |
| DGNVSSCGDH PSEGCFCPPD KVMLEGSCVP EEACTQCIGE DGVQHQFLEA WVPDHQPCQI | 2280 |
| CTCLSGRKVN CTTQPCPTAK APTCGLCEVA RLRQNADQCC PEYECVCDPV SCDLPPVPHC | 2340 |
| ERGLQPTLTN PGECRPNFTC ACRKEECKRV SPPSCPPHRL PTLRKTQCCD EYECACNCVN | 2400 |
| STVSCPLGYL ASTATNDCGC TTTTCLPDKV CVHRSTIYPV GQFWEEGCDV CTCTDMEDAV | 2460 |
| NGLRVAQCSQ KPCEDSCRSG FTYVLHEGEC CGRCLPSACE VVTGSPRGDS QSSWKSVGSQ | 2520 |
| WASPENPCLI NECVRVKEEV FIQQRNVSCP QLEVPVCPSG FQLSCKTSAC CPSCRCERME | 2580 |
| ACMLNGTVIG PGKTVMIDVC TTCRCMVQVG ISGFKLECR KTTCNPCPLG YKEENNTGEC | 2640 |
| CGRCLPTACT IQLRGGQIMT LKRDETLQDG CDTHFCKVNE RGEYFWEKRV TGCPPFDEHK | 2700 |
| CLAEGGKIMK IPGTCCDTCE EPECNDITAR LQYVKVGSCK SEVEVDIHYC QGKCASKAYIY | 2760 |
| SIDINDVQDQ CSCCSPTRTE PMQVALHCTN GSVVYHEVLN AMECKCSPRK CSK | |

AAC7 protein sequence:
Gene name: KIAA1294 protein
Probeset Accession #: AA432248
Protein Accession #: BAA92532
Cellular localization: predicted nuclear protein
PFAM prediction: 22-153 Band 41 domain (underlined seq). A number of
cytoskeletal-associated proteins that associate with various proteins
at the interface between the plasma membrane and the cytoskeleton con-
tain a conserved N-terminal domain of about 150 amino-acid residues.

| | |
|---|---|
| MAVQLVPDSA LGLLMMTEGR RCQVHLLDDR KLELLVQPKL LAKELLDLVA SHFNLKEKEY | 60 |
| FGIAFTDETG HLNWLQLDRR VLEHDFPKKS GPVVLYFCVR FYIESISYLK DNATIELFFL | 120 |
| NAKSCIYKEL IDVDSEVVFE LASYILQEAK GDFSSNEVVR SDLKKLPALP TQALKEHPSL | 180 |
| AYCEDRVIEH YKKLNGQTRG QAIVNYMSIV ESLPTYGVHY YAVKDKQGIP WWLGLSYKGI | 240 |
| FQYDYHDKVK PRKIFQWRQL ENLYFREKKF SVEVHDPRRA SVTRRTFGHS GIAVHTWYAC | 300 |
| PALIKSIWAM AISQHQFYLD RKQSKSKIHA ARSLSEIAID LTETGTLKTS KLCLMGSKGK | 360 |
| IISGSSGSLL SSGSQESDSS QSAKKDMLAA LKSRQEALEE TLRQRLEELK KLCLREAELT | 420 |
| GKLPVEYPLD PGEEPPIVRR RIGTAFKLDE QKILPKGEEA ELERLEREFA IQSQITEAAR | 480 |
| RLASDPNVSK KLKKQRKTSY LNALKKLQEI ENAINENRIK SGKKPTQRAS LIIDDGNIAS | 540 |
| EDSSLSDALV LEDEDSQVTS TISPLHSPHK GLPPRPPSHN RPPPPQSLEG LRQMHYHRND | 600 |
| YDKSPIKPKM WSESSLDEPY EKVKKRSSHS HSSSHKRFPS TGSCAEAGGG SNSLQNSPIR | 660 |
| GLPHWNSQSS MPSTPDLRVR SPHYVHSTRS VDISPTRLHS LALHFRHRSS SLESQGKLLG | 720 |
| SENDTGSPDF YTPRTRSSNG SDPMDDCSSC TSHSSSEHYY PAQMNANYST LAEDSPSKAR | 780 |
| QRQRQRQRAA GALGSASSGS MPNLAARGGA GGAGGAGGGV YLHSQSQPSS QYRIKEYPLY | 840 |
| IEGGATPVVV RSLESDQECH YSVKAQFKTS NSYTAGGLFK ESWRGGGGDE GDTGRLTPSR | 900 |
| SQILRTPSLG REGAHDKGAG RAAVSDELRQ WYQRSTASHK EHSRLSHTSS TSSDSGSQYS | 960 |
| TSSQSTFVAH SRVTRMPQMC KATSAALPQS QRSSTPSSEI GATPPSSPHH ILTWQTGEAT | 1020 |
| ENSPILDGSE SPPHQSTDE | |

ACG8 Protein sequence:
Gene name: ubiquitin E3 ligase SMURF2
Unigene number: Hs.21806 (3′UTR only)
Probeset Accession #: AA398243
Protein Accession #: AF301463_1
Cellular Localization: predicted cytoplasmic
Summary: Smurf 2 Is a Ubiquitin E3 Ligase Mediating Proteasome-dependent
Degradation of Smad2 in Transforming Growth Factor-beta Signaling

| | |
|---|---|
| MSNPGGRRNG PVKLRLTVLC AKNLVKKDFF RLPDPFAKVV VDGSGQCHST DTVKNTLDPK | 60 |
| WNQHYDLYIG KSDSVTISVW NHKKIHKKQG AGFLGCVRLL SNAINRLKDT GYQRLDLCKL | 120 |
| GPNDNDTVRG QIVVSLQSRD RIGTGGQVVD CSRLFDNDLP DGWEERRTAS GRIQYLNHIT | 180 |
| RTTQWERPTR PASEYSSPGR PLSCFVDENT PISGTNGATC GQSSDPRLAE RRVRSQRHRN | 240 |
| YMSRTHLHTP PDLPEGYEQR TTQQGQVYFL HTQTGVSTWH DPRVPRDLSN INCEELGPLP | 300 |
| PGWEIRNTAT GRVYFVDHNN RTTQFTDPRL SANLHLVLNR QNQLKDQQQQ QVVSLCPDDT | 360 |
| ECLTVPRYKR DLVQKLKILR QELSQQQPQA GHCRIEVSRE EIFEESYRQV MKMRPKDLWK | 420 |
| RLMIKFRGEE GLDYGGVARE WLYLLSHEML NPYYGLFQYS RDDIYTLQIN PDSAVNPEHL | 480 |
| SYFHFVGRIM GMAVFHGEYI DGGFTLPFYK QLLGKSITLD DMELVDPDLH NSLVWILEND | 540 |
| ITGVLDHTFC VEHNAYGEII QHELKPNGKS IPVNEENKKE YVRLYVNWRF LRGIEAQFLA | 600 |
| LQKGFNEVIP QHLLKTFDEK ELELIICGLG KIDVNDWKVN TRLKECTPDS NIVKWFWKAV | 660 |
| EFFDEERRAR LLQFVTGSSR VPLQGFKALQ GAAGPRLFTI HQIDACTNNL PKAHTCFNRI | 720 |
| DIPPYESYEK LYEKLLTAIE ETCGFAVE | |

ACE1 Protein sequence:
Gene name: EST
Unigene number: Hs.30089
Probeset Accession #: AA410480
CAT cluster#: cluster 96816_1
Summary: predicted open reading frame

| | |
|---|---|
| PLWTEPPLSC CLPATYPADR GPAEPCSCAG VILGFLLFRG HNSQPTMTQT SSSQGGLGGL | 60 |
| SLTTEPVSSN PGYIPSSEAN RPSHLSSTGT PGAGVPSSGR DGGTSRDTFQ TTPPNSTTMS | 120 |
| LSMREDATIL PSPTSETVLT VAAFGVISFI VILVVVVIIL VGVVSLRFKC RKSKESGDPQ | 180 |
| KPGEREEKVG HRREPYPWN | |

ACJ2 Protein sequence:
Gene name: Complement component Clq receptor
Unigene number: Hs.97199
Probeset Accession #: AA487558
Protein Accession #: NP_036204
Signal sequence: 1-17 (first underlined sequence)
Transmemrane domain: 589-605 (second underlined sequence)
Cellular localization: This gene encodes a predicted type I membrane
protein. Summary: This protein acts as a receptor for complement pro-
tein Clq, mannose-binding lectin, and pulmonary surfactant protein A.
This protein is a functional receptor involved in ligand-mediated
enhancement of phagocytosis.

| | |
|---|---|
| MATSMGLLLL LLLLLTQPGA GTGADTEAVV CVGTACYTAH SGKLSAAEAQ NHCNQNGGNL | 60 |
| ATVKSKEEAQ HVQRVLAQLL RREAALTARM SKFWIGLQRE KGKCLDPSLP LKGFSWVGGG | 120 |
| EDTPYSNWHK ELRNSCISKR CVSLLLDLSQ PLLPNRLPKW SEGPCGSPGS PGSNIEGFVC | 180 |
| KFSFKGMCRP LALGGPGQVT YTTPFQTTSS SLEAVPFASA ANVACGEGDK DETQSHYFLC | 240 |
| KEKAPDVFDW GSSGPLCVSP KYGCNFNNGG CHQDCFEGGD GSFLCGCRPG FRLLDDLVTC | 300 |
| ASRNPCSSSP CRGGATCVLG PHGKNYTCRC PQGYQLDSSQ LDCVDVDECQ DSPCAQECVN | 360 |
| TPGGFRCECW VGYEPGGPGE GACQDVDECA LGRSPCAQGC TNTDGSFHCS CEEGYVLAGE | 420 |
| DGTQCQDVDE CVGPGGPLCD SLCFNTQGSF HCGCLPGWVL APNGVSCTMG PVSLGPPSGP | 480 |
| PDEEDKGEKE GSTVPRAATA SPTRGPEGTP KATPTTSRPS LSSDAPITSA PLKMLAPSGS | 540 |
| SGVWREPSIH HATAASGPQE PAGGDSSVAT QNNDGTDGQK LLLFYILGTV VAILLLLALA | 600 |
| LGLLVYRKRR AKREEKKEKK PQNAADSYSW VPERAESRAM ENQYSPTPGT DC | |

ACJ3 Protein sequence:
Gene name: FLT1/vascular endothelial growth factor receptor
Unigene number: Hs.138671
Probeset Accession #: AA047437
Transmettlbrane domain: predicted 764-780 (underlined sequence)
Cellular Localization: predicted cell surface tyrosine kinase

| | |
|---|---|
| MVSYWDTGVL LCALLSCLLL TGSSSGSKLK DPELSLKGTQ HIMQAGQTLH LQCRGEAAHK | 60 |
| WSLPEMVSKE SERLSITKSA CGRNGKQFCS TLTLNTAQAN HTGFYSCKYL AVPTSKKKET | 120 |
| ESAIYIFISD TGRPFVEMYS EIPEIIHMTE GRELVIPCRV TSPNITVTLK KFPLDTLIPD | 180 |
| GKRIIWDSRK GFIISNATYK EIGLLTCEAT VNGHLYKTNY LTHRQTNTII DVQISTPRPV | 240 |
| KLLRGHTLVL NCTATTPLNT RVQMTWSYPD EKNKRASVRR RIDQSNSHAN IFYSVLTIDK | 300 |
| MQNKDKGLYT CRVRSGPSFK SVNTSVHIYD KAFITVKHRK QQVLETVAGK RSYRLSMKVK | 360 |
| AFPSPEVVWL KDGLPATEKS ARYLTRGYSL IIKDVTEEDA GNYTILLSIK QSNVFKNLTA | 420 |
| TLIVNVKPQI YEKAVSSFPD PALYPLGSRQ ILTCTAYGIP QPTIKWFWHP CNHNHSEARC | 480 |
| DFCSNNEESF ILDADSNMGN RIESITQRMA IIEGKNKMAS TLVVADSRIS GIYICIASNK | 540 |
| VGTVGRNISF YITDVPNGFH VNLEKMPTEG EDLKLSCTVN KFLYRDVTWI LLRTVNNRTM | 600 |
| HYSISKQKMA ITKEHSITLN LTIMNVSLQD SGTYACRARN VYTGEEILQK KEITIRDQEA | 660 |
| PYLLRNLSDH TVAISSSTTL DCHANGVPEP QITWFKNNHK IQQEPGIILG PGSSTLFIER | 720 |
| VTEEDEGVYH CKATNQKGSV ESSAYLTVQG TSDKSNLELI TLTCTCVAAT LFWLLLTLLI | 780 |
| RKMKRSSSEI KTDYLSIIMD PDEVPLDEQC ERLPYDASKW EFARERLKLG KSLGRGAFGK | 840 |
| VVQASAFGIK KSPTCRTVAV KMLKEGATAS EYKALMTELK ILTHIGHHLN VVNLLGACTK | 900 |
| QGGPLMVIVE YCKYGNLSNY LKSKRDLFFL NKDAALHMEP KKEKMEPGLE QGKKPRLDSV | 960 |
| TSSESFASSG FQEDKSLSDV EEEEDSDGFY KEPITMEDLI SYSFQVARGM EFLSSRKCIE | 1020 |
| RDLAARNILL SENNVVKICD FGLARDIYKN PDYVRKGDTR LPLKWMAPES IFDKIYSTKS | 1080 |
| DVWSYGVLLW EIFSLGGSPY PGVQMDEDFC SRLREGMRMR APEYSTPEIY QIMLDCWHRD | 1140 |
| PKERPRFAEL VEKLGDLLQA NVQQDGKDYI PINAILTGNS GFTYSTPAFS EDFFKESISA | 1200 |
| PKFNSGSSDD VRYVNAFKFM SLERIKTFEE LLPNATSMFD DYQGDSSTLL ASPMLKRFTW | 1260 |
| TDSKPKASLK IDLRVTSKSK ESGLSDVSRP SFCHSSCGHV SEGKRRFTYD HAELERKIAC | 1320 |
| CSPPPDYNSV VLYSTPPI | |

ACJ9 Protein sequence:
Gene name: Purine nucleoside phosphorylase
Unigene number: Hs.75514
Probeset Accession #: K02574
Protein Accession #: CAA25320
Cellular Localization: predicted cytoplasmic
Summary: likely to catalyze the reversible phosphorolytic cleavage of
purine ribonucleosides and 2′-deoxyribonucleosides

| | |
|---|---|
| MENGYTYEDY KNTAEWLLSH TKHRPQVAII CGSGLGGLTD KLTQAQIFDY SEIPNFPRST | 60 |
| VPGHAGRLVF GFLNGRACVM MQGRFHMYEG YPLWKVTFPV RVFHLLGVDT LVVTNAAGGL | 120 |
| NPKFEVGDIM LIRDHINLPG FSGQNPLRGP NDERFGDRFP AMSDAYDRTM RQRALSTWKQ | 180 |
| MGEQRELQEG TYVMVAGPSF ETVAECRVLQ KLGADAVGMS TVPEVIVARH CGLRVFGFSL | 240 |
| ITNKVIMDYE SLEKANHEEV LAAGKQAAQK LEQFVSILMA SIPLPDKAS | |

ACK4 Protein sequence
Gene name: EST
Probeset Accession #: R68763
Predicted amino acid seg: FGENESH exon prediction on BAC clone AC009414
Predicted nuclear target motifs: from 25 (4) RRRP (underlined); 176 (5)
RRRR (underlined); 177 (5) RRRR (underlined; 239 (5) KRKK (underlined);
399 (4) PPRARRT (underlined); 400 (5) PRARRTE (underlined)
Cellular localization: predicted nuclear

| | |
|---|---|
| MPPEQHHQPN KVSPKLCSAQ PAPRGRRRPG GRGPAAGGRT FANARFVLGE GVAIERGADD | 60 |
| TTQPPVAGSV NPEGAAAALV PLAGARVAAA ADALHDAPRA VPGLLALGLV TGQADQRPGA | 120 |
| GARQQQQQPQ QRDQEVPAAG QPPVPRHQVH PPAPPPPPPR SRAGSGAGAL PCAGHTRRRR | 180 |
| RTSSPRSSPP LSGPPGRASP RGARPPPLLR AAPTPSPRAL APAAASPPPP PPPPGREGEK | 240 |
| RKKFPPGSSG STQTSGAAAA VAAALGSSPG RRRLLPLLLR VGRPRSGAAS GPVPASRAAE | 300 |
| WARWRSTRSA ASAPRAPLAS LLRRSSGRLF MAGASAARAA PSPILPPPPD LPPTPTRRAP | 360 |
| LIGCPPSPAR PAPSASPSPS RAAGPFLPPS HASTSSRSPP PRARRTEPAV PPSCGSGPGA | 420 |
| AGALRMGLGR TQRAARVAVS RALAGTVAAA AGLGARRARR LHLRGQIGVR RVAGTPEARG | 480 |
| RGDGCSLGRV SPDRTPGKGS KGMEPPHTG | |

AAA8 Protein sequence:
Gene name: ETL protein, with extended open reading frame
Unigene number: Hs.57958
Probeset Accession #: D58024
Protein Accession #: AAG33021
Transmembrane domains: predicted 454-470, 486-502, 511-527, 528-544,
556-572, 600-616, 642-661, 672-689 (underlined sequences) Extended
sequence: Residues 1-564 were added to the sequence in AAG33021
Cellular Localization: predicted cell surface serpentine receptor

| | |
|---|---|
| MKTAALTPPR SPPPPPLRPP PMKRLPLLVV FSTLLNCSYT QNCTKTPCLP NAKCEIRNGI | 60 |
| EACYCNMGFS GNGVTICEDD NECGNLTQSC GENANCTNTE GSYYCMCVPG FRSSSNQDRF | 120 |
| ITNDGTVCIE NVNANCHLDN VCIAANINKT LTKIRSIKEP VALLQEVYRN SVTDLSPTDI | 180 |
| ITYIEILAES SSLLGYKNNT ISAKDTLSNS TLTEFVKTVN NFVQRDTFVV WDKLSVNHRR | 240 |
| THLTKLMHTV EQATLRISQS FQKTTEFDTN STDIALKVFF FDSYNMKHIH PHMNMDGDYI | 300 |
| NIFPKRKAAY DSNGNVAVAF LYYKSIGPLL SSSDNFLLKP QNYDNSEEEE RVISSVISVS | 360 |
| MSSNPPTLYE LEKITFTLSH RKVTDRYRSL CAFWNYSPDT MNGSWSSEGC ELTYSNETHT | 420 |
| SCRCNHLTHF AILMSSGPSI GIKDYNILTR ITQLGIIISL ICLAICIFTF WFFSEIQSTR | 480 |
| TTIHKNLCCS LFLAELVFLV GINTNTNKLX SVSIIAGLLH YFFLAAFAWM CIEGIHLYLI | 540 |
| VVGVIYNKGF LHKNFYIFGY LSPAVVVGFS AALGYRYYGT TKVCWLSTET HFIWSFIGPA | 600 |
| CLIILVNLLA FGVIIYKVFR HTAGLKPEVS CFENIRSCAR GALALLFLLG TTWIFGVLHV | 660 |
| VHASVVTAYL FTVSNAFQGM FIFLFLCVLS RKIQEEYYRL FKNVPCCFGC LR | |

AAC6 Protein sequence:
Gene name: EST
Unigene number: H5.134797
Probeset Accession #: AA025351
Protein accession #: BAB14599
Signal sequence: predicted 1-24 (first underlined sequence)
extended sequence: second underlined sequence

| | |
|---|---|
| MILSLLFSLG GPLGWGLLGA WAQASSTSLS DLQSSRTPGV WKAEAEDTSK DPVGRNWCPY | 60 |
| PMSKLVTLLA LCKTEKFLIH SQQPCPQGAP DCQKVKVMYR MAHKPVYQVK QKVLTSLAWR | 120 |
| CCPGYTGPNC EHHDSMAIPE PADPGDSHQE PQDGPVSFKP GHLAAVINEV EVQQEQQEHL | 180 |
| LGDLQNDVHR VADSLPGLWK ALPGNLTAAV MEANQTGHEF PDRSLEQVLL PHVDTFLQVH | 240 |
| FSPIWRSFNQ SLHSLTQAIR NLSLDVEANR QAISRVQDSA VARADFQELG AKFEAKVQEN | 300 |
| TQRVGQLRQD VEDRLHAQHF TLHRSISELQ ADVDTKLKRL HKAQEAPGTN GSLVLATPGA | 360 |
| GARPEPDSLQ ARLGQLQRL SELHMTTARR EEELQYTLED MRATLTRHVD EIKELYSESD | 420 |
| ETFDQISKVE RQVEELQVH TALRELRVIL MEKSLIMEEN KEEVERQLLE LNLTLQHLQG | 480 |
| GHADLIKYVK DCNCQKLYLD LDVIREGQRD ATRALEETQV SLDERRQLDG SSLQALQNAV | 540 |
| DAVSLAVDAH KAEGERARAA TSRLRSQVQA LDDEVGALKA AAAEARHEVR QLHSAFAALL | 600 |
| EDALRHEAVL AALFGEEVLE EMSEQTPGPL PLSYEQIRVA LQDAASGLQE QALGWDELAA | 660 |
| RVTALEQASE PPRPAEHLEP SHDAGREEAA TTALAGLARE LQSLSNDVKN VGRCCEAEAG | 720 |
| AGAASLNASL DGLHNALFAT QRSLEQHQRL FHSLFGNFQG LMEANVSLDL GKLQTMLSRK | 780 |
| GKKQQKDLEA PRKRDKKEAE PLVDIRVTGP VPGALGAALW EASPVAFYAS FSEGTAALQT | 840 |
| VKFNTTYINI GSSYFPEHGY FRAPERGVYL FAVSVEFGPG PGTGQLVFGG HHRTPVCTTG | 900 |
| QGSGSTATVF AMAELQKGER VWFELTQGSI TKRSLSGTAF GGFLMFKT | |

ACH7 Protein sequence:
Gene name: EST
Unigene number: Hs.3807
Probeset Accession #: AA292694
BAC Accession #: AL161751
FGENESH predicted aa seg: 1-647; based on BAC clone AL161751

| | |
|---|---|
| MGKDFMTKTP KAFATKAKID KWDLIKLKSF CTAKETIIRV NSQPTDWQKT FAIYPSDKGV | 60 |
| IARIYKELEQ IYKKKKPTKT LRTHFLSRPK GNCWPLGPRG DSWQLGGPSG ARAEGKGGGT | 120 |
| GLGKPAVEGG DRAPDTALRP RAGQIQVGSS SACGASENEA GVRPVPPLAG ALARAGRRRT | 180 |
| PHCRPCWLLG LGGLLQPAPR YHEAAGGRGG LHPARWGAQH RACGRRAARC ARAPAGRPRA | 240 |
| RRGLQRPAVL GRTGAQAFPL HPGERAFAGF LLAVLRPRRS RKRHAAVGGG APTLLHRAEM | 300 |
| RGTPGHRWGR ARSWKEMRCH LRANGYLCKY QFEVLCPAPR PGAASNLSYR APFQLESAAL | 360 |
| DFSPPGTEVS ALCRGQLPIS VTCIADEIGA RWDKLSGDVL CPCPGRYLRA GKCAELPNCL | 420 |
| DDLGGFACEC ATGFELGKDG RSCVTSGEGQ PTLGGTGVPT RRPPATATSP VPQRTWPIRV | 480 |
| DEKLGETPLV PEQDNSVTSI PEIPRWGSQS TMSTLQMSLQ AESKATITPS GSVISKFNST | 540 |
| TSSATPQAFD SSSAVVFIFV STAVVVLVIL TMTVLGLVKL CFHESPSSQP RKESMGPPGL | 600 |
| ESDPEPAALG SSSAHCTNNG VKVGDCDLRD RAEGALLAES PLGSSDA | |

AAD4 Protein sequence
Gene name: ERG
Unigene number: Hs.45514
Probeset Accession #: R32894
Protein Accession #: AAA52398
Signal sequence: none
Transmembrane domains: none
PFAM domains: predicted Ets-domain 294-373; SAM_PNT: 122-206
Summary: ERG2 is a sequence-specific DNA-binding protein.

| | |
|---|---|
| MIQTVPDPAA HIKEALSVVS EDQSLFECAY GTPHLAKTEM TASSSSDYGQ TSKMSPRVPQ | 60 |
| QDWLSQPPAR VTIKMECNPS QVNGSRNSPD ECSVAKGGKM VGSPDTVGMN YGSYMEEKHM | 120 |
| PPPNMTTNER RVIVPADPTL WSTDHVRQWL EWAVKEYGLP DVNILLFQNI DGKELCKMTK | 180 |
| DDFQRLTPSY NADILLSHLH YLRETPLPHL TSDDVDKALQ NSPRLMHARN TDLPYEPPRR | 240 |
| SAWTGHGHPT PQSKAAQPSP STVPKTEDQR PQLDPYQILG PTSSRLANPG SGQIQLWQFL | 300 |
| LELLSDSSNS SCITWEGTNG EFKMTDPDEV ARRWGERKSK PNMNYDKLSR ALRYYYDKNI | 360 |
| MTKVHGKRYA YKFDFHGIAQ ALQPHPPESS LYKYPSDLPY MGSYHARPQK MNFVAPHPPA | 420 |
| LPVTSSSFFA APNPYWNSPT GGIYPNTRLP TSHMPSELGT YY | 462 |

AAD5 Protein sequence
Gene name: activin A receptor type Il-like 1 (ALK-1)
Unigene number: Hs.172670
Probeset Accession #: T57112
Protein Accession #: NP_000011
Signal sequence: predicted 1-21
Transmembrane domain: predicted 119-135
PFAM domains: predicted pkinase 204-489
Summary: Type Ia membrane protein; receptor tyrosine kinase

| | |
|---|---|
| MTLGSPRKGL LMLLMALVTQ GDPVKPSRGP LVTCTCESPH CKGPTCRGAW CTVVLVREEG | 60 |
| RHPQEHRGCG NLHRELCRGR PTEFVNHYCC DSHLCNHNVS LVLEATQPPS EQPGTDGQLA | 120 |
| LILGPVLALL ALVALGVLGL WHVRRRQEKQ RGLHSELGES SLILKASEQG DTMLGDLLDS | 180 |
| DCTTGSGSGL PFLVQRTVAR QVALVECVGK GRYGEVWRGL WHGESVAVKI FSSRDEQSWF | 240 |
| RETEIYNTVL LRHDNILGFI ASDMTSRNSS TQLWLITHYH EHGSLYDFLQ RQTLEPHLAL | 300 |
| RLAVSAACGL AHLHVEIFGT QGKPAIAHRD FKSRNVLVKS NLQCCIADLG LAVMHSQGSD | 360 |
| YLDIGNNPRV GTKRYMAPEV LDEQIRTDCF ESYKWTDA FGLVLWEIAR RTIVNGIVED | 420 |
| YRPPFYDVVP NDPSFEDMKK VVCVDQQTPT IPNRLAADPV LSGLAQMMRE CWYPNPSARL | 480 |
| TALRIKKTLQ KISNSPEKPK VIQ | |

AAD8 Protein sequence
Gene name: ESTs
Unigene number: Hs.144953
Probeset Accession #: AA404418
Protein Accession #: n/a
Signal sequence: n/a
Transmembrane domains: n/a
PFAM domains: n/a
Summary: no ORF identified; possible frameshifts. Nearby to PCTAIRE
protein kinase 2 (PCTK2) on the genome (within 100 kb).
ACA2 Protein sequence
Gene name: EST
Unigene number: Hs.16450
Probeset Accession #: AA478778
Protein Accession #: n/a
Signal sequence: n/a
Transmembrane domains: n/a
PFAM domains: n/a
Summary: no ORF identified, possible frameshifts; although a match was
found to the HTGS genomic sequence, the sequence does not extend far
enough upstream to predict coding exons.
ACA4 Protein sequence
Gene name: alpha satellite junction DNA sequence
Unigene number: Hs.247946
Probeset Accession #: M21305
Protein Accession #: AAA88020
Signal sequence: none
Transmembrane domains: none
PFAM domains; none
MEWNGMAWNR IKWNGINSSG MEWNGMEWNA VQCNRNEWNE LELTGMEWNG MHLN
ACG6 Protein sequence
Gene name: intercellular adhesion molecule 2 (ICAM2)
Unigene number: Hs.83733
Probeset Accession #: M32334
Protein Accession #: NP_000864
Signal sequence: predicted 1-21
Transmembrane domain: predicted 224-248
PFAM domains: predicted 41-98, 127-197; immunoglobulin-like C2-type
domains Summary: a predicted Type Ia membrane protein; it plays a role
in cell adhesion and is the ligand for the LFA-1 protein. ICAM2 is also
called CD102.

| | |
|---|---|
| MSSFGYRTLT VALFTLICCP GSDEKVFEVH VRPKKLAVEP KGSLEVNCST TCNQPEVGGL | 60 |
| ETSLNKILLD EQAQWKHYLV SNISHDTVLQ CHFTCSGKQE SNNSNVSVYQ PPRQVILTLQ | 120 |
| PTLVAVGKSF TIECRVPTVE PLDSLTLFLF RGNETLHYET FGKAAPAPQE ATATFNSTAD | 180 |
| REDGRRNFSC LAVLDLMSRG GNIFHKHSAP KMLEIYEPVS DSQMVIIVTV VSVLLSLFVT | 240 |
| SVLLCFIFGQ HLRQQRMGTY GVRAAWRRLP QAFRP | |

ACG7 Protein sequence
Gene name: Cadherin 5, VE-cadherin (CDH5)
Unigene number: Hs.76206
Probeset Accession #: X79981
Protein Accession #: NP_001786
Signal sequence: predicted 1-27
Transmembrane domain: predicted 604-620
PFAM domains: Cadherin domains predicted 53-141, 156-249, 263-364, 377-
470, and 487-576
Summary: Likely a Type I membrane protein. Cadherins are calc. m-
dependent adhesive proteins that mediate cell-to-cell interaction. VE-
cadherin is associated with intercellular junctions.

| | |
|---|---|
| MQRLMMLLAT SGACLGLLAV AAVAAAGANP AQRDTHSLLP THRRQKRDWI WNQMHIDEEK | 60 |
| NTSLPHHVGK IKSSVSRKNA KYLLKGEYVG KVFRVDAETG DVFAIERLDR ENISEYHLTA | 120 |
| VIVDKDTGEN LETPSSFTIK VHDVNDNWPV FTHRLFNASV PESSAVGTSV ISVTAVDADD | 180 |
| PTVGDHASVM YQILKGKEYF AIDNSGRIIT ITKSLDREKQ ARYEIVVEAR DAQGLRGDSG | 240 |
| TATVLVTLQD INDNFPFFTQ TKYTFVVPED TRVGTSVGSL FVEDPDEPQN RMTKYSILRG | 300 |
| DYQDAFTIET NPAHNEGIIK PMKPLDYEYI QQYSFIVEAT DPTIDLRYMS PPAGNRAQVI | 360 |
| INITDVDEPP IFQQPFYHFQ LKENQKKPLI GTVLAMDPDA ARHSIGYSIR RTSDKGQFFR | 420 |
| VTKKGDIYNE KELDREVYPW YNLTVEAKEL DSTGTPTGKE SIVQVHIEVL DENDNAPEFA | 480 |
| KPYQPKVCEN AVHGQLVLQI SAIDKDITPR NVKFKFTLNT ENNFTLTDNH DNTANITVKY | 540 |
| GQFDREHTKV HFLPVVISDN GMPSRTGTST LTVAVCKCNE QGEFTFCEDM AAQVGVSIQA | 600 |
| VVAILLCILT ITVITLLIFL RRRLRKQARA HGKSVPEIHE QLVTYDEEGG GEMDTTSYDV | 660 |
| SVLNSVRRGG AKPPRPALDA RPSLYAQVQK PPRHAPGAHG GPGEMAANIE VKKDEADHDG | 720 |
| DGPPYDTLHI YGYEGSESIA ESLSSLGTDS SDSDVDYDFL NDWGPRFKML AELYGSDPRE | 780 |
| ELLY | |

ACG9 Protein sequence
Gene name: lysyl oxidase-like 2 (LOXL2)
Unigene number: Hs.83354
Probeset Accession #: U89942
Protein Accession #: NP_002309
Signal sequence: predicted 1-2
Transmembrane domains: none predicted
PFAM domains: scavenger receptor cysteine-rich domains predicted 68-
159, 203-238, 336-425, 439-528; Lysyl oxidase predicted 548-749.
Summary: Likely a secreted protein. Lysyl oxidase is a copper-dependent
amine oxidase that belongs to a heterogeneous family of enzymes that
oxidize primary amine substrates to reactive aldehydesm, acting on the
extracellular matrix substrates, e.g., collagen and elastin.

| | |
|---|---|
| MERPLCSHLC SCLAMLALLS PLSLAQYDSW PHYPEYFQQP APEYHQPQAP ANVAKIQLRL | 60 |
| AGQKRKHSEG RVEVYYDGQW GTVCDDDFSI HAAHVVCREL GYVEAKSWTA SSSYGKGEGP | 120 |
| IWLDNLHCTG NEATLAACTS NGWGVTDCKH TEDVGVVCSD KRIPGFKFDN SLINQIENLN | 180 |
| IQVEDIRIRA ILSTYRKRTP VMEGYVEVKE GKTWKQICDK HWTAKNSRVV CGMFGFPGER | 240 |
| TYNTKVYKMF ASRRKQRYWP FSMDCTGTEA HISSCKLGPQ VSLDPMIGNT CENGLPAVVS | 300 |
| CVPGQVFSPD GPSRFRKAYK PEQPLVRLRG GAYIGEGRVE VLKNGEWGTV CDDKWDLVSA | 360 |
| SVVCRELGFG SAKEAVTGSR LGQGIGPIHL NEIQCTGNEK SIIDCKFNAE SQGCNHEEDA | 420 |
| GVRCNTPAMG LQKKLRLNGG RNPYEGRVEV LVERNGSLVW GMVCGQNWGI VEAMVVCRQL | 480 |
| GLGFASNAFQ ETWYWHGDVN SNKVVMSGVK CSGTELSLAH CRHDGEDVAC PQGGVQYGAG | 540 |
| VACSETAPDL VLNAEMVQQT TYLEDRPMFM LQCAMEENCL SASAAQTDPT TGYRRLLRFS | 600 |
| SQIHNNGQSD FRPKNGRHAW IWHDCHRHYH SMEVFTHYDL LNLNGTKVAE GHKASFCLED | 660 |
| TECEGDIQKN YECANFGDQG ITMGCWDMYR HDIDCQWVDI TDVPPGDYLF QVVINPNFEV | 720 |
| AESDYSNNIM KCRSRYDGHR IWMYNCHIGG SFSEETEKKF EHFSGLLNNQ LSPQ | |

ACH2 Protein sequence
Gene name: TIE tyrosine-protein kinase
Unigene number: Hs.78824
Probeset Accession #: X60957
Protein Accession #: NP_005415
Signal sequence: predicted 1-21
Transmembrane domain: predicted 770-786
PFAM domains: laminin-EGF predicted 234-267; FN3 predicted 460-520, 548-
632, and 644-729; tyrosine_kinase predicted 839-1107
Summary: Likely a Type Ia membrane protein; TIE is a tyrosine-kinase
receptor with an unknown ligand; its expression is likely necessary for
normal blood vessel development.

| | |
|---|---|
| MVWRVPPFLL PILFLASHVG AAVDLTLLAN LRLTDPQRFF LTCVSGEAGA GRGSDAWGPP | 60 |
| LLLEKDDRIV RTPPGPPLRL ARNGSHQVTL RGFSKPSDLV GVFSCVGGAG ARRTRVIYVH | 120 |
| NSPGAHLLPD KVTHTVNKGD TAVLSARVHK EKQTDVIWKS NGSYFYTLDW HEAQDGRFLL | 180 |
| QLPNVQPPSS GIYSATYLEA SPLGSAFFRL IVRGCGAGRW GPGCTKECPG CLHGGVCHDH | 240 |
| DGECVCPPGF TGTRCEQACR EGRFGQSCQE QCPGISGCRG LTFCLPDPYG CSCGSGWRGS | 300 |
| QCQPCAPGH FGADCRLQCQ CQNGGTCDRF SGCVCPSGWH GVHCEKSDRI PQILNMASEL | 360 |
| EFNTMPRI NCAAAGNPFP VRGSIELRKP DGTVLLSTKA IVEPEKTTAE FEVPRLVLAD | 420 |
| SGFWECRVST SGGQDSRRFK VNVKVPPVPL AAPRLLTKQS RQLVVSPLVS FSGDGPISTV | 480 |
| RLHYRPQDST MDWSTIVVDP SENVTLMNLR PKTGYSVRVQ LSRPGEGGEG AWGPPTLMTT | 540 |
| DCPEPLLQPW LEGWHVEGTD RLRVSWSLPL VPGPLVGDGF LLRLWDGTRG QERRENVSSP | 600 |
| QARTALLTGL TPGTHYQLDV QLYHCTLLGP ASPPAHVLLP PSGPPAPRHL HAQALSDSEI | 660 |
| QLTWKHPEAL PGPISKYVVE VQVAGGAGDP LWIDVDRPEE TSTIIRGLNA STRYLFRMRA | 720 |
| SIQGLGDWSN TVEESTLGNG LQAEGPVQES RAAEEGLDQQ LILAVVGSVS ATCLTILAAL | 780 |
| LTLVCIRRSC LHRRRTFTYQ SGSGEETILQ FSSGTLTLTR RPKLQPEPLS YPVLEWEDIT | 840 |
| FEDLIGEGNF GQVIRAMIKK DGLKMNAAIK MLKEYASEND HRDFAGELEV LCKLGHHPNI | 900 |
| INLLGACKNR GYLYIAIEYA PYGNLLDFLR KSRVLETDPA FAREHGTAST LSSRQLLRFA | 960 |
| SDAANGMQYL SEKQFIHRDL AARNVLVGEN LASKIADFGL SRGEEVYVKK TMGRLPVRWM | 1020 |
| AIESLNYSVY TTKSDVWSFG VLLWEIVSLG GTPYCGMTCA ELYEKLPQGY RMEQPRNCDD | 1080 |
| EVYELMRQCW RDRPYERPPF AQIALQLGRM LEARKAYVNM SLFENFTYAG IDATAEEA | |

ACH3 Protein sequence
Gene name: placental growth factor (PGF; PlGF1; VEGF-related protein)
Unigene number: Hs.2894
Probeset Accession #: X54936
Protein Accession #: NP_002623
Signal sequence: predicted 1-21
Transmembrane domain: none predicted
PFAM domains: PDGF predicted 52-130
Summary: Likely a secreted protein; likely regulates angiogenesis by
interacting with FLT1 and FLK1.

| | |
|---|---|
| MPVMRLFPCF LQLLAGLALP AVPPQQWALS AGNGSSEVEV VPFQEVWGRS YCRALERLVD | 60 |
| VVSEYPSEVE HMFSPSCVSL LRCTGCCGDE NLHCVPVETA NVTMQLLKIR SGDRPSYVEL | 120 |
| TFSQHVRCEC RPLREKMKPE RCGDAVPRR | |

ACH4 Protein sequence
Gene name: nidogen 2 (NID2)
Unigene number: Hs.82733
Probeset Accession #: D86425
Protein Accession #: NP_031387
Signal sequence: predicted 1-30
Transmembrane domain: none predicted
PFAM domains: EGF-like_domains predicted 489-524, 764-800,
806-843, 853-891, and 897-930; thyroglobulin_repeats pre-
dicted 941-1006, and 1020-1085; LDL_receptor_repeats
predicted 1155-1197, 1199-1240, and 1242-1285. Summary: A secreted pro-
tein; NID2 likely interacts with collagens I and IV and laminin-1 to pro-
mote cell adhesion to the basement membrane.

| | |
|---|---|
| MEGDRVAGRP VLSSLPVLLL LQLLMLRAAA LHPDELFPHG ESWWDQLLQE GDDVKLSRGE | 60 |
| AGESPALLTK PDSATSTWAP TASSPLRTSP GKRSMWTMIS PPTSRPSPLF WRTSTRATAE | 120 |
| AESCTERTPP PQCWAWPPAM CALASRALRA FYPHPRLPGH LGAGRRLRGG QTRALPSGEL | 180 |
| NTFQAVLASD GSDSYALFLY PANGLQFLGT RPKESYNVQL QLPARVGFCR GEADDLKSEG | 240 |
| PYFSLTSTEQ SVKNLYQLSN LGIPGVWAFH IGSTSPLDNV RPAAVGDLSA AHSSVPLGRS | 300 |
| FSHATALESD YNEDNLDYYD VNEEEAEYLP GEPEEALNGH SSIDVSFQSK VDTKPLEESS | 360 |
| TLDPHTKEGT SLGEVGGPDL KGQVEPWDER ETRSPAPPEV DRDSLAPSWE TPPPYPENGS | 420 |
| IQPYPDGGPV PSEMDVPPAH PEEEIVLRSY PASGHTTPLS RGTYEVGLED NIGSNTEVFT | 480 |
| YNAANKETCE HNHRQCSRHA FCTDYATGFC CHCQSKFYGN GKHCLPEGAP HRVNGKVSGH | 540 |
| LHVGHTPVHF TDVDLHAYIV GNDGRAYTAI SHIPQPAAQA LLPLTPIGGL FGWLFALEKP | 600 |
| GSENGFSLAG AAFTHDMEVT FYPGEETVRI TQTAEGLDPE NYLSIKTNIQ GQVPYVPANF | 660 |
| TAHISPYKEL YHYSDSTVTS TSSRDYSLTF GAINQTWSYR IHQNITYQVC RHAPRHPSFP | 720 |
| TTQQLNVDRV FALYNDEERV LRFAVTNQIG PVKEDSDPTP VNPCYDGSHM CDTTARCHPG | 780 |
| TGVDYTCECA SGYQGDGRNC VDENECATGF HRCGPNSVCI NLPGSYRCEC RSGYEFADDR | 840 |
| HTCILITPPA NPCEDGSHTC APAGQARCVH HGGSTFSCAC LPGYAGDGHQ CTDVDECSEN | 900 |
| RCHPAATCYN TPGSFSCRCQ PGYYGDGFQC IPDSTSSLTP CEQQQRHAQA QYAYPGARFH | 960 |
| IPQCDEQGNF LPLQCHGSTG FCWCVDPDGH EVPGTQTPPG STPPHCGPSP EPTQRPPTIC | 1020 |
| ERWRENLLEH YGGTPRDDQY VPQCDDLGNF IPLQCHGKSD FCWCVDKDGR EVQGTRSQPG | 1080 |
| TTPACIPTVA PPMVRPTPRP DVTPPSVGTF LLYTQGQQIG YLPLNGTRLQ ITAAKTLLSL | 1140 |
| HGSIIVGIDY DCRERMVYWT DVAGRTISPA GLELGAEPET IVNSGLISPE GLAIDHIRRT | 1200 |
| MYWTDSVLDK IESALLDGSE RKVLFYTDLV NPRAIAVDPI RGNLYWTDWN REAPKIETSS | 1260 |
| LDGENRRILI NTDIGLPNGL TFDPFSKLLC WADAGTKKLE CTLPDGTGRR VIQNNLKYPF | 1320 |
| SIVSYADHFY HTDWRRDGVV SVNKHSGQFT DEYLPEQRSH LYGITAVYPY CPTGRK | |

ACH5 Protein sequence
Gene name: SNL (singed-like; sea urchin fascin homolog-like)
Unigene number: Hs.118400
Probeset Accession #: U03057
Protein Accession #: NP_003079
Signal sequence: none identified
Transmembrane domain: none identified
PFAM domains: none identified
Summary: a cytoplasmic, actin-bundling protein that is likely to be
involved in the assembly of actin filament bundles present in micro-
spikes, membrane ruffles, and stress fibers

| | |
|---|---|
| MTANGTAEAV QIQFGLINCG NKYLTAEAFG FKVNASASSL KKKQIWTLEQ PPDEAGSAAV | 60 |
| CLRSHLGRYL AADKDGNVTC EREVPGPDCR FLIVAHDDGR WSLQSEARRR YFGGTEDRLS | 120 |
| CFAQTVSPAE KWSVHIAMHP QVNIYSVTRK RYAHLSARPA DEIAVDRDVP WGVDSLITLA | 180 |
| FQDQRYSVQT ADHRFLRHDG RLVARPEPAT GYTLEFRSGK VAFRDCEGRY LAPSGPSGTL | 240 |
| KAGKATKVGK DELFALEQSC AQVVLQAANE RNVSTRQGMD LSANQDEETD QETFQLEIDR | 300 |
| DTKKCAFRTH TGKYWTLTAT GGVQSTASSK NASCYFDIEW RDRRITLRAS NGKFVTSKKN | 360 |
| GQLAASVETA GDSELFLMKL INRPIIVFRG EHGFIGCRKV TGTLDANRSS YDVFQLEFND | 420 |
| GAYNIKDSTG KYWTVGSDSA VTSSGDTPVD FFFEFCDYNK VAIKVGGRYL KGDHAGVLKA | 480 |
| SAETVDPASL WEY | |

ACH6 Protein sequence
Gene name: endothelial protein C receptor (EPCR; PROCR)
Unigene number: Hs.82353
Probeset Accession #: L35545
Protein Accession #: NP_006395
Signal sequence: predicted 1-17
Transmembrane domain: predicted 211-227
PFAM domains: none identified
Summary: a Type Ia membrane protein, EPCR likely binds to [thrombin]-
activated Protein C, a vitamin K-dependent serine protease zymogen
necessary for blood coagulation.

| | |
|---|---|
| MLTTLLPILL LSGWAFCSQD ASDGLQRLHM LQISYFRDPY HVWYQGNASL GGHLTHVLEG | 60 |
| PDTNTTIIQL QPLQEPESWA RTQSGLQSYL LQFEGLVRLV HQERTLAFPL TIRCFLGCEL | 120 |
| PPEGSRAHVF FEVAVNGSSF VSFRPERALW QADTQVTSGV VTFTLQQLNA YNRTRYELRE | 180 |
| FLEDTCVQYV QKHISAENTK GSQTSRSYTS LVLGVLVGGF IIAGVAVGIF LCTGGRRC | |

ACH8 Protein sequence
Gene name: melanoma adhesion molecule (MCAM; MUC18)
Unigene number: Hs.211579
Probeset Accession #: D51069
Protein Accession #: NP_006491
Signal sequence: predicted 1-17
Transmembrane domain: predicted 559-575
PFAM domains: immunoglobulin_domains predicted 264-324, and
356-410. Summary: a Type Ia membrane protein, associated with tumor
progression and the development of metastasis in human malignant mel-
anoma, and may play a role in neural crest cells during embryonic
development.

| | |
|---|---|
| MGLPRLVCAF LLAACCCCPR VAGVPGEAEQ PAPELVEVEV GSTALLKCGL SQSQGNLSHV | 60 |
| DWFSVHKEKR TLIFRVRQGQ GQSEPGEYEQ RLSLQDRGAT LALTQVTPQD ERIFLCQGKR | 120 |
| PRSQEYRIQL RVYKAPEEPN IQVNPLGIPV NSKEPEEVAT CVGRNGYPIP QVIWYKNGRP | 180 |
| LKEEKNRVHI QSSQTVESSG LYTLQSILKA QLVKEDKDAQ FYCELNYRLP SGNHMKESRE | 240 |
| VTVPVFYPTE KVWLEVEPVG MLKEGDRVEI RCLADGNPPP HFSISKQNPS TREAEEETTN | 300 |
| DNGVLVLEPA RKEHSGRYEC QAWNLDTMIS LLSEPQELLV NYVSDVRVSP AAPERQEGSS | 360 |
| LTLTCEAESS QDLEFQWLRE ETDQVLERGP VLQLHDLKRE AGGGYRCVAS VPSIPGLNRT | 420 |
| QLVKLAIFGP PWMAFKERKV WVKENMVLNL SCEASGHPRP TISWNVNGTA SEQDQDPQRV | 480 |
| LSTLNVLVTP ELLETGVECT ASNDLGKNTS ILFLELVNLT TLTPDSNTTT GLSTSTASPH | 540 |
| TRANSTSTER KLPEPESRGV VIVAVIVCIL VLAVLGAVLY FLYKKGKLPC RRSGKQEITL | 600 |
| PPSRKTELVV EVKSDKLPEE MGLLQGSSGD KRAPGDQGEK YIDLRH | |

ACH9 Protein sequence
Gene name: endothelin-1 (EDN1)
Unigene number: Hs.2271
Probeset Accession #: J05008
Protein Accession #: NP_001946
Signal sequence: predicted 1-17
Transmembrane domain: none predicted
PFAM domains: Endothelin domains predicted 59-73, and 108-129.
Summary: a secreted zymogen; the active protein is likely a 26-amino
acid peptide with potent mammalian vasoconstrictor activity; it is
necessary for normal vessel development.

| | |
|---|---|
| MDYLLMIFSL LFVACQGAPE TAVLGAELSA VGENGGEKPT PSPPWRLRRS KRCSCSSLMD | 60 |
| KECVYFCHLD IIWVNTPEHV VPYGLGSPRS KRALENLLPT KATDRENRCQ CASQKDKKCW | 120 |
| NFCQAGKELR AEDIMEKDWN NHKKGKDCSK LGKKCIYQQL VRGRKIRRSS EEHLRQTRSE | 180 |
| TMRNSVKSSF HDPKLKGKPS RERYVTHNPA HW | |

ACJ1 Protein sequence
Gene name: BMX non-receptor tyrosine kinase
Unigene number: Hs.27372
Probeset Accession #: X83107
Protein Accession #: NP_001712
Signal sequence: none identified
Transmembrane domain: none identified
PFAM domains: plektrin_homology_domain predicted 6-111;
SH2_domain predicted 294-383; protein_kinase_domain
predicted 417-663 Summary: a cytoplasmic protein, it likely plays a
role in the growth and differentiation of hematopoietic cells; it is
known to also be expressed in endothelial cells.

| | |
|---|---|
| MDTKSILEEL LLKRSQQKKK MSPNNYKERL FVLTKTNLSY YEYDKMKRGS RKGSIEIKKI | 60 |
| RCVEKVNLEE QTPVERQYPF QIVYKDGLLY VYASNEESRS QWLKALQKEI RGNPHLLVKY | 120 |
| HSGFFVDGKF LCCQQSCKAA PGCTLWEAYA NLHTAVNEEK HRVPTFPDRV LKIPRAVPVL | 180 |
| KMDAPSSSTT LAQYDNESKK NYGSQPPSSS TSLAQYDSNS KKIYGSQPNF NMQYIPREDF | 240 |
| PDWWQVRKLK SSSSSEDVAS SNQKERNVNH TTSKISWEFP ESSSSEEEEN LDDYDWFAGN | 300 |
| ISRSQSEQLL RQKGKEGAFM VRNSSQVGMY TVSLFSKAVN DKKGTVKHYH VHTNAENKLY | 360 |
| LAENYCFDSI PKLIHYHQHN SAGMITRLRH PVSTKANKVP DSVSLGNGIW ELKREEITLL | 420 |
| KELGSGQFGV VQLGKWKGQY DVAVKMIKEG SMSEDEFFQE AQTMMKLSHP KLVKFYGVCS | 480 |
| KEYPIYIVTE YISNGCLLNY LRSHGKGLEP SQLLEMCYDV CEGMAFLESH QFIHRDLAAR | 540 |
| NCLVDRDLCV KVSDFGMTRY VLDDQYVSSV GTKFPVKWSA PEVFHYFKYS SKSDVWAFGI | 600 |
| LMWEVFSLGK QPYDLYDNSQ VVLKVSQGHR LYRPHLASDT IYQIMYSCWH ELPEKRPTFQ | 660 |
| QLLSSIEPLR EKDKH | |

ACJ4 Protein sequence
Gene name: prostaglandin G/H synthase 2 (COX-2; PGES-2)
Unigene number: Hs.196384
Probeset Accession #: D28235
Protein Accession #: NP_000954
Signal sequence: predicted 1-17
Transmembrane domain: none identified
PFAM domains: EGF-like_domain predicted 18-55.
Summary: a microsomal enzyme; COX-2 is the therapeutic target of the
nonsteroidal anti-inflammatory drugs (NSAIDs), such as aspirin.

| | |
|---|---|
| MLARALLLCA VLALSHTANP CCSHPCQNRG VCMSVGFDQY KCDCTRTGFY GENCSTPEFL | 60 |
| TRIKLFLKPT PNTVHYILTH FKGFWNVVNN IPFLRNAIMS YVLTSRSHLI DSPPTYNADY | 120 |
| GYKSWEAFSN LSYYTRALPP VPDDCPTPLG VKGKKQLPDS NEIVEKLLLR RKFIPDPQGS | 180 |
| NMMFAFFAQH FTHQFFKTDH KRGPAFTNGL GHGVDLNHIY GETLARQRKL RLFKDGKMKY | 240 |
| QIIDGEMYPP TVKDTQAEMI YPPQVPEHLR FAVGQEVFGL VPGLMMYATI WLREHNRVCD | 300 |
| VLKQEHPEWG DEQLFQTSRL ILIGETIKIV IEDYVQHLSG YHFKLKFDPE LLFNKQFQYQ | 360 |
| NRIAAEFNTL YHWHPLLPDT FQIHDQKYNY QQFIYNNSIL LEHGITQFVE SFTRQIAGRV | 420 |
| AGGRNVPPAV QKVSQASIDQ SRQMKYQSFN EYRKRFMLKP YESFEELTGE KEMSAELEAL | 480 |
| YGDIDAVELY PALLVEKPRP DAIFGETMVE VGAPFSLKGL MGNVICSPAY WKPSTFGGEV | 540 |
| GFQIINTASI QSLICNNVKG CPFTSFSVPD PELIKTVTIN ASSSRSGLDD INPTVLLKER | 600 |
| STEL | |

ACJ6 Protein sequence
Gene name: SEC14-like-1
Unigene number: Hs.75232
Probeset Accession #: D67029
Protein Accession #: NP_002994
Signal sequence: none identified
Transmembrane domain: none identified
PFAM domains: none identified
Summary: a cytoplasmic protein

| | |
|---|---|
| MVQKYQSPVR VYKYPFELIM AAYERRFPTC PLIPMFVGSD TVSEFKSEDG AIHVIERRCK | 60 |
| LDVDAPRLLK KIAGVDYVYF VQKNSLNSRE RTLHIEAYNE TFSNRVIINE HCCYTVHPEN | 120 |
| EDWTCFEQSA SLDIKSFFGF ESTVEKIAMK QYTSNIKKGK EIIEYYLRQL EEEGITFVPR | 180 |
| WSPPSITPSS ETSSSSSKKQ AASMAVVIPE AALKEGLSGD ALSSPSAPEP VVGTPDDKLD | 240 |
| ADHIKRYLGD LTPLQESCLI RLRQWLQETH KGKIPKDEHI LRFLRARDFN IDKAREIMCQ | 300 |
| SLTWRKQHQV DYILETWTPP QVLQDYYAGG WHHHDKDGRP LYVLRLGQMD TKGLVRALGE | 360 |
| EALLRYVLSV NEERLRRCEE NTKVFGRPIS SWTCLVDLEG LNMRHLWRPG VKALLRIIEV | 420 |
| VEANYPETLG RLLILRAPRV FPVLWTLVSP FIDDNTRRKF LIYAGNDYQG PGGLLDYIDK | 480 |
| EIIPDFLSGE CMCEVPEGGL VPKSLYRTAE ELENEDLKLW TETIYQSASV FKGAPHEILI | 540 |
| QIVDASSVIT WDFDVCKGDI VFNIYHSKRS PQPPKKDSLG AHSITSPGGN NVQLIDKVWQ | 600 |
| LGRDYSMVES PLICKEGESV QGSHVTRWPG FYILQWKFHS MPACAASSLP RVDDVLASLQ | 660 |
| VSSHKCKVMY YTEVIGSEDF RGSMTSLESS HSGFSQLSAA TTSSSQSHSS SMISR | |

ACJ8 Protein sequence
Gene name: intercellular adhesion molecule 1 (ICAM1; CD54)
Unigene number: Hs.168383
Probeset Accession #: M24283
Protein Accession #: NP_000192
Signal sequence: predicted 1-27
Transmembrane domain: predicted 481-497
PFAM domains: immunoglobulin_domains predicted 128-188, and
325-373. Summary: a Type 1a membrane protein; ICAM1 is typically
expressed on endothelial cells and cells of the immune system; ICAM2.
binds to integrins of type CD11a/CD18, or CD11b/CD18; ICAM1 is also ex-
ploited by Rhinovirus as a receptor.

| | |
|---|---|
| MAPSSPRPAL PALLVLLGAL FPGPGNAQTS VSPSKVILPR GGSVLVTCST SCDQPKLLGI | 60 |
| ETPLPKKELL LPGNNRKVYE LSNVQEDSQP MCYSNCPDGQ STAKTFLTVY WTPERVELAP | 120 |
| LPSWQPVGKN LTLRCQVEGG APRANLTVVL LRGEKELKRE PAVGEPAEVT TTVLVRRDHH | 180 |
| GANFSCRTEL DLRPQGLELF ENTSAPYQLQ TFVLPATPPQ LVSPRVLEVD TQGTVVCSLD | 240 |
| GLFPVSEAQV HLALGDQRLN PTVTYGNDSF SAKASVSVTA EDEGTQRLTC AVILGNQSQE | 300 |
| TLQTVTIYSF PAPNVILTKP EVSEGTEVTV KCEAHPRAKV TLNGVPAQPL GPRAQLLLKA | 360 |
| TPEDNGRSFS CSATLEVAGQ LIHKNQTREL RVLYGPRLDE RDCPGNWTWP ENSQQTPMCQ | 420 |
| AWGNPLPELK CLKDGTFPLP IGESVTVTRD LEGTYLCRAR STQGEVTREV TVNVLSPRYE | 480 |
| IVIITVVAAA VIMGTAGLST YLYNRQRKIK KYRLQQAQKG TPMKPNTQAT PP | |

ACK3 Protein sequence
Gene name: angiopoietin 1 receptor (TIE-2; TEK)
Unigene number: Hs.89640
Probeset Accession #: L06139
Protein Accession #: NP_000450
Signal sequence: predicted 1-18
Transmembrane domain: predicted 746-770
PFAM domains: immunoglobulin_domains predicted 44-102, 370-424;
EGF_like_domains predicted 210-252, 254-299, and 301-
341; FN3_domains predicted 444-536, 541-634, and 638-732; pro-
tein_kinase_domain predicted 824-1096.
Summary: a Type 1a membrane protein; it is expressed almost exclusively
in endothelial cells in mice, rats, and humans; the ligand for this re-
ceptor is angiopoietin-1; defects in TEK are associated with inherited
venous malformations; the TEK signaling pathway appears to be critical
for endothelial cell-smooth muscle cell communication in venous morpho-
genesis.

| | |
|---|---|
| MDSLASLVLC GVSLLLSGTV EGAMDLILIN SLPLVSDAET SLTCIASGWR PEEPITIGRD | 60 |
| FEALMNQHQD PLEVTQDVTR EWAKKVVWKR EKASKINGAY FCEGRVRGEA IRIRTMKMRQ | 120 |
| QASFLPATLT MTVDKGDNVN ISFKKVLIKE EDAVIYKNGS FIHSVPRHEV PDILEVELPH | 180 |
| AQPQDAGVYS ARYIGGNLFT SAFTRLIVRR CEAQKWGPEC NHLCTACMNN GVCHEDTGEC | 240 |
| ICPPGFMGRT CEKACELHTF GRTCKERCSG QEGCKSYVFC LPDPYGCSCA TGWKGLQCNE | 300 |
| ACHPGFYGPD CKLRCSCNNG EMCDRFQGCL CSPGWQGLQC EREGIPRMTP KIVDLPDHIE | 360 |
| VNSGKFNPIC KASGWPLPTN EEMTLVKPDG TVLHPKDFNH TDHFSVAIFT IHRILPPDSG | 420 |
| VWVCSVNTVA GMVEKPFNIS VKVLPKPLNA PNVIDTGHNF AVINISSEPY FGDGPIKSKK | 480 |
| LLYKPVNHYE AWQHIQVTNE IVTLNYLEPR TEYELCVQLV RRGEGGEGHP GPVRRFTTAS | 540 |
| IGLPPPRGLN LLPKSQTTLN LTWQPIFPSS EDDFYVEVER RSVQKSDQQN IKVPGNLTSV | 600 |
| LLNNLHPREQ YVVRARVNTK AQGEWSEDLT AWTLSDILPP QPENIKISNI THSSAVISWT | 660 |
| ILDGYSISSI TIRYKVQGKN EDQHVDVKIK NATIIQYQLK GLEPETAYQV DIFAENNIGS | 720 |
| SNPAFSHELV TLPESQAPAD LGGGKMLLIA ILGSAGMTCL TVLLAFLIIL QLKRANVQRR | 780 |
| MAQAFQNVRE EPAVQFNSGT LALNRKVKNN PDPTIYPVLD WNDIKFQDVI GEGNFGQVLK | 840 |
| ARIKKDGLRM DAAIKRMKEY ASKDDHRDFA GELEVLCKLG HHPNIINLLG ACEHRGYLYL | 900 |
| AIEYAPHGNL LDFLRKSRVL ETDPAFAIAN STASTLSSQQ LLHFAADVAR GMDYLSQKQF | 960 |
| IHRDLAARNI LVGENYVAKI ADFGLSRGQE VYVKKTMGRL PVRWMAIESL NYSVYTTNSD | 1020 |
| VWSYGVLLWE IVSLGGTPYC GMTCAELYEK LPQGYRLEKP LNCDDEVYDL MRQCWREKPY | 1080 |
| ERPSFAQILV SLNRMLEERK TYVNTTLYEK FTYAGIDCSA EEAA | |

PZA6 Protein sequence
Gene name: prostate differentiation factor (PLAB; MIC-1)
Unigene number: Hs.116577
Probeset Accession #: AB000584
Protein Accession #: NP_004855
Signal sequence: predicted 1-29
Transmembrane domain: none identified
PFAM domains: TGF-beta _domain predicted 211-308.
Summary: a secreted protein; its exact function is unclear; it inhibits
proliferation of primitive hematopoietic progenitors; it inhibits acti-
vation of macrophages; it is highly expressed in placenta and in serum
of pregnant women; it may promote fetal survival by suppressing the pro-
duction of maternally-derived proinflammatory cytokines within the
uterus.

| | |
|---|---|
| MPGQELRTVN GSQMLLVLLV LSWLPHGGAL SLAEASRASF PGPSELHSED SRFRELRKRY | 60 |
| EDLLTRLRAN QSWEDSNTDL VPAPAVPILT PEVRLGSGGH LHLRISRAAL PEGLPEASRL | 120 |
| HRALFRLSPT ASRSWDVTRP LRRQLSLARP QAPALHLRLS PPPSQSDQLL AESSSARPQL | 180 |
| ELHLRPQAAR GRRRARARNG DDCPLGPGRC CRLHTVRASL EDLGWADWVL SPREVQVTNC | 240 |
| IGACPSQFRA ANMHAQIKTS LHRLKPDTEP APCCVPASYN PMVLIQKTDT GVSLQTYDDL | 300 |
| LAKDCHCI | |

AAD2 Protein sequence:
Gene name: Thrombospondin-1
Unigene number: Hs.87409
Probeset Accession #: AA232645
Protein Accession #: NP_003237.1
Signal sequence: predicted 1-18 (first underlined sequence)
Transmembrane Domain: none identified
Summary: Thrombospondin is a large modular glycoprotein component of the
extracellular matrix and contains a variety of distinct domains, includ-
ing three repeating subunits (types I, II, and III) that share homology
to an assortment of other proteins.

| | |
|---|---|
| MGLAWGLGVL FLMRVCGTNR IPESGGDNSV FDIFELTGAA RKGSGRRLVK GPDPSSPAFR | 60 |
| IEDANLIPPV PDDKFQDLVD AVRAEKGFLL LASLRQMKKT RGTLLALERK DHSGQVFSVV | 120 |
| SNGKAGTLDL SLTVQGKQHV VSVEEALLAT GQWKSITLFV QEDRAQLYID CEKMENAELD | 180 |
| VPIQSVFTRD LASIARLRIA KGGVNDNFQG VLQNVRFVFG TTPEDILRNK GCSSSTSVLL | 240 |
| TLDNNVVNGS SPAIRTNYIG HKTKDLQAIC GISCDELSSM VLELRGLRTI VTTLQDSIRK | 300 |
| VTEENKELAN ELRRPPLCYH NGVQYRNNEE WTVDSCTECH CQNSVTICKK VSCPIMPCSN | 360 |
| ATVPDGECCP RCWPSDSADD GWSPWSEWTS CSTSCGNGIQ QRGRSCDSLN NRCEGSSVQT | 420 |
| RTCHIQECDK RFKQDGGWSH WSPWSSCSVT CGDGVITRIR LCNSPSPQMN GKPCEGEARE | 480 |
| TKACKKDACP INGGWGPWSP WDICSVTCGG GVQKRSRLCN NPAPQFGGKD CVGDVTENQI | 540 |
| CNKQDCPIDG CLSNPCFAGV KCTSYPDGSW KCGACPPGYS GNGIQCTDVD ECKEVPDACF | 600 |
| NHNGEHRCEN TDPGYNCLPC PPRFTGSQPF GQGVEHATAN KQVCKPRNPC TDGTHDCNKN | 660 |
| AKCNYLGHYS DPMYRCECKP GYAGNGIICG EDTDLDGWPN ENLVCVANAT YHCKKDNCPN | 720 |
| LPNSGQEDYD KDGIGDACDD DDDNDKIPDD RDNCPFHYNP AQYDYDRDDV GDRCDNCPYN | 780 |
| HNPDQADTDN NGEGDACAAD IDGDGILNER DNCQYVYNVD QRDTDMDGVG DQCDNCPLEH | 840 |
| NPDQLDSDSD RIGDTCDNNQ DIDEDGHQNN LDNCPYVPNA NQADHDKDGK GDACDHDDDN | 900 |
| DGIPDDKDNC RLVPNPDQKD SDGDGRGDAC KDDFDHDSVP DIDDICPENV DISETDFRRF | 960 |
| QMIPLDPKGT SQNDPNWVVR HQGKELVQTV NCDPGLAVGY DEFNAVDFSG TFFINTERDD | 1020 |
| DYAGFVFGYQ SSSRFYVVMW KQVTQSYWDT NPTRAQGYSG LSVKVVNSTT GPGEHLRNAL | 1080 |
| WHTGNTPGQV RTLWHDPRHI GWKDFTAYRW RLSHRPKTGF IRVVMYEGKK IMADSGPIYD | 1140 |
| KTYAGGRLGL FVFSQEMVFF SDLKYECRDP | |

AAD9 protein sequence
Gene name: LIM homeobox protein cofactor (CLIM-1)
Unigene number: Hs.4980
Probeset Accession #: F13782
Protein Accession #: AAC83552
Pfam: LIM bind
Transmembrane Domain: none identifed
Summary: The LIM homeodomain (LIM-HD) proteins, which contain two tan-
dem LIM domains followed by a homeodomain, are critical transcriptional
regulators of embryonic development. The LIM domain is a conserved
cysteine-rich zinc-binding motif found in LIM-HD proteins, cytoskeletal
components, LIM kinases, and other proteins. LIM domains are protein-pro-
tein interaction motifs, can inhibit binding of LIM-HD proteins to DNA,
and can negatively regulate LIM-HD protein function.

| | |
|---|---|
| MSSTPHDPFY SSPFGPFYRR HTPYMVQPEY RIYEMNKRLQ SRTEDSDNLW WDAFATEFFE | 60 |
| DDATLTLSFC LEDGPKRYTI GRTLIPRYFS TVFEGGVTDL YYILKHSKES YHNSSITVDC | 120 |
| DQCTMVTQHG KPMFTKVCTE GRLILEFTFD DLMRIKTWHF TIRQYRELVP RSILANHAQD | 180 |
| PQVLDQLSKN ITRMGLTNFT LNYLRLCVIL EPMQELMSRH KTYNLSPRDC LKTCLFQKWQ | 240 |
| RMVAPPAEPT RQPTTKRRKR KNSTSSTSNS SAGNNANSTG SKKKTTAANL SLSSQVPDVM | 300 |
| VVGEPTLMGG EFGDEDERLI TRLENTQYDA ANGMDDEEDF NNSPALGNNS PWNSKPPATQ | 360 |
| ETKSENPPPQ ASQ | |

AAE1 protein seanence
Gene name: guanine nucleotide binding protein 11
Unigene number: Hs.83381
Probeset Accession #: U31384
Protein Accession #: NP_004117.1
Pfam: G-gamma; CAAX motif (farnesylation site) prediction underlined
Summary: The G gamma proteins are a component of the trimeric G-proteins
that interact with cell surface receptors. The G protein beta and gamma
subunits directly regulate the activities of various enzymes and ion
channels after receptor ligation. Unlike most of the other known gamma
subunits, gamma 11 is modified by a farnesyl group and is not capable
of interacting with beta 2.

| | |
|---|---|
| MPALHIEDLP EKEKLIG4EVE QLRKEVKLQR QQVSKCSEEI KNYIEERSGE DPLVKGIPED | 60 |
| KNPFKEKGSC VIS | |

AAE2 protein sequence
Gene name: Transcription factor 4 (Immunoglobulin transcription factor
2) (ITF-2) CSL3-3 Enhancer factor 2) (SEF-2) Unigene number: Hs.289068
Probeset Accession #: M74719 Protein Accession #: NP_003190.1
Pfam: HLH domain prediction underlined Summary: Transcription factor 4
is a helix-loop-helix (HLH) protein which belongs to a family of nu-
clear proteins, designated SL3-3 enhancer factors 2 (SEF2), that inter-
act with an Ephrussi box-like motif within the glucocorticoid response
element in the enhancer of the murine leukemia virus SL3-3. Various cell
types display differences both in the sets of SEF2-DNA complexes formed
and in their amounts. Molecular analysis of cDNA clones show the exist-
ence of multiple related mRNA species containing alternative coding
regions, which are most probably a result of differential splicing.

| | |
|---|---|
| MHHQQRMAAL GTDKELSDLL DFSAMFSPPV SSGKNGPTSL ASGHFTGSNV EDRSSSGSWG | 60 |
| NGGHPSPSRN YGDGTPYDHM TSRDLGSHDN LSPPFVNSRI QSKTERGSYS SYGRESNLQG | 120 |
| CHQQSLLGGD MDMGNPGTLS PTKPGSQYYQ YSSNNPRRRP LHSSAMEVQT KKVRKVPPGL | 180 |
| PSSVYAPSAS TADYNRDSPG YPSSKPATST FPSSFFMQDG HHSSDPWSSS SGMNQPGYAG | 240 |
| MLGNSSHIPQ SSSYCSLEPH ERLSYPSHSS ADINSSLPPM STFHRSGTNH YSTSSCTPPA | 300 |
| NGTDSIMANR GSGAAGSSQT GDALGKALAS IYSPDHTNNS FSSNPSTPVG SPPSLSAGTA | 360 |
| VWSRNGGQAS SSPNYEGPLH SLQSRIEDRL ERLDDAIHVL RNHAVGPSTA MGGHGDMHG | 420 |
| IIGPSHNGAM GGLGSGYGTG LLSANRHSLM VGTHREDGVA LRGSHSLLPN QVPVPQLPVQ | 480 |
| SATSPDLNPP QDPYRGMPPG LQGQSVSSGS SEIKSDDEGD ENLQDTKSSE DKKLDDDKKD | 540 |
| IKSITSNNDD EDLTPEQKAE REKERRMANN ARERLRVRDI NEAFKELGRM VQLHLKSDKP | 600 |
| QTKLLILHQA VAVILSLEQQ VRERNLNPKA ACLKRREEEK VSSEPPPLSL AGPHPGMGDA | 660 |
| SNHMGQM | |

AAE4 protein sequence
Gene name: phosphatidyicholine 2-acylhydrolase
Unigene number: Hs.211587
Probeset Accession #: M68874
Protein Accession #: AAA60105.1
Pfam: PLA2 B, C2 domain prediction underlined
Summary: Phospholipases A2 (PLA2s) play a key role in inflammatory pro-
cesses through production of precursors of eicosanoids and platelet-acti-
vating factor. PLA2 is a 100 kd protein that contains a structural
element homologous to the C2 region of protein kinase C.

| | |
|---|---|
| MSFIDPYQHI IVEHQYSHKF TVVVLRATKV TKGAFGDMLD TPDPYVELFI STTPDSRKRT | 60 |
| RHFNNDINPV WNETFEFILD PNQENVLEIT LMDANYVMDE TLGTATFTVS SMKVGEKKEV | 120 |
| PFIFNQVTEM VLEMSLEVCS CPDLRFSMAL CDQEKTFRQQ RKEHIRESMK KLLGPKNSEG | 180 |
| LHSARDVPVV AILGSGGGFR AMVGFSGVMK ALYESGILDC ATYVAGLSGS TWYMSTLYSH | 240 |
| PDFPEKGPEE INEELMKNVS HNPLLLLTPQ KVKRYVESLW KKKSSGQPVT FTDIFGMLIG | 300 |
| ETLIHNRMNT TLSSLKEKVN TAQCPLPLFT CLHVKPDVSE LMFADWVEFS PYEIGMAKYG | 360 |
| TFMAPDLFGS KFFMGTVVKK YEENPLHFLM GVWGSAFSIL FNRVLGVSGS QSRGSTMEEE | 420 |
| LENITTKHIV SNDSSDSDDE SHEPKGTENE DAGSDYQSDN QASWIHRMIM ALVSDSALFN | 480 |
| TREGRAGKVH NFMLGLNLNT SYPLSPLSDF ATQDSFDDDE LDAAVADPDE FERIYEPLDV | 540 |
| KSKKIHVVDS GLTFNLPYPL ILRPQRGVDL IISFDFSARP SDSSPPFKEL LLAEKWAKMN | 600 |
| KLPFPKIDPY VFDREGLKEC YVFKPKNPDM EKDCPTIIHF VLANINFRKY KAPGVPRETE | 660 |
| EEKEIADFDI FDDPESPFST FNFQYPNQAF KRLHDLMHFN TLNNIDVIKE AMVESIEYRR | 720 |
| QNPSRCSVSL SNVEARRFFN KEFLSKPKA | |

ACA1 protein sequence
Gene name: tissue factor pathway inhibitor 2 TFPI2, placental protein 5
(PP5)
Unigene number: Hs.78045
Probeset Accession #: D29992
Protein Accession #: BAA06272.1
Pfam: Kunitz BPTI
Signal sequence: underlined
Summary: ACA1 is a serine proteinase inhibitor that was originally puri-
fied from conditioned medium of the human glioblastoma cell line T98G.
ACA1 is identical to placental protein 5 (PP5) and TFPI2, a placenta-
derived glycoprotein with serine proteinase inhibitor activity. PP5 be-
longs to the Kunitz-type serine proteinase inhibitor family, having
three putative Kunitz-type inhibitor domains.

| | |
|---|---|
| MDPARPLGLS ILLLFLTEAA LGDAAQEPTG NNAEICLLPL DYGPCRALLL RYYYDRYTQS | 60 |
| CRQFLYGGCE GNANNFYTWE ACDDACWRIE KVPKVCRLQV SVDDQCEGST EKYFFNLSSM | 120 |
| TCEKFFSGGC HRNRIENRFP DEATCMGFCA PKKIPSFCYS PKDEGLCSAN VTRYYFNPRY | 180 |
| RTCDAFTYTG CGGNDNNFVS REDCKRACAK ALKKKKKMPK LRFASRIRKI RKKQF | |

ACB8 protein sequence
Gene name: myosin X
Unigene number: Hs.61638
Probeset Accession #: N77151
Protein Accession #: NP_036466
Pfam: myosin head, IQ (calmodulin binding motif), PH, MyTH4
Summary: Myosins are molecular motors that move along filamentous actin.
Seven classes of myosin are expressed in vertebrates: conventional
myosin, or myosin-II, as well as the 6 unconventional myosin classes-I,
-V, -VI, -VII, -IX, and -X.

| | |
|---|---|
| MDNFFTEGTR VWLRENGQHF PSTVNSCAEG IVVFRTDYGQ VFTYKQSTIT HQKVTAMHPT | 60 |
| NEEGVDDMAS LTELHGGSIM YNLFQRYKRN QIYTYIGSIL ASVNPYQPIA GLYEPATMEQ | 120 |
| YSRRHLGELP PHIFAIANEC YRCLWKRYDN QCILISGESG AGKTESTKLI LKFLSVISQQ | 180 |
| SLELSLKEKT SCVERAILES SPIMEAFGNA KTVYNNNSSR FGKFVQLNIC QKGNIQGGRI | 240 |
| VDYLLEKNRV VRQNPGERNY HIFYALLAGL EHEEREEFYL STPENYHYLN QSGCVEDKTI | 300 |
| SDQESFREVI TANDVMQFSK EEVREVSRLL AGILHLGNIE FITAGGAQVS FKTALGRSAE | 360 |
| LLGLDPTQLT DALTQRSMFL RGEEILTPLN VQQAVDSRDS LAMALYACCF EWVIKKINSR | 420 |
| IKGNEDFKSI GILDIFGFEN FEVNHFEQFN INYANEKLQE YFNKHIFSLE QLEYSREGLV | 460 |
| WEDIDWIDNG ECLDLIEKKL GLLALINEES HFPQATDSTL LEKLHSQHAN NHFYVKPRVA | 540 |
| VNNFGVKHYA GEVQYDVRGI LEKNRDTFRD DLLNLLRESR FDFIYDLFEH VSSRNNQDTL | 600 |
| KCGSKHRRPT VSSQFKDSLH SLMATLSSSN PFFVRCIKPN MQKMPDQFDQ AVVLNQLRYS | 660 |
| GMLETVRIRK AGYAVRRPFQ DFYKRYKVLM RNLALPEDVR GKCTSLLQLY DASNSEWQLG | 720 |
| KTKVFLRESL EQKLEKRREE EVSHAAMVIR AHVLGFLARK QYRKVLYCVV IIQKNYRAFL | 780 |
| LRRRFLHLKK AAIVFQKQLR GQIARRVYRQ LLAEKREQEE KKKQEEEEKK KREEEERERE | 840 |
| RERREAELRA QQEEETRKQQ ELEALQKSQK EAELTRELEK QKENKQVEEI LRLEKEIEDL | 900 |
| QRMKEQQELS LTEASLQKLQ ERRDQELRRL EEEACRAAQE FLESLNFDEI DECVRNIERS | 960 |
| LSVGSEFSSE LAESACEEKP NFNFSQPYPE EEVDEGFEAD DDAFKDSPNP SEHGHSDQRT | 1020 |
| SGIRTSDDSS EEDPYNNDTV VPTSPSADST VLLAPSVQDS GSLHNSSSGE STYCMPQNAG | 1080 |
| DLPSPDGDYD YDQDDYEDGA ITSGSSVTFS NSYGSQWSPD YRCSVGTYNS SGAYRFSSEG | 1140 |
| AQSSFEDSEE DFDSRFDTDD ELSYRRDSVY SCVTLPYFHS FLYMKGGLMN SWKRRWCVLK | 1200 |
| DETFLWFRSK QEALKQGWLH KKGGGSSTLS RRNWKKRWFV LRQSKLMYFE NDSEEKLKGT | 1260 |
| VEVRTAKEII DNTTKENGID IIMADRTFHL IAESPEDASQ WFSVLSQVHA STDQEIQEMH | 1320 |
| DEQANPQNAV GTLDVGLIDS VCASDSPDRP NSFVIITANR VLHCNADTPE EMHHWITLLQ | 1380 |
| RSKGDTRVEG QEFIVRGWLH KEVKNSPKMS SLKLKKRWFV LTHNSLDYYK SSEKNALKLG | 1440 |
| TLVLNSLCSV VPPDEKIFKE TGYWNVTVYG RKHCYRLYTK LLNEATRWSS AIQNVTDTKA | 1500 |
| PIDTPTQQLI QDIKENCLNS DVVEQIYKRN PILRYTHHPL HSPLLPLPYG DINLNLLKDK | 1560 |
| GYTTLQDEAI KIFNSLQQLE SMSDPIPIIQ GILQTGHDLR PLRDELYCQL IKQTNKVPHP | 1620 |
| GSVGNLYSWQ ILTCLSCTFL PSRGILKYLK FHLKRIREQF PGTEMEKYAL FTYESLKKTK | 1680 |
| CREFVPSRDE IEALIHRQEM TSTVYCHGGG SCKITINSHT TAGEVVEKLI RGLAMEDSRN | 1740 |
| MFALFEYNGH VDKAIESRTV VADVLAKFEK LAATSEVGDL PWKFYFKLYC FLDTDNVPKD | 1800 |
| SVEFAFMFEQ AHEAVIHGHH PAPEENLQVL AALRLQYLQG DYTLHAAIPP LEEVYSLQRL | 1860 |
| KARISQSTKT FTPCERLEKR RTSFLEGTLR RSFRTGSVVR QKVEEEQMLD MWIKEEVSSA | 1920 |
| RASIIDKWRK FQGNNQEQAM AKYMALIKEW PGYGSTLFDV ECKEGGFPQE LWLGVSADAV | 1980 |
| SVYKRGEGRP LEVFQYEHIL SFGAPLANTY KIVVDERELL FETSEVVDVA KLMKAYISMI | 2040 |
| VKKRYSTTRS ASSQGSSR | |

ACC3 protein sequence
Gene name: calcitonin receptor-like (CALCRL)
Unigene number: Hs.152175
Probeset Accession #: L76380
Protein Accession #: NP_005786.1
Pfam: 7TM 2 (7 transmembrane receptor (Secretin family))
Transmembrane domains: predictions underlined
Signal sequence: first underlined region
Summary: Calcitonin gene-related peptide (CGRP) is a neuropeptide with
diverse biological effects including potent vasodilator activity. The
human CGRP1 receptor shares significant peptide sequence homology with
the human calcitonin receptor, a member of the G-protein-coupled recept-
or superfamily. Stable expression in 293 (HEK 293) cells produces spec-
ific, high affinity binding sites for CGRP. Exposure of these cells to
CGRP results in a 60-fold increase in cAMP production.

| | |
|---|---|
| MEKKCTLYFL VLLPFFMILV TAELEESPED SIQLGVTRNK IMTAQYECYQ KIMQDPIQQA | 60 |
| EGVYCNRTWD GWLCWNDVAA GTESMQLCPD YFQDFDPSEK VTKICDQDGN WFRHPASNRT | 120 |
| WTNYTQCNVN THEKVKTALN LFYLTIIGHG LSIASLLISL GIFFYFKSLS CQRITLHKNL | 180 |
| FFSFVCNSVV TIIHLTAVAN NQALVATNPV SCKVSQFIHL YLMGCNYFWM LCEGIYLHTL | 240 |
| IVVAVFAEKQ HLMWYYFLGW GFPLIPACIH AIARSLYYND NCWISSDTHL LYIIHGPICA | 300 |
| ALLVNLFFLL NIVRVLITKL KVTHQAESNL YMKAVRATLI LVPLLGIEFV LIPWRPEGKI | 360 |
| AEEVYDYIMH ILMHFQGLLV STIFCFFNGE VQAILRRNWN QYKIQFGNSF SNSEALRSAS | 420 |
| YTVSTISDGP GYSHDCPSEH L&GKSIHDIE NVLLKPENLY N | |

ACC5 protein sequence
Gene name: Selectin E (endothelial adhesion molecule 1)
Unigene number: Hs.89546
Probeset Accession #: M24736
Protein Accession #: NP_000441.1
Pfam: lectin c, EGF like domain, sushi (SCR domain)
Signal sequence: first underlined region
Transmembrane domain: second underlined region
Summary: Focal adhesion of leukocytes to the blood vessel lining is a
key step in inflammation and certain vascular disease processes. Endo-
thelial leukocyte adhesion molecule-1 (ELAM-1), a cell surface glyco-
protein expressed by cytokine-activated endothelial, mediates the ad-
hesion of blood neutrophils. The primary sequence of ELAM-1 predicts
an amino-terminal lectin-like domain, an EGF domain, and six tandem re-
petitive motifs (about 60 amino acids each) related to those found
in complement regulatory proteins. A similar domain structure is also
found in the MEL-14 lymphocyte cell surface homing receptor, and in gran-
ule-membrane protein 140, a membrane glycoprotein of platelet and endo-
thelial secretory granules that can be rapidly mobilized (less than 5
minutes) to the cell surface by thrombin and other stimuli. Thus, ELAM-1
may be a member of a nascent gene family of cell surface molecules in-
volved in the regulation of inflammatory and immunological events at the
interface of vessel wall and blood.

| | |
|---|---|
| MIASQFLSAL TLVLLIKESG AWSYNTSTEA MTYDEASAYC QQRYTHLVAI QNKEEIEYLN | 60 |
| SILSYSPSYY WIGIRKVNNV WVWVGTQKPL TEEAKNWAPG EPNNRQKDED CVEIYIKREK | 120 |
| DVGMWNDERC SKKKLALCYT AACTNTSCSG HGECVETINN YTCKCDPGFS GLKCEQIVNC | 180 |
| TALESPEHGS LVCSHPLGNF SYNSSCSISC DRGYLPSSME TMQCMSSGEW SAPIPACNVV | 240 |
| ECDAVTNPAN GFVECFQNPG SFPWNTTCTF DCEEGFELMG AQSLQCTSSG NWDNEKPTCK | 300 |
| AVTCRAVRQP QNGSVRCSHS PAGEFTFKSS CNFTCEEGFM LQGPAQVECT TQGQWTQQIP | 360 |
| VCEAFQCTAL SNPERGYMNC LPSASGSFRY GSSCEFSCEQ GFVLKGSKRL QCGPTGEWDN | 420 |
| EKPTCEAVRC DAVHQPPKGL VRCAHSPIGE FTYKSSCAFS CEEGFELYGS TQLECTSQGQ | 480 |
| WTEEVPSCQV VKCSSLAVPG KINMSCSGEP VFGTVCKFAC PEGWTLNGSA ARTCGATGHW | 540 |
| SGLLPTCEAP TESNIPLVAG LSAAGLSLLT LAPFLLWLRK CLRKAKKFVP ASSCQSLESD | 600 |
| GSYQKPSYIL | |

ACC8 protein sentience
Gene name: Chemokine (C-X-C motif), receptor 4 (fusin)
Unigene number: Hs.89414
Probeset Accession #: L06797
Protein Accession #: NP_003458.1
Pfam: 7TM 1 (7 transmembrane receptor (rhodopsin family))
Signal sequence: none identified
Transmembrane domains: predictions underlined
Summary: The chemokine receptor CXCR4 (also designated fusin and LESTR)
is a cofactor for fusion and entry of T cell-tropic strains of HIV-1.

| | |
|---|---|
| MEGISIYTSD NYTEEMGSGD YDSMKEPCFR EENANFNKIF LPTIYSIIFL TGIVGNGLVI | 60 |
| LVMGYQKKLR SMTDKYRLHL SVADLLFVIT LPFWAVDAVA NWYFGNFLCK AVHVIYTVNL | 120 |
| YSSVLILAFI SLDRYLAIVH ATNSQRPRKL LAEKVVYVGV WIPALLLTIP DFIFANVSEA | 180 |
| DDRYICDRFY PNDLWVVVFQ FQHIMVGLIL PGIVILSCYC IIISKLSHSK GHQKRKALKT | 240 |
| TVILILAFFA CWLPYYIGIS IDSFILLEII KQGCEFENTV HKWISITEAL AFFHCCLNPI | 300 |
| LYAFLGAKFK TSAQHALTSV SRGSSLKILS KGKRGGHSSV STESESSSFH SS | |

ACF2 protein sequence
Gene name: Endothelial cell-specific molecule 1
Unigene number: Hs.41716
Probeset Accession #: X89426
Protein Accession #: NP_008967.1
Signal sequence: underlined
Pfam: IGFBP (Insulin-like growth factor binding proteins)
Summary: Human endothelial cell-specific molecule (called ESM-1) was
cloned from a human umbilical vein endothelial cell (HUVEC) cDNA library.
Constitutive ESM-1 gene expression is seen in HUVECs but not in the
other human cell lines. The cDNA sequence contains an open reading frame
of 552 nucleotides and a 1398-nucleotide 3′-untranslated region including
several domains involved in mRNA instability and five putative polyadenyl-
ation consensus sequences. The deduced 184-amino acid sequence defines a
cysteine-rich protein with a functional NH2-terminal hydrophobic signal
sequence.

| | |
|---|---|
| MKSVLLLTTL LVPAHLVAAW SNNYAVDCPQ HCDSSECKSS PRCKRTVLDD CGCCRVCAAG | 60 |
| RGETCYRTVS GMDGMKCGPG LRCQPSNGED PFGEEFGICK DCPYGTFGMD CRETCNCQSG | 120 |
| ICDRGTGKCL KFPFFQYSVT KSSNRFVSLT EHDMASGDGN IVREEVVKEN AAGSPVMRKW | 180 |
| LNPR | |

ACF4 protein sequence
Gene name: P53-responsive gene 2 similar to D.melanogaster peroxidasin
(U11052)
Unigene number: Hs.118893
Probeset Accession #: D86983
Protein Accession #: BAA13219
Pfam: LRRNT (Leucine rich repeat N-terminal domain), LRR (Leucine Rich
Repeat), LRRCT (Leucine rich repeat C-terminal domain), Ig (immunoglo-
bulin domain), Peroxidase, VWC (von Willebrand factor type C domain)
Summary: ACF4 is a gene originally identified from KG-1 cell and brain
cDNA libraries.

| | |
|---|---|
| SRPWWLRASE RPSAPSAMAK RSRGPGRRCL LALVLFCAWG TLAVVAQKPG AGCPSRCLCF | 60 |
| RTTVRCMHLL LEAVPAVAPQ TSILDLRFNR IREIQPGAFR RLRNLNTLLL NNNQIKRIPS | 120 |
| GAFEDLENLK YLYLYKNEIQ SIDRQAFKGL ASLEQLYLHF NQIETLDPDS FQHLPKLERL | 180 |
| FLHNNRITHL VPGTFNHLES MKRLRLDSNT LHCDCEILWL ADLLKTYAES GNAQAAAICE | 240 |
| YPRRIQGRSV ATITPEELNC ERPRITSEPQ DADVTSGNTV YFTCRAEGNP KPEIIWLRNN | 300 |
| NELSMKTDSR LNLLDDGTLM IQNTQETDQG IYQCMAKNVA GEVKTQEVTL RYFGSPARPT | 360 |
| FVIQPQNTEV LVGESVTLEC SATGHPPPRI SWTRGDRTPL PVDPRVNITP SGGLYIQNW | 420 |
| QGDSGEYACS ATNNIDSVHA TAFIIVQALP QFTVTPQDRV VIEGQTVDFQ CEAKGNPPPV | 480 |
| IAWTKGGSQL SVDRRHLVLS SGTLRISGVA LHDQGQYECQ AVNIIGSQKV VAHLTVQPRV | 540 |
| TPVFASIPSD TTVEVGANVQ LPCSSQGEPE PAITWNKDGV QVTESGKFHI SPEGFLTIND | 600 |
| VGPADAGRYE CVARNTIGSA SVSMVLSVNV PDVSRNGDPF VATSIVEAIA TVDRAINSTR | 660 |
| THLFDSRPRS PNDLLALFRY PRDPYTVEQA RAGEIFERTL QLIQEHVQHG LMVDLNGTSY | 720 |
| HYNDLVSPQY LNLIANLSGC TAHRRVNNCS DMCFHQKYRT HDGTCNNLQH PMWGASLTAF | 780 |
| ERLLKSVYEN GFNTPRGINP HRLYNGHALP MPRLVSTTLI GTETVTPDEQ FTHMLMQWGQ | 840 |
| FLDHDLDSTV VALSQARFSD GQHCSNVCSN DPPCFSVMIP PNDSRARSGA RCMFFVRSSP | 900 |
| VCGSGMTSLL MNSVYPREQI NQLTSYIDAS NVYGSTEHEA RSIRDLASHR GLLRQGIVQR | 960 |
| SGKPLLPFAT GPPTECMRDE NESPIPCFLA GDHRANEQLG LTSMHTLWFR EENRIATELL | 1020 |
| KLNPHWDGDT IYYETRKIVG AEIQHITYQH WLPKILGEVG MRTLGEYHGY DPGINAGIFN | 1080 |
| AFATAAFRFG HTLVNPLLYR LDENFQPIAQ DHLPLHKAFF SPFRIVNEGG IDPLLRGLFG | 1140 |
| VAGKMRVPSQ LLNTELTERL FSMAHTVALD LAAINIQRGR DHGIPPYHDY RVYCNLSAAH | 1200 |
| TFEDLKNEIK NPEIREKLKR LYGSTLNIDL FPALVVEDLV PGSRLGPTLM CLLSTQFKRL | 1260 |
| RDGDRLWYEN PGVFSPAQLT QIKQTSLARI LCDNADNITR VQSDVFRVAE FPHGYGSCDE | 1320 |
| IPRVDLRVWQ DCCEDCRTRG QFNAFSYHFR GRRSLEFSYQ EDKPTKKTRP RKIPSVGRQG | 1380 |
| EHLSNSTSAF STRSDASGTN DFREFVLEMQ KTITDLRTQI KKLESRLSTT ECVDAGGESH | 1440 |
| ANNTKWKKDA CTICECKDGQ VTCFVEACPP ATCAVPVNIP GACCPVCLQK RAEEKP | |

ACF5 protein sequence
Gene name: Mitogen-activated protein kinase kinase kinase kinase 4
Unigene number: Hs.3628
Probeset Accession #: N54067
Protein Accession #: NP_004825.1
Pfam: pkinase (Eukaryotic protein kinase domain), CNH domain
Summary: The yeast serine/threonine kinase STE20 activates a signaling
cascade that includes STE11 (mitogen-activated protein kinase kinase
kinase), STE7 (mitogen-activated protein kinase kinase), and FUS3/KSS1
(mitogen-activated protein kinase) in response to signals from both Cdc42
and the heterotrimeric G proteins associated with transmembrane pheromone
receptors. ACF5 is a human cDNA encoding a protein kinase homologous to
STE20. This protein kinase, also designated HPK/GCK-like kinase CHGK),
has nucleotide sequences that encode an open reading frame of 1165 amino
acids with 11 kinase subdomains. HGK is a serine/threonine protein kinase
that specifically activated the c-Jun N-terminal kinase (JNK) signaling
pathway when transfected into 293T cells, but does not stimulate either
the extracellular signal-regulated kinase or p38 kinase pathway. HGK also
increased AP-1-mediated transcriptional activity in vivo. HGK may be a
novel activator of the JNK pathway. The cascade may look like this:HGK
-> TAK1 -> MKK4, MKK7 -> JNK kinase cascade, which may
mediate the TNF-alpha signaling pathway.

| | |
|---|---|
| MANDSPAKSL VDIDLSSLRfl PAGIFELVEV VGNGTYGQVY KGRHVKTGQL AAIKVMDVTE | 60 |
| DEEEEIKLEI NMLKKYSHWR NIATYYGAFI KKSPPGHDDQ LWLVMEFCGA GSITDLVKMT | 120 |
| KGNTLKEDWI AYISREILRG LAHLHIHHVI HRDIKGQNVL LTENAEVKLV DFGVSAQLDR | 180 |
| TVGRRNTFIG TPYWMAPEVI ACDENPDATY DYRSDLWSCG ITAIEMAEGA PPLCDMHPMR | 240 |
| ALFLIPRNPP PRLKSKKWSK KFFSFIEGCL VKNYMQRPST EQLLKHPFIR DQPNERQVRI | 300 |
| QLKDHIDRTR KKRGEKDETE YEYSGSEEEE EEVPEQEGEP SSIVNVPGES TLRRDFLRLQ | 360 |
| QENKERSEAL RRQQLLQEQQ LREQEEYKRQ LLAERQKRIE QQKEQRRRLE EQQRREREAR | 420 |
| RQQEREQRRR EQEEKRRLEE LERRRKEEEE RRRAEEEKRR VEREQEYIRR QLEEEQRHLE | 480 |
| VLQQQLLQEQ AMLLHDERRP HPQHSQQPPP PQQERSKPSF HAPEPKAHYE PADRAREVPV | 540 |
| RTTSRSPVLS RRDSPLQGSG QQNSQAGQRN STSIEPRLLW ERVEKLVPRP GSGSSSGSSN | 600 |
| SGSQPGSHPG SQSGSGERFR VRSSSKSEGS PSQRLENAVK KPEDKKEVFR PLKPAGEV | 660 |
| TALAKELRAV EDVRPPHKVT DYSSSSEESG TTDEEDDDVE QEGADESTSG PEDTRAASSE | 720 |
| NLSNGETESV KTMIVNDDVE SEPAMTPSKE GTLIVRQTQS ASSTLQKHKS SSSFTPFIDP | 780 |
| RLLQISPSSG TTVTSVVGFS CDGMRPEAIR QDPTRKGSVV NVNPTNTRPQ SDTPEIRKYK | 840 |
| KRFNSEILCA ALWGVNLLVG TESGLMLLDR SGQGKVYPLI NRRRFQQMDV LEGLNVLVTI | 900 |
| SGKKDKLRVY YLSWLRNKIL HNDPEVEKKQ GWTTVGDLEG CVHYKVVKYE RIKFLVIALK | 960 |
| SSVEVYAWAP KPYHKFMAFK SFGELVHKPL LVDLTVEEGQ RLKVIYGSCA GFHAVDVDSG | 1020 |
| SVYDIYLPTH VRKNPHSMIQ CSIKPHAIII LPNTDGMELL VCYEDEGVYV NTYGRITKDV | 1080 |
| VLQWGEMPTS VAYIRSNQTM GWGEKAIEIR SVETGHLDGV FMHKRAQRLK FLCERNDKVF | 1140 |
| FASVRSGGSS QVYFMTLGRT SLLSW | |

ACF8 protein sequence
Gene name: Phospholipase A2, group IVC (cytosolic, calcium-independent)
Unigene number: Hs.18858
Probeset Accession #: AA054087
Protein Accession #: NP_003697.1
Pfam: none identified
Summary: ACF8 is a membrane-bound, calcium-independent PLA2, named cPLA2-
gamma. The sequence encodes a 541-amino acid protein containing a domain
with significant homology to the catalytic domain of the 85-kDa cPLA2
(cPLA2-alpha). cPLA2-gamma does not contain the regulatory calcium-depen-
dent lipid binding (caLB) domain found in cPLA2-alpha. cPLA2-gamma does
contain two consensus motifs for lipid modification, a prenylation motif
(-CCLA) at the C terminus and a myristoylation site at the N terminus.
cPLA2-gamma demonstrates a preference for arachidonic acid at the sn-2
position of phosphatidylcholine as compared with palmitic acid. cPLA2-
gamma encodes a 3-kilobase message, which is highly expressed in heart
and skeletal muscle, suggesting a specific role in these tissues.

| | |
|---|---|
| MGSSEVSIIP GLQKEEKAAV ERRRLHVLKA LKKLRIEADE APVVAVLGSG GGLRAHIACL | 60 |
| GVLSEMKEQG LLDAVTYLAG VSGSTWAISS LYTNDGDMEA LEADLKHRFT RQEWDLAKSL | 120 |
| QKTIQAARSE NYSLTDFWAY MVISKQTREL PESHLSNMKK PVEEGTLPYP IFAAIDNDLQ | 180 |
| PSWQEARAPE TWFEFTPHHA GFSALGAFVS ITHFGSKFKK GRLVRTHPER DLTFLRGLWG | 240 |
| SALGNTEVIR EYIFDQLRNL TLKGLWRRAV ANAKSIGHLI FARLLRLQES SQGEHPPPED | 300 |
| EGGEPEHTWL TEMLENWTRT SLEKQEQPHE DPERKGSLSN LMDFVKKTGI CASKWEWGTT | 360 |
| HNFLYKHGGI RDKIMSSRKH LHLVDAGLAI NTPFPLVLPP TREVHLILSF DFSAGDPFET | 420 |
| IFATTDYCRR HKIPFPQVEE AELDLWSKAP ASCYILKGET GPVVIHFPLF NIDACGGDIE | 480 |
| AWSDTYDTFK LADTYTLDVV VLLLALAKKN VRENKKKILR ELMNVAGLYY PKDSARSCCL | 540 |
| A | |

ACG1 protein sequence
Gene name: carbohydrate (chondroitin 6/keratan) sulfotransferase 1
Unigene number: Hs.104576
Probeset Accession #: AA868063
Protein Accession #: NP_003645.1
Pfam: none identified
Summary: Chondroitin 6-sulfotransferase (C6ST) is the key enzyme in the
biosynthesis of chondroitin 6-sulfate, a glycosaminoglycan implicated in
chondrogenesis, neoplasia, atherosclerosis, and other processes. C6ST
catalyzes the transfer of sulfate from 3′-phosphoadenosine 5′-phospho-
sulfate to carbon 6 of the N-acetylgalactosamine residues of chondroitin.

| | |
|---|---|
| MQCSWKAVLL LALASIAIQY TAIRTFTAKS FHTCPGLAEA GLAERLCEES PTFAYNLSRK | 60 |
| THILILATTR SGSSFVGQLF NQHLDVFYLF EPLYHVQNTL IPRFTQGKSP ADRRVMLGAS 120 | |
| RflLLRSLYDC DLYFLENYIK PPPVNHTTDR IFRRGASRVL CSRPVCDPPG PAflLVLEEGD 180 | |
| CVRKCGLLNL TVAAEACRER SHVAIKTVRV PEVNDLRALV EDPRLNLKVI QLVRflPRGIL 240 | |
| ASRSETFPflT YRLWRLWYGT GRKPYNLDVT QLTTVCEDFS NSVSTGLMRP PWLKGKYMLV 300 | |
| RYEDLARNPM KKTEEIYGFL GIPLDSHVAR WIQNNTRGDP TLGKHKYGTV RNSAATAEKW 360 | |
| RFRLSYDIVA FAQNACQQVL AQLGYKIAAS EEEL~G~PSVS LVEERDFRPF S | |

ACG5 protein sequence
Gene name: Multimerin
Unigene number: Hs.268107
Probeset Accession #: U27109
Protein Accession #: AAC52065
Sign sequence: prediction underlined
Pfam. EGF-like domain, Clq domain
Summary: Multimerin is a massive, soluble protein found in platelets and
in the endothelium of blood vessels. Multimerin is composed of varying
sized, disulfide-linked multimers, the smallest of which is a homotrimer.
Multimerin is a factor V/Va-binding protein and may function as a carrier
protein for platelet factor V. Northern analyses show a 4.7-kilobase tran-
script in cultured endothelial cells, a megakaryocytic cell line, plate-
lets, and highly vascular tissues. The multimerin cDNA can encode a pro-
tein of 1228 amino acids with the probable signal peptide cleavage site
between amino acids 19 and 20. The protein is predicted to be hydro-
philic and to contain 23 N-glycosylation sites. The adhesive motif RGDS
(Arg-Gly-Asp-Ser) and an epidermal growth factor-like domain were identi-
fied. Multimerin contains a probable coiled-coil structures in the
central portion of its sequence. Additionally, the carboxyl-terminal re-
gion of multimerin resembles the globular, non-collagen-like, carboxyl-
terminal domains of several other trimeric proteins, including complement
C1q and collagens type VIII and X.

| | |
|---|---|
| MKGARLFVLL SSLWSGGIGL NNSKHSWTIP EDGNSQKTMP SASVPPNKIQ SLQILPTTRV | 60 |
| MSAEIATTPE ARTSEDSLLK STLPPSETSA PAEGVRNQTL TSTEKAEGW KLQNLTLPTN | 120 |
| ASIKFNPGAE SVVLSNSTLK FLQSFARKSN EQATSLNTVG GTGGIGGVGG TGGVGNRAPR | 180 |
| ETYLSRGDSS SSQRTDYQKS NFETTRGKNW CAYVHTRLSP TVTLDNQVTY VPGGKGPCGW | 240 |
| TGGSCPQRSQ KISNPVYRMQ HKIVTSLDWR CCPGYSGPKC QLRAQEQQSL IHTNQAESHT | 300 |
| AVGRGVAEQQ QQQGCGDPEV MQKMTDQVNY QAMKLTLLQK KIDNISLTVN DVRNTYSSLE | 360 |
| GKVSEDKSRE FQSLLKGLKS KSINVLIRDI VREQFKIFQN DMQETVAQLF KTVSSLSEDL | 420 |
| ESTRQIIQKV NESVVSIAAQ QKFVLVQENR PTLTDIVELR NHIVNVRQEM TLTCEKPIKE | 480 |
| LEVKQTELEG ALEQEHSRSI LYYESLNKTL SKLKEVHEQL LSTEQVSDQK NAPAAESVSN | 540 |
| NVTEYMSTLH ENIKKQSLMM LQMFEDLHIQ ESKINNLTVS LEMEKESLRG ECEDMLSKCR | 600 |
| NDFKFQLIQT EENLHVLNQT LAEVLFPMDN KMDKMSEQLN DLTYDMEILQ PLLEQGASLR | 660 |
| QTMTYEQPKE AIVIRKKIEN LTSAVNSLNF IIKELTKRHN LLRNEVQGRD DALERRINEY | 720 |
| ALEMEDGLNK TMTIINNAID FIQDNYALKE TLSTIKDNSE IHHKCTSDME TILTFIPQFH | 780 |
| RLNDSIQTLV NDNQRYNFVL QVAKTLAGIP RDEKLNQSNF QKMYQMFNET TSQVRKYQQN | 840 |
| MSHLEEKLLL TTKISKNFET RLQDIESKVT QTLIPYYISV KKGSVVTNER DQALQLQVLN | 900 |
| SRFKALEAKS IHLSINFFSL NKTLHEVLTM CHNASTSVSE LNATIPKWIK HSLPDIQLLQ | 960 |
| KGLTEFVEPI IQIKTQAALS NSTCCIDRSL PGSLANVVKS QKQVKSLPKK INALKKPTVN | 1020 |
| LTTVLIGRTQ RNTDNIIYPE EYSSCSRHPC QNGGTCINGR TSFTCACRHP FTGDNCTIKL | 1080 |
| VEENALAPDF SKGSYRYAPM VAFFASHTYG MTIPGPILFN NLDVNYGASY TPRTGKFRIP | 1140 |
| YLGVYVFKYT IESFSAHISG FLVVDGIDKL AFESENINSE IHCDRVLTGD ALLELNYGQE | 1200 |
| VWLRILAKGTI PAKFPPVTTF SGYLLYRT | |

ACC6 protein sequence
Gene name: Homo sapiens cDNA FLJ11502 fis, clone HEMBA10021O2, weakly
similar to ANKRYIN
Unigene number: Hs.213194
Probeset Accession #: AA187101
Protein Accession #: none
Pfam: ankyrin repeats

| | |
|---|---|
| VAARPPVSRM EPRAADGCFL GDVGFWVERT PVHEAAQRGE SLQLQQLIES GACVNQVTVD | 60 |
| SITPLHAASL QGQARCVQLL LAAGAQVDAR NIDGSTPLCD ACASGSIECV KLLLSYGAKV | 120 |
| NPPLYTASPL HEASFPRLLS TLASTPWIN | |

ACC7 protein sequence
Gene name: Human PAL A gene
Unigene number: Hs.6906
Probeset Accession #: AA083572 cluster
Protein Accession #: P11233
Pfam: ras
Features: CAAX motif is underlined
Summary: The RALA gene encodes a low molecular mass ras-like GTP-binding
protein that shares about 50% similarity with the ras proteins. GTP-
binding proteins mediate the transmembrane signaling initiated by the
occupancy of certain cell surface receptors. The RALA gene maps to 7p22-
p15.

| | |
|---|---|
| MAANKPKGQN SLALHKVIMV GSGGVGKSAL TLQFMYDEFV EDYEPTKADS YRKKVVLDGE | 60 |
| EVQIDILDTA GQEDYAAIRD NYFRSGEGFL CVFSITEMES FAATADFREQ ILRVKEDENV | 120 |
| PFLLVGNKSD LEDKRQVSVE EAKNRAEQWN VNYVETSAKT RANVDKVFFD LMREIRARKM | 180 |
| EDSKEKNGKK KRKSLAKRIR ERCC | |

ACC9 protein sequence
Gene name: KIAA0955 protein
Unigene number: Hs.10031
Probeset Accession #: AA027168
Protein Accession #: BAA76799.1
Pfam: CARD (Caspase recruitment domain)
Summary: Gene was originally isolated as a brain cDNA. The coding region
contains a CARD domain, suggesting involvement in apoptotic signaling
pathways.

| | |
|---|---|
| MMRQRQSHYC SVLFLSVNYL GGTFPGDICS EENQIVSSYA SKVCFEIEED YKNRQFLGPE | 60 |
| GNVDVELIDK STNRYSVWFP TAGWYLWSAT GLGFLVRDEV TVTIAFGSWS QHLALDLQHH | 120 |
| EQWLVGGPLF DVTAEPEEAV AEIHLPHFIS LQGEVDVSWF LVARFKNEGM VLEHPARVEP | 180 |
| FYAVLESPSF SLMGILLRIA SGTRLSIPIT SNTLIYYHPH PEDIKFHLYL VPSDALLTKA | 240 |
| IDDEEDRFHG VRLQTSPPME PLNFGSSYIV SNSANLKVMP KELKLSYRSP GEIQHFSKFY | 300 |
| AGQMKEPIQL EITEKRHGTL VWDTEVKPVD LQLVAASAPP PFSGAAFVKE NHRQLQARMG | 360 |
| DLKGVLDDLQ DNEVLTENEK ELVEQEKTRQ SKNEALLSMV EKKGDLALDV LFRSISERDP | 420 |
| YLVSYLRQQN L | |

ACF6 Protein sequence
Gene name: Homo sapiens cDNA FLJ10669 fis, clone NT2RP2006275, weakly
similar to Microtubule-associated protein 1B [CONTAINS: LIGHT CHAIN
LC1]
Unigene number: Hs.66048
Probeset Accession #: AA609717
Protein Accession #: BAA91743.1
Pfam: none identified
Summary: The cDNA for FLJ10669 was originally isolated from NT2 neuronal
precursor cells (teratocarcinoma cell line) after 2-weeks of retinoic
acid (RA) treatment. The protein sequence has similarity to microtubule-
associated protein 1B (MAP-1B), suggesting a function for ACFE in the reg-
ulating the cytoskeleton.

| | |
|---|---|
| MGVGRLDMYV LHPPSAGAER TLASVCALLV WHPAGPGEKV VRVLFPGCTP PACLLDGLVR | 60 |
| LQHLRFLREP VVTPQDLEGP GRAESKESVG SRDSSKREGL LATHPRPGQE RPGVARKEPA | 120 |
| RAEAPRKTEK EAKTPRELKK DPKPSVSRTQ PREVRRAASS VPNLKKTNAQ AAPKPRKAPS | 180 |
| TSHSGFPPVA NGPRSPPSLR CGEASPPSAA CGSPASQLVA TPSLELGPIP AGEEKALELP | 240 |
| LAASSIPRPR TPSPESHRSP AEGSERLSLS PLRGGEAGPD ASPTVTTPTV TTPSLPAEVG | 300 |
| SPHSTEVDES LSVSFEQVLP PSAPTSEAGL SLPLRGPRAR RSASPHDVDL CLVSPCEFEH | 360 |
| RKAVPMAPAP ASPGSSNDSS ARSQERAGGL GAEETPPTSV SESLPTLSDS DPVPLAPGAA | 420 |
| DSDEDTEGFG VPRHDPLPDP LKVPPPLPDP SSICMVDPEM LPPKTARQTE NVSRTRKPLA | 480 |
| RPNSRAAAPK ATPVAAAKTK GLAGGDRASR PLSARSEPSE KGGRAPLSRK SSTPKTATRG | 540 |
| PSGSASSRPG VSATPPKSPV YLDLAYLPSG SSAHLVDEEF FQRVRALCYV ISGQDQRKEE | 600 |
| GMRAVLDALL ASKQHWDRDL QVTLIPTFDS VAMHTWYAET HARHQALGIT VLGSNGMVSM | 660 |
| QDDAFPACKV EF | |

What is claimed is:

1. A method of detecting an angiogenesis-associated transcript in a cell in a patient, the method comprising contacting a biological sample from the patient with a polynucleotide that selectively hybridized to a sequence at least 80% identical to a sequence as shown in Table 1.

2. The method of claim 1, wherein the biological sample is a tissue sample.

3. The method of claim 1, wherein the biological sample comprises isolated nucleic acids.

4. The method of claim 3, wherein the nucleic acids are mRNA.

5. The method of claim 3, further comprising the step of amplifying nucleic acids before the step of contacting the biological sample with the polynucleotide.

6. The method of claim 1, wherein the polynucleotide comprises a sequence as shown in Table 1.

7. The method of claim 1, wherein the polynucleotide is labeled.

8. The method of claim 7, wherein the label is a fluorescent label.

9. The method of claim 1, wherein the polynucleotide is immobilized on a solid surface.

10. The method of claim 1, wherein the patient is undergoing a therapeutic regimen to treat a disease associated with angiongenesis.

11. The method of claim 1, wherein the patient is suspected of having cancer.

12. An isolated nucleic acid molecule consisting of a polynucleotide sequence as shown in Table 1.

13. The nucleic acid molecule of claim 12, which is labeled.

14. The nucleic acid of claim 13, wherein the label is a fluorescent label

15. An expression vector comprising the nucleic acid of claim 12.

16. A host cell comprising the expression vector of claim 15.

17. An isolated nucleic acid molecule which encodes a polypeptide having an amino acid sequence as shown in Table 2.

18. An isolated polypeptide which is encoded by a nucleic acid molecule having polynucleotide sequence as shown in Table 1.

19. An isolated polypeptide having an amino acid sequence as shown in Table 2.

20. An antibody that specifically binds a polypeptide of claim 19.

21. The antibody of claim 20, further conjugated to an effector component.

22. The antibody of claim 21, wherein the effector component is a fluorescent label.

23. The antibody of claim 21, wherein the effector component is a radioisotope.

24. The antibody of claim 21, which is an antibody fragment.

25. The antibody of claim 21, which is a humanized antibody

26. A method of detecting a cell undergoing angiogenesis in a biological sample from a patient, the method comprising contacting the biological sample with an antibody of claim 20.

27. The method of claim 26, wherein the antibody is further conjugated to an effector component.

28. The method of claim 27, wherein the effector component is a fluorescent label.

29. The method of detecting antibodies specific to angiogenesis in a patient, the method comprising contacting a biological sample from the patient with a polypeptide comprising a sequence as shown in Table 2.
